(19) United States
(12) Patent Application Publication
(10) Pub. No.: US 2021/0213006 A1
(43) Pub. Date: Jul. 15, 2021

(54) METHODS OF TREATING NEURODEGENERATIVE DISEASES

(71) Applicant: Seal Rock Therapeutics, Inc., Seattle, WA (US)

(72) Inventors: Artur PLONOWSKI, Cupertino, CA (US); Kathleen Ann ELIAS, San Francisco, CA (US); Samuel David BROWN, San Carlos, CA (US); Terence Graham PORTER, Chicago, IL (US); Neil Dwayne MCDONNELL, Seattle, WA (US)

(21) Appl. No.: 17/271,883

(22) PCT Filed: Aug. 27, 2019
(86) PCT No.: PCT/US2019/048388

§ 371 Date: Feb. 26, 2021

Related U.S. Application Data
(60) Provisional application No. 62/724,574, filed on Aug. 29, 2018.

(51) Int. Cl. A61K 31/4439; A61P 25/28

(52) U.S. Cl. CPC A61K 31/4439; A61P 25/28

(57) ABSTRACT

Described herein are methods of treating or preventing an ASK1 or DYRK1A associated disease, disorder, or condition comprising administering to a subject in need thereof a dual inhibitor of ASK1 and DYRK1A; including administering pharmaceutically acceptable salts and solvates thereof.

### CROSS-REFERENCE TO RELATED APPLICATIONS

This application claims the benefit of U.S. Provisional Application No. 62/724,574, filed Aug. 29, 2018, which is incorporated herein by reference in its entirety.

### BACKGROUND OF THE INVENTION

Alzheimer's disease (AD) is the most common neurodegenerative disorder, which affects 5.5 million people in the U.S. and 40 million worldwide. At present, there is no curative therapy for AD. Alzheimer's disease is complex and can be considered a disease of four major pathological processes. The four processes are: 1) vascular hypoperfusion of the brain associated with mitochondrial dysfunction, 2) destructive protein inclusion, 3) uncontrolled oxidative stress, and 4) pro-inflammatory immune process secondary to microglial and astrocytic dysfunction in the brain. The complexity of AD and other neurodegenerative disorders dictates that a polypharmacological approach is most likely to achieve the greatest impact in treating AD. Two enzymes, ASK1 and DYRK1A have been implicated in all four of the pathological processes associated with AD. Therefore dual inhibitors of ASK1 and DYRK1A show promise as a therapeutic treatments for AD and other neurodegenerative diseases.

### SUMMARY OF THE INVENTION

In one aspect described herein are methods for the treatment or prevention of an ASK1 or DYRK1A associated condition, comprising administering to a subject in need thereof, a therapeutically effective amount of a dual inhibitor of ASK1 and DYRK1A.

In some embodiments, the dual inhibitor of ASK1 and DYRK1A is a compound having the structure of Formula (IV), or a pharmaceutically acceutable salt or solvate thereof:

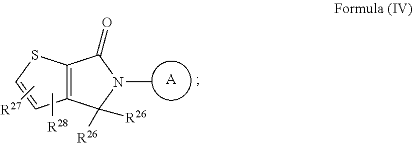

wherein:

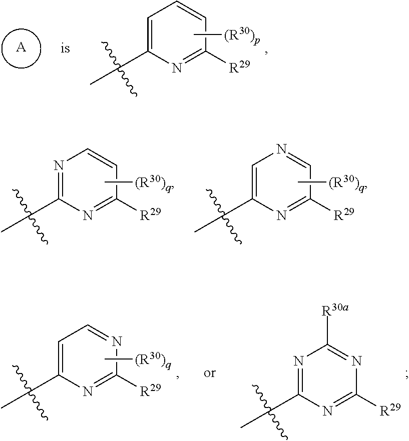

each R²⁶ is independently selected from a group consisting of hydrogen, halogen, and C₁-C₆alkyl;
R²⁷ is selected from a group consisting of hydrogen, halogen, —CN, —OH, —OR⁶, —SR⁶, —S(═O)R⁷, —NO₂, —N(R³¹)₂, —S(═O)₂R³², —NHS(═O)₂R³², —S(═O)₂N(R³¹)₂, —C(═O)R³², —C(═O)OR³¹, —OC(═O)R³², —C(═O)N(R³¹)₂, —OC(═O)N(R³¹)₂, —NR³¹C(═O)N(R³¹)₂, —NR³¹C(═O)R³², —NR³¹C(═O)OR³¹, C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉ heterocycle, C₆₋₁₀aryl, C₁₋₉heteroaryl, and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂—N(R³⁸)₂, —N(R³⁸)₂, —N(R³⁸)C(═O)R³⁹, and —N(R³⁸)S(═O)₂R³⁸;
R²⁸ is selected from a group consisting of hydrogen, halogen, —CN, and C₁₋₆alkyl; or R²⁷ and R²⁸ are combined to form a phenyl ring optionally substituted with one, two, or three R³³ substituents;
R²⁹ is selected from a group consisting of hydrogen, halogen, —CN, —OH, —OR³¹, —SR³¹, —S(═O)R³², —NO₂, —N(R³¹)₂, —S(═O)₂R³², —NHS(═O)₂R³², —S(═O)₂N(R³¹)₂, —C(═O)R³², —C(═O)OR³¹, —OC(═O)R³², —C(═O)N(R³¹)₂, —OC(═O)N(R³¹)₂, —NR³¹C(═O)N(R³¹)₂, —NR³¹C(═O)R³², —NR³¹C(═O)OR³¹, C₁₋₆alkYl, C₂₋₆alkenyl, C₂₋₆alkynyl, C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂—N(R³⁸)₂, —N(R³⁸)₂, —N(R³⁸)C(═O)R³⁹, and —N(R³⁸)S(═O)₂R³⁸;
each R³° is independently selected from a group consisting of halogen, —CN, and C₁₋₆alkyl;
R^{30a} is selected from the group consisting of hydrogen and C₁-C₆alkyl;
each R³¹ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle; or two R³¹ on the same heteroatom are taken together with that heteroatom to which they are attached to form a C₂₋₉heterocycle or a C₂₋₉heteroaryl;
each R³² is independently selected from the group consisting of C₁-C₆alkyl, C₃-C₈cycloalkyl, and C₂₋₉heterocycle;
each R³³ is independently selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸;
each R³⁸ is independently selected from the group consisting of hydrogen, C₁-C₆alkyl, and C₃-C₈cycloalkyl; or two R³⁸ on the same heteroatom are taken together with that heteroatom to which they are attached to form a C₂₋₉heterocycle;
each R³⁹ is independently selected from the group consisting of C₁-C₆alkyl and C₃-C₈cycloalkyl;
p is 0, 1, 2, or 3; and
q is 0, 1, or 2.

In some embodiments, the dual inhibitor having the structure of Formula (IV) has the structure of Formula (IVa), or a pharmaceutically acceptable salt or solvate thereof:

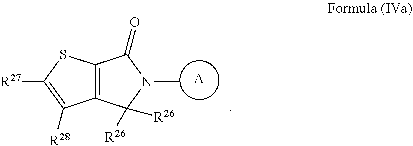

In some embodiments, the dual inhibitor having the structure of Formula (IV) has the structure of Formula (IVb), or a pharmaceutically acceptable salt or solvate thereof:

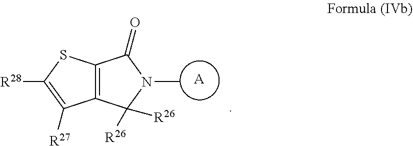

In some embodiments, the dual inhibitor of ASK1 and DYRK1A is a compound having the structure of Formula (IVc), or a pharmaceutically acceptable salt or solvate thereof:

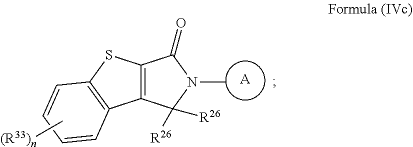

wherein R²⁶ and R³³ are defined in embodiments; and
n is 0, 1, 2, or 3.

In some embodiments, the dual inhibitor of ASK1 and DYRK1A is a compound having the structure of Formula (V), or a pharmaceutically acceptable salt or solvate thereof:

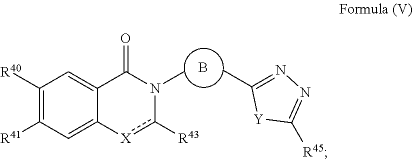

wherein:
[symbol] is a single bond or a double bond;
X is selected from the group consisting of C(R⁴²), CH(R⁴²), N, and N(R⁴²);
Y is N(R⁴⁴) or O;

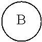

is selected from the group consisting of phenyl and a 5- to 6-membered heteroaryl;
R⁴⁰ is a 5- to 10-membered heteroaryl optionally substituted with one, two, or three R⁴⁶ groups;
R⁴¹ is hydrogen, halogen, —OH, or —NH₂, or is selected from the group consisting of C₁₋₆ alkyl, C₁₋₃ heteroalkyl, a 5- to 6-membered heterocycloalkyl, phenyl, and a 5- to 6-membered heteroaryl, optionally substituted with one, two, or three R⁴⁶ groups;
   R⁴² is selected from the group consisting of hydrogen, halogen, —OH, and —NH₂;
R⁴³ is selected from the group consisting of hydrogen, C₁₋₃alkyl, and C₁₋₃alkoxy;
R⁴⁴ is selected from the group consisting of H, C₁₋₈alkyl, C₃₋₇cycloalkyl, and a 3- to 6-membered heterocycloalkyl, optionally substituted with one, two, or three R⁴⁶ groups;
R⁴⁵ is selected from the group consisting of hydrogen and C₁₋₆alkyl;
or R⁴⁴ and R⁴⁵ are joined together to form a 5- to 6-membered ring;
R⁴⁶ is selected from the group consisting of hydrogen, halogen, —OH, —NH₂, NH₂—(C═O)—, C₁₋₃alkyl, C₁₋₃alkoxy, C₁₋₃alkyl-NH—(C═O)—, C₁₋₃alkyl-S(═O)₂—, C₃₋₆cycloalkyl, a 3- to 6-membered heterocycloalkyl, and phenyl; the “hetero” moieties of the 5- to 10-membered heteroaryl, C₁₋₃ heteroalkyl, 5- to 6-membered heterocycloalkyl, 5- to 6-membered heteroaryl, and 3- to 6-membered heterocycloalkyl are each independently selected from the group consisting of —NH—, N, —O—, —S—, —S(═O)₂—, and —NH—C(═O)—; and
the numbers of the heteroatoms or heteroatom groups in any of the above cases are each independently one, two, or three.

In some embodiments, the dual inhibitor of ASK1 and DYRK1A is a compound having the structure of Formula (VI), or a pharmaceutically acceptable salt or solvate thereof:

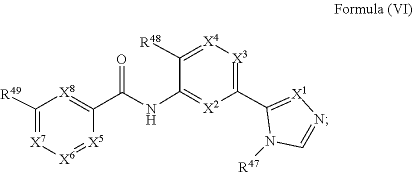

wherein:
R⁴⁷ is alkyl, alkenyl, alkynyl, cycloalkyl, aryl, heteroaryl, or heterocyclyl, all of which are optionally substituted with one, two, or three substituents selected from halogen, oxo, alkyl, cycloalkyl, heterocyclyl, aryl, aryloxy, —NO₂, —R⁵², —C(O)—R⁵², —OC(O)—R⁵²—C(O)—O—R⁵², —C(O)—N(R⁵²)(R⁵³), —OC(O)—N(R⁵²)(R⁵³), —S—R⁵², —S(═O)—R⁵², —S(═O)₂R⁵², —S(═O)₂—N(R⁵²)(R⁵³), —S(═O)₂—O—R⁵², —N(R⁵²)(R⁵³), —N(R⁵²)—C(O)—R⁵³, —N(R⁵²)—C(O)—O—R⁵³, —N(R⁵²)—C(O)—N(R⁵²)(R⁵³), —N(R⁵²)—S(═O)₂—R⁵², —CN, and —O—R⁵², wherein alkyl, cycloalkyl, heterocyclyl, phenyl, and phenoxy are optionally substituted with one, two, or three substituents selected from alkyl, cycloalkyl, alkoxy, hydroxyl, and halogen;
wherein R⁵² and R⁵³ are independently selected from the group consisting of hydrogen, C₁₋₁₅alkyl, cycloalkyl, heterocyclyl, aryl, and heteroaryl, all of which are optionally substituted with one, two, or three substituents selected from halo, alkyl, mono- or dialkylamino, alkyl or aryl or heteroaryl amide, —CN, alkoxy, —CF₃, aryl, and heteroaryl;
or R⁵² and R⁵³ when taken together with the nitrogen to which they are attached form a heterocycle;
R⁴⁸ is hydrogen, halogen, —CN, alkoxy, or alkyl optionally substituted by halogen;
R⁴⁹ is aryl, heteroaryl, or heterocyclyl, all of which are optionally substituted with one, two, or three substituents selected from alkyl, alkoxy, cycloalkyl, cycloalkylalkyl, aryl, arylalkyl, heteroaryl, heteroarylalkyl, heterocyclyl, heterocyclylalkyl, halogen, oxo, —NO₂, haloalkyl, haloalkoxy, —CN, —O—R⁵², —O—C(O)—R⁵², —O—C(O)—N(R⁵²)(R⁵³), —S—R⁵², —N(R⁵²)(R⁵³), —S(═O)—R⁵², —S(═O)₂R⁵², —S(═O)₂—N(R⁵²)(R⁵³), —S(═O)₂—O—R⁵², —N(R⁵²)—C(O)—R⁵³, —N(R⁵²)—C(O)—O—R⁵³, —N(R⁵²)—C(O)—N(R⁵²)(R⁵³), —C(O)—R⁵², —C(O)—O—R⁵², —C(O)—N(R⁵²)(R⁵³), and —N(R⁵²)—S(═O)₂—R⁵³, wherein the alkyl, alkoxy, cycloalkyl, aryl, heteroaryl, or heterocyclyl is optionally substituted with one, two, or three substituents selected from halogen, oxo, —NO₂, alkyl, haloalkyl, haloalkoxy, —N(R⁵²)(R⁵³), —C(O)—R⁵², —C(O)—O—R⁵², —C(O)—N(R⁵²)(R⁵³), —CN, —O—R⁵², cycloalkyl, aryl, heteroaryl, and heterocyclyl;
with the proviso that the heteroaryl or heterocyclyl moiety includes at least one ring nitrogen atom; X¹, X², X³, X⁴, X⁵, X⁶, X⁷, and X⁸ are independently C(R⁵⁰) or N, wherein
each R⁵⁰ is independently hydrogen, alkyl, alkoxy, cycloalkyl, aryl, heteroaryl, heterocyclyl, halogen, —NO₂, haloalkyl, haloalkoxy, —CN, —O—R⁵², —S—R⁵², —N(R⁵²)(R⁵³), —S(═O)—R⁵², —S(═O)₂R⁵², —S(═O)₂—N(R⁵²)(R⁵³), —S(═O)₂—O—R⁵², —N(R⁵²)—C(O)—R⁵³, —N(R⁵²)—C(O)—O—R⁵³, —N(R⁵²)—C(O)—N(R⁵²)(R⁵³), —C(O)—R⁵², —C(O)—O—R⁵², —C(O)—N(R⁵²)(R⁵³), or —N(R⁵²)—S(═O)₂—R⁵³, wherein the alkyl, cycloalkyl, aryl, heteroaryl, and heterocyclyl is further optionally substituted with one, two, or three substituents selected from halogen, oxo, —NO₂, —CF₃, —O—CF₃, —N(R⁵²)(R⁵³), —C(O)—R⁵², —C(O)—O—R⁵³, —C(O)—N(R⁵²)(R⁵³), —CN, —O—R⁵²; or X⁵ and X⁶ or X⁶ and X⁷ are joined to provide optionally substituted fused aryl or optionally substituted fused heteroaryl; and
with the proviso that at least one of X², X³, and X⁴ is C(R⁵⁰); at least two of X⁵, X⁶, X⁷, and X⁸ are C(R⁵⁰); and at least one of X², X³, X⁴, X⁵, X⁶, X⁷, and X⁸ is N.

In some embodiments, the dual inhibitor of ASK1 and DYRK1A is a compound having the structure of Formula (VII), or a pharmaceutically acceptable salt or solvate thereof:

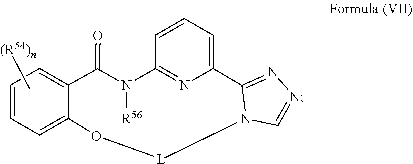

wherein:
L is selected from C₃₋₅alkylene and C₃₋₅alkenylene, wherein C₃₋₅alkylene and C₃₋₅alkenylene are optionally substituted with one or two R⁵⁵ groups;
each R⁵⁴ is independently selected from C₁₋₆alkyl, C₂₋₆alkenyl, C₂₋₆alkynyl, carbocyclyl, heterocyclyl, halo, —CN, —C(O)R^{54a}, —C(O)₂R^{54a}, —C(O)N(R^{54a}), —N(R^{54a}), —N(R^{54a})C(O)R^{54a}, —N(R^{54a})C(O)₂R^{54a}, —N(R^{54a})C(O)N(R^{54a}), —N(R^{54a})S(O)₂R^{54a}, —OR^{54a}, —OC(O)R^{54a}, —OC(O)N(R^{54a})₂, —SR^{54a}, —S(O)R^{54a}, —S(O)₂R^{54a}, —S(O)N(R^{54a})₂, and —S(O)₂N(R^{54a})₂, wherein C₁₋₆alkyl, C₂₋₆alkenyl, C₂₋₆alkynyl, carbocyclyl, and heterocyclyl, are optionally substituted with one or more R⁵⁸ groups;
each R^{54a} is independently selected from H, C₁₋₆alkyl, C₂₋₆alkenyl, C₂₋₆alkynyl, carbocyclyl, and heterocyclyl, wherein C₁₋₆alkyl, C₂₋₆alkenyl, C₂₋₆alkynyl, carbocyclyl, and heterocyclyl are each optionally and independently substituted with one or more R⁵⁸ groups;
each R⁵⁸ is independently selected from C₁₋₆alkyl, C₂₋₆alkenyl, C₂₋₆alkynyl, carbocyclyl, heterocyclyl, halo, —CN, —C(O)R^{58a}, —C(O)₂R^{58a}, —C(O)N(R^{58a})₂, —N(R^{58a})₂, —N(R^{58a})C(O)R^{58a}, —N(R^{58a})C(O)₂R^{58a}, —N(R^{58a})C(O)N(R^{58a})₂, —N(R^{58a})S(O)₂R^{58a}, —OR^{58a}, —OC(O)R^{58a}, —OC(O)N(R^{58a})₂, —SR^{8a}, —S(O)R^{58a}, —S(O)₂R^{58a}, —S(O)N(R^{58a})₂, and —S(O)₂N(R^{58a})₂, wherein C₁₋₆alkyl, C₂₋₆alkenyl, C₂₋₆alkynyl, carbocyclyl, and heterocyclyl are optionally and independently substituted with one or more groups selected from halo, —CN, —C(O)R^{58a}, —C(O)₂R^{58a}, —C(O)N(R^{58a})₂, —N(R^{58a}), —N(R^{58a})C(O)R^{58a}, —N(R^{58a})C(O)₂R^{58a}, —N(R^{58a})C(O)N(R^{58a})₂, —N(R^{58a})S(O)₂R^{58a}, —OR^{58a}, —OC(O)R^{58a}, —OC(O)N(R^{58a})₂, —SR^{58a}, —S(O)R^{58a}, —S(O)₂R^{58a}, —S(O)N(R^{58a})₂, and —S(O)₂N(R^{58a})₂;
each R^{58a} is independently selected from H, C₁₋₆alkyl, C₂₋₆alkenyl, C₂₋₆alkynyl, carbocyclyl, and heterocyclyl;
each R⁵⁵ is independently selected from C₁₋₆alkyl, —CN, —C(O)R^{55a}, —C(O)₂R^{55a}, —C(O)N(R^{55a})₂, —NO₂, —N(R^{55a})₂, —N(R^{55a})C(O)R^{55a}, —N(R^{55a})C(O)₂R^{55a}, —N(R^{55a})C(O)N(R^{55a})₂, —N(R^{55a})S(O)₂R^{55a}, —OR^{55a}, —OC(O)R^{55a}, —OC(O)N(R^{55a})₂, —SR^{55a}, —S(O)R^{55a}, —S(O)₂R^{55a}, —S(O)N(R^{55a})₂, and —S(O)₂N(R⁵⁴)₂, wherein C₁₋₆alkyl is optionally substituted with one or more R⁵⁹ groups;
each R^{55a} is independently selected from H, C₁₋₆alkyl, C₂₋₆alkenyl, C₂₋₆alkynyl, carbocyclyl, and heterocyclyl wherein C₁₋₆alkyl, C₂₋₆alkenyl, C₂₋₆alkynyl, carbocyclyl, and heterocyclyl is optionally and independently substituted with one or more R⁵⁹ groups; and
each R⁵⁹ is independently selected from C₁₋₆alkyl, halo, and —OR^{59a};
each R^{59a} is independently selected from H, C₁₋₆alkyl, C₂₋₆alkenyl, C₂₋₆alkynyl, carbocyclyl, and heterocyclyl;
R⁵⁶ is H or C₁₋₄alkyl; and
n is selected from 0, 1, and 2.

In some embodiments, the dual inhibitor of ASK1 and DYRK1A is a compound having the structure of Formula (VIII), or a pharmaceutically acceptable salt or solvate thereof:

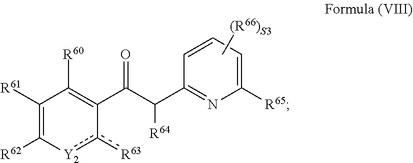

wherein:

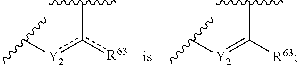

wherein:
Y² is N or CR^{Y2};
R⁶³ is H, halo, —CN, —OR⁶⁷, —SR⁶⁷, —S(═O)R⁶⁹, —NO₂, —NR⁶⁷R⁶⁸, —S(═O)₂R⁶⁹, —NR⁶⁷S(═O)₂R⁶⁹, —S(═O)₂NR⁶⁷R⁶⁸, —C(═O)R⁶⁹, —OC(═O)R⁶⁹, —CO₂R⁶⁷, —OCO₂R⁶⁷, —C(═O)NR⁶⁷R⁶⁸, —OC(═O)NR⁶⁷R⁶⁸, —NR⁶⁷C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl;
R^{Y2} is H, halo, —CN, —OR⁶⁷, —SR⁶⁷, —S(═O)R⁶⁹, —NO₂, —NR⁶⁷R⁶⁸, —S(═O)₂R⁶⁹, —NR⁶⁷S(═O)₂R⁶⁹, —S(═O)₂NR⁶⁷R⁶⁸, —C(═O)R⁶⁹, —OC(═O)R⁶⁹, —CO₂R⁶⁷, —OCO₂R⁶⁷, —C(═O)NR⁶⁷R⁶⁸, —OC(═O)NR⁶⁷R⁶⁸, —NR⁶⁷C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl; or

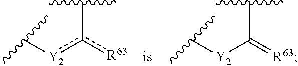

wherein:
Y² is NR^{Y3};
R⁶³ is O or S; and
R^{Y3} is H or optionally substituted alkyl;
R⁶⁰, R⁶¹, and R⁶² are independently H, halo, —CN, —OR⁶⁷, —SR⁶⁷, —S(═O)R⁶⁹, —NO₂, —NR⁶⁷R⁶⁸, S(═O) R⁶⁹—NR⁶⁷S(═O)₂R⁶⁹, —S(═O)₂NR⁶⁷R⁶⁸, —C(═O)R⁶⁹, —OC(═O)R⁶⁹, —CO₂R⁶⁷, —OCO₂R⁶⁷, —C(═O)NR⁶⁷R⁶⁸, —OC(═O)N R⁶⁷R⁶⁸, —NR⁶⁷C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl;
R⁶⁴ is H, optionally substituted alkyl, optionally substituted alkenyl, optionally substituted alkynyl, optionally substituted heteroalkyl, optionally substituted cycloalkyl, optionally substituted heterocycloalkyl, optionally substituted aryl, or optionally substituted heteroaryl;
or R⁶³ and R⁶⁴ are taken together with the atoms to which they are attached to form an optionally substituted heterocycloalkyl or an optionally substituted heteroaryl;
R⁶⁵ is optionally substituted fused bicyclic heterocycloalkyl or optionally substituted fused bicyclic heteroaryl;
each R⁶⁶ is independently halo, —CN, —OR⁶⁷, —SR⁶⁷, —S(═O)R⁶⁹, —NO₂, —NR⁶⁷R⁶⁸, —S(═O)₂R⁶⁹, —NR⁶⁷S(═O)₂R⁶⁹, —S(═O)₂NR⁶⁷R⁶⁸, —C(═O)R⁶⁹, —OC(═O)R⁶⁹, —CO₂R⁶⁷, —OCO₂R⁶⁷, —C(═O)NR⁶⁷R⁶⁸, —OC(═O)N R⁶⁷R⁶⁸, —NR⁶⁷C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl;
each R⁶⁷ and R⁶⁸ is independently H, optionally substituted —CN, —OR⁶⁷, —SR⁶⁷, —S(═O)R⁶⁹, —NO₂, —NR⁶⁷R⁶⁸, —S(═O) R⁶⁹-NR⁶⁷S(═O)₂R⁶⁹, —S(═O)₂NR⁶⁷R⁶⁸, —C(═O)R⁶⁹, —OC(═O)R⁶⁹, —CO₂R⁶⁷, —OCO₂R⁶⁷, —C(═O)NR⁶⁷R⁶⁸, —OC(═O)NR⁶⁷R⁶⁸, —NR⁶⁷C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl;
or R⁶⁷ and R⁶⁸, together with the nitrogen atom to which they are attached, form an optionally substituted heterocycloalkyl or optionally substituted heteroaryl;
R⁶⁹ is optionally substituted alkyl, optionally substituted alkynyl, optionally substituted heteroalkyl, optionally substituted cycloalkyl, optionally substituted heterocycloalkyl, optionally substituted aryl, or optionally substituted heteroaryl; and
s3 is 0-3.

In some embodiments, the dual inhibitor of ASK 1 and DYRK1A is a compound having the structure of Formula (IX), or a pharmaceutically acceptable salt or solvate thereof:

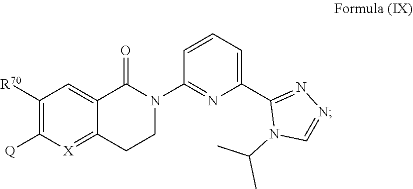

wherein:
X is selected from the group consisting of CH and N;
Q is selected from the group consisting of CH₃ and H; and
R⁷⁰ is selected from the group consisting of

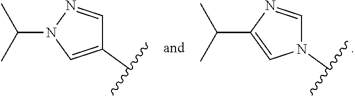

In some embodiments, the dual inhibitor of ASK 1 and DYRK1A is a compound having the structure of Formula (X), or a pharmaceutically acceptable salt or solvate thereof:

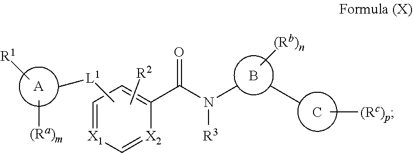

wherein:
Ring C is phenyl, 6-membered hetroaryl, or a 5-membered heteroaryl;
each R^{a} is independently hydrogen, deuterium, halogen, —CN, —OR⁵, —SR⁵, —S(═O)R⁴, —S(═O)₂R⁴, —S(═O)₂N(R⁵)₂, —R⁵S(═O)₂R⁴, —C(═O)R⁴, —OC(═O)R⁴, —CO₂R⁵, —OCO₂R⁴, —N(R⁵)₂, —OC(═O)N(R⁵)₂, —C(═O)N(R⁵)₂, —R⁵C(═O)R₄, —R⁵C(═O)OR⁴, —R⁵C(═O)N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, substituted or unsubstituted C₃-C₆cycloalkyl;
m is 0, 1, 2, or 3;
R¹ is hydrogen, deuterium, halogen, —CN, —OR⁵, —SR⁵, —S(═O)R⁴, —S(═O)₂R⁴, —N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, or substituted or unsubstituted —C₁-C₄alkylene-N(R⁵)₂;
L¹ is linker that is —X²—, L², -L²-X²—, —X²-L³-, or -L²-X²-L³-;
   X² is —O—, —S—, —S(═O)—, —S(═O)₂—, —S(═O)₂NR⁶—, —C(═O)—, —C(═O)O—, —C(═O)NR⁶—, —OC(═O)NR⁶—, —NR⁶C(═O)O, —NR⁶C(═O)NR⁶—, —OC(═O)—, —NR⁶C(═O)—, —NR⁶S(═O)₂—, or —NR⁶—; R⁶ is hydrogen, C₁-C₆alkyl, C₁-C₆fluoroalkyl, or C₁-C₆deuteroalkyl;
L² is substituted or unsubstituted C₁-C₄alkylene, substituted or unsubstituted C₂-C₄alkenylene or substituted or unsubstituted C₂-C₄alkynylene;
L³ is C₁-C₄alkylene;
X¹ is CR² or N;
X² is CR² or N;
each R² is independently hydrogen, deuterium, halogen, —CN, —OR⁵, —SR⁵, —S(═O)R⁴, —S(═O)₂R⁴, —N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, substituted or unsubstituted C₃-C₆cycloalkyl;
R³ is hydrogen, substituted or unsubstituted C₁-C₆alkyl, substituted or unsubstituted C₁-C₆fluoroalkyl, or substituted or unsubstituted C₁-C₆deuteroalkyl;
Ring D is a 6-membered heteroaryl, phenyl, or a 5-membered heteroaryl;
each R^{b} is independently hydrogen, deuterium, halogen, —CN, —OR⁵, —SR⁵, —S(═O)R⁴, —S(═O)₂R⁴, —S(═O)₂N(R⁵)₂, —R⁵S(═O)₂R⁴, —C(═O)R⁴, —OC(═O)R⁴, —CO₂R⁵, —OCO₂R⁴, —N(R⁵)₂, —OC(═O)N(R⁵)₂, —R⁵C(═O)R⁴, —R²C(═O)OR⁴, —C(═O)N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, substituted or unsubstituted C₃-C₆cycloalkyl;
n is 0, 1, 2, 3, or 4;
Ring E is a 5-membered heteroaryl;
each R^{c} is independently hydrogen, deuterium, halogen, —CN, —OR⁵, —SR^{S}, —S(═O)R⁴, —S(═O)₂R⁴, —S(═O)₂N(R⁵)₂, —NR⁵S(═O)₂R⁴, —C(═O)R⁴, —OC(═O)R⁴, —CO₂R⁵, —OCO₂R⁴, —N(R⁵)₂, —OC(═O)N(R⁵)₂, —NR⁵C(═O)R⁴, —NR⁵C(═O)OR⁴, —C(═O)N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, or substituted or unsubstituted C₃-C₆cycloalkyl;
p is 0, 1, 2, or 3;
each R⁴ is independently selected from C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, substituted or unsubstituted benzyl and substituted or unsubstituted heteroaryl;
each R⁵ is independently selected from hydrogen, C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, substituted or unsubstituted benzyl and substituted or unsubstituted heteroaryl;
or two R⁵ on the same N atom are taken together with the N atom to which they are attached to a substituted or unsubstituted N-containing heterocycle.

In some embodiments, the dual inhibitor of ASK1 and DYRK1A is a compound having the structure of Formula (XI), or a pharmaceutically acceptable salt or solvate thereof:

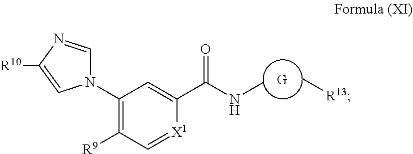

wherein:
Ring G is selected from

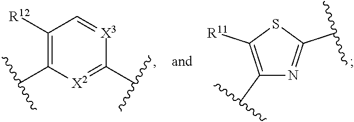

X¹, X² and X³ are each independently selected from N or)C(R¹⁰);
R¹⁰, R¹¹ and R¹² are each independently selected from the group consisting of: hydrogen, halogen, cyano, optionally substituted —C₁-C₆ alkyl, optionally substituted —C₃-C₈ cycloalkyl, optionally substituted 3- to 8-membered heterocycloalkyl, and optionally substituted —C₁-C₆ alkoxyl;
R¹³ is selected from:

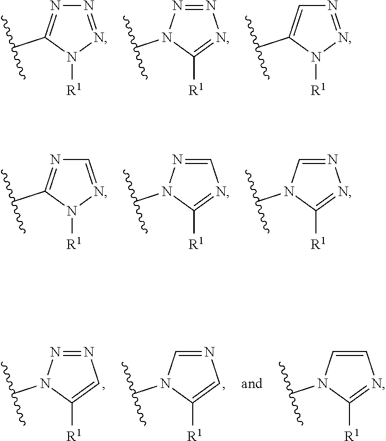

each of which is optionally substituted when possible;
R¹ is selected from the group consisting of: hydrogen, optionally substituted —C₁-C₆ alkyl, optionally substituted —C₂-C₈ alkenyl, optionally substituted —C₂-C₈ alkynyl, optionally substituted —C₃-C₈ cycloalkyl, optionally substituted aryl, optionally substituted arylalkyl, optionally substituted 3- to 8-membered heterocycloalkyl, optionally substituted heteroaryl, optionally substituted heteroarylalkyl, and —N(R¹⁶)(R¹⁷);
provided that when R¹³ is

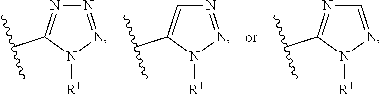

R¹ is not —N(R¹⁶)(R¹⁷);
R⁹ is selected from the group consisting of: hydrogen, halogen, cyano, optionally substituted —C₁-C₆ alkyl, optionally substituted —C₂-C₈ alkenyl, optionally substituted —C₂-C₈ alkynyl, optionally substituted —C₃-C₈ cycloalkyl, optionally substituted aryl, optionally substituted arylalkyl, optionally substituted 3- to 8-membered heterocycloalkyl, optionally substituted heteroaryl, optionally substituted heteroarylalkyl, —N(R¹⁶)(R¹⁷), —S(O)₂N(R¹⁶)(R¹⁷), —N(R¹⁶)C(O)(R¹⁷), and —N(R¹⁶)S(O)₂(R¹⁷); wherein
R¹⁶ and R¹⁷ are independently selected from the group consisting of hydrogen, —C₁-C₁₅ alkyl, cycloalkyl, heterocycloalkyl, aryl, and heteroaryl, wherein each alkyl, cycloalkyl, heterocycloalkyl, aryl and heteroaryl is optionally substituted with 1-3 substituents independently selected from halo, alkyl, alkylamino, dialkylamino, alkyl-C(O)NH—, aryl-C(O)NH-, heteroaryl-C(O)-NH, —CN, alkoxy, —CF₃, aryl, and heteroaryl,
or R¹⁷ and R¹⁶ are taken together with the nitrogen to which they are attached to form a heterocyclic.

In a further aspect described herein, is a method for the treatment or prevention of an ASK1 or DYRK1A associated condition, comprising administering to a subject in need thereof, a dual inhibitor of ASK1 and DYRK1A, wherein the dual inhibitor is a pharmaceutical composition comprising a compound of Formula (IV), (IVa), (IVb), (IVc), (V), (VI), (VII), (VIII), (IX), (X), or (XI), or a pharmaceutically acceptable salt or solvate thereof, and at least one pharmaceutically acceptable excipient. In another aspect described herein, is a method for the treatment or prevention of an ASK1 or DYRK1A associated condition, comprising administering to a subject in need thereof a therapeutic amount of a dual inhibitor of ASK1 and DYRK1A, wherein the dual inhibitor is a pharmaceutical composition comprising a compound of Formula (IV), (IVa), (IVb), (IVc), (V), (VI), (VII), (VIII), (IX), (X), (XI), or a pharmaceutically acceptable salt or solvate thereof and at least one pharmaceutically acceptable excipient.

In another aspect described herein, the ASK1 or DYRK1A associated condition is a cognitive impairment. In another aspect described herein, the cognitive impairment is a neurodegenerative disease or a neurodevelopmental disorder.

In another aspect described herein, is a method for the treatment or prevention of a neurodegenerative disease in a subject in need thereof, comprising administering to the subject a therapeutically effective amount of a dual inhibitor of ASK1 and DYRK1A, wherein the dual inhibitor is a compound of Formula (IV), (IVa), (IVb), (IVc), (V), (VI), (VII), (VIII), (IX), (X), or (XI), or a pharmaceutically acceptable salt or solvate thereof In another aspect described herein, is a method of treating a neurodegenerative disease in a subject in need thereof, comprising administering to the subject a therapeutically effective amount of a dual inhibitor of ASK1 and DYRK1A, wherein the dual inhibitor is a pharmaceutical composition comprising a compound of Formula (IV), (IVa), (IVb), (IVc), (V), (VI), (VII), (VIII), (IX), (X), or (XI), or a pharmaceutically acceptable salt or solvate thereof, and at least one pharmaceutically acceptable excipient.

In another aspect described herein, the neurodegenerative disease is an Alzheimer's disease (AD), an Alexander disease, an Alper's disease, an amyotrophic lateral sclerosis (ALS), an ataxia telangiectasia, a Canavan disease, a chronic traumatic encephalopathy, a Cockayne syndrome, a corticobasal degeneration, a Creutzfeld-Jakob disease, a dementia, a Guillain-Barre Syndrome, a Huntington disease (HD), a Kennedy's disease, a Krabbe disease, a Lewy body dementia, a Parkinson's disease (PD), a multiple sclerosis, a Pelizaeus-Merzbacher disease, a Pick's disease, a Refsum's disease, a Sandhoff disease, a Schilder's disease, a spinal cord injury, a Steele-Richardson-Olszewski disease, a tabes dorsalis, and/or a traumatic brain injury.

In another aspect described herein, the neurodegenerative disease is Alzheimer's disease (AD). In another aspect described herein, is a method for the treatment or prevention of Alzheimer's disease comprising administering to a subject in need thereof a therapeutic amount of a dual inhibitor of ASK1 and DYRK1A, wherein the dual inhibitor is a compound of Formula (IV), (IVa), (IVb), (IVc), (V), (VI), (VII), (VIII), (IX), (X), (XI), or a pharmaceutically acceptable salt or solvate thereof

In another aspect described herein, is a method for the treatment or prevention of a neurodevelopmental disorder in a subject in need thereof, comprising administering to the subject a therapeutically effective amount of a dual inhibitor of ASK1 and DYRK1A, wherein the dual inhibitor is a compound of Formula (IV), (IVa), (IVb), (IVc), (V), (VI), (VII), (VIII), (IX), (X), or (XI), or a pharmaceutically acceptable salt or solvate thereof In another aspect described herein is a method of treating a neurodevelopmental disorder in a subject in need thereof, comprising administering to the subject a therapeutically effective amount of a dual inhibitor of ASK1 and DYRK1A, wherein the dual inhibitor is a pharmaceutical composition comprising a compound of Formula (IV), (IVa), (IVb), (IVc), (V), (VI), (VII), (VIII), (IX), (X), or (XI), or a pharmaceutically acceptable salt or solvate thereof, and at least one pharmaceutically acceptable excipient.

In another aspect described herein, the neurodevelopmental disorder is Down syndrome (DS). In another aspect described herein is a method for the treatment or prevention of Down syndrome comprising administering to a subject in need thereof a therapeutic amount of a dual inhibitor of ASK1 and DYRK1A, wherein the dual inhibitor is a compound of Formula (IV), (IVa), (IVb), (IVc), (V), (VI), (VII), (VIII), (IX), (X), or (XI), or a pharmaceutically acceptable salt or solvate thereof or a pharmaceutical excipient.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Apoptosis signal-regulating kinase 1 (ASK1), a ubiquitously expressed 1375 amino acid serine/threonine-selective kinase, is a member of the mitogen-activated protein kinases (MAPKs) family.

The MAPK pathway is one of the intracellular signaling systems that regulate essential cellular functions, such as proliferation, motility, differentiation, stress response and apoptosis. Each MAPK pathway consists of three classes of protein kinases: MAPK kinase (MAP3K), MAPK kinase (MAP2K), and MAPK. MAP3K phosphorylates and thereby activates MAP2K, and activated MAP2K, in turn, phosphorylates and activates MAPK. Activated MAPK then translocate to the cell nucleus and regulates the activities of transcription factors to control gene expression.

Apoptosis signal-regulating kinase 1 (ASK1) is a membrane-proximal MAP3K (MAP-kinase-kinase-kinase) upstream of pathways which play important roles in the cellular response to environmental stresses (e.g. the c-Jun and p38 pathways, which are known to be responsive to UV and oxidative damage). A positive regulator of mitochondrial apoptosis, ASK1 is tightly regulated and activated by cellular damage signals as diverse as receptor-acting inflammatory cytokines (e.g. TNFa and LPS), calcium and intracellular sensors (e.g. the redox sensor thioredoxin, and the ER-stress-responsive IRE1). The activation of ASK1 also leads to abnormal phosphorylation of tau which causes extracellular deposition of senile plaques composed of beta-amyloid (Abeta) and the formation of intracellular neurofibrillary tangles (NFT).

Dual specificity tyrosine phosphorylation regulated kinase-1A (DYRK1A) is a member of the DYRK family. DYRK1A catalyzes autophosphorylation on serine/threonine and tyrosine resides.

The gene for DYRK1A is located within the Down syndrome critical region on human chromosome 21. DYRK1A is up-regulated during the early stages of embryonic development. Mice hemizygous for DYRK1A, show significant phenotypic effects including decreased neonatal viability, a reduced number of neurons, alternations in motor development and function, and impairment in the development of learning strategies. The diversity of phenotypes resulting from differential DYRK1A gene dosage suggests that DYRK1A activity is tightly regulated during the neuronal development processes. Moreover, lack of DYRK1A activity is associated with mental retardation and poor cognitive function.

DYRK1A targets a multitude of exogenous protein substrates, including transcription factors, splicing factors, cytoskeletal targets and synaptic proteins. DYRK1A phosphorylates the intracellular domain of the Notch receptor, regulating Notch-dependent biological processes such as angiogenesis, differentiation and transcription. By phosphorylating amyloid precursor protein (APP), tau, presenillinl, Asf, and setin-4 (all proteins involved in either neurofibrillary degeneration or β-amyloidosis), DYRK1A is an important factor in neuronal cell death and thereby is implicated the pathology of cognitive decline.

Neurodegenerative diseases are characterized by the progressive loss of neurons. Among the pathways strongly impacting the pathogenesis of neurodegenerative diseases, oxidative stress, a condition that occurs because of an imbalance in oxidant and antioxidant levels, has been known to play a vital role in the pathophysiology of neurodegenerative diseases. One of the molecules activated by oxidative stress is apoptosis signal-regulating kinase 1 (ASK1), which has been shown to play a role in neurodegenerative disease. ASK1 activation is regulated by multiple steps, including oligomerization, phosphorylation, and protein-protein interactions. In the oxidative stress state, reactive oxygen species (ROS) induce the dissociation of thioredoxin, a protein regulating cellular reduction and oxidation (redox), from the N-terminal region of ASK1, and ASK 1 is subsequently activated by the oligomerization and phosphorylation of a critical threonine residue, leading to cell death. ASK1 is also associated with insulin signal transduction, an important signaling component in cognitive decline. The inhibition of ASK1 induces tyrosine phosphorylation of IRS-1 and prevents cognitive decline in the brain. Thus, there is an association between ASK1 and neurodegeneration.

Evidence supports the critical role for DYRK1A in neurodegeneration and Alzheimer's disease (AD). Key DYRK1A serine/threonine phosphorylation targets including tau protein, amyloid precursor protein (APP), and presenilin point to clear mechanisms through which elevated DYRK1A activity contributes to AD progression.

Parkinson's disease (PD) is a disorder of the nervous system that results from the loss of cells in various parts of the brain, including in the substantia nigra. The substantia nigra cells produce dopamine, a chemical messenger responsible for transmitting signals within the brain that allow for coordination and movement. Loss of dopamine causes neurons to fire without normal control, leaving patients less able to direct their movement. In the mitochondrial complex 1 model of MPTP (1-methyl-4-phenyl-1,2,3,6-tetrahyropyridine) model of dopaminergic cell loss, ASK1 deficient mice were shown to be relatively resistant to MPTP lesions. Moreover, MPTP-induced dopamine neuron toxicity and motor impairment was also attenuated in ASK1 knock-out mice.

DYRK1A and ASK1 have been implicated in brain development. Overexpression of DYRK1A has been associated with decreased cognitive development and has been associated with Down syndrome (DS) and autism disorders. ASK1 and DYRK1A show promise as viable targets for a multitude of central nervous system based cognitive disorders and diseases.

### Certain Terminology

Unless otherwise stated, the following terms used in this application, including the specification and claims, have the definitions given below. It must be noted that, as used in the specification and the appended claims, the singular forms “a,” “an”, and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. In this application, the use of “or” or “and” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

An “alkyl” group refers to an aliphatic hydrocarbon group. The alkyl moiety may be branched or straight chain. The “alkyl” group may have 1 to 15 carbon atoms (whenever it appears herein, a numerical range such as “1 to 15” refers to each integer in the given range; e.g., “1 to 15 carbon atoms” means that the alkyl group may consist of 1 carbon atom, 2 carbon atoms, 3 carbon atoms, etc., up to and including 15 carbon atoms, although the present definition also covers the occurrence of the term “alkyl” where no numerical range is designated). In one aspect the alkyl is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, sec-butyl, tertiary butyl, pentyl, neopentyl, hexyl, and the like.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkynyl group has the formula —C≡C—R, wherein R refers to the remaining portions of the alkynyl group. In some embodiments, R is H or an alkyl. Non-limiting examples of an alkynyl group include —C≡CH, —C≡CCH₃—C≡CCH₂CH₃, —CH₂C≡CH.

The term “cycloalkyl” refers to a monocyclic or polycyclic aliphatic, non-aromatic radical, wherein each of the atoms forming the ring (i.e. skeletal atoms) is a carbon atom. Cycloalkyls may be saturated, or partially unsaturated. Cycloalkyls may be fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, and cyclooctyl. Cycloalkyl groups may be substituted or unsubstituted. Depending on the structure, a cycloalkyl group can be a monoradical or a diradical (i.e., an cycloalkylene group, such as, but not limited to, cyclopropan-1,1-diyl, cyclobutan-1,1-diyl, cyclopentan-1,1-diyl, cyclohexan-1,1-diyl, cyclohexan-1,4-diyl, cycloheptan-1,1-diyl, and the like). In one aspect, a cycloalkyl is a C₃-C₆cycloalkyl.

The term “aromatic” refers to a planar ring having a delocalized π-electron system containing 4n+2π electrons, where n is an integer. Aromatics are optionally substituted. The term “aromatic” includes both cycloalkyl aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups. The term “aryl” refers to an aromatic ring wherein each of the atoms forming the ring is a carbon atom. Aryl groups are optionally substituted. Depending on the structure, an aryl group can be a monoradical or a diradical (i.e., an arylene group).

The terms “heteroaryl” or, alternatively, “heteroaromatic” refers to an aryl group that includes one or more ring heteroatoms selected from nitrogen, oxygen and sulfur. Illustrative examples of heteroaryl groups include the following moieties:

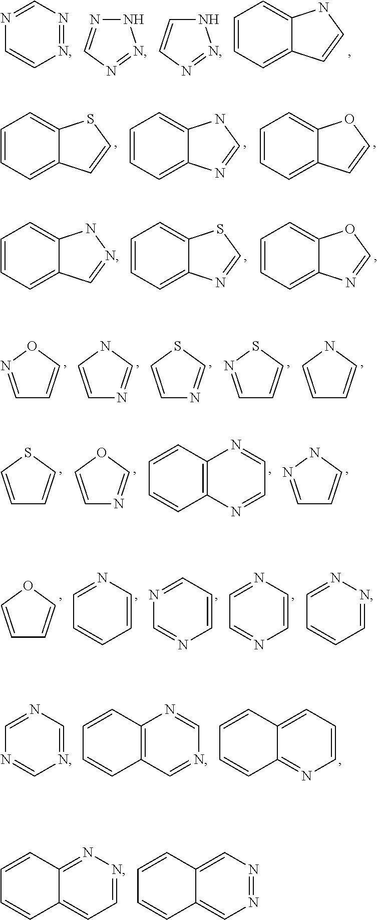

and the like. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. In some embodiments, a heteroaryl contains 0-3 N atoms in the ring. In some embodiments, a heteroaryl contains 1-3 N atoms in the ring. In some embodiments, a heteroaryl contains 0-3 N atoms, 0-1 O atoms, and 0-1 S atoms in the ring. In some embodiments, a heteroaryl is a monocyclic or bicyclic heteroaryl. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl. Depending on the structure, a heteroaryl group can be a monoradical or a diradical (i.e., a heteroarylene group).

A “heterocycle” or “heterocycloalkyl” group refers to a cycloalkyl group wherein at least one of the carbon atoms of the cycloalkyl is replaced with nitrogen (unsubstituted or substituted, e.g. —NH—, —N(alkyl)-), oxygen (—O—), or sulfur (e.g. —S—, —S(═O)— or —S(═O)₂—). The radicals may be fused with an aryl or heteroaryl. In some embodiments, the heterocycloalkyl is selected from oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, and indolinyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-3 N atoms in the ring. In some embodiments, a heterocycloalkyl contains 0-3 N atoms, 0-3 O atoms and 0-1 S atoms in the ring.

The term “halo” or, alternatively, “halogen” or “halide” means fluoro (F), chloro (Cl), bromo (Br) or iodo (I). The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, nitro, haloalkyl, fluoroalkyl, fluoroalkoxy, and amino, including mono- and di-substituted amino groups, and the protected derivatives thereof. In some embodiments, optional substituents are independently selected from halogen, —CN, —NH₂, —NH(CH₃), —N(CH₃)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some embodiments, optional substituents are independently selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic, saturated or unsaturated carbon atoms, excluding aromatic carbon atoms) includes oxo (═O).

In certain embodiments, the compounds presented herein possess one or more stereocenters and each center independently exists in either the R or S configuration. The compounds presented herein include all diastereomeric, enantiomeric, and epimeric forms as well as the appropriate mixtures thereof. Stereoisomers are obtained, if desired, by methods such as, stereoselective synthesis and/or the separation of stereoisomers by chiral chromatographic columns. In some embodiments, halogen is F, Cl, Br, or I. In some embodiments, halogen is F or Cl. In some embodiments, halogen is F.

The methods and formulations described herein include the use of N-oxides (if appropriate), crystalline forms (also known as polymorphs), or pharmaceutically acceptable salts of compounds having the structure of Formula (IV), (IVa), (IVb), (IVc), (V), (VI), (VII), (VIII), (IX), or (X), as well as active metabolites of these compounds having the same type of activity. In some situations, compounds may exist as tautomers. All tautomers are included within the scope of the compounds presented herein. In specific embodiments, the compounds described herein exist in solvated forms with pharmaceutically acceptable solvents such as water, ethanol, and the like. In other embodiments, the compounds described herein exist in unsolvated form.

The term “acceptable” with respect to a formulation, composition or ingredient, as used herein, means having no persistent detrimental effect on the general health of the subject being treated.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula (IV), (IVa), (IVb), (IVc), (V), (VI), (VII), (VIII), (IX), or (X), or a pharmaceutically acceptable salt thereof, and a co-agent, are both administered to a subject simultaneously in the form of a single entity or dosage. The term “non-fixed combination” means that the active ingredients, e.g. a compound of Formula (IV), (IVa), (IVb), (IVc), (V), (VI), (VII), (VIII), (IX), or (X), or a pharmaceutically acceptable salt thereof, and a co-agent, are administered to a subject as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

An “ASK1 inhibitor” refers to a compound (e.g., compounds described herein) that reduces the activity of ASK1 when compared to a control, such as absence of the compound or a compound with known inactivity.

A “DYRK1A inhibitor” refers to a compound (e.g., compounds described herein) that reduces the activity of DYRK lA when compared to a control, such as absence of the compound or a compound with known inactivity.

A “dual inhibitor of ASK1 and DYRK1A” refers to a compound displaying less than 10-fold selectivity between ASK1 and DYRK1A and/or simultaneously acting on ASK1 and DYRK1A with an IC₅₀ lower than 100 nM on each.

As defined herein, the term “inhibition,” “inhibit,” “inhibiting,” and the like, in reference to a protein-inhibitor interaction means negatively affecting (e.g. decreasing) the activity or function of the protein relative to the activity or function of the protein in the absence of the inhibitor. In embodiments, inhibition means negatively affecting (e.g. decreasing) the concentration or levels of the protein relative to the concentration or level of the protein in the absence of the inhibitor. In embodiments, inhibition refers to reduction of a disease or symptoms of disease. In certain embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In certain embodiments, inhibition refers to a reduction of activity of a target protein resulting from a direct interaction (e.g. an inhibitor binds to the target protein). In certain embodiments, inhibition refers to a reduction of activity of a target protein from an indirect interaction (e.g. an inhibitor binds to a protein that activates the target protein, thereby preventing target protein activation).

The terms “condition”, “disease”, or “disorder” refer to a state of being or health status of a patient or subject capable of being treated with the compounds or methods provided herein. The condition may be a cognitive impairment. The disease may be a neurodegenerative disease. The disorder may be a neurodevelopmental disorder.

As used herein, the term “cognitive impairment” refers to the brain, central nervous system or peripheral nervous system being diminished, weakened, or damaged especially in functions associated with cognition such as, but not limited to, thinking, learning, speaking, memory, coordination, and/or muscle memory and control. Cognitive impairment can be the result of a progressive neurodegenerative process. Cognitive impairment can also result from incomplete and/or aberrant neurodevelopment and/or insufficient development of the central nervous system and the brain.

As used herein, the term “cognitive dysfunction” refers to the brain, central nervous system or peripheral nervous system being diminished, weakened, or damaged especially in functions associated with cognition such as, but not limited to, thinking, learning, speaking, memory, coordination, and/or muscle memory and control. The terms “cognitive dysfunction” and “cognitive impairment” are synonymous.

As used herein, the terms “neurodegeneration” and/or “neurodegenerative disease” refer to a disease or condition involving the progressive loss of structure or function of neurons, including the death of neurons. Many neurodegenerative diseases—including but not limited to amyotrophic lateral sclerosis, Parkinson's, Alzheimer's, Huntington's, traumatic brain injury (TBI), and chronic traumatic encephalopathy (CTE)—occur as a result of neurodegenerative processes. Such diseases result in progressive degeneration and/or death of neuron cells. Neurodegeneration can be found in many different levels of neuronal circuitry ranging from molecular to systemic.

As used herein, the term “neurodevelopmental disorder” refers to a disorder or condition involving insufficient neuron and/or brain development. This process results from a multitude of causes including but not limited to environmental or genetic factors. Many neurodevelopmental disorders—including but not limited to Down syndrome (DS) and mental retardation—are characterized by poor neuronal development leading to impaired cognition and brain function.

The dual inhibitors described herein can be used in combination with one another or with other active agents or therapies known to be useful in the treatment or prevention of neurodegenerative disease (e.g. Alzheimer's disease or Parkinson's disease).

The dual inhibitors described herein can be used in combination with one another or with other active agents or therapies known to be useful in the treatment or prevention of neurodevelopmental disorder (e.g. Down syndrome).

### Compounds

In one aspect, presented herein is a method for treating a cognitive impairment in a subject in need thereof, comprising administering to the subject, a dual inhibitor of ASK1 and DYRK1A, wherein the dual inhibitor is a compound having the structure of Formula (IV), or a pharmaceutically acceptable salt or solvate thereof:

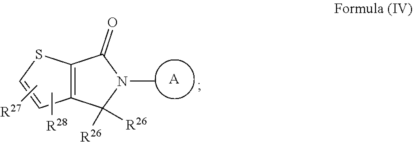

wherein:

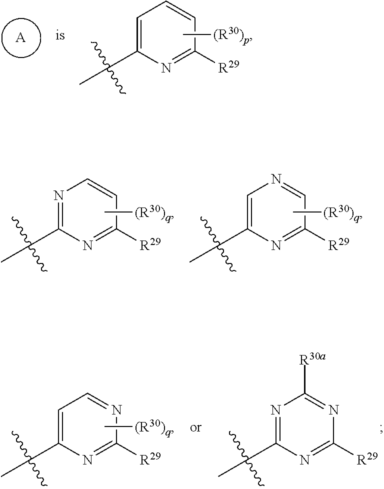

each R²⁶ is independently selected from a group consisting of hydrogen, halogen, and C₁-C₆alkyl;
R²⁷ is selected from a group consisting of hydrogen, halogen, —CN, —OH, —OR³¹, —SR³¹, —S(═O)R³², —NO₂, —N(R³¹)₂, —S(═O)₂R³², —NHS(═O)₂R³², —S(═O)₂N(R³¹)₂, —C(═O)R³², —C(═O)OR³¹, —OC(═O)R³², —C(═O)N(R⁶)₂, —OC(═O)N(R³¹)₂, —NR³¹C(═O)N(R³¹)₂, —NR³¹C(═O)R³², —NR³¹C(═O)OR³¹, C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂-N(R³⁹)₂, —N(R³⁸)₂, —N(R³⁹)C(═O)R³⁹, and —N(R³⁸)S(═O)₂R³⁸;
R²⁸ is selected from a group consisting of hydrogen, halogen, —CN, and C₁₋₆alkyl; or R²⁷ and R²⁸ are combined to form a phenyl ring optionally substituted with one, two, or three R³³ substituents;
R²⁹ is selected from a group consisting of hydrogen, halogen, —CN, —OH, —OR³¹, —SR³¹, —S(═O)R³², —NO₂, —N(R³¹)₂, —S(═O)₂R³², —NHS(═O)₂R³², —S(═O)₂N(R³¹)₂, —C(═O)R³², —C(═O)OR³¹, —OC(═O)R³², —C(═O)N(R³¹)₂, —OC(═O)N(R³¹)₂, —NR³¹C(═O)N(R³¹)₂, —NR³¹C(═O)R³², —NR³¹C(═O)OR³¹, C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂—N(R³⁸)₂, —N(R³⁸)₂, —N(R³⁸)C(═O)R³⁹, and —N(R³⁸)S(═O)₂R³⁸;
each R³⁰ is independently selected from a group consisting of halogen, —CN, and C₁₋₆alkyl;
R^{30a} is selected from the group consisting of hydrogen and C₁-C₆alkyl;
each R³¹ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle; or two R³¹ on the same heteroatom are taken together with that heteroatom to which they are attached to form a C₂₋₉heterocycle or a C₂₋₉heteroaryl;
each R³² is independently selected from the group consisting of C₁-C₆alkyl, C₃-C₈cycloalkyl, and C₂₋₉heterocycle;
each R³³ is independently selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸;
each R³⁸ is independently selected from the group consisting of hydrogen, C₁-C₆alkyl, and C₃-C₈cycloalkyl; or two R³⁸ on the same heteroatom are taken together with that heteroatom to which they are attached to form a C₂₋₉heterocycle;
each R³⁹ is independently selected from the group consisting of C₁-C₆alkyl and C₃-C₈cycloalkyl;
p is 0, 1, 2, or 3; and q is 0, 1, or 2.

In some embodiments, the dual inhibitor of Formula (IV), has the structure of Formula (IVa), or a pharmaceutically acceptable salt or solvate thereof:

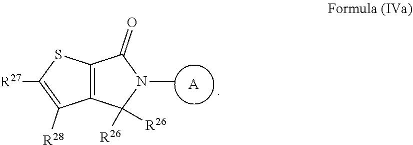

In some embodiments, the dual inhibitor of Formula (IV), has the structure of Formula (IVb), or a pharmaceutically acceptable salt or solvate thereof:

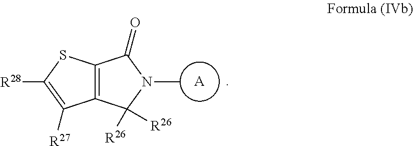

In some embodiments of the dual inhibitor of Formula (IV), (IVa), or (IVb), or a pharmaceutically acceptable solvate or salt thereof, R²⁷ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋ₘaryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), or (IVb), or a pharmaceutically acceptable salt or solvate thereof, R²⁷ is selected from a group consisting of C₂₋₉heterocycle and C₁₋₉heteroaryl; wherein C₂₋₉heterocycle and C₁₋₉heteroaryl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), or (IVb), or a pharmaceutically acceptable salt or solvate thereof, R²⁷ is selected from a group consisting of C₂₋₉heterocycle and C₁₋₉heteroaryl; wherein C₂₋₉heterocycle and C₁₋₉heteroaryl are optionally substituted with one or two substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), or (IVb), or a pharmaceutically acceptable salt or solvate thereof, R²⁷ is selected from a group consisting of pyrazole, imidazole, thiazole, and pyridine; wherein pyrazole, imidazole, thiazole, and pyridine are optionally substituted with one or two substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), or (IVb), or a pharmaceutically acceptable salt or solvate thereof, R²⁷ is selected from a group consisting of pyrazole, imidazole, thiazole, and pyridine; wherein pyrazole, imidazole, thiazole, and pyridine are optionally substituted with one or two substituents selected from the group consisting of halogen, C₁₋₆alkyl, and C₃₋₈cycloalkyl. In some embodiments of the dual inhibitor of Formula (IV), (IVa), or (IVb), or a pharmaceutically acceptable salt or solvate thereof, R²⁷ is

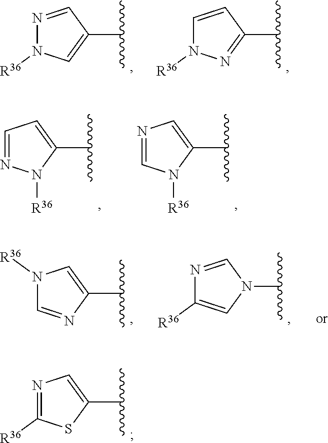

wherein R³⁶ is C₁-C₆alkyl or C₃-C₆cycloalkyl. In some embodiments of the dual inhibitor of Formula (IV), (IVa), or (IVb), or a pharmaceutically acceptable salt or solvate thereof, R²⁷ is

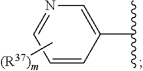

wherein each R³⁷ is independently hydrogen, halogen, CN, C₁-C₆alkyl, or C₃-C₆cycloalkyl; and m is 1 or 2. In some embodiments of the dual inhibitor of Formula (IV), (IVa), or (IVb), or a pharmaceutically acceptable salt or solvate thereof, R²⁷ is selected from a group consisting of unsubstituted pyrazole, unsubstituted imidazole, unsubstituted thiazole, and unsubstituted pyridine. In some embodiments of the dual inhbitor of Formula (IV), (IVa), or (IVb), or a pharmaceutically acceptable salt or solvate thereof, R²⁸ is unsubstituted pyridyl. In some embodiments of the dual inhbitor of Formula (IV), (IVa), or (IVb), or a pharmaceutically acceptable salt or solvate thereof, R²⁷ is C₆₋₁₀aryl optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (W), (IVa), or (IVb), or a pharmaceutically acceptable salt or solvate thereof, R²⁷ is phenyl optionally substituted with one or two substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual of Formula (IV), (IVa), or (IVb), or a pharmaceutically acceptable salt or solvate thereof, R²⁷ is —C(═O)N(R³¹)₂ and each R³¹ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle; or two R³¹ on the same heteroatom are taken together with that heteroatom to which they are attached, to form a C₂₋₉heterocycle or a C₂₋₉heteroaryl. In some embodiments of the dual inhibitor of Formula (IV), (IVa), or (IVb), or a pharmaceutically acceptable salt or solvate thereof, R²⁷ is

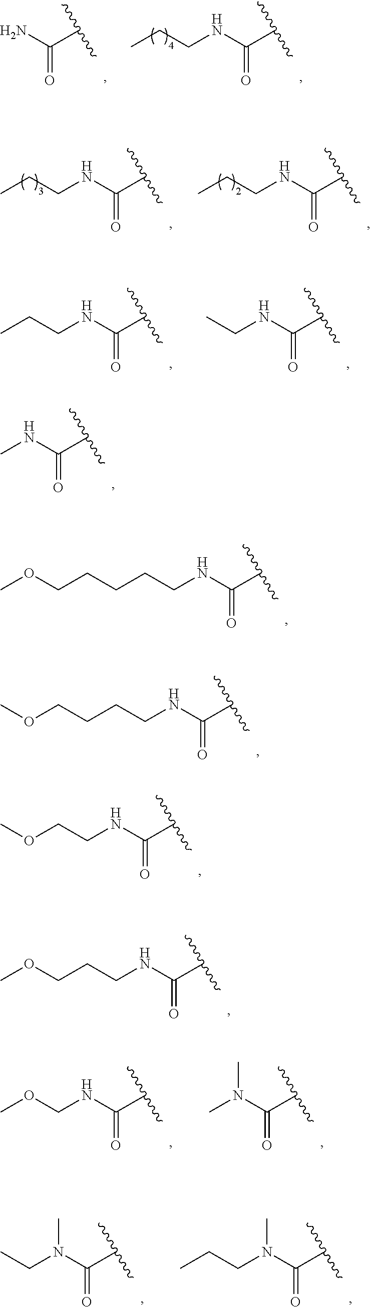

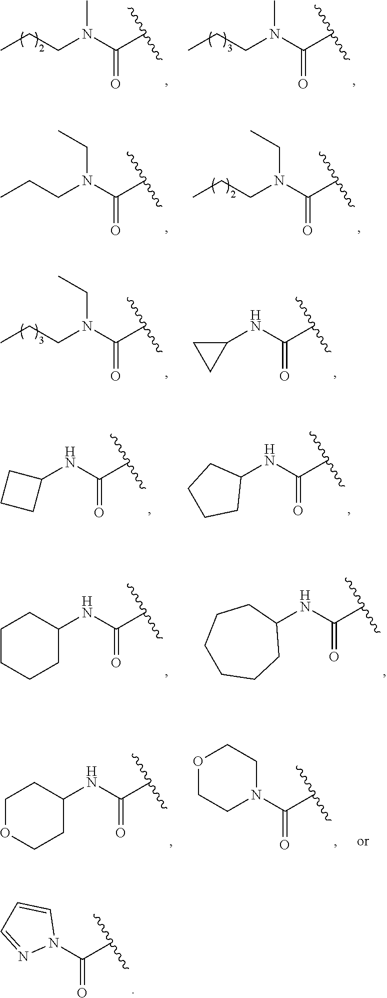

In some embodiments of the dual inhibitor of Formula (IV), (IVa), or (IVb), or a pharmaceutically acceptable salt or solvate thereof, R²⁷ is

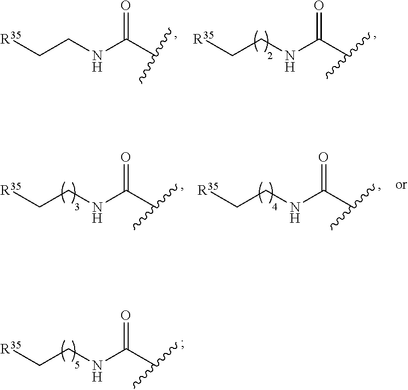

wherein R³⁵ is C₁₋₉heteroaryl. In some embodiments of the dual inhibitor of Formula (IV), (IVa), or (IVb), or a pharmaceutically acceptable salt or solvate thereof, R²⁷ is —NHC(═O)R³². In some embodiments of the dual inhibitor of Formula (IV), (IVa), or (IVb), or a pharmaceutically acceptable salt or solvate thereof, R²⁸ is unsubstituted pyrazolyl. In some embodiments of the dual inhibitor of Formula (IV), (IVa), or (IVb), or a pharmaceutically acceptable salt or solvate thereof, R²⁷ is

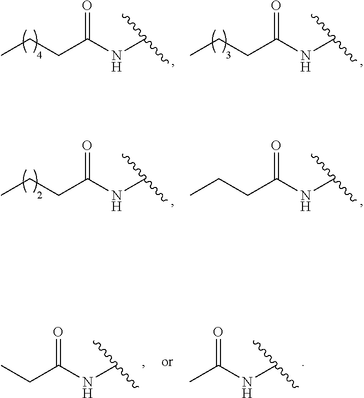

In some embodiments of the dual inhibitor of Formula (IV), (IVa), or (IVb), or a pharmaceutically acceptable salt or solvate thereof, R²⁷ is —C(═O)R³². In some embodiments of the dual inhibitor of Formula (IV), (IVa), or (IVb), or a pharmaceutically acceptable salt or solvate thereof, R²⁷ is

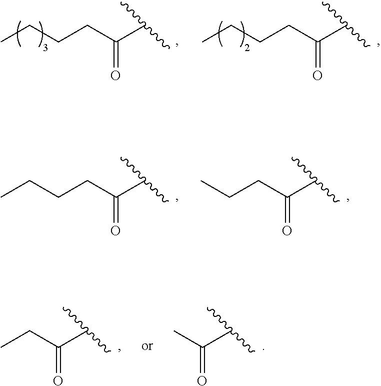

In some embodiments of the dual inhibitor of Formula (IV), (IVa), or (IVb), or a pharmaceutically acceptable salt or solvate thereof, R²⁸ is hydrogen. In some embodiments of the dual inhibitor of Formula (IV), (IVa), or (IVb), or a pharmaceutically acceptable salt or solvate thereof R²⁸ is C₁-C₆alkyl.

In one aspect presented herein, is a method for treating cognitive impairment comprising administering to the subject, a dual inhibitors of ASK1 and DYRK1A, wherein the dual inhibitor is a compound having the structure of Formula (IVc), or a pharmaceutically acceptable salt or solvate thereof:

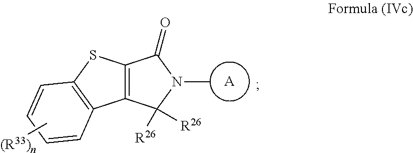

wherein each R²⁶ is defined in embodiments; R³³ is independently selected from a group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂—N(R³⁸)₂, —N(R³⁸)₂, —N(R³⁸)C(═O)R³⁹, and —N(R³⁸)S(═O)₂R³⁸; and n is 0, 1, or 2.

In some embodiments of the dual inhibitor of Formula (IV), (IVa), (IVb), or (IVc), or a pharmaceutically acceptable salt or solvate thereof,

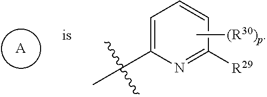

In some embodiments of the dual inhibitor of Formula (IV), (IVa), (IVb), or (IVc), or a pharmaceutically acceptable salt or solvate thereof,

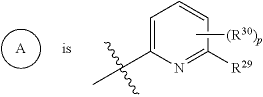

and p is 2. In some embodiments of the dual inhibitor of Formula (IV), (IVa), (IVb), or (IVc), or a pharmaceutically acceptable salt or solvate thereof,

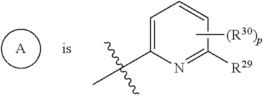

and p is 1. In some embodiments of the dual inhibitor of Formula (IV), (IVa), (IVb), or (IVc), or a pharmaceutically acceptable salt or solvate thereof,

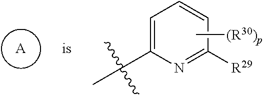

and p is 0. In some embodiments is the dual inhibitor of Formula (IV), (IVa), (IVb), or (IVc), or a pharmaceutically acceptable salt or solvate thereof,

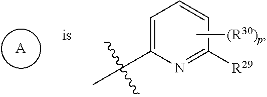

p is 0, and R²⁹ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

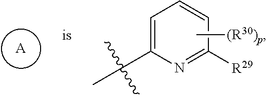

p is 0, and R²⁹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂—N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

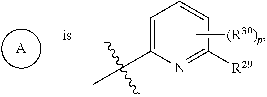

p is 0, and R²⁹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one or two substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

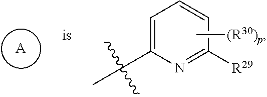

p is 0, and R²⁹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

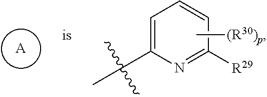

p is 0, and R²⁹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halogen, C₁₋₆alkyl, and C₃₋₈cycloalkyl. In some embodiments the dual inhibitor of Formula (IV), (IVa), (IVb), or (IVc), or a pharmaceutically acceptable salt or solvate thereof,

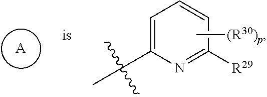

### p is 0, and R²⁹ is

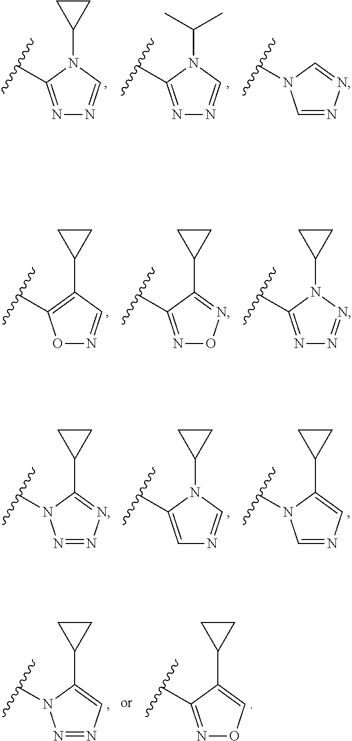

In some embodiments of the dual inhibitor of Formula (IV), (IVa), (IVb), or (IVc), or a pharmaceutically acceptable salt or solvate thereof, wherein

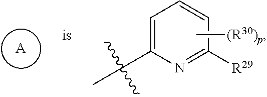

### p is 0, and R²⁹ is

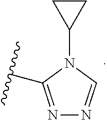 [SMILES: *c1nncn1C1CC1]

In some embodiments is the dual inhibitor of Formula (IV), (IVa), (IVb), or (IVc), or a pharmaceutically acceptable salt or solvate thereof, wherein

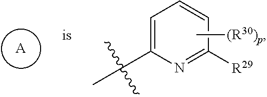

### p is 0, and R²⁹ is

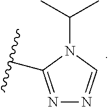 [SMILES: *c1nncn1C(C)C]

In some embodiments of the dual inhibitor of Formula (IV), (IVa), (IVb) or (IVc), or a pharmaceutically acceptable salt or solvate thereof, wherein

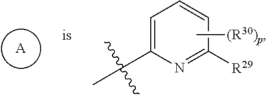

### p is 0, and R²⁹ is

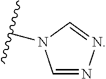 [SMILES: *n1cnnc1]

In some embodiments the dual inhibitor of Formula (IV), (IVa), (IVb) or (IVc), or a pharmaceutically acceptable salt or solvate thereof, wherein

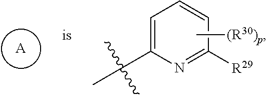

### p is 0, and R²⁹ is

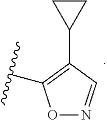 [SMILES: *c1oncc1C1CC1]

In some embodiments of the dual inhibitor of Formula (IV), (IVa), (IVb) or (IVc), or a pharmaceutically acceptable salt or solvate thereof, wherein

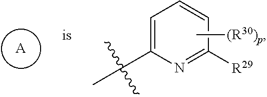

### p is 0, and R²⁹ is

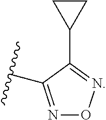 [SMILES: *c1nonc1C1CC1]

In some embodiments of the dual inhibitor of Formula (IV), (IVa), (IVb) or (IVc), or a pharmaceutically acceptable salt or solvate thereof

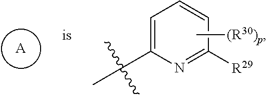

### p is 0, and R²⁹ is

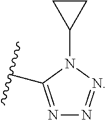 [SMILES: *c1nnnn1C1CC1]

In some embodiments of a dual inhibitor of Formula (IV), (IVa), (IVb) or (IVc), or a pharmaceutically acceptable salt or solvate thereof, wherein

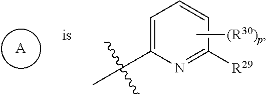

### p is 0, and R²⁹ is

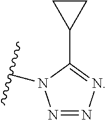 [SMILES: *n1nnnc1C1CC1]

In some embodiments of the dual inhibitor of Formula (IV), (IVa), (IVb) or (IVc), or a pharmaceutically acceptable salt or solvate thereof, wherein

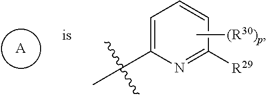

### p is 0, and R²⁹ is

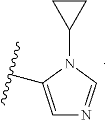 [SMILES: *c1cncn1C1CC1]

In some embodiments of the dual inhibitor of Formula (IV), (IVa), (IVb) or (IVc), or a pharmaceutically acceptable salt or solvate thereof, wherein

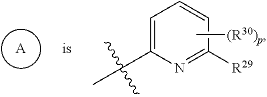

### s, p is 0, and R²⁹ is

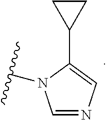 [SMILES: *n1cncc1C1CC1]

In some embodiments of the dual inhibitor of Formula (IV), (IVa), (IVb) or (IVc), or a pharmaceutically acceptable salt or solvate thereof, wherein

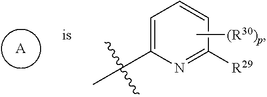

### p is 0, and R²⁹ is

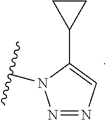 [SMILES: *n1nncc1C1CC1]

In some embodiments of the dual inhibitor of Formula (IV), (IVa), (IVb) or (IVc), or a pharmaceutically acceptable salt or solvate thereof, wherein

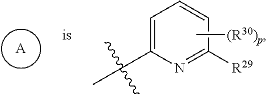

### p is 0, and R²⁹ is

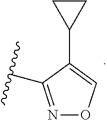 [SMILES: *c1nocc1C1CC1]

In some embodiments of the dual inhibitor of Formula (IV), (IVa), (IVb) or (IVc), or a pharmaceutically acceptable salt or solvate thereof, wherein

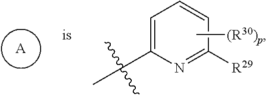

### p is 0, and R²⁹ is

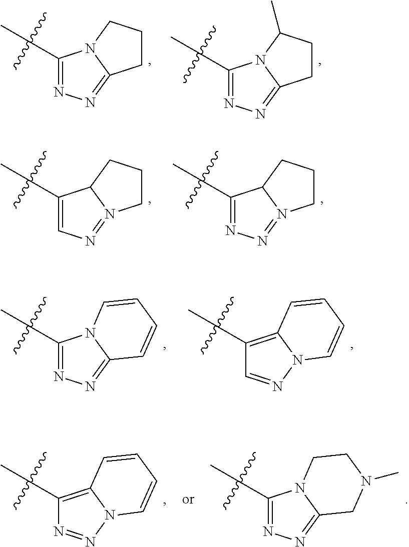

In some embodiments of the dual inhibitor of Formula (IV), (IVa), (IVb) or (IVc), or a pharmaceutically acceptable salt or solvate thereof, wherein

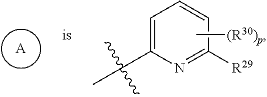

### p is 0, and R²⁹ is

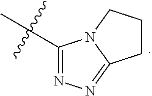 [SMILES: *c1nnc2n1CCC2]

In some embodiments of the dual inhibitor of Formula (IV), (IVa), (IVb) or (IVc), or a pharmaceutically acceptable salt or solvate thereof, wherein

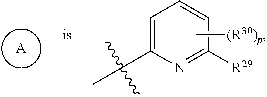

### p is 0, and R²⁹ is

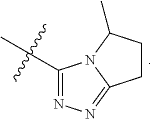 [SMILES: *c1nnc2n1C(C)CC2]

In some embodiments of the dual inhibitor of Formula (IV), (IVa), (IVb) or (IVc), or a pharmaceutically acceptable salt or solvate thereof, wherein

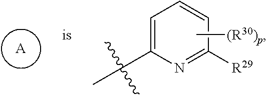

### p is 0, and R²⁹ is

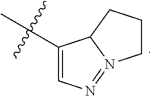

In some embodiments of the dual inhibitor of Formula (IV), (IVa), (IVb) or (IVc), or a pharmaceutically acceptable salt or solvate thereof, wherein

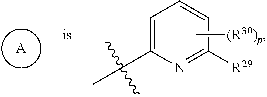

### p is 0, and R²⁹ is

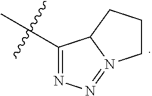

In some embodiments of the dual inhibitor of Formula (IV), (IVa), (IVb) or (IVc), or a pharmaceutically acceptable salt or solvate thereof, wherein

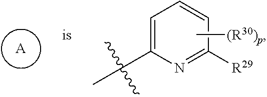

### p is 0, and R²⁹ is

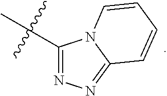 [SMILES: *c1nnc2ccccn12]

In some embodiments of the dual inhibitor of Formula (IV), (IVa), (IVb) or (IVc), or a pharmaceutically acceptable salt or solvate thereof, wherein

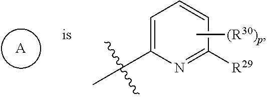

### p is 0, and R²⁹ is

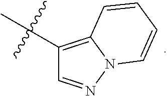 [SMILES: *c1cnn2ccccc12]

In some embodiments of the dual inhibitor of Formula (IV), (IVa), (IVb) or (IVc), or a pharmaceutically acceptable salt or solvate thereof, wherein

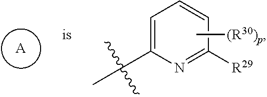

### p is 0, and R²⁹ is

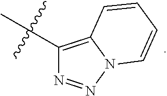 [SMILES: *c1nnn2ccccc12]

In some embodiments of the dual inhibitor of Formula (IV), (IVa), (IVb), or (IVc), or a pharmaceutically acceptable salt or solvate thereof, wherein

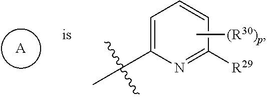

### p is 0, and R²⁹ is

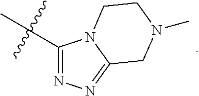 [SMILES: *c1nnc2n1CCN(C)C2]

In some embodiments of the dual inhibitor of Formula (IV), (IVa), (IVb), or (IVc), or a pharmaceutically acceptable salt or solvate thereof, wherein

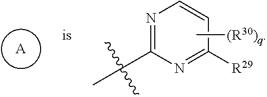

In some embodiments of the dual inhibitor of Formula (IV), (IVa), (IVb), or (IVc), or a pharmaceutically acceptable salt or solvate thereof, wherein

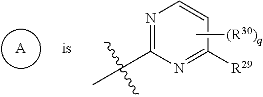

and q is 2. In some embodiments of the dual inhibitor of Formula (IV), (IVa), (IVb), or (IVc), or a pharmaceutically acceptable salt or solvate thereof, wherein

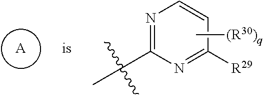

and q is 1. In some embodiments is the dual inhibitor of Formula (IV), (IVa), (IVb), or (IVc), or a pharmaceutically acceptable salt or solvate thereof, wherein

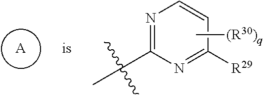

and q is 0. I n some embodiments of the dual inhibitor of Formula (IV), (IVa), (IVb), or (IVc), or a pharmaceutically acceptable salt or solvate thereof, wherein

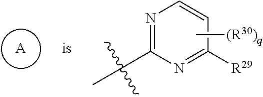

q is 0, and R²⁹ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂, —N(R³⁸)₂, —-N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof, wherein

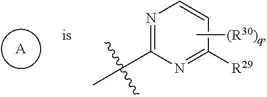

q is 0, and R²⁹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

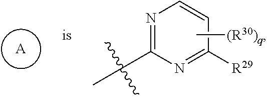

q is 0, and R²⁹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one or two substituents selected from the group consisting of halogen, —CN, C₁₋₆alkyl, —C₁₋₆alkyl-OH, C₁₋₆haloalkyl, C₃₋₈cycloalkyl, C₂₋₉ heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂—N(R¹⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

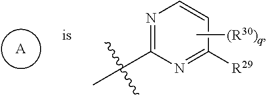

q is 0, and R²⁹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂—N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

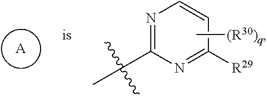

q is 0, and R²⁹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halogen, C₁₋₆alkyl, and C₃₋₈cycloalkyl. In some embodiments of the dual inhibitor of Formula (IV), (IVa), (IVb), or (IVc), or a pharmaceutically acceptable salt or solvate thereof,

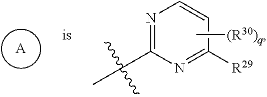

### q is 0, and R²⁹ is

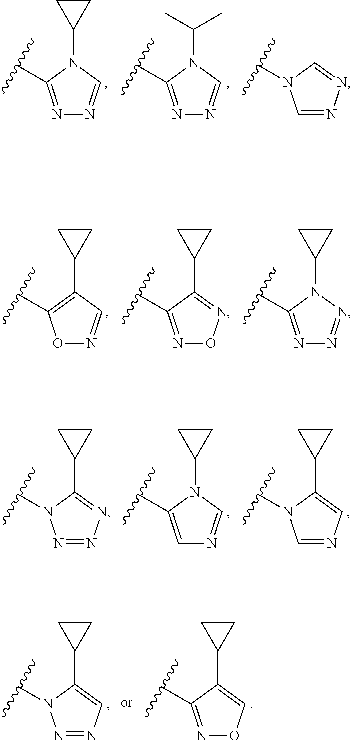

In some embodiments of the dual inhibitor of Formula (IV), (IVa), (IVb), or (IVc), or a pharmaceutically acceptable salt or solvate thereof,

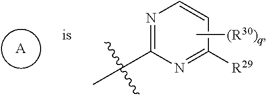

### q is 0, and R²⁹ is

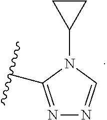 [SMILES: *c1nncn1C1CC1]

In some embodiments is the dual inhibitor of Formula (IV), (IVa), (IVb), or (IVc), or a pharmaceutically acceptable salt or solvate thereof,

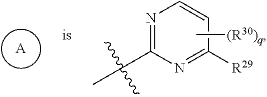

### q is 0, and R²⁹ is

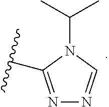 [SMILES: *c1nncn1C(C)C]

In some embodiments of the dual inhibitor of Formula (IV), (IVa), (IVb), or (IVc), or a pharmaceutically acceptable salt or solvate thereof, wherein

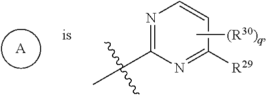

### q is 0, and R²⁹ is

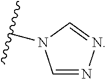 [SMILES: *n1cnnc1]

In some embodiments of the dual inhibitor of Formula (IV), (IVa), (IVb), or (IVc), or a pharmaceutically acceptable salt or solvate thereof,

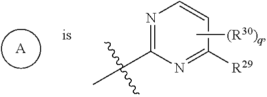

### q is 0, and R²⁹ is

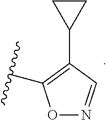 [SMILES: *c1oncc1C1CC1]

In some embodiments of the dual inhibitor of Formula (IV), (IVa), (IVb), or (IVc), or a pharmaceutically acceptable salt or solvate thereof,

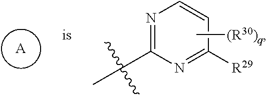

### q is 0, and R²⁹ is

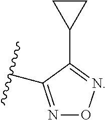 [SMILES: *c1nonc1C1CC1]

In some embodiments of the dual inhibitor of Formula (IV), (IVa), (IVb), or (IVc), or a pharmaceutically acceptable salt or solvate thereof,

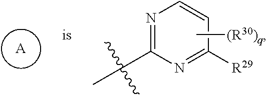

### q is 0, and R²⁹ is

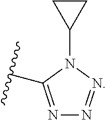 [SMILES: *c1nnnn1C1CC1]

In some embodiments of the dual inhibitor of Formula (IV), (IVa), (IVb), or (IVc), or a pharmaceutically acceptable salt or solvate thereof,

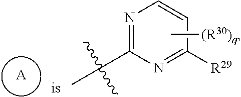

### q is 0, and R²⁹ is

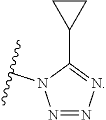 [SMILES: *n1nnnc1C1CC1]

In some embodiments of the dual inhibitor of Formula (IV), (IVa), (IVb), or (IVc), or a pharmaceutically acceptable salt or solvate thereof,

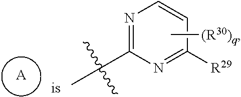

### q is 0, and R²⁹ is

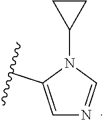 [SMILES: *c1cncn1C1CC1]

In some embodiments the dual inhibitor of Formula (IV), (IVa), (IVb), or (IVc), or a pharmaceutically acceptable salt or solvate thereof,

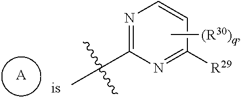

### q is 0, and R²⁹ is

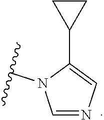 [SMILES: *n1cncc1C1CC1]

In some embodiments of the dual inhibitor of Formula (IV), (IVa), (IVb), or (IVc), or a pharmaceutically acceptable salt or solvate thereof,

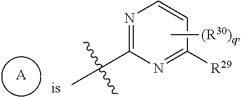

### q is 0, and R²⁹ is

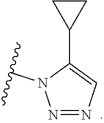 [SMILES: *n1nncc1C1CC1]

In some embodiments of the dual inhibitor of Formula (IV), (IVa), (IVb), or (IVc), or a pharmaceutically acceptable salt or solvate thereof,

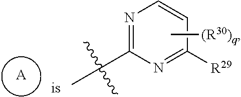

### q is 0, and R²⁹ is

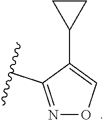 [SMILES: *c1nocc1C1CC1]

In some embodiments of the dual inhibitor of Formula (IV), (IVa), (IVb), or (IVc), or a pharmaceutically acceptable salt or solvate thereof,

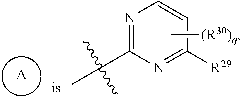

### q is 0, and R²⁹ is

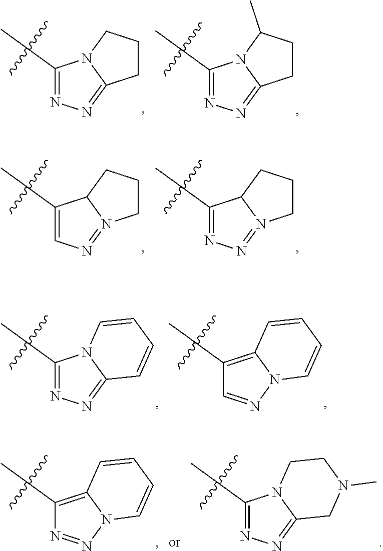

In some embodiments of the dual inhibitor of Formula (IV), (IVa), (IVb), or (IVc), or a pharmaceutically acceptable salt or solvate thereof, wherein

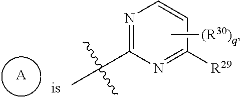

### q is 0, and R²⁹ is

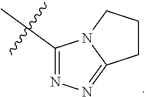 [SMILES: *c1nnc2n1CCC2]

In some embodiments of the dual inhibitor of Formula (IV), (IVa), (IVb), or (IVc), or a pharmaceutically acceptable salt or solvate thereof,

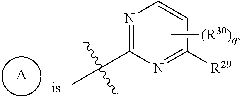

### q is 0, and R²⁹ is

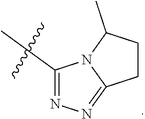 [SMILES: *c1nnc2n1C(C)CC2]

In some embodiments the dual inhibitor of Formula (IV), (IVa), (IVb), or (IVc), or a pharmaceutically acceptable salt or solvate thereof,

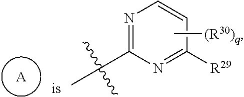

### q is 0, and R²⁹ is

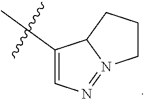

In some embodiments of the dual inhibitor of Formula (IV), (IVa), (IVb), or (IVc), or a pharmaceutically acceptable salt or solvate thereof,

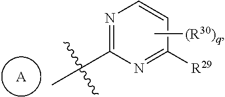

### is q is 0, and R²⁹ is

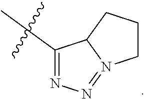

In some embodiments of the dual inhibitor of Formula (IV), (IVa), (IVb), or (IVc), or a pharmaceutically acceptable salt or solvate thereof,

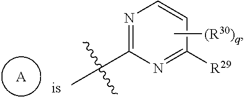

### q is 0, and R²⁹ is

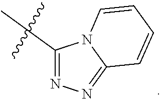 [SMILES: *c1nnc2ccccn12]

In some embodiments of the dual inhibitor of Formula (IV), (IVa), (IVb), or (IVc), or a pharmaceutically acceptable salt or solvate thereof,

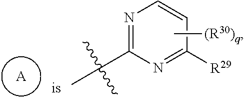

### q is 0, and R²⁹ is

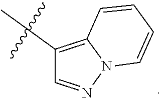 [SMILES: *c1cnn2ccccc12]

In some embodiments of the dual inhibitor of Formula (IV), (IVa), (IVb), or (IVc), or a pharmaceutically acceptable salt or solvate thereof,

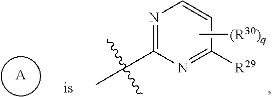

### q is 0, and R²⁹ is

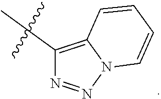 [SMILES: *c1nnn2ccccc12]

In some embodiments of the dual inhibitor of Formula (IV), (IVa), (IVb), or (IVc), or a pharmaceutically acceptable salt or solvate thereof,

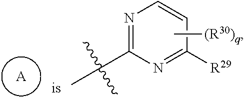

### q is 0, and R²⁹ is

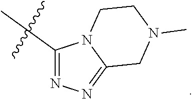 [SMILES: *c1nnc2n1CCN(C)C2]

In some embodiments the dual inhibitor of Formula (IV), (IVa), (IVb), or (IVc), or a pharmaceutically acceptable salt or solvate thereof,

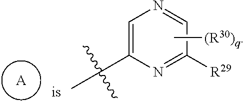

In some embodiments of the dual inhibitor of Formula (IV), (IVa), (IVb), or (IVc), or a pharmaceutically acceptable salt or solvate thereof,

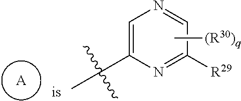

and q is 2. In some embodiments of the dual inhibitor of Formula (IV), (IVa), (IVb), or (IVc), or a pharmaceutically acceptable salt or solvate thereof,

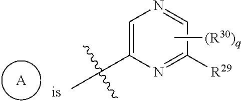

and q is 1. In some embodiments of the dual inhibitor of Formula (IV), (IVa), (IVb), or (IVc), or a pharmaceutically acceptable salt or solvate thereof,

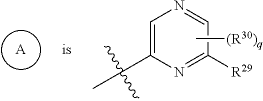

and q is 0. In some embodiments of the dual inhibitor of Formula (IV), (IVa), (IVb), or (IVc), or a pharmaceutically acceptable salt or solvate thereof,

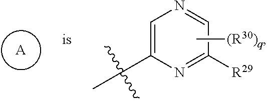

q is 0, and R²⁹ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

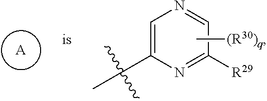

q is 0, and R²⁹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂—N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

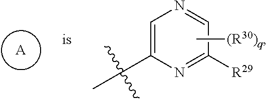

q is 0, and R²⁹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one or two substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

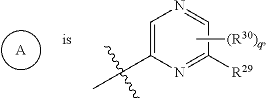

q is 0, and R²⁹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

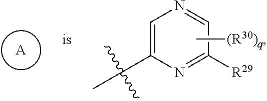

q is 0, and R²⁶ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halogen, C₁₋₆alkyl, and C₃₋₈cycloalkyl. In some embodiments of the dual inhibitor of Formula (IV), (IVa), (IVb), or (IVc), or a pharmaceutically acceptable salt or solvate thereof,

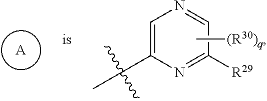

### q is 0, and R²⁶ is

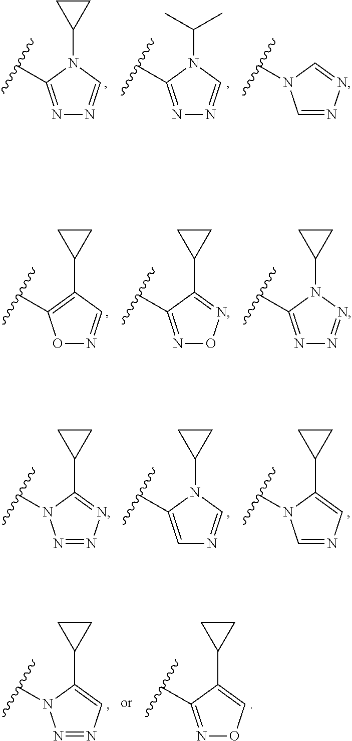

In some embodiments of the dual inhibitor of Formula (IV), (IVa), (IVb), or (IVc), or a pharmaceutically acceptable salt or solvate thereof,

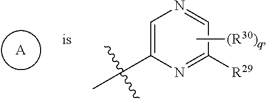

### q is 0, and R²⁶ is

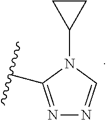 [SMILES: *c1nncn1C1CC1]

In some embodiments of the dual inhibitor of Formula (IV), (IVa), (IVb), or (IVc), or a pharmaceutically acceptable salt or solvate thereof,

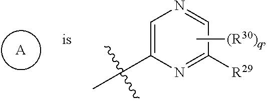

### q is 0, and R²⁶ is

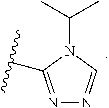 [SMILES: *c1nncn1C(C)C]

In some embodiments of the dual inhibitor of Formula (IV), (IVa), (IVb), or (IVc), or a pharmaceutically acceptable salt or solvate thereof,

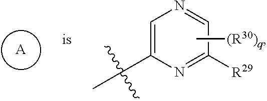

### q is 0, and R²⁶ is

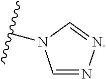 [SMILES: *n1cnnc1]

In some embodiments of the dual inhibitor of Formula (IV), (IVa), (IVb), or (IVc), or a pharmaceutically acceptable salt or solvate thereof,

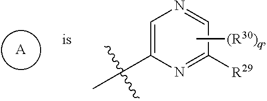

### q is 0, and R²⁶ is

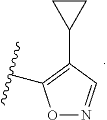 [SMILES: *c1oncc1C1CC1]

In some embodiments of the dual inhibitor of Formula (IV), (IVa), (IVb), or (IVc), or a pharmaceutically acceptable salt or solvate thereof,

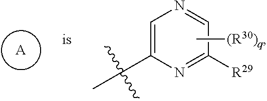

### q is 0, and R²⁹ is

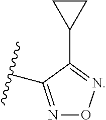 [SMILES: *c1nonc1C1CC1]

In some embodiments of the dual inhibitor of Formula (IV), (IVa), (IVb), or (IVc), or a pharmaceutically acceptable salt or solvate thereof,

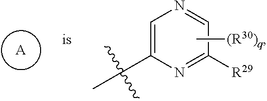

### q is 0, and R²⁹ is

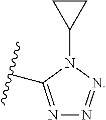 [SMILES: *c1nnnn1C1CC1]

In some embodiments of the dual inhibitor of Formula (IV), (IVa), (IVb), or (IVc), or a pharmaceutically acceptable salt or solvate thereof,

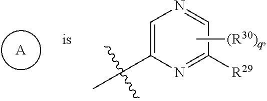

### q is 0, and R²⁹ is

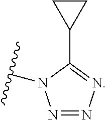 [SMILES: *n1nnnc1C1CC1]

In some embodiments of the dual inhibitor of Formula (IV), (IVa), (IVb), or (IVc), or a pharmaceutically acceptable salt or solvate thereof,

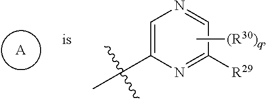

### q is 0, and R²⁹ is

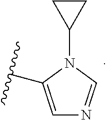 [SMILES: *c1cncn1C1CC1]

In some embodiments of the dual inhibitors of Formula (IV), (IVa), (IVb), or (IVc), or a pharmaceutically acceptable salt or solvate thereof,

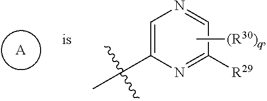

### q is 0, and R²⁹ is

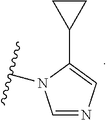 [SMILES: *n1cncc1C1CC1]

In some embodiments of the dual inhibitor of Formula (IV), (IVa), (IVb), or (IVc), or a pharmaceutically acceptable salt or solvate thereof,

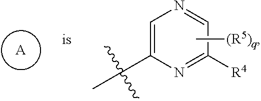

### q is 0, and R²⁹ is

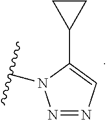 [SMILES: *n1nncc1C1CC1]

In some embodiments of the dual inhibitor of Formula (IV), (IVa), (IVb), or (IVc), or a pharmaceutically acceptable salt or solvate thereof,

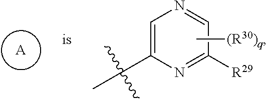

### q is 0, and R²⁹ is

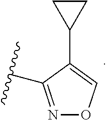 [SMILES: *c1nocc1C1CC1]

In some embodiments of the dual inhibitor of Formula (IV), (IVa), (IVb), or (IVc), or a pharmaceutically acceptable salt or solvate thereof,

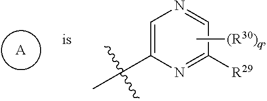

### q is 0, and R²⁹ is

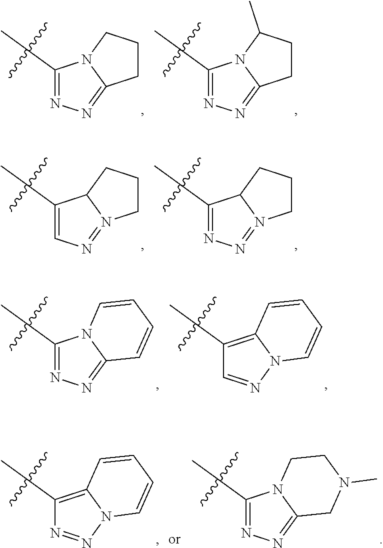

In some embodiments of the dual inhibitor of Formula (IV), (IVa), (IVb), or (IVc), or a pharmaceutically acceptable salt or solvate thereof,

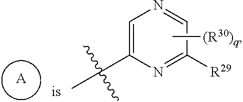

### q is 0, and R²⁹ is

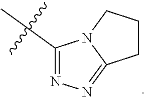 [SMILES: *c1nnc2n1CCC2]

In some embodiments of the dual inhibitor of Formula (IV), (IVa), (IVb), or (IVc), or a pharmaceutically acceptable salt or solvate thereof,

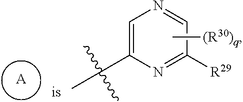

### q is 0, and R²⁹ is

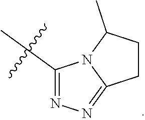 [SMILES: *c1nnc2n1C(C)CC2]

In some embodiments of the dual inhibitor of Formula (IV), (IVa), (IVb), or (IVc), or a pharmaceutically acceptable salt or solvate thereof,

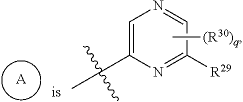

### q is 0, and R²⁹ is

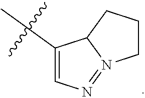

In some embodiments of the dual inhibitor of Formula (IV), (IVa), (IVb), or (IVc), or a pharmaceutically acceptable salt or solvate thereof,

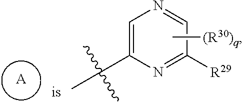

### q is 0, and R²⁹ is

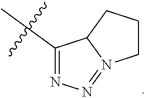

In some embodiments of the dual inhibitor of Formula (IV), (IVa), (IVb), or (IVc), or a pharmaceutically acceptable salt or solvate thereof,

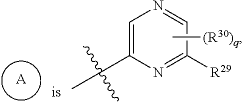

### q is 0, and R²⁹ is

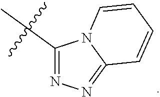 [SMILES: *c1nnc2ccccn12]

In some embodiments of the dual inhibitor of Formula (IV), (IVa), (IVb), or (IVc), or a pharmaceutically acceptable salt or solvate thereof,

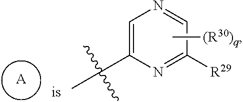

### q is 0, and R²⁹ is

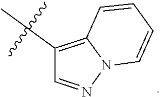 [SMILES: *c1cnn2ccccc12]

In some embodiments of the dual inhibitor of Formula (IV), (IVa), (IVb), or (IVc), or a pharmaceutically acceptable salt or solvate thereof,

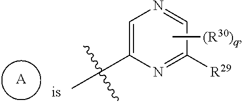

### q is 0, and R⁴ is

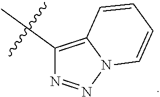 [SMILES: *c1nnn2ccccc12]

In some embodiments of the dual inhibitor of Formula (IV), (IVa), (IVb), or (IVc), or a pharmaceutically acceptable salt or solvate thereof,

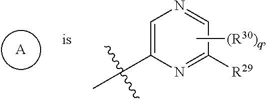

### q is 0, and R²⁹ is

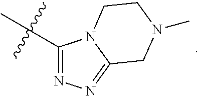 [SMILES: *c1nnc2n1CCN(C)C2]

In some embodiments of the dual inhibitor of Formula (IV), (IVa), (IVb), or (IVc), or a pharmaceutically acceptable salt or solvate thereof,

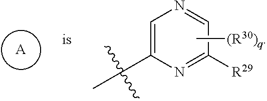

In some embodiments of the dual inhibitor of Formula (IV), (IVa), (IVb), or (IVc), or a pharmaceutically acceptable salt or solvate thereof,

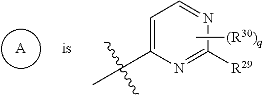

and q is 2. In some embodiments of the dual inhibitor of Formula (IV), (IVa), (IVb), or (IVc), or a pharmaceutically acceptable salt or solvate thereof,

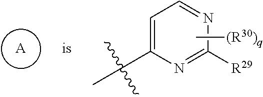

and q is 1. In some embodiments of the dual inhibitor of Formula (IV), (IVa), (IVb), or (IVc), or a pharmaceutically acceptable salt or solvate thereof,

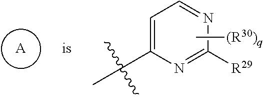

and q is 0. In some embodiments of the dual inhibitor of Formula (IV), (IVa), (IVb), or (IVc), or a pharmaceutically acceptable salt or solvate thereof,

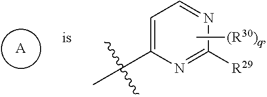

q is 0, and R²⁹ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

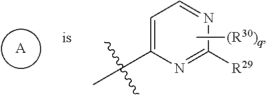

q is 0, and R²⁹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂—N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

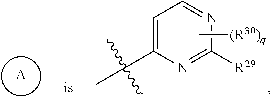

q is 0, and R²⁹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one or two substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

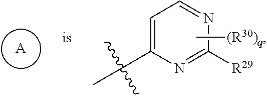

q is 0, and R²⁹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

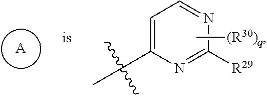

q is 0, and R²⁹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halogen, C₁₋₆alkyl, and C₃₋₈cycloalkyl. In some embodiments of the dual inhibitor of Formula (IV), (IVa), (IVb), or (IVc), or a pharmaceutically acceptable salt or solvate thereof,

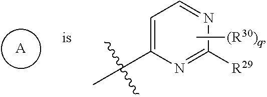

### q is 0, and R²⁹ is

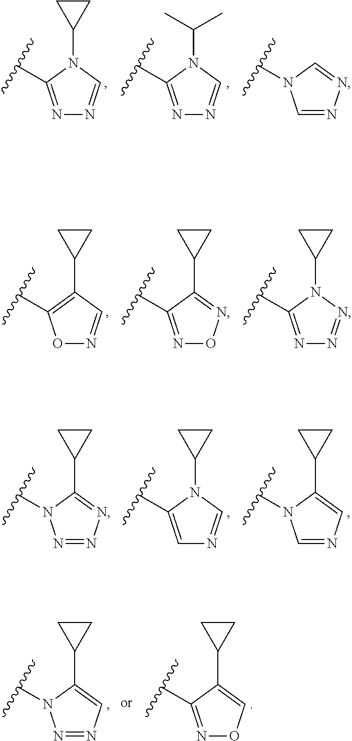

In some embodiments of the dual inhibitor of Formula (IV), (IVa), (IVb), or (IVc), or a pharmaceutically acceptable salt or solvate thereof,

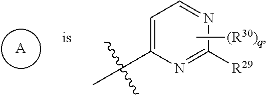

### q is 0, and R²⁹ is

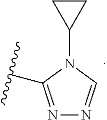 [SMILES: *c1nncn1C1CC1]

In some embodiments of the dual inhibitor of Formula (IV), (IVa), (IVb), or (IVc), or a pharmaceutically acceptable salt or solvate thereof,

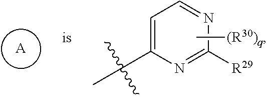

### q is 0, and R²⁹ is

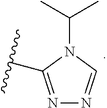 [SMILES: *c1nncn1C(C)C]

In some embodiments of the dual inhibitor of Formula (IV), (IVa), (IVb), or (IVc), or a pharmaceutically acceptable salt or solvate thereof,

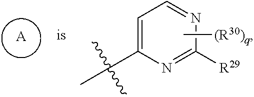

### q is 0, and R²⁹ is

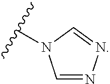 [SMILES: *n1cnnc1]

In some embodiments of the dual inhibitor of Formula (IV), (IVa), (IVb), or (IVc), or a pharmaceutically acceptable salt or solvate thereof,

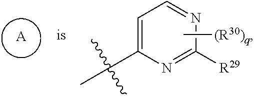

### q is 0, and R²⁹ is

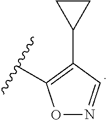 [SMILES: *c1oncc1C1CC1]

In some embodiments of the dual inhibitor of Formula (IV), (IVa), (IVb), or (IVc), or a pharmaceutically acceptable salt or solvate thereof,

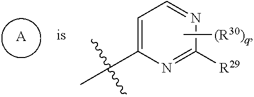

### q is 0, and R²⁹ is

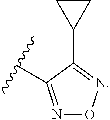 [SMILES: *c1nonc1C1CC1]

In some embodiments of the dual inhibitor of Formula (IV), (IVa), (IVb), or (IVc), or a pharmaceutically acceptable salt or solvate thereof,

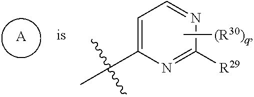

### q is 0, and R²⁹ is

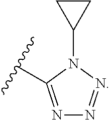 [SMILES: *c1nnnn1C1CC1]

In some embodiments of the dual inhibitor of Formula (IV), (IVa), (IVb), or (IVc), or a pharmaceutically acceptable salt or solvate thereof,

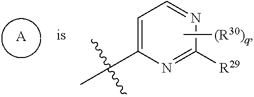

### q is 0, and R²⁹ is

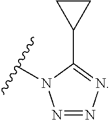 [SMILES: *n1nnnc1C1CC1]

In some embodiments of the dual inhibitor of Formula (IV), (IVa), (IVb), or (IVc), or a pharmaceutically acceptable salt or solvate thereof,

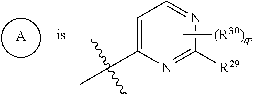

### q is 0, and R²⁹ is

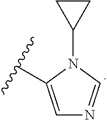 [SMILES: *c1cncn1C1CC1]

In some embodiments of the dual inhibitor of Formula (IV), (IVa), (IVb), or (IVc), or a pharmaceutically acceptable salt or solvate thereof,

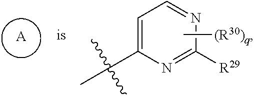

### q is 0, and R²⁹ is

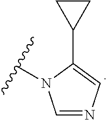 [SMILES: *n1cncc1C1CC1]

In some embodiments of the dual inhibitor of Formula (IV), (IVa), (IVb), or (IVc), or a pharmaceutically acceptable salt or solvate thereof,

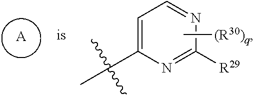

### q is 0, and R²⁹ is

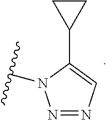 [SMILES: *n1nncc1C1CC1]

In some embodiments of the dual inhibitor of Formula (IV), (IVa), (IVb), or (IVc), or a pharmaceutically acceptable salt or solvate thereof,

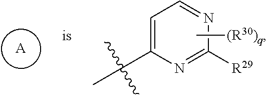

### q is 0, and R²⁹ is

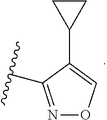 [SMILES: *c1nocc1C1CC1]

In some embodiments of the dual inhibitor of Formula (IV), (IVa), (IVb), or (IVc), or a pharmaceutically acceptable salt or solvate thereof,

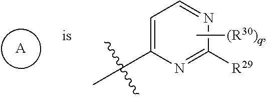

### q is 0, and R²⁹ is

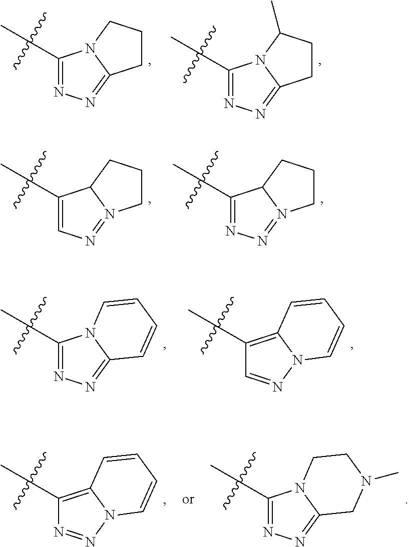

In some embodiments of the dual inhibitor of Formula (IV), (IVa), (IVb), or (IVc), or a pharmaceutically acceptable salt or solvate thereof,

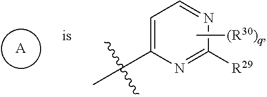

### q is 0, and R²⁹ is

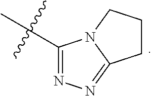 [SMILES: *c1nnc2n1CCC2]

In some embodiments of the dual inhibitor of Formula (IV), (IVa), (IVb), or (IVc), or a pharmaceutically acceptable salt or solvate thereof,

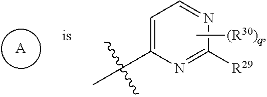

### q is 0, and R²⁹ is

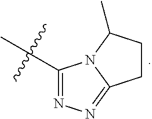 [SMILES: *c1nnc2n1C(C)CC2]

In some embodiments of the dual inhibitor of Formula (IV), (IVa), (IVb), or (IVc), or a pharmaceutically acceptable salt or solvate thereof,

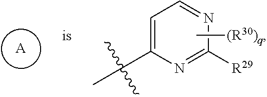

### q is 0, and R²⁹ is

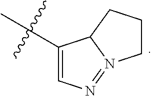

In some embodiments of the dual inhibitor of Formula (IV), (IVa), (IVb), or (IVc), or a pharmaceutically acceptable salt or solvate thereof,

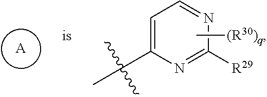

### q is 0, and R²⁹ is

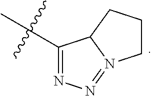

In some embodiments of the dual inhibitor of Formula (IV), (IVa), (IVb), or (IVc), or a pharmaceutically acceptable salt or solvate thereof,

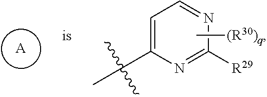

### q is 0, and R²⁹ is

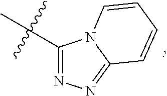 [SMILES: *c1nnc2ccccn12]

In some embodiments of the dual inhibitor of Formula (IV), (IVa), (IVb), or (IVc), or a pharmaceutically acceptable salt or solvate thereof,

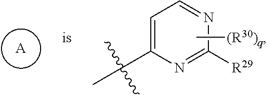

### q is 0, and R²⁹ is

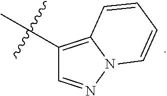 [SMILES: *c1cnn2ccccc12]

In some embodiments of the dual inhibitor of Formula (IV), (IVa), (IVb), or (IVc), or a pharmaceutically acceptable salt or solvate thereof,

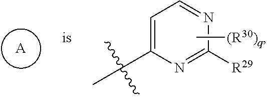

### q is 0, and R²⁹ is

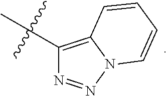 [SMILES: *c1nnn2ccccc12]

In some embodiments of the dual inhibitor of Formula (IV), (IVa), (IVb), or (IVc), or a pharmaceutically acceptable salt or solvate thereof,

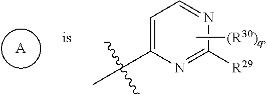

### q is 0, and R²⁹ is

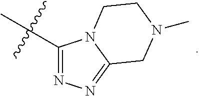 [SMILES: *c1nnc2n1CCN(C)C2]

In some embodiments of the dual inhibitor of Formula (IV), (IVa), (IVb), or (IVc), or a pharmaceutically acceptable salt or solvate thereof,

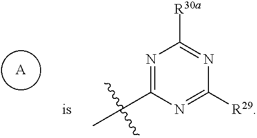

In some embodiments of the dual inhibitor of Formula (IV), (IVa), (IVb), or (IVc), or a pharmaceutically acceptable salt or solvate thereof,

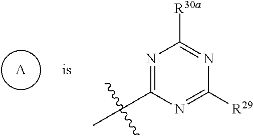

and q is 2. In some embodiments of the dual inhibitor of Formula (IV), (IVa), (IVb), or (IVc), or a pharmaceutically acceptable salt or solvate thereof,

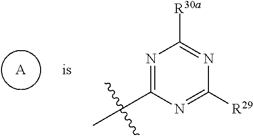

and q is 1. In some embodiments of the dual inhibitor of Formula (IV), (IVa), (IVb), or (IVc), or a pharmaceutically acceptable salt or solvate thereof,

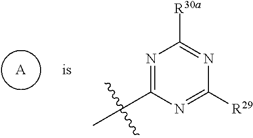

and q is 0. In some embodiments of the dual inhibitor of Formula (W), (IVa), (IVb), or (IVc), or a pharmaceutically acceptable salt or solvate thereof,

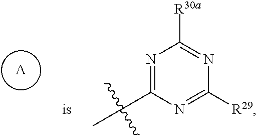

R^{30a} is hydrogen, and R²⁹ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (W), (IVa), (IVb), or (IVc), or a pharmaceutically acceptable salt or solvate thereof,

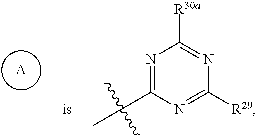

R^{30a} is hydrogen, and R²⁹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one, two, or three substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂—N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

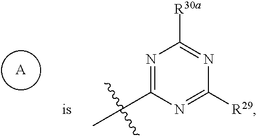

R^{30a} is hydrogen, and R²⁹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one or two substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. I n some embodiments of the dual inhibitor of Formula (IV), (IVa), (IVb), or (IVc), or a pharmaceutically acceptable salt or solvate thereof,

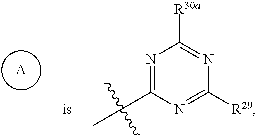

R^{30a} is hydrogen, and R²⁹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halogen, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁸, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸. In some embodiments of the dual inhibitor of Formula (IV), (IVa), (IVb), or (IVc), or a pharmaceutically acceptable salt or solvate thereof,

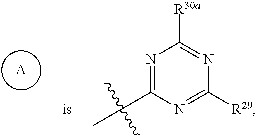

R^{30a} is hydrogen, and R²⁹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halogen, C₁₋₆alkyl, and C₃₋₈cycloalkyl. In some embodiments of the dual inhibitor of Formula (IV), (IVa), (IVb), or (IVc), or a pharmaceutically acceptable salt or solvate thereof,

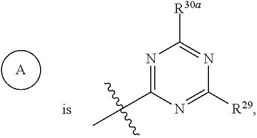

R^{30a} is hydrogen, and R²⁹ is

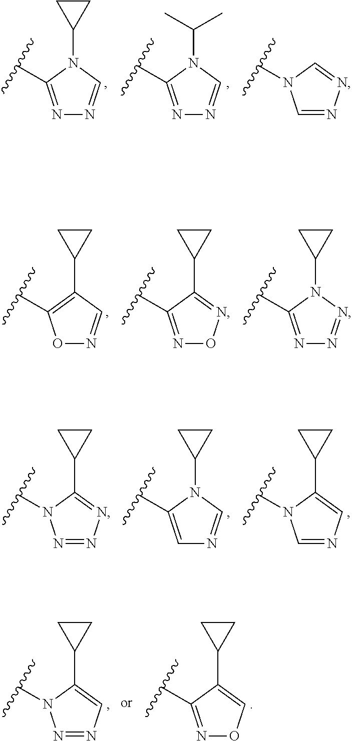

In some embodiments of the dual inhibitor of Formula (IV), (IVa), (IVb), or (IVc), or a pharmaceutically acceptable salt or solvate thereof,

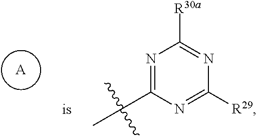

R^{30a} is hydrogen, and R²⁹ is

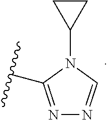 [SMILES: *c1nncn1C1CC1]

In some embodiments of the dual inhibitor of Formula (IV), (IVa), (IVb), or (IVc), or a pharmaceutically acceptable salt or solvate thereof,

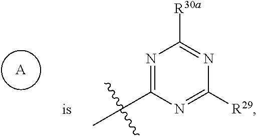

R^{30a} is hydrogen, and R²⁹ is

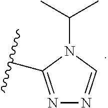 [SMILES: *c1nncn1C(C)C]

In some embodiments of the dual inhibitor of Formula (IV), (IVa), (IVb), or (IVc), or a pharmaceutically acceptable salt or solvate thereof,

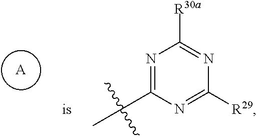

R^{30a} is hydrogen, and R²⁹ is

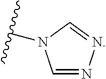 [SMILES: *n1cnnc1]

In some embodiments of the dual inhibitor of Formula (IV), (IVa), (IVb), or (IVc), or a pharmaceutically acceptable salt or solvate thereof,

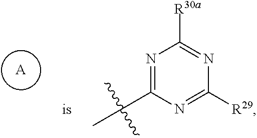

R^{30a} is hydrogen, and R²⁹ is

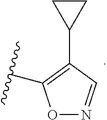 [SMILES: *c1oncc1C1CC1]

In some embodiments of the dual inhibitor of Formula (IV), (IVa), (IVb), or (IVc), or a pharmaceutically acceptable salt or solvate thereof,

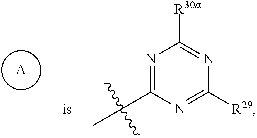

R^{30a} is hydrogen, and R²⁹ is

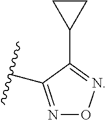 [SMILES: *c1nonc1C1CC1]

In some embodiments of the dual inhibitor of Formula (IV), (IVa), (IVb), or (IVc), or a pharmaceutically acceptable salt or solvate thereof,

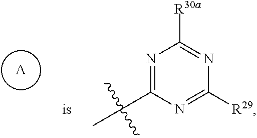

R^{30a} is hydrogen, and R²⁹ is

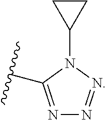 [SMILES: *c1nnnn1C1CC1]

In some embodiments of the dual inhibitor of Formula (IV), (IVa), (IVb), or (IVc), or a pharmaceutically acceptable salt or solvate thereof, wherein

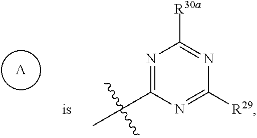

R^{30a} is hydrogen, and R²⁹ is

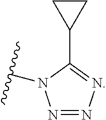 [SMILES: *n1nnnc1C1CC1]

In some embodiments of the dual inhibitor of Formula (IV), (IVa), (IVb), or (IVc), or a pharmaceutically acceptable salt or solvate thereof,

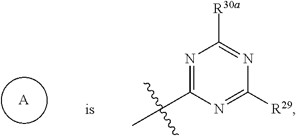

R^{30a} is hydrogen, and R²⁹ is

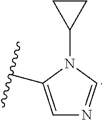 [SMILES: *c1cncn1C1CC1]

In some embodiments of the dual inhibitor of Formula (IV), (IVa), (IVb), or (IVc), or a pharmaceutically acceptable salt or solvate thereof,

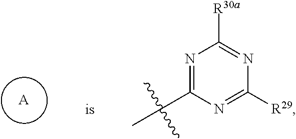

R^{30a} is hydrogen, and R²⁹ is

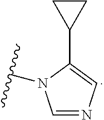 [SMILES: *n1cncc1C1CC1]

In some embodiments of the dual inhibitor of Formula (IV), (IVa), (IVb), or (IVc), or a pharmaceutically acceptable salt or solvate thereof,

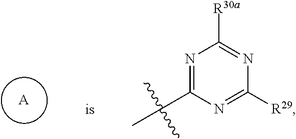

R^{5a} is hydrogen, and R⁴ is

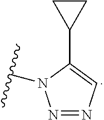 [SMILES: *n1nncc1C1CC1]

In some embodiments of the dual inhibitor of Formula (IV), (IVa), (IVb), or (IVc), or a pharmaceutically acceptable salt or solvate thereof,

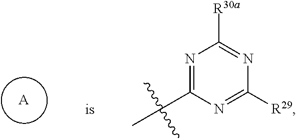

R^{5a} is hydrogen, and R²⁹ is

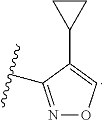 [SMILES: *c1nocc1C1CC1]

In some embodiments of the dual inhibitor of Formula (IV), (IVa), (IVb), or (IVc), or a pharmaceutically acceptable salt or solvate thereof,

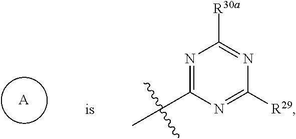

R^{30a} is hydrogen, and R²⁹ is

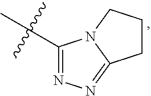 [SMILES: *c1nnc2n1CCC2]

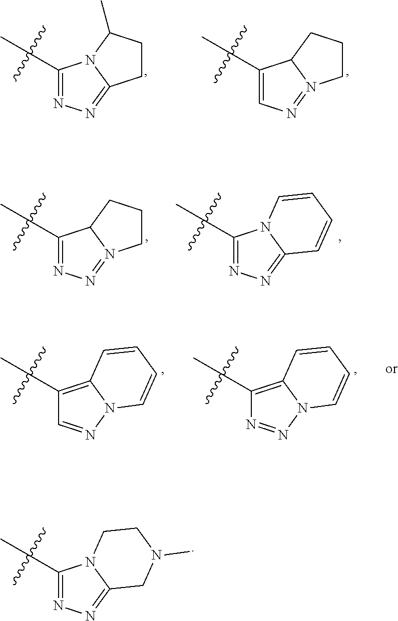

In some embodiments of the dual inhibitor of Formula (IV), (IVa), (IVb), or (IVc), or a pharmaceutically acceptable salt or solvate thereof,

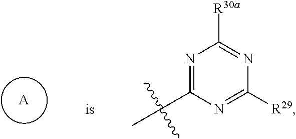

R^{30a} is hydrogen, and R²⁹ is

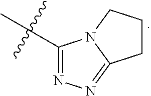 [SMILES: *c1nnc2n1CCC2]

In some embodiments of the dual inhibitor of Formula (IV), (IVa), (IVb), or (IVc), or a pharmaceutically acceptable salt or solvate thereof,

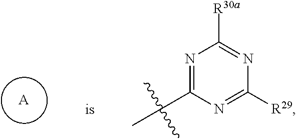

R^{30a} is hydrogen, and R⁴ is

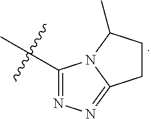 [SMILES: *c1nnc2n1C(C)CC2]

In some embodiments of the dual inhibitor of Formula (IV), (IVa), (IVb), or (IVc), or a pharmaceutically acceptable salt or solvate thereof,

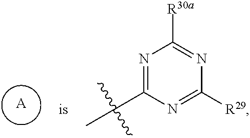

R^{30a} is hydrogen, and R²⁹ is

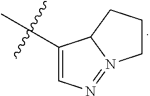

In some embodiments of the dual inhibitor of Formula (IV), (IVa), (IVb), or (IVc), or a pharmaceutically acceptable salt or solvate thereof,

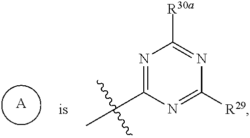

R^{30a} is hydrogen, and R²⁹ is

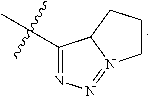

In some embodiments of the dual inhibitor of Formula (IV), (IVa), (IVb), or (IVc), or a pharmaceutically acceptable salt or solvate thereof, wherein

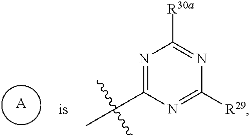

R^{30a} is hydrogen, and R²⁹ is

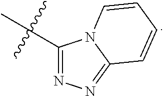 [SMILES: *c1nnc2ccccn12]

In some embodiments of the dual inhibitor of Formula (IV), (IVa), (IVb), or (IVc), or a pharmaceutically acceptable salt or solvate thereof,

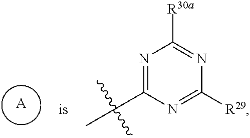

R^{30a} is hydrogen, and R²⁹ is

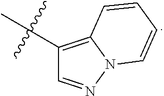 [SMILES: *c1cnn2ccccc12]

In some embodiments of the dual inhibitor of Formula (IV), (IVa), (IVb), or (IVc), or a pharmaceutically acceptable salt or solvate thereof,

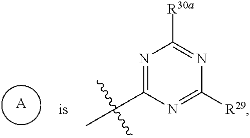

R^{30a} is hydrogen, and R²⁹ is

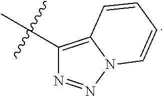 [SMILES: *c1nnn2ccccc12]

In some embodiments of the dual inhibitor of Formula (IV). (IVa). (IVb), or (IVc), or a pharmaceutically acceptable salt or solvate thereof,

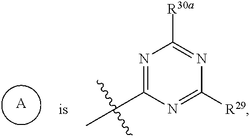

R^{30a} is hydrogen, and R²⁹ is

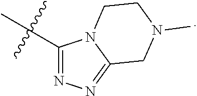 [SMILES: *c1nnc2n1CCN(C)C2]

In some embodiments, the dual inhibitor of ASK1 and DYRK1A is:

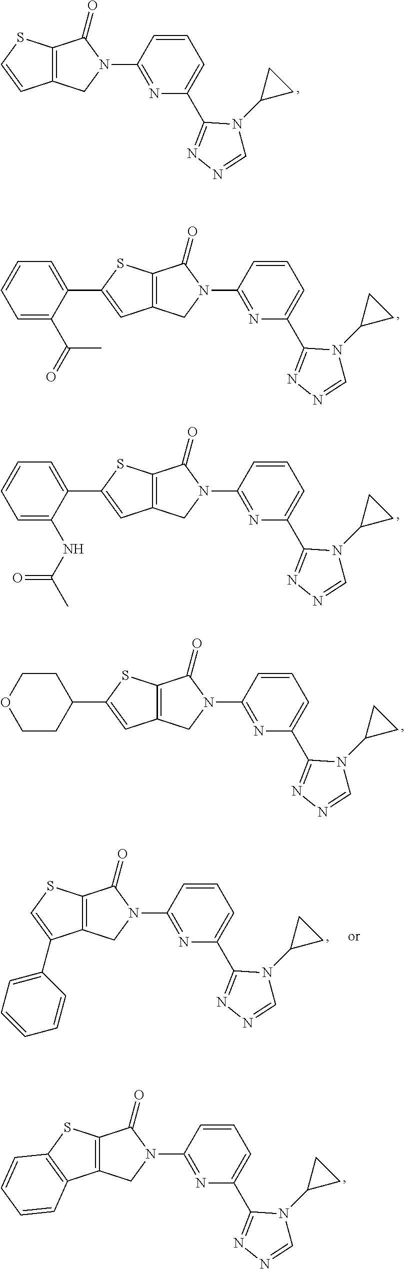 [SMILES: O=C1c2sccc2CN1c1cccc(-c2nncn2C2CC2)n1 ; CC(=O)c1ccccc1-c1cc2c(s1)C(=O)N(c1cccc(-c3nncn3C3CC3)n1)C2 ; CC(=O)Nc1ccccc1-c1cc2c(s1)C(=O)N(c1cccc(-c3nncn3C3CC3)n1)C2 ; O=C1c2sc(C3CCOCC3)cc2CN1c1cccc(-c2nncn2C2CC2)n1 ; O=C1c2scc(-c3ccccc3)c2CN1c1cccc(-c2nncn2C2CC2)n1 ; O=C1c2sc3ccccc3c2CN1c1cccc(-c2nncn2C2CC2)n1]

or a pharmaceutically acceptable salt or solvate thereof

In some embodiments, the dual inhibitor of ASK1 and DYRK1A is:

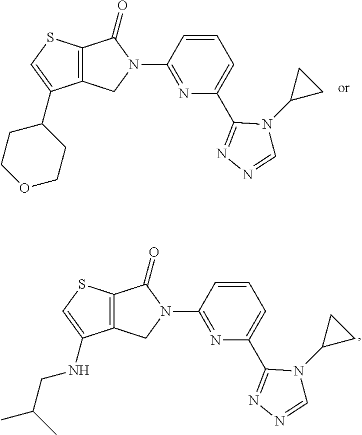 [SMILES: O=C1c2scc(C3CCOCC3)c2CN1c1cccc(-c2nncn2C2CC2)n1 ; CC(C)CNc1csc2c1CN(c1cccc(-c3nncn3C3CC3)n1)C2=O]

or a pharmaceutically acceptable salt or solvate thereof.

In some embodiments, the dual inhibitor of ASK1 and DYRK1A is:

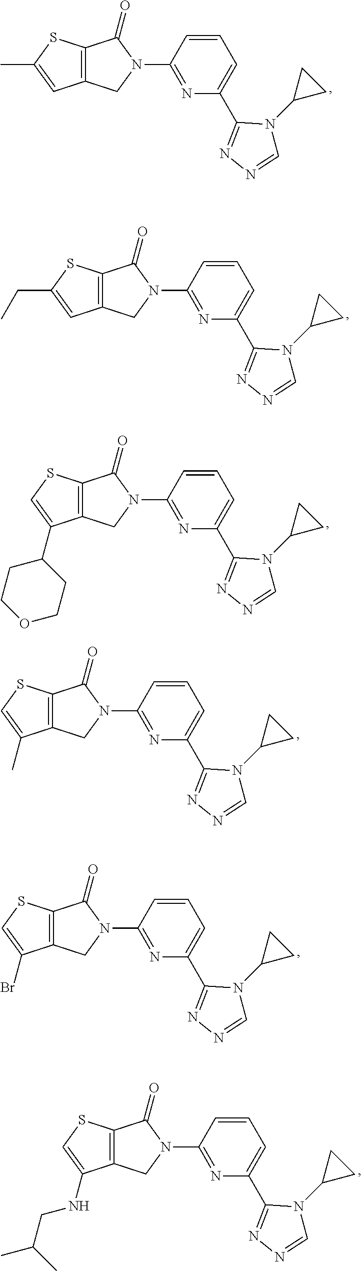 [SMILES: Cc1cc2c(s1)C(=O)N(c1cccc(-c3nncn3C3CC3)n1)C2 ; CCc1cc2c(s1)C(=O)N(c1cccc(-c3nncn3C3CC3)n1)C2 ; O=C1c2scc(C3CCOCC3)c2CN1c1cccc(-c2nncn2C2CC2)n1 ; Cc1csc2c1CN(c1cccc(-c3nncn3C3CC3)n1)C2=O ; O=C1c2scc(Br)c2CN1c1cccc(-c2nncn2C2CC2)n1 ; CC(C)CNc1csc2c1CN(c1cccc(-c3nncn3C3CC3)n1)C2=O]

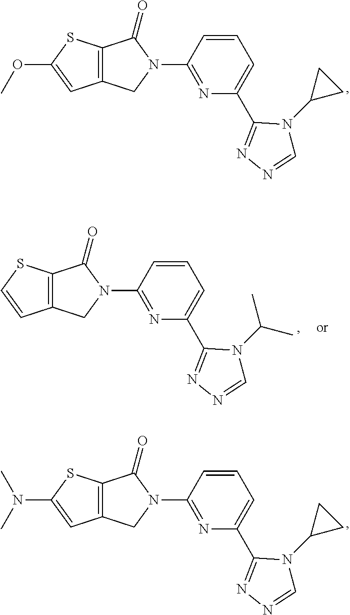

or a pharmaceutically acceptable salt or solvate thereof

In one aspect presented herein, is a method for treating a cognitive impairment in a subject in need thereof, comprising administering to the subject, a dual inhibitor of ASK1 and DYRK1A, wherein the dual inhibitor is a compound having the structure of Formula (V), or a pharmaceutically acceptable salt or solvate thereof:

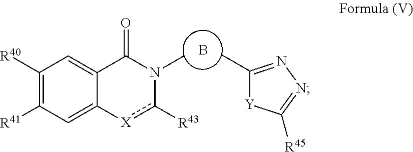

wherein:
[symbol] is a single bond or a double bond;
X is selected from a group consisting of —C(R⁴²), —CH(R⁴²), N, and —N(R⁴²);
Y is selected from a group consisting of N(R⁴⁴) and O;

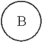

is selected from a group consisting of phenyl and a 5- to 6-membered heteroaryl;
R⁴⁰ is a 5- to 10-membered heteroaryl, optionally substituted with one, two, or three R⁴⁶ groups;
R⁴¹ is hydrogen, F, Cl, Br, I, OH, or NH₂, or is selected from the group consisting of C₁₋₆ alkyl, C₁₋₃ heteroalkyl, a 5- to 6-membered heterocycloalkyl, phenyl ,and a 5- to 6-membered heteroaryl, optionally substituted with one, two, or three R⁴⁶ groups;
R⁴² is selected from the group consisting of hydrogen, F, Cl, Br, I, OH, and NH₂;
R⁴³ is selected from a group consisting of hydrogen, C₁₋₃ alkyl, and C₁₋₃ alkoxy;
R⁴⁴ is selected from the group consisting of hydrogen, C₁₋₈ alkyl, C₃₋₇ cycloalkyl, and a 3- to 6-membered heterocycloalkyl, optionally substituted with one, two, or three R⁴⁶ groups;
R⁴⁵ is selected from a group consisting of hydrogen and C₁₋₆ alkyl;
or R⁴⁴ and R⁴⁵ are joined together to form a 5- to 6-membered ring;
R⁴⁶ is selected from the group consisting of hydrogen, F, Cl, Br, I, OH, NH₂, NH₂—(C═O)—, C₁₋₃ alkyl, C₁₋₃ alkoxy, C₁₋₃alkyl-NH—(C═O)—, C₁₋₃alkyl-S(═O)₂—, C₃-₆ cycloalkyl, a 3- to 6-membered heterocycloalkyl, and phenyl; the “hetero” moieties of the 5- to 10-membered heteroaryl, C₁₋₃ heteroalkyl, 5- to 6-membered heterocycloalkyl, 5- to 6-membered heteroaryl, and 3- to 6-membered heterocycloalkyl are each independently selected from the group consisting of —NH—, N, —O—, —S—, —S(═O)₂—, and —NH—C(═O)—; and the numbers of the heteroatoms or heteroatom groups in any of the above cases are each independently one, two, or three.

In some embodiments of the dual inhibitor of Formula (V), or a pharmaceutically acceptable salt or solvate thereof, R⁴⁶ is selected from the group consisting of hydrogen, F, Cl, Br, I, —OH, —NH₂, —CH₃, —CH₂CH₃, isopropyl, —OCH₃, —C(═O)NH₂, —C(═O)NHCH₃, —S(═O)₂CH₃, cycloprolyl, phenyl, and

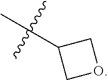 [SMILES: *C1COC1]

In some embodiments of the dual inhibitor of Formula (V), or a pharmaceutically acceptable salt or solvate thereof, R⁴⁰ is imidazolyl, 4,5,6,7-tetrahydro-1H-benzokilimidazolyl, or pyridinyl, each optionally substituted with one, two, or three groups selected from R⁴⁶.

In some embodiments of the dual inhibitor of Formula (V), or a pharmaceutically acceptable salt or solvate thereof, R⁴⁰ is

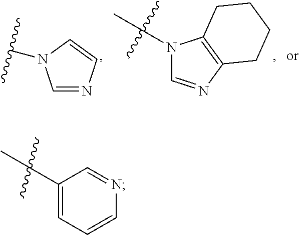 [SMILES: *n1ccnc1 ; *n1cnc2c1CCCC2 ; *c1cccnc1]

each optionally substituted with one, two, or three R⁴⁶ groups; R⁴⁶ is selected from the group consisting of hydrogen, F, Cl, Br, I, OH, NH₂, NH₂—(C═O)—, C₁₋₃ alkyl, C₁₋₃ alkoxy, C₁₋₃alkyl-NH—(C═O)—, C₁₋₃alkyl-S(═O)₂—, C₃₋₆ cycloalkyl, a 3- to 6-membered heterocycloalkyl, and phenyl.

In some embodiments of the dual inhibitor of Formula (V), or a pharmaceutically acceptable salt or solvate thereof, R⁴⁰ is

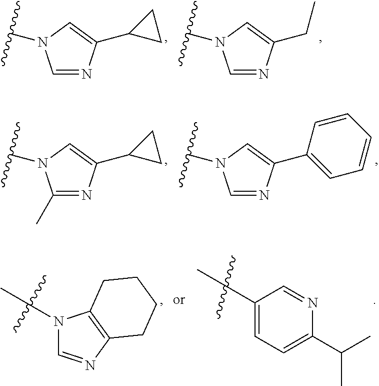

In some embodiments of the dual inhibitor of Formula (V), or a pharmaceutically acceptable salt or solvate thereof, R⁴¹ is hydrogen, F, Cl, Br, I, OH, or NH₂, or is selected from the group consisting of C₁₋₃alkyl, C₁₋₃alkylamino, C₁₋₃alkoxy, morpholinyl, phenyl, pyridinyl, and thienyl, each optionally substituted with one, two, or three R⁴⁶ groups; R⁴⁶ is selected from the group consisting of hydrogen, F, Cl, Br, I, OH, NH₂, NH₂—(C═O)—, C₁₋₃ alkyl, C₁₋₃ alkoxy, C₁₋₃alkyl-NH—(C═O)—, C₁₋₃alkyl-S(═O)₂—, C₃₋₆ cycloalkyl, a 3- to 6-membered heterocycloalkyl, and phenyl.

In some embodiments of the dual inhibitor of Formula (V), or a pharmaceutically acceptable salt or solvate thereof, R⁴¹ is hydrogen, F, Cl, Br, I, OH, or NH₂, or is selected from the group consisting of —CH₃, —CH₂CH₃, isopropyl, —NHCH₃, —OCH₃, —O—CH₂CH₃, —O(CH₂)₂CH₃, phenyl, 3-pyridyl,

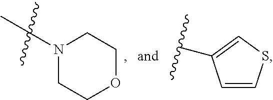 [SMILES: *N1CCOCC1 ; *c1ccsc1]

each optionally substituted with one, two, or three R⁴⁶ groups.

In some embodiments of the dual inhibitor of Formula (V), or a pharmaceutically acceptable salt or solvate thereof, R⁴¹ selected from the group consisting of hydrogen, F, Cl, Br, I, OH, NH₂, —CH₃, —CH₂CH₃, isopropyl, —N(CH₃)₂, —O—CH₃, phenyl, 3-pyridyl,

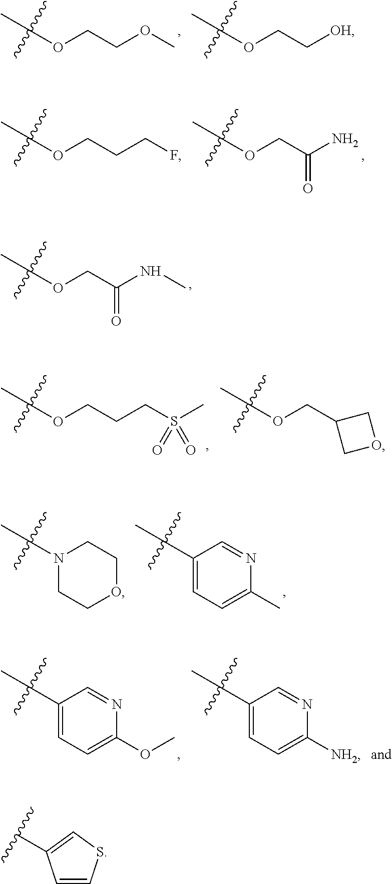

In some embodiments of the dual inhibitor of Formula (V), or a pharmaceutically acceptable salt or solvate thereof, R⁴³ is selected from hydrogen, —CH₃, —CH₂CH₃, and —OCH₃.

In some embodiments of the dual inhibitor of Formula (V), or a pharmaceutically acceptable salt or solvate thereof, R⁴⁴ is selected from a group consisting of hydrogen, C₁₋₆alkyl, C₃₋₆cycloalkyl, and a 5- to 6-membered heterocycloalkyl, optionally substituted with one, two, or three R⁴⁶ groups; each R⁴⁶ is selected from the group consisting of hydrogen, F, Cl, Br, I, OH, NH₂, NH₂—(C═O)—, C₁₋₃ alkyl, C₁₋₃ alkoxy, C₁₋₃alkyl-NH—(C═O)—, C₁₋₃alkyl-S(═O)₂—, C₃₋₆ cycloalkyl, a 3- to 6-membered heterocycloalkyl, and phenyl.

In some embodiments of the dual inhibitor of Formula (V), or a pharmaceutically acceptable salt or solvate thereof, R⁴⁴ is selected from a group consisting of hydrogen, methyl, ethyl, isopropyl, cyclopropyl, tert-butyl, sec-butyl, cyclopentyl, cyclohexyl, and

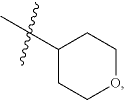 [SMILES: *C1CCOCC1]

each optionally substituted with one, two, or three R⁴⁶ groups; each R⁴⁶ is selected from the group consisting of hydrogen, F, Cl, Br, I, OH, NH₂, NH₂—(C—O)—, C₁₋₃ alkyl, C₁₋₃ alkoxy, Cₗ₋₃alkyl-S(═O)₂—, C₃₋₆ cycloalkyl, a 3- to 6-membered heterocycloalkyl, and phenyl.

In some embodiments of the dual inhibitor of Formula (V), or a pharmaceutically acceptable salt or solvate thereof, R⁴⁴ is selected from a group consisting of hydrogen, methyl, ethyl, isopropyl, cyclopropyl, tert-butyl, sec-butyl, —CH₂CF₃,

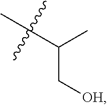 [SMILES: *C(C)CO]

cyclopentyl, cyclohexyl,

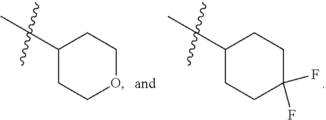 [SMILES: *C1CCOCC1 ; *C1CCC(F)(F)CC1]

In some embodiments of the dual inhibitor of Formula (V), or a pharmaceutically acceptable salt or solvate thereof, R⁴⁵ is selected from the group consisting of hydrogen and C₁₋₃alkyl. In some embodiments of the dual inhibitor of Formula (V), or a pharmaceutically acceptable salt or solvate thereof, R⁴⁵ is selected from the group consisting of hydrogen, methyl, and ethyl.

In some embodiments of the dual inhibitor of Formula (V), or a pharmaceutically acceptable salt or solvate thereof,

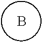

is selected from the group consisting of phenyl, pyridinyl, thienyl, and thiazolyl. In some embodiments of the dual inhibitor of Formula (V), or a pharmaceutically acceptable salt or solvate thereof,

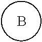

is selected from the group consisting of phenyl,

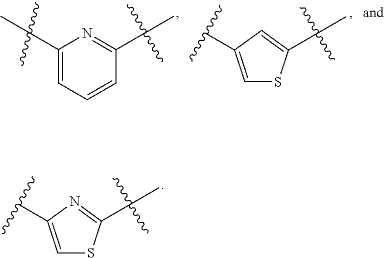 [SMILES: *c1cccc(*)n1 ; *c1csc(*)c1 ; *c1csc(*)n1]

In some embodiments of the dual inhibitor of Formula (V), or a pharmaceutically acceptable salt or solvate thereof, the structural unit

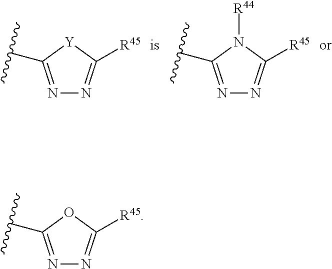

In some embodiments of the dual inhibitor of Formula (V), or a pharmaceutically acceptable salt or solvate thereof, R⁴⁴ and R⁴⁵ are joined together to form a 5- to 6-membered ring. In some embodiments of the dual inhibitor of Formula (V), of a pharmaceutically acceptable salt or solvate thereof, the structural unit

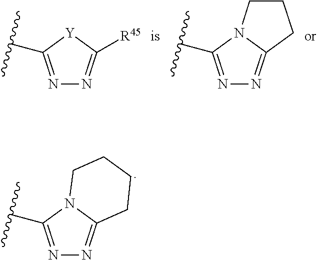

In some embodiments of the dual inhibitor of Formula (V), or a pharmaceutically acceptable salt or solvate thereof, the structural unit

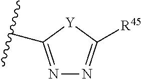

is selected from the group consisting of

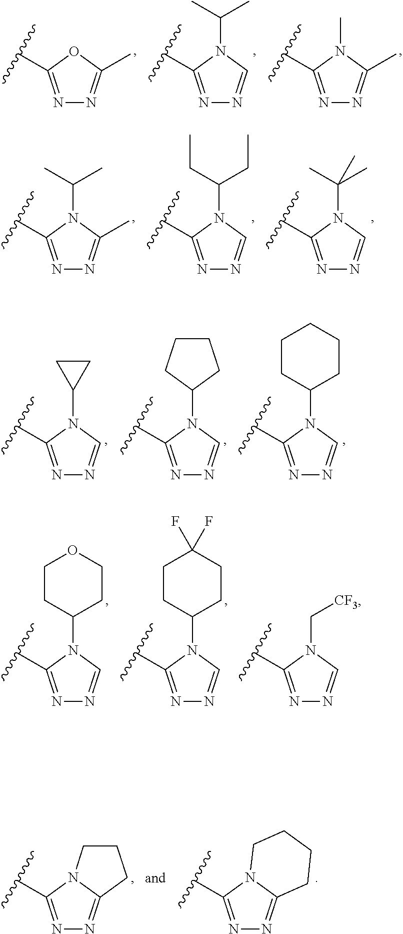

In some embodiments, the dual inhibitor having the structure of Formula (V), has the structure of Formula (Va), (Vb), or (Vc), or a pharmaceutically acceptable salt or solvate thereof:

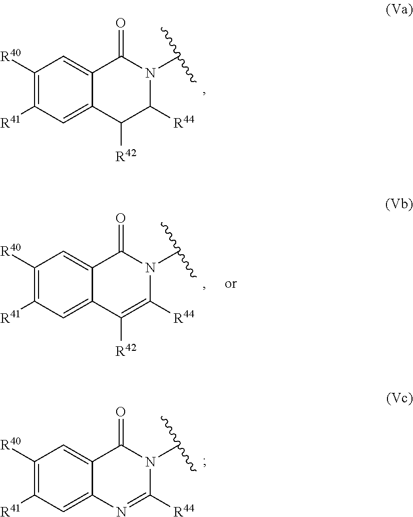

wherein R⁴⁰, R⁴¹, R⁴², R⁴³, and R⁴⁴ are described in embodiments.

In some embodiments, the dual inhibitor of ASK1 and DYRK1A is:

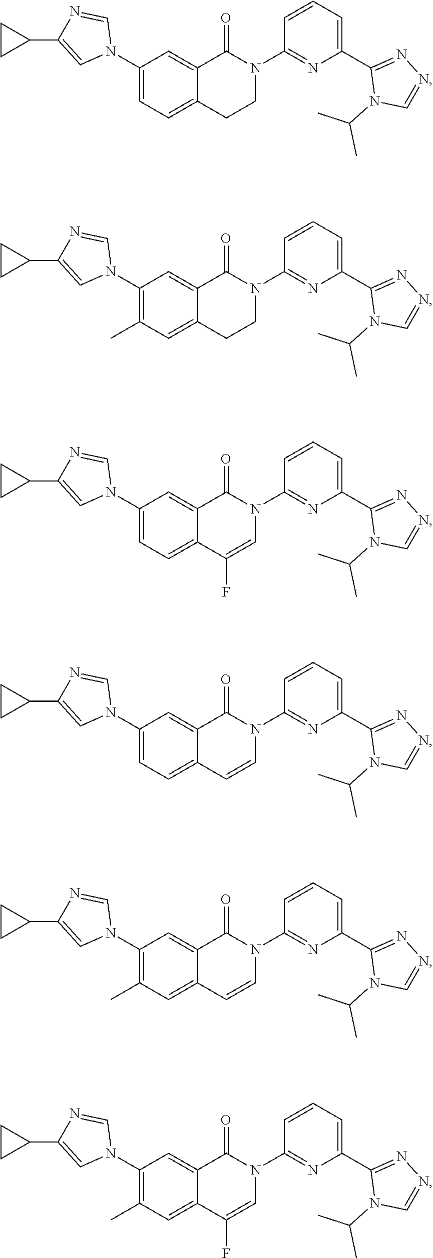 [SMILES: CC(C)n1cnnc1-c1cccc(N2CCc3ccc(-n4cnc(C5CC5)c4)cc3C2=O)n1 ; Cc1cc2c(cc1-n1cnc(C3CC3)c1)C(=O)N(c1cccc(-c3nncn3C(C)C)n1)CC2 ; CC(C)n1cnnc1-c1cccc(-n2cc(F)c3ccc(-n4cnc(C5CC5)c4)cc3c2=O)n1 ; CC(C)n1cnnc1-c1cccc(-n2ccc3ccc(-n4cnc(C5CC5)c4)cc3c2=O)n1 ; Cc1cc2ccn(-c3cccc(-c4nncn4C(C)C)n3)c(=O)c2cc1-n1cnc(C2CC2)c1 ; Cc1cc2c(F)cn(-c3cccc(-c4nncn4C(C)C)n3)c(=O)c2cc1-n1cnc(C2CC2)c1]

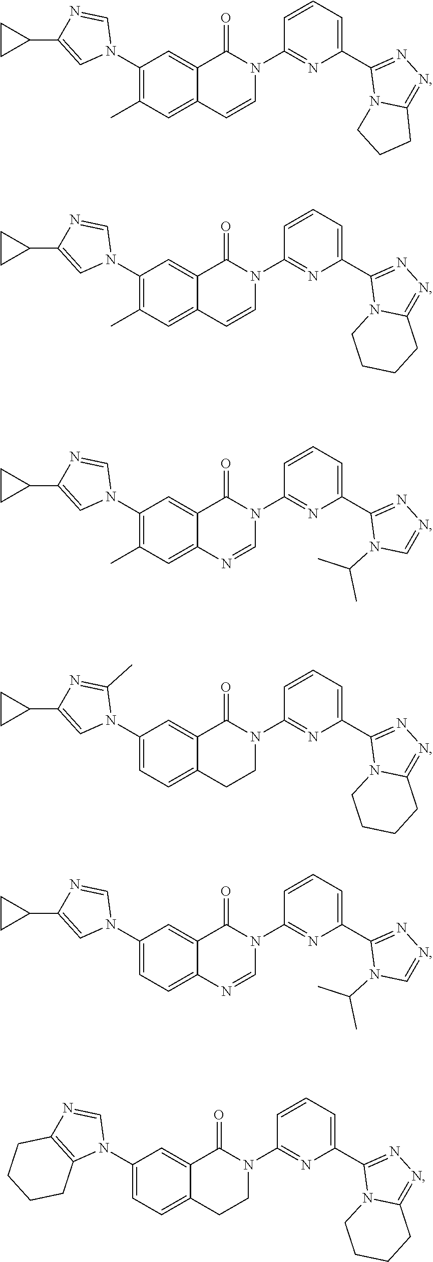 [SMILES: Cc1cc2ccn(-c3cccc(-c4nnc5n4CCC5)n3)c(=O)c2cc1-n1cnc(C2CC2)c1 ; Cc1cc2ccn(-c3cccc(-c4nnc5n4CCCC5)n3)c(=O)c2cc1-n1cnc(C2CC2)c1 ; Cc1cc2ncn(-c3cccc(-c4nncn4C(C)C)n3)c(=O)c2cc1-n1cnc(C2CC2)c1 ; Cc1nc(C2CC2)cn1-c1ccc2c(c1)C(=O)N(c1cccc(-c3nnc4n3CCCC4)n1)CC2 ; CC(C)n1cnnc1-c1cccc(-n2cnc3ccc(-n4cnc(C5CC5)c4)cc3c2=O)n1 ; O=C1c2cc(-n3cnc4c3CCCC4)ccc2CCN1c1cccc(-c2nnc3n2CCCC3)n1]

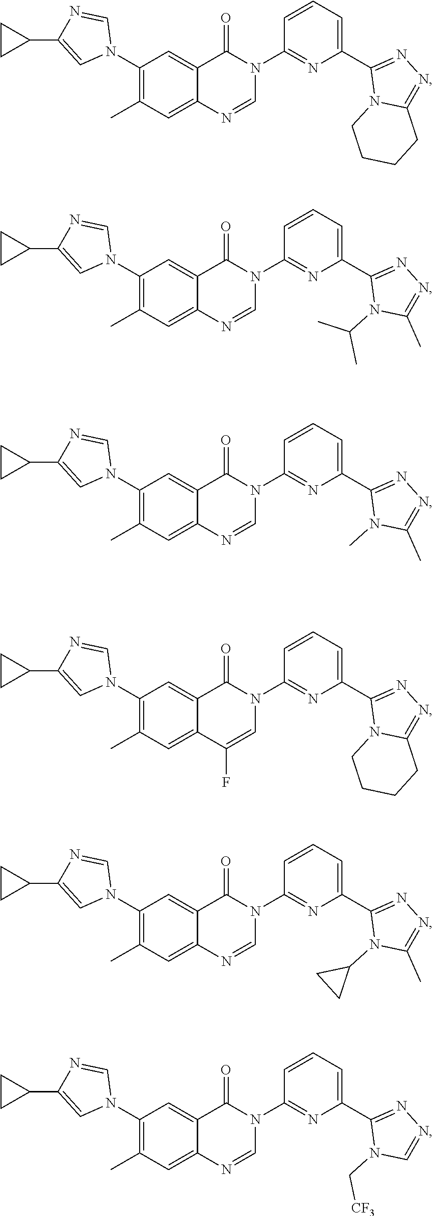 [SMILES: Cc1cc2ncn(-c3cccc(-c4nnc5n4CCCC5)n3)c(=O)c2cc1-n1cnc(C2CC2)c1 ; Cc1cc2ncn(-c3cccc(-c4nnc(C)n4C(C)C)n3)c(=O)c2cc1-n1cnc(C2CC2)c1 ; Cc1cc2ncn(-c3cccc(-c4nnc(C)n4C)n3)c(=O)c2cc1-n1cnc(C2CC2)c1 ; Cc1cc2c(F)cn(-c3cccc(-c4nnc5n4CCCC5)n3)c(=O)c2cc1-n1cnc(C2CC2)c1 ; Cc1cc2ncn(-c3cccc(-c4nnc(C)n4C4CC4)n3)c(=O)c2cc1-n1cnc(C2CC2)c1 ; Cc1cc2ncn(-c3cccc(-c4nncn4CC(F)(F)F)n3)c(=O)c2cc1-n1cnc(C2CC2)c1]

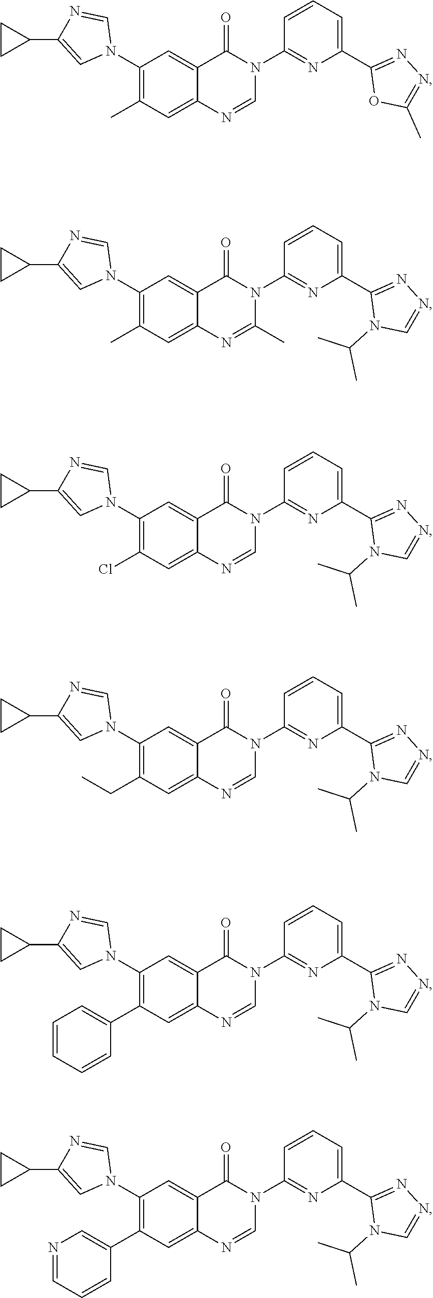 [SMILES: Cc1nnc(-c2cccc(-n3cnc4cc(C)c(-n5cnc(C6CC6)c5)cc4c3=O)n2)o1 ; Cc1cc2nc(C)n(-c3cccc(-c4nncn4C(C)C)n3)c(=O)c2cc1-n1cnc(C2CC2)c1 ; CC(C)n1cnnc1-c1cccc(-n2cnc3cc(Cl)c(-n4cnc(C5CC5)c4)cc3c2=O)n1 ; CCc1cc2ncn(-c3cccc(-c4nncn4C(C)C)n3)c(=O)c2cc1-n1cnc(C2CC2)c1 ; CC(C)n1cnnc1-c1cccc(-n2cnc3cc(-c4ccccc4)c(-n4cnc(C5CC5)c4)cc3c2=O)n1 ; CC(C)n1cnnc1-c1cccc(-n2cnc3cc(-c4cccnc4)c(-n4cnc(C5CC5)c4)cc3c2=O)n1]

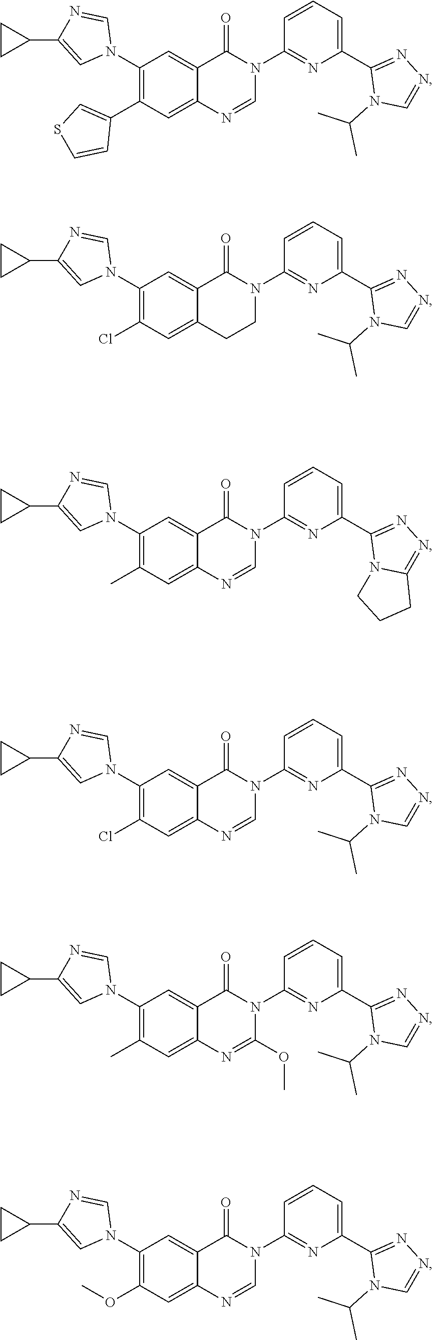 [SMILES: CC(C)n1cnnc1-c1cccc(-n2cnc3cc(-c4ccsc4)c(-n4cnc(C5CC5)c4)cc3c2=O)n1 ; CC(C)n1cnnc1-c1cccc(N2CCc3cc(Cl)c(-n4cnc(C5CC5)c4)cc3C2=O)n1 ; Cc1cc2ncn(-c3cccc(-c4nnc5n4CCC5)n3)c(=O)c2cc1-n1cnc(C2CC2)c1 ; CC(C)n1cnnc1-c1cccc(-n2cnc3cc(Cl)c(-n4cnc(C5CC5)c4)cc3c2=O)n1 ; COc1nc2cc(C)c(-n3cnc(C4CC4)c3)cc2c(=O)n1-c1cccc(-c2nncn2C(C)C)n1 ; COc1cc2ncn(-c3cccc(-c4nncn4C(C)C)n3)c(=O)c2cc1-n1cnc(C2CC2)c1]

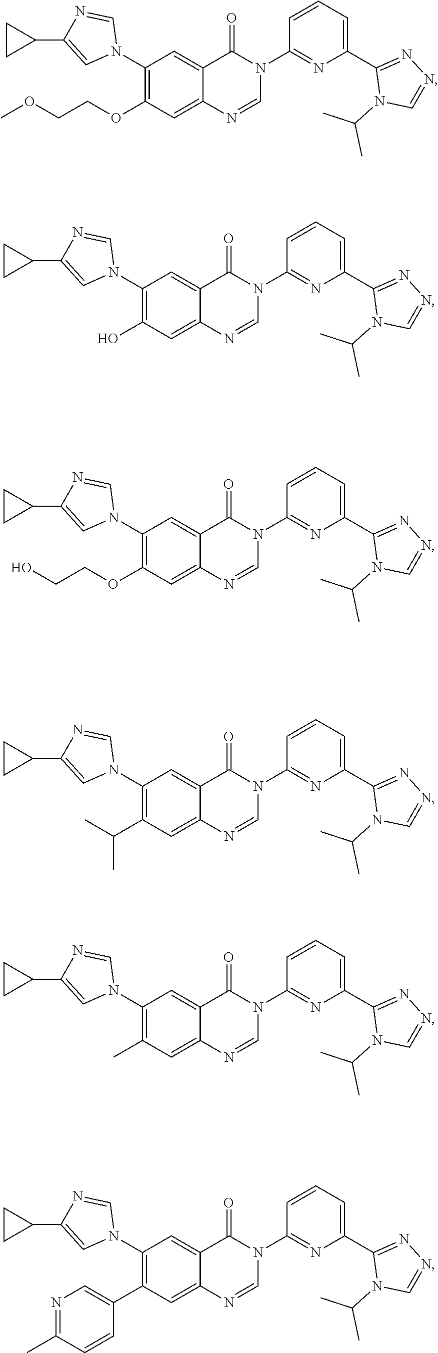 [SMILES: COCCOc1cc2ncn(-c3cccc(-c4nncn4C(C)C)n3)c(=O)c2cc1-n1cnc(C2CC2)c1 ; CC(C)n1cnnc1-c1cccc(-n2cnc3cc(O)c(-n4cnc(C5CC5)c4)cc3c2=O)n1 ; CC(C)n1cnnc1-c1cccc(-n2cnc3cc(OCCO)c(-n4cnc(C5CC5)c4)cc3c2=O)n1 ; CC(C)c1cc2ncn(-c3cccc(-c4nncn4C(C)C)n3)c(=O)c2cc1-n1cnc(C2CC2)c1 ; Cc1cc2ncn(-c3cccc(-c4nncn4C(C)C)n3)c(=O)c2cc1-n1cnc(C2CC2)c1 ; Cc1ccc(-c2cc3ncn(-c4cccc(-c5nncn5C(C)C)n4)c(=O)c3cc2-n2cnc(C3CC3)c2)cn1]

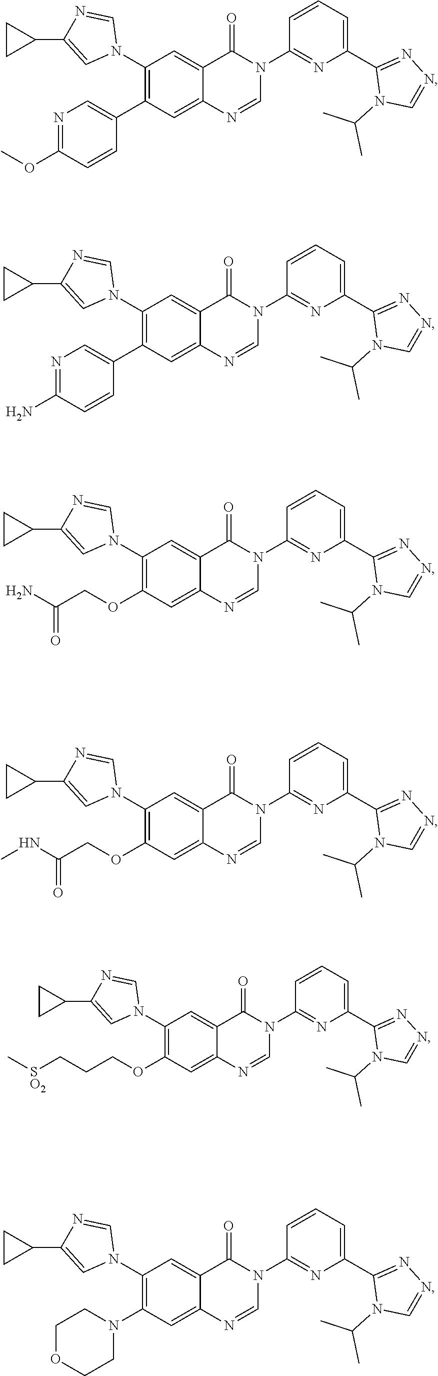 [SMILES: COc1ccc(-c2cc3ncn(-c4cccc(-c5nncn5C(C)C)n4)c(=O)c3cc2-n2cnc(C3CC3)c2)cn1 ; CC(C)n1cnnc1-c1cccc(-n2cnc3cc(-c4ccc(N)nc4)c(-n4cnc(C5CC5)c4)cc3c2=O)n1 ; CC(C)n1cnnc1-c1cccc(-n2cnc3cc(OCC(N)=O)c(-n4cnc(C5CC5)c4)cc3c2=O)n1 ; CNC(=O)COc1cc2ncn(-c3cccc(-c4nncn4C(C)C)n3)c(=O)c2cc1-n1cnc(C2CC2)c1 ; CC(C)n1cnnc1-c1cccc(-n2cnc3cc(OCCCS(C)(=O)=O)c(-n4cnc(C5CC5)c4)cc3c2=O)n1 ; CC(C)n1cnnc1-c1cccc(-n2cnc3cc(N4CCOCC4)c(-n4cnc(C5CC5)c4)cc3c2=O)n1]

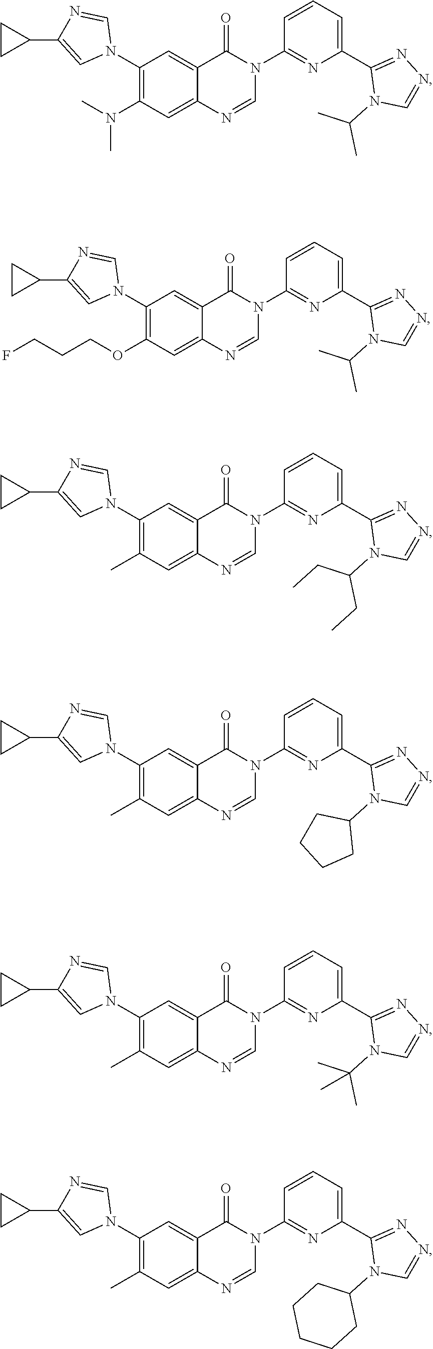 [SMILES: CC(C)n1cnnc1-c1cccc(-n2cnc3cc(N(C)C)c(-n4cnc(C5CC5)c4)cc3c2=O)n1 ; CC(C)n1cnnc1-c1cccc(-n2cnc3cc(OCCCF)c(-n4cnc(C5CC5)c4)cc3c2=O)n1 ; CCC(CC)n1cnnc1-c1cccc(-n2cnc3cc(C)c(-n4cnc(C5CC5)c4)cc3c2=O)n1 ; Cc1cc2ncn(-c3cccc(-c4nncn4C4CCCC4)n3)c(=O)c2cc1-n1cnc(C2CC2)c1 ; Cc1cc2ncn(-c3cccc(-c4nncn4C(C)(C)C)n3)c(=O)c2cc1-n1cnc(C2CC2)c1 ; Cc1cc2ncn(-c3cccc(-c4nncn4C4CCCCC4)n3)c(=O)c2cc1-n1cnc(C2CC2)c1]

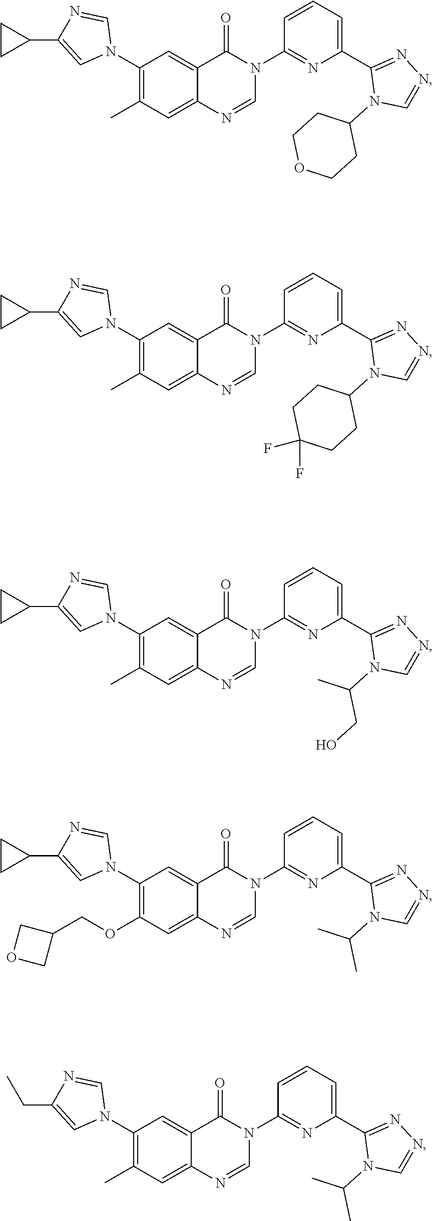 [SMILES: Cc1cc2ncn(-c3cccc(-c4nncn4C4CCOCC4)n3)c(=O)c2cc1-n1cnc(C2CC2)c1 ; Cc1cc2ncn(-c3cccc(-c4nncn4C4CCC(F)(F)CC4)n3)c(=O)c2cc1-n1cnc(C2CC2)c1 ; Cc1cc2ncn(-c3cccc(-c4nncn4C(C)CO)n3)c(=O)c2cc1-n1cnc(C2CC2)c1 ; CC(C)n1cnnc1-c1cccc(-n2cnc3cc(OCC4COC4)c(-n4cnc(C5CC5)c4)cc3c2=O)n1 ; CCc1cn(-c2cc3c(=O)n(-c4cccc(-c5nncn5C(C)C)n4)cnc3cc2C)cn1]

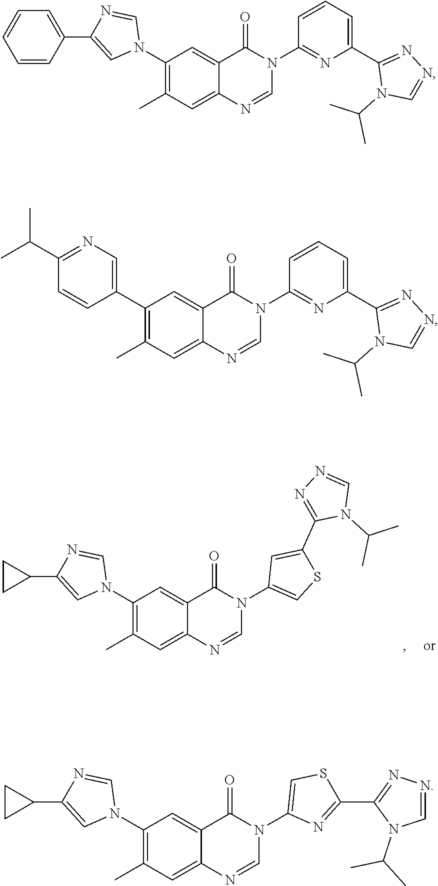 [SMILES: Cc1cc2ncn(-c3cccc(-c4nncn4C(C)C)n3)c(=O)c2cc1-n1cnc(-c2ccccc2)c1 ; Cc1cc2ncn(-c3cccc(-c4nncn4C(C)C)n3)c(=O)c2cc1-c1ccc(C(C)C)nc1 ; Cc1cc2ncn(-c3csc(-c4nncn4C(C)C)c3)c(=O)c2cc1-n1cnc(C2CC2)c1 ; Cc1cc2ncn(-c3csc(-c4nncn4C(C)C)n3)c(=O)c2cc1-n1cnc(C2CC2)c1]

or a pharmaceutically acceptable salt or solvate thereof.

In another aspect presented herein, is a method for treating a cognitive impairment in a subject in need thereof, comprising administering to the subject a dual inhibitor of ASK1 and DYRK1A, wherein the dual inhibitor is a compound having the structure of Formula (VI), or a pharmaceutically acceptable salt or solvate thereof:

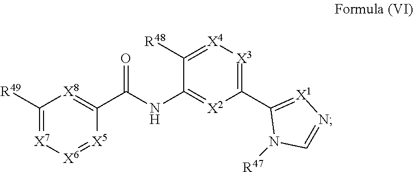

wherein:
R⁴⁷ is alkyl, alkenyl, alkynyl, cycloalkyl, aryl, heteroaryl, or heterocyclyl, all of which are optionally substituted with one, two, or three substituents selected from halo, oxo, alkyl, cycloalkyl, heterocyclyl, aryl, aryloxy, —NO₂, R⁵², —C(O)—R⁵², —OC(O)—R⁵²-C(O)—O—R⁵², —C(O)—N(R⁵²)(R⁵³), —OC(O)—N(R⁵²)(R⁵³), —S—R⁵², —S(═O)—R⁵², —S(═O)₂R⁵², —S(═O)₂—N(R⁵²)(R⁵³), —S(═O)₂—O—R⁵², —N(R⁵²)(R⁵³), —N(R⁵²)—C(O)—R⁵³, —N(R⁵²)—C(O)—O—R⁵³, —N(R⁵²)—C(O)—N(R⁵²)(R⁵³), —N(R⁵²)—S(═O)₂—R⁵², —CN, and —O—R⁵², wherein alkyl, cycloalkyl, heterocyclyl, phenyl, and phenoxy are optionally substituted by one, two, or three substituents selected from alkyl, cycloalkyl, alkoxy, hydroxyl, and halo;
   wherein R⁵² and R⁵³ are independently selected from the group consisting of hydrogen, C₁-C₁₅ alkyl, cycloalkyl, heterocyclyl, aryl, and heteroaryl, all of which are optionally substituted with one, two, or three substituents selected from halo, alkyl, mono- or dialkylamino, alkyl or aryl or heteroaryl amide, —CN, alkoxy, —CF₃, aryl, and heteroaryl;
   or R⁵² and R⁵³ when taken together with the nitrogen to which they are attached form a heterocycle;
R⁴⁸ is hydrogen, halogen, cyano, alkoxy, or alkyl optionally substituted by halo;
R⁴⁹ is aryl, heteroaryl, or heterocyclyl, all of which are optionally substituted with one or more substituents selected from alkyl, alkoxy, cycloalkyl, cycloalkylalkyl, aryl, arylalkyl, heteroaryl, heteroarylalkyl, heterocyclyl, heterocyclylalkyl, halo, oxo, —NO₂, haloalkyl, haloalkoxy, —CN, —O—R⁵², —O—C(O)—R⁵², —O—C(O)—N(R⁵²)(R⁵³), —S—R⁵², —N(R⁵²)(R⁵³), —S(═O)—R⁵², —S(═O)₂R⁵², —S(═O)₂—N(R⁵²)(R⁵³), —S(═O)₂—O—R⁵², —N(R⁵²)—C(O)—R⁵³, —N(R⁵²)—C(O)—
R⁵³, —N(R⁵²)—C(O)—N(R⁵²)(R⁵³), —C(O)—R⁵², —C(O)—O—R⁵², —C(O)—N(R⁵²)(R⁵³), and —N(R⁵²)—S(═O)₂—R⁵³, wherein the alkyl, alkoxy, cycloalkyl, aryl, heteroaryl or heterocyclyl is further optionally substituted with one or more substituents selected from halo, oxo, —NO₂, alkyl, haloalkyl, haloalkoxy, —N(R⁵²)(R⁵³), —C(O)—R⁵², —C(O)—O—R⁵², —C(O)—N(R⁵²)(R⁵³), —CN, —O—R⁵², cycloalkyl, aryl, heteroaryl and heterocyclyl;
with the proviso that the heteroaryl or heterocyclyl moiety includes at least one ring nitrogen atom; X¹, X², X³, X⁴, X⁵, X⁶, X⁷ and X⁸ are independently C(R⁵⁰) or N,
   wherein each R⁵⁰ is independently hydrogen, alkyl, alkoxy, cycloalkyl, aryl, heteroaryl, heterocyclyl, halo, —NO₂, haloalkyl, haloalkoxy, —CN, —O—R⁵², —S—R⁵², —N(R⁵²)(R⁵³), —S(═O)—R⁵², —S(═O)₂R⁵², —S(═O)₂—N(R⁵²)(R⁵³), —S(═O)₂—O—R⁵², —N(R⁵²)—C(O)—R⁵³, —N(R⁵²)—C(O)—O—R⁵³, —N(R⁵²)—C(O)—N(R⁵²)(R⁵³), —C(O)—R⁵², —C(O)—O—R⁵², —C(O)—N(R⁵²)(R⁵³), or —N(R⁵²)—S(═O)₂—R⁵³, wherein the alkyl, cycloalkyl, aryl, heteroaryl, and heterocyclyl is further optionally substituted with one or more substituents selected from halo, oxo, —NO₂, —CF₃, —O—CF₃, —N(R⁵²)(R⁵³), —C(O)—R⁵², —C(O)—O—R⁵³, —C(O)—N(R⁵²)(R⁵³), —CN, —O—R⁵²;
or X⁵ and X⁶ or X⁶ and X⁷ are joined to provide optionally substituted fused aryl or optionally substituted fused heteroaryl; and with the proviso that at least one of X², X³, and X⁴ is C(R⁵⁰); at least two of X⁵, X⁶, X⁷, and X⁸ are C(R⁵⁰); and at least one of X², X³, X⁴, X⁵, X⁶, X⁷ and X⁸ is N.

In some embodiments, the dual inhibitor of ASK1 and DYRK1A is selonsertib (GS4997), or a pharmaceutically acceptable salt or solvate thereof.

In another aspect presented herein, is a method for treating a cognitive impairment, in a subject in need thereof, comprising administering to the subject a dual inhibitor of ASK1 and DYRK1A, wherein the dual inhibitor is a compound having the structure of Formula (VII), or a pharmaceutically acceptable salt or solvate thereof:

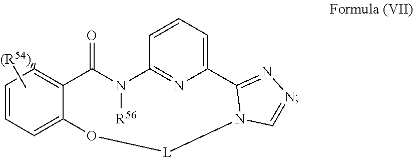

wherein:
L is selected from C₃₋₅alkylene and C₃₋₅alkenylene, wherein C₃₋₅alkylene and C₃₋₅alkenylene are optionally substituted with one or two R⁵⁵ groups;
each R⁵⁴ is independently selected from C₁₋₆alkyl, C₂₋₆alkenyl, C₂₋₆alkynyl, carbocyclyl, heterocyclyl, halogen, —CN, —C(O)R^{54a}, —C(O)₂R^{54a}, —C(O)N(R^{54a})₂, —N(R^{54a})₂, —N(R^{54a})C(O)R^{54a}, —N(R^{54a})C(O)₂R^{54a}, —N(R^{54a})C(O)N(R^{54a})₂, —N(R^{54a})S(O)₂R^{54a}, —OR^{54a}, —OC(O)R^{54a}, —OC(O)N(R^{54a})₂, —SR^{54a}, —S(O)R^{54a}, —S(O)₂R^{54a}, —S(O)N(R^{54a})₂, and —S(O)₂N(R^{54a})₂, wherein C₁₋₆alkyl, C₂₋₆alkenyl, C₂₋₆alkynyl, carbocyclyl, and heterocyclyl, are optionally substituted with one or more R⁵⁸ groups;
each R^{54a} is independently selected from hydrogen, C₁₋₆alkyl, C₂₋₆alkenyl, C₂₋₆alkynyl, carbocyclyl, and heterocyclyl, wherein C₁₋₆alkyl, C₂₋₆alkenyl, C₂₋₆alkynyl, carbocyclyl, and heterocyclyl, wherein each are optionally and independently substituted with one or more R⁵⁸ groups;
each R⁵⁸ is independently selected from C₁₋₆alkyl, C₂₋₆alkenyl, C₂₋₆alkynyl, carbocyclyl, heterocyclyl, halogen, —CN, —C(O)R^{58a}, —C(O)₂R^{58a}, —C(O)N(R^{58a})₂, —N(R^{58a})₂, —N(R^{58a})C(O)R^{58a}, —N(R^{58a})C(O)₂R^{58a}, —N(R^{58a})C(O)N(R^{58a})₂, —N(R^{58a})S(O)₂R^{58a}, —OR^{58a}, —OC(O)R^{58a}, —OC(O)N(R^{58a})₂, —SR^{58a}, —S(O)R^{58a}, —S(O)₂R^{58a}, —S(O)N(R^{58a})₂, and —S(O)₂N(R^{58a})₂, wherein C₁₋₆alkyl, C₂₋₆alkenyl, C₂₋₆alkynyl, carbocyclyl, and heterocyclyl are optionally and independently substituted with one or more groups selected from halogen, —CN, —C(O)R^{58a}, —C(O)₂R^{58a}, —C(O)N(R^{58a})₂, —N(R^{58a})₂, —N(R^{58a})C(O)R^{58a}, —N(R^{58a})C(O)₂R^{58a}, —N(R^{58a})C(O)N(R^{58a})₂, —N(R^{58a})S(O)₂R^{58a}, —OR^{58a}, —OC(O)R^{58a}, —OC(O)N(R^{58a})₂, —SR^{58a}, —S(O)R^{58a}, —S(O)₂R^{58a}, —S(O)N(R^{58a})₂, and —S(O)₂N(R^{58a})₂;
each R^{58a} is independently selected from hydrogen, C₁₋₆alkyl, C₂₋₆alkenyl, C₂₋₆alkynyl, carbocyclyl, and heterocyclyl;
each R⁵⁵ is independently selected from C₁₋₆alkyl, —CN, —C(O)R^{55a}, —C(O)₂R^{55a}, —C(O)N(R^{55a})₂, —NO₂, —N(R^{55a})₂, —N(R^{55a})C(O)R^{55a}, —N(R^{55a})C(O)₂R^{55a}, —N(R^{55a})C(O)N(R^{55a})₂, —N(R^{55a})S(O)₂R^{55a}, —OR^{55a}, —OC(O)R^{55a}, —OC(O)N(R^{55a})₂, —SR^{55a}, —S(O)R^{55a}, —S(O)₂R^{55a}, —S(O)N(R^{55a})₂, and —S(O)₂N(R⁵⁴)₂, wherein C₁₋₆alkyl is optionally substituted with one or more R⁵⁹ groups;
each R^{55a} is independently selected from hydrogen, C₁₋₆alkyl, C₂₋₆alkenyl, C₂₋₆alkynyl, carbocyclyl, and heterocyclyl, wherein C₁₋₆alkyl, C₂₋₆alkenyl, C₂₋₆alkynyl, carbocyclyl, and heterocyclyl is optionally and independently substituted with one or more R⁵⁹ groups; and
each R⁵⁹ is independently selected from C₁₋₆alkyl, halogen, and —OR^{59a};
each R^{59a} is independently selected from hydrogen, C₁₋₆alkyl, C₂₋₆alkenyl, C₂₋₆alkynyl, carbocyclyl, and heterocyclyl;
R⁵⁶ is hydrogen or C₁₋₄alkyl; and
n is selected from 0, 1, and 2.

In some embodiments of the dual inhibitor of Formula (VII), or a pharmaceutically acceptable salt or solvate thereof, L is C₃₋₅alkylene, optionally substituted with one or two R⁵⁵. In some embodiments, L is C₄alkylene, optionally substituted with one or two R⁵⁵. In some embodiments of the dual inhibitor of Formula (VII), L is C₄alkenylene, optionally substituted with one or two R⁵⁵.

In some embodiments of the dual inhibitor of Formula (VII), or a pharmaceutically acceptable salt or solvate thereof, wherein R⁵⁵ is selected from a group consisting of C₁₋₆alkyl, —CN, —C(O)R^{55a}, —C(O)₂R^{55a}, —C(O)N(R^{55a})₂, —NO₂, —N(R^{55a})₂, —N(R^{55a})C(O)R^{55a}, —N(R^{55a})C(O)₂R^{55a}, —OR^{55a}, —OC(O)R^{55a}, and —OC(O)N(R)^{55a}, wherein C₁₋₆alkyl is optionally substituted with one, two, three, or four R⁵⁹; wherein each R^{55a} is independently selected from hydrogen or C₁₋₆alkyl, wherein each C₁₋₆alkyl is optionally substituted with one, two, or three R⁵⁹; R⁵⁹ is independently halogen or —OR^{59a}; and each R^{59a} is independently hydrogen or C₁₋₄alkyl.

In some embodiments of the dual inhibitor of Formula (VII), or a pharmaceutically acceptable salt or solvate thereof, wherein R⁵⁵ is C₁₋₄alkyl. In some embodiments, R⁵⁵ is methyl.

In some embodiments of the dual inhibitor or Formula (VII), or a pharmaceutically acceptable salt or solvate thereof, L is —(CH₂)₃—, —(CH₂)₄—, —CH(CH₃)—CH₂—CH₂—CH₂—, —CH₂—CH(CH₃)—CH₂—CH₂—, —CH₂—CH₂—CH(CH₃)—CH₂——-CH₂—CH₂—CH₂—CH(CH₃)—, —(CH₂)₅—, —CH₂—CH═CH—CH₂—, or —CH₂—CH₂—CH═CH—CH₂—. In one embodiment of the dual inhibitor of Formula (V) or a pharmaceutically acceptable salt or solvate thereof, L is —(CH₂)₄—, —CH₂—CH(CH₃)—CH₂—CH₂—, —CH₂—CH₂—CH₂—CH(CH₃)—, —(CH₂)₅—, —(CH₂)₃—, or —CH₂—CH═CH—CH₂.

In some embodiments of the dual inhibitor of Formula (VII), or a pharmaceutically acceptable salt or solvate thereof, R⁵⁴ is independently selected from C₁₋₆alkyl, C₂₋₆alkenyl, C₂₋₆alkynyl, 4- to 7-membered monocyclic non-aromatic heterocyclyl, 5- to 6-membered N-containing heteroaryl, 6- to 8-membered spiro or bridged bicyclic heterocyclyl, halogen, —CN, —C(O)R⁵⁴, —C(O)₂R^{54a}, —C(O)N(R^{54a})₂, —N(R^{54a}), —OR^{54a}, —S(O)₂R^{54a}, and —S(O)₂N(R^{54a})₂; wherein C₁₋₆alkyl, C₂₋₆alkenyl, 4- to 7-membered monocyclic non-aromatic heterocyclyl, 5- to 6-membered N-containing heteroaryl, and 6- to 8-membered spiro or bridged bicyclic heterocyclyl, are each optionally substituted with one, two, three, or four R⁵⁷groups. In some embodiments of the dual inhibitor of Formula (VII), or a pharmaceutically acceptable salt or solvate thereof, each R⁵⁴ is independently selected from hydrogen, C₁₋₆alkyl, and 4- to 7-membered monocyclic N-containing non-aromatic heterocyclyl, wherein C₁₋₆alkyl, and 4- to 7-membered monocyclic N-containing non-aromatic heterocyclyl are optionally and independently substituted with one, two, or three R⁵⁷; and each R⁵⁷ is independently selected from C₁₋₆alkyl, halogen, —N(R⁵⁷)₂, —OR⁵⁷ , —CN, C₃₋₆cycloalkyl, 4- to 7-membered monocyclic non-aromatic heterocyclyl, wherein said C₁₋₆alkyl, C₃₋₆cyloalkyl, and 4- to 7-membered monocyclic non-aromatic heterocyclyl are optionally substituted with one, two, three, four, or five substituents selected from halogen, —CN, —C(O)R^{57a}, —C(O)₂R^{57a}, —C(C)N(R^{57a}), —N(R^{57a}), —N(R^{57a})C(O)R^{57a}, —N(R^{57a})C(O)₂R^{57a}, —N(R^{57a})C(C)N(R^{57a}), —N(R^{57a})S(O)₂R^{57a}, —OR^{57a}, —OC(O)R^{57a}, —OC(O)N(R^{57a})₂, —SR^{57a}, —S(O)R^{57a}, —S(O)₂R^{57a}, —S(O)N(R^{57a}), and —S(O)₂N(R^{57a})₂; and each R^{57a} is independently hydrogen or C₁₋₄alkyl.

In some embodiments of the dual inhibitor of Formula (VII), or a pharmaceutically acceptable salt or solvate thereof, R⁵⁴ is independently selected from C₁₋₆alkyl, C₂₋₆alkenyl, 4- to 7-membered monocyclic non-aromatic heterocyclyl, 5- to 6-membered N-containing heteroaryl, 6-to 8-membered spiro or bridged bicyclic heterocyclyl, halogen, —CN, —C(O)R⁵⁴ , —C(O)₂R⁵⁴ , —C(O)N(R^{54a})₂, —S(O)₂R^{54a}, and —S(O)₂N(R^{54a})₂, wherein C₁₋₆alkyl, C₂₋₆alkenyl, 4- to 7-membered monocyclic non-aromatic heterocyclyl, 5- to 6-membered N-containing heteroaryl, and 6- to 8-membered spiro or bridged bicyclic heterocyclyl, are optionally substituted with one, two, three or four R⁵⁷; R^{54a} is independently selected from hydrogen, C₁₋₆alkyl, and 4- to 7-membered monocyclic N-containing non-aromatic heterocyclyl, wherein said C₁₋₆alkyl, and 4- to 7-membered monocyclic N-containing non-aromatic heterocyclyl in each occurrence are optionally and independently substituted with one or three R⁵⁷; and R⁵⁷ is independently selected from C₁₋₆alkyl, halogen, and C₃₋₆cyloalkyl.

In some embodiments of the dual inhibitor of Formula (VII), or a pharmaceutically acceptable salt or solvate thereof, R⁵⁴ is selected from C₁₋₆alkyl, C₂₋₆alkenyl,C₂₋₆alkynyl, C₃₋₆cycloalkyl selected from cyclopropyl, cyclobutyl, cyclopentyl, and cyclohexyl, 4- to 7-membered monocyclic heterocyclyl selected from azetidinyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, dihydropyranyl, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, tetrahydropyridinyl, pyranyl, thiopyranyl, pyrazinyl, pyrimidinyl, pyridazinyl, oxazinyl, thiazinyl, dioxinyl, dithiinyl, oxathianyl, triazinyl, tetrazinyl, azepinyl, oxepinyl, thiepinyl, diazepinyl, and thiazepinyl, 6- to 8-membered spiro or bridged bicyclic heterocyclyl selected from 3-azabicyclo[3.1.0]hexanyl, 3-azabicyclo[3.1.1]heptanyl, 2-azaspiro[3.3]heptanyl, 2-oxa-6-azaspiro[3.3]heptanyl, 3-oxa-8-azabicyclo[3.2.1]octanyl, 6-oxa-3-azabicyclo[3.1.1]heptanyl, 8-oxa-3-azabicyclo[3.2.1]octanyl, 3-oxa-6-azabicyclo[3.1. l]heptanyl, and 5-azaspiro[2.3]hexanyl, 4,5,6,7-tetrahydropyrazolo[1,5-a]pyrazinyl, halogen, —CN, —OR^{54a}, —NHR^{54a}, —C(O)R^{54a}, and —S(O)₂R^{54a}, wherein C₁₋₆alkyl, C₂₋₆alkenyl, C₃₋₆cycloalkyl, 4- to 7-membered monocyclic heterocyclyl, and 6- to 8-membered Spiro or bridged bicyclic heterocyclyl are optionally substituted with one, two, three or four R⁵⁷; R^{54a} is hydrogen, C₁₋₆alkyl or 4- to 7-membered monocyclic heterocyclyl selected from azetidinyl, pyrrolidinyl, imidazolidinyl, pyrazolidinyl, oxazolidinyl, isoxazolidinyl, thiazolidinyl, isothiazolidinyl, piperidinyl, piperazinyl, morpholinyl, and azepinyl, wherein C₁₋₆alkyl or 4- to 7-membered monocyclic heterocyclyl is independently optionally substituted with one, two, or three R⁵⁷; and R⁵⁷ is independently selected from C₁₋₆alkyl, C₃₋₆cycloalkyl selected from cyclopropyl, cyclobutyl, cyclopentyl, and cyclohexyl, oxatanyl, —OR^{57a}, —N(R^{57a})₂ and halogen; R^{57a} is hydrogen or C₁₋₄alkyl.

In some embodiments of the dual inhibitor of Formula (VII), or pharmaceutically acceptable salt thereof, R⁵⁴ is independently selected from C₁₋₆alkyl, C₂₋₆alkenyl, C₃₋₆cycloalkyl selected from cyclopropyl, cyclobutyl, cyclopentyl, and cyclohexyl, 4- to 7-membered monocyclic heterocyclyl selected from azetidinyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, dihydropyranyl, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyranyl, thiopyranyl, pyrazinyl, pyrimidinyl, pyridazinyl, oxazinyl, thiazinyl, dioxinyl, dithiinyl, oxathianyl, triazinyl, tetrazinyl, azepinyl, oxepinyl, thiepinyl, diazepinyl, and thiazepinyl, 6- to 8-membered spiro or bridged bicyclic heterocyclyl selected from 3-azabicyclo[3.1.0]hexanyl, 3-azabicyclo[3.1.1]heptanyl, 2-azaspiro[3.3]heptanyl, 2-oxa-6-azaspiro[3.3]heptanyl, 3-oxa-8-azabicyclo[3.2.1]octanyl, 6-oxa-3-azabicyclo[3.1.1]heptanyl, 8-oxa-3-azabicyclo[3.2.1]octanyl, 3-oxa-6-azabicyclo[3. 1. l]heptanyl, and 5-azaspiro[2.3]hexanyl, halogen, —CN, —C(O)R⁵⁴ , and —S(O)₂R⁵⁴ , wherein C₁₋₆alkyl, C₂₋₆alkenyl, C₃₋₆cycloalkyl, 4- to 7-membered monocyclic heterocyclyl, and 6- to 8-membered spiro or bridged bicyclic heterocyclyl are optionally substituted with one, two, three, or four R⁵⁷; R^{54a} in each occurrence is hydrogen, C₁₋₆alkyl or 4- to 7-membered monocyclic heterocyclyl selected from azetidinyl, pyrrolidinyl, imidazolidinyl, pyrazolidinyl, oxazolidinyl, isoxazolidinyl, thiazolidinyl, isothiazolidinyl, piperidinyl, piperazinyl, morpholinyl, and azepanyl; and R⁵⁷ in each occurrence is independently selected from C₁₋₆alkyl, C₃₋₆cycloalkyl selected from cyclopropyl, cyclobutyl, cyclopentyl, and cyclohexyl, and halogen.

In some embodiments of the dual inhibitor of Formula (VII), or a pharmaceutically acceptable salt or solvate thereof, n is 1; R⁵⁴ is independently selected from —CH₃, —CF₃, —C≡CH, —CH₂NH₂, —CH₂N(CH₃)₂, —C(CH₃)₂OH, —C(CH₃)₂OCH₃, —C(CH₃)₂CN, —CH═CH₂, heterocyclyl selected from azetidinyl, pyrrolidinyl, tetrahydrofuranyl, piperidinyl, tetrahydropyranyl, piperazinyl, morpholinyl, dihydrofuranyl, dihydropyranyl, imidazolyl, pyrazolyl, triazolyl, tetrahydropyridinyl, pyridinyl, pyrimidinyl, pyridazinyl, 2-oxa-6-azaspiro[3.3]heptanyl, 3-oxa-8-azabicyclo[3.2.1]octanyl, 6-oxa-3-azabicyclo[3.1.1]heptanyl, 8-oxa-3-azabicyclo[3.2.1]octanyl, and 3-oxa-6-azabicyclo[3.1.1]heptanyl, 4,5,6,7-tetrahydropyrazolo[1,5-a]pyrazinyl, —Br, —F, —CN, —OR^{54a}, —NHR^{54a}, —C(O)R^{54a}, and —S(O)₂R^{54a}, wherein the heterocyclyl is optionally substituted with one, two, or three R⁵⁷ and —C≡CH is optionally substituted one R⁵⁷; R^{54a} is hydrogen, —CH₃, —CH₂CH₂OCH₃, —CH₂CH₂N(CH₃)₂, or pyrrolidinyl; and R⁵⁷ is independently selected from —CH₂CH₃, —CH(CH₃)₂, —CF₃, cyclopropyl, cyclobutyl, oxatanyl, 3-methyloxetan-3-yl, —CH₂CH₂N(CH₃)₂, and —F.

In some embodiments of the dual inhibitor of Formula (VII), or pharmaceutically acceptable salt thereof, n is 1; R⁵⁴ is independently selected from —CH₃, —CF₃, —CH═CH₂, heterocyclyl selected from azetidinyl, pyrrolidinyl, tetrahydrofuranyl, piperidinyl, tetrahydropyranyl, piperazinyl, morpholinyl, dihydrofuranyl, dihydropyranyl, imidazolyl, pyrazolyl, triazolyl, pyridinyl, 2-oxa-6-azaspiro[3.3]heptanyl, 3-oxa-8-azabicyclo[3.2.1]octanyl, 6-oxa-3-azabicyclo[3.1.1]heptanyl, 8-oxa-3-azabicyclo[3.2.1]octanyl, and 3-oxa-6-azabicyclo[3.1.1]heptanyl, —Br, —F, —CN, —C(O)R^{54a}, and —S(O)₂R^{54a}, wherein the heterocyclyl is optionally substituted with one or two R⁵⁷; R^{54a} in each occurrence is hydrogen, —CH₃ or pyrrolidinyl; R⁵⁷ in each occurrence is independently selected from —CH₃, —CH₂CH₃, cyclopropyl, and —F.

In some embodiments, the dual inhibitor having the structure of Formula (VII), has the structure of (VIIa), or a pharmaceutically acceptable salt or solvate thereof:

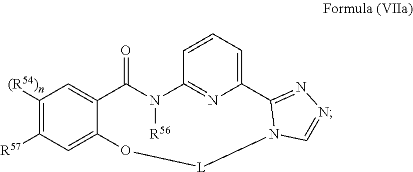

wherein:
L is —(CH₂)₄—, —CH₂—CH₂—CH₂—CH(CH₃)—, or —CH(CH₃)—CH₂—CH₂—CH₂—;
R⁵⁴ is —CN, halogen, or heterocyclyl, selected from imidazolyl, pyrazolyl, pyridine, piperazine, 1,2,3,6-tetrahydropyridine, piperidine, pyrimidine, and 6-oxa-3-azabicyclo[3.1.1]heptanyl, wherein each heterocycle is optionally substituted with one R⁵⁸ group;
R⁵⁶ is hydrogeen;
R⁵⁷ is halogen;
R⁵⁸ is C₁₋₄alkyl or C₃₋₆cycloalkyl, wherein Cₕ₄alkyl is optionally substituted with one —N(R^{58a})₂;
R^{58a} is hydrogen or C₁₋₄alkyl; and
n is 0 or 1.

In some embodiments of the dual inhibitor of Formula (VII) or (VIIa), or a pharmaceutically acceptable salt or solvate thereof, L is —(CH₂)₄— or —CH₂—CH₂—CH₂—CH(CH₃)—; n is 0 or 1; R⁵⁴ is heterocyclyl selected from imidazolyl, pyrazolyl, and 6-oxa-3-azabicyclo[3.1.1]heptanyl, wherein the heterocyclyl is optionally substituted with one R⁵⁷; and R⁵⁷ is C₁₋₄alkyl or C₃₋₆cycloalkyl.

In some embodiments of the dual inhibitor of Formula (VII) or (VIIa), or a pharmaceutically acceptable salt or solvate thereof, R⁵⁴ is —CN, —F, or a heterocyclyl selected from the following

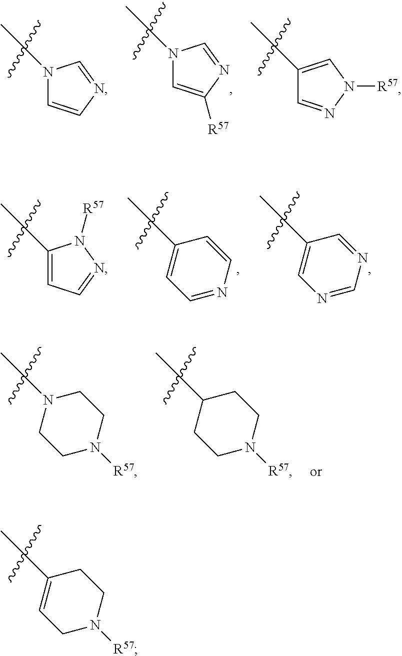

R^{54a} is hydrogen or —F; and R⁵⁷ is R⁵⁷ is —CH₃, —CH₂CH₂N(CH₃)₂, or cyclopropyl.

In some embodiments of the dual inhibitor of ASK1 and DYRK1A is:

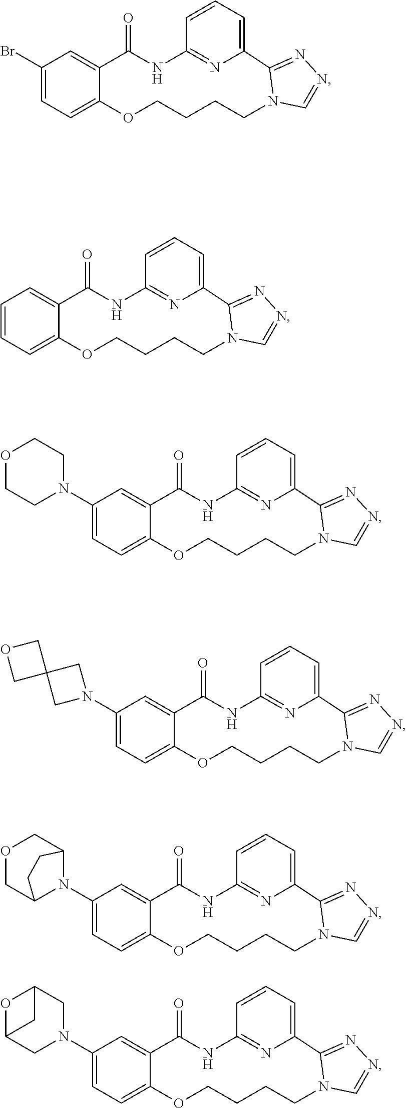

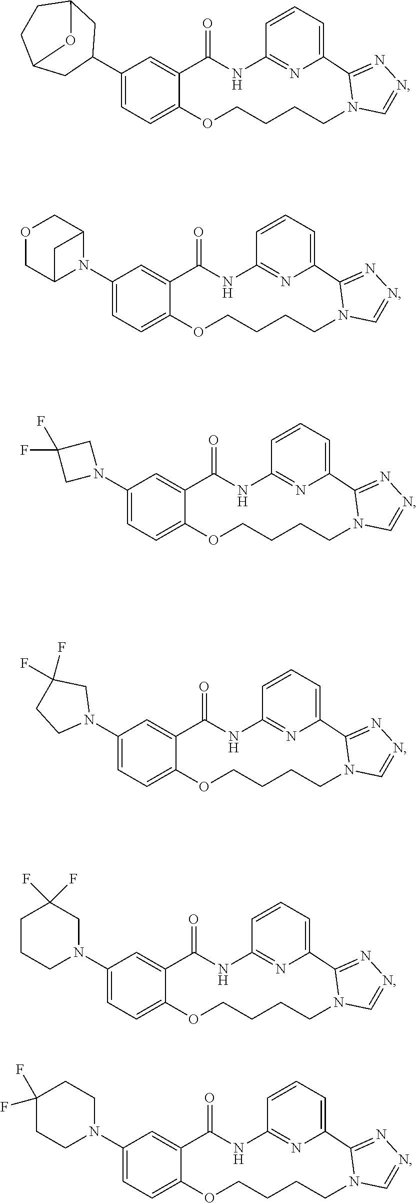 [SMILES: O=C1Nc2cccc(n2)-c2nncn2CCCCOc2ccc(C3CC4CCC(C3)O4)cc21 ; O=C1Nc2cccc(n2)-c2nncn2CCCCOc2ccc(N3C4COCC3C4)cc21 ; O=C1Nc2cccc(n2)-c2nncn2CCCCOc2ccc(N3CC(F)(F)C3)cc21 ; O=C1Nc2cccc(n2)-c2nncn2CCCCOc2ccc(N3CCC(F)(F)C3)cc21 ; O=C1Nc2cccc(n2)-c2nncn2CCCCOc2ccc(N3CCCC(F)(F)C3)cc21 ; O=C1Nc2cccc(n2)-c2nncn2CCCCOc2ccc(N3CCC(F)(F)CC3)cc21]

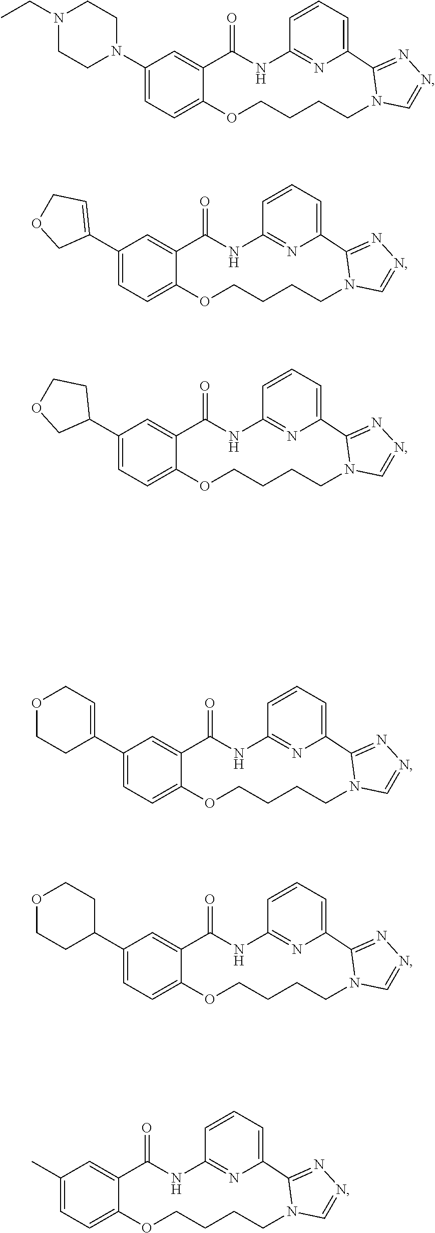 [SMILES: CCN1CCN(c2ccc3c(c2)C(=O)Nc2cccc(n2)-c2nncn2CCCCO3)CC1 ; O=C1Nc2cccc(n2)-c2nncn2CCCCOc2ccc(C3=CCOC3)cc21 ; O=C1Nc2cccc(n2)-c2nncn2CCCCOc2ccc(C3CCOC3)cc21 ; O=C1Nc2cccc(n2)-c2nncn2CCCCOc2ccc(C3=CCOCC3)cc21 ; O=C1Nc2cccc(n2)-c2nncn2CCCCOc2ccc(C3CCOCC3)cc21 ; Cc1ccc2c(c1)C(=O)Nc1cccc(n1)-c1nncn1CCCCO2]

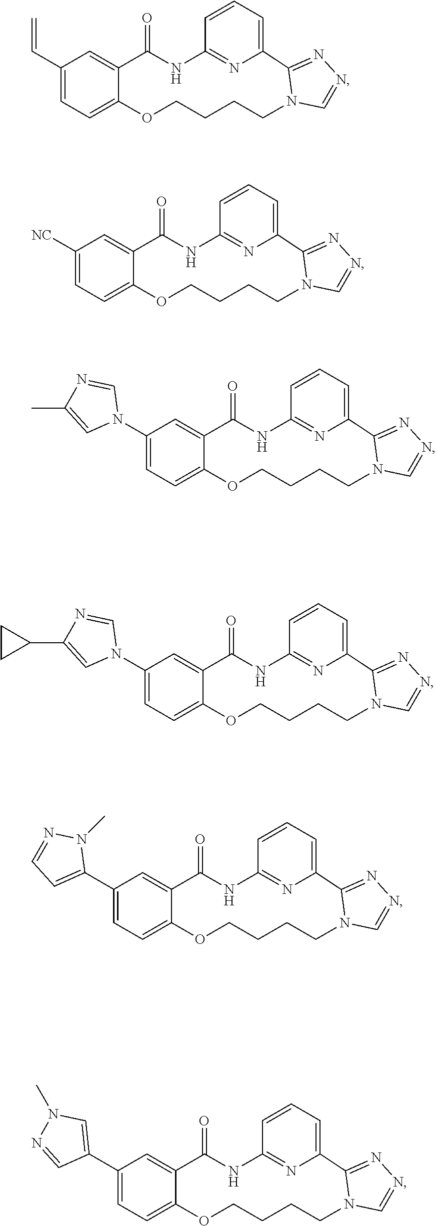 [SMILES: C=Cc1ccc2c(c1)C(=O)Nc1cccc(n1)-c1nncn1CCCCO2 ; N#Cc1ccc2c(c1)C(=O)Nc1cccc(n1)-c1nncn1CCCCO2 ; Cc1cn(-c2ccc3c(c2)C(=O)Nc2cccc(n2)-c2nncn2CCCCO3)cn1 ; O=C1Nc2cccc(n2)-c2nncn2CCCCOc2ccc(-n3cnc(C4CC4)c3)cc21 ; Cn1nccc1-c1ccc2c(c1)C(=O)Nc1cccc(n1)-c1nncn1CCCCO2 ; Cn1cc(-c2ccc3c(c2)C(=O)Nc2cccc(n2)-c2nncn2CCCCO3)cn1]

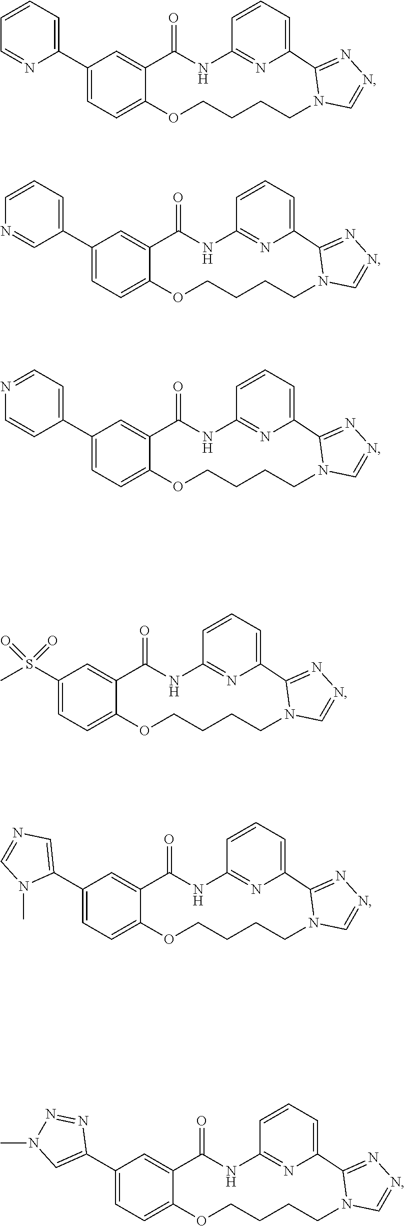 [SMILES: O=C1Nc2cccc(n2)-c2nncn2CCCCOc2ccc(-c3ccccn3)cc21 ; O=C1Nc2cccc(n2)-c2nncn2CCCCOc2ccc(-c3cccnc3)cc21 ; O=C1Nc2cccc(n2)-c2nncn2CCCCOc2ccc(-c3ccncc3)cc21 ; CS(=O)(=O)c1ccc2c(c1)C(=O)Nc1cccc(n1)-c1nncn1CCCCO2 ; Cn1cncc1-c1ccc2c(c1)C(=O)Nc1cccc(n1)-c1nncn1CCCCO2 ; Cn1cc(-c2ccc3c(c2)C(=O)Nc2cccc(n2)-c2nncn2CCCCO3)nn1]

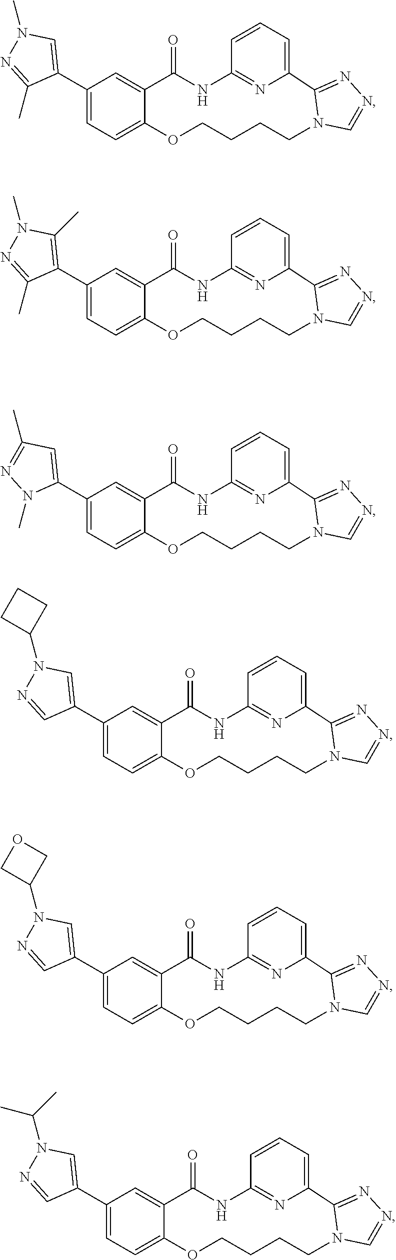 [SMILES: Cc1nn(C)cc1-c1ccc2c(c1)C(=O)Nc1cccc(n1)-c1nncn1CCCCO2 ; Cc1nn(C)c(C)c1-c1ccc2c(c1)C(=O)Nc1cccc(n1)-c1nncn1CCCCO2 ; Cc1cc(-c2ccc3c(c2)C(=O)Nc2cccc(n2)-c2nncn2CCCCO3)n(C)n1 ; O=C1Nc2cccc(n2)-c2nncn2CCCCOc2ccc(-c3cnn(C4CCC4)c3)cc21 ; O=C1Nc2cccc(n2)-c2nncn2CCCCOc2ccc(-c3cnn(C4COC4)c3)cc21 ; CC(C)n1cc(-c2ccc3c(c2)C(=O)Nc2cccc(n2)-c2nncn2CCCCO3)cn1]

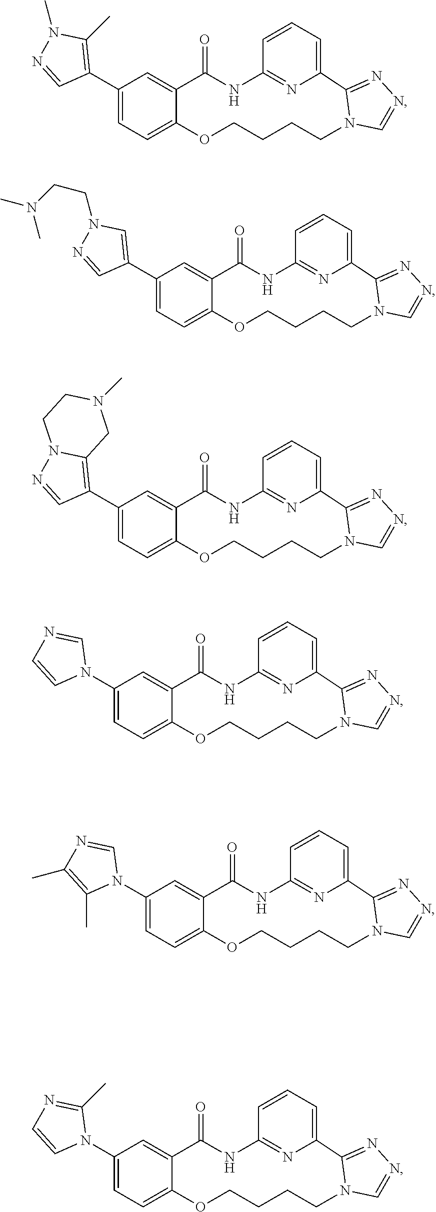 [SMILES: Cc1c(-c2ccc3c(c2)C(=O)Nc2cccc(n2)-c2nncn2CCCCO3)cnn1C ; CN(C)CCn1cc(-c2ccc3c(c2)C(=O)Nc2cccc(n2)-c2nncn2CCCCO3)cn1 ; CN1CCn2ncc(-c3ccc4c(c3)C(=O)Nc3cccc(n3)-c3nncn3CCCCO4)c2C1 ; O=C1Nc2cccc(n2)-c2nncn2CCCCOc2ccc(-n3ccnc3)cc21 ; Cc1ncn(-c2ccc3c(c2)C(=O)Nc2cccc(n2)-c2nncn2CCCCO3)c1C ; Cc1nccn1-c1ccc2c(c1)C(=O)Nc1cccc(n1)-c1nncn1CCCCO2]

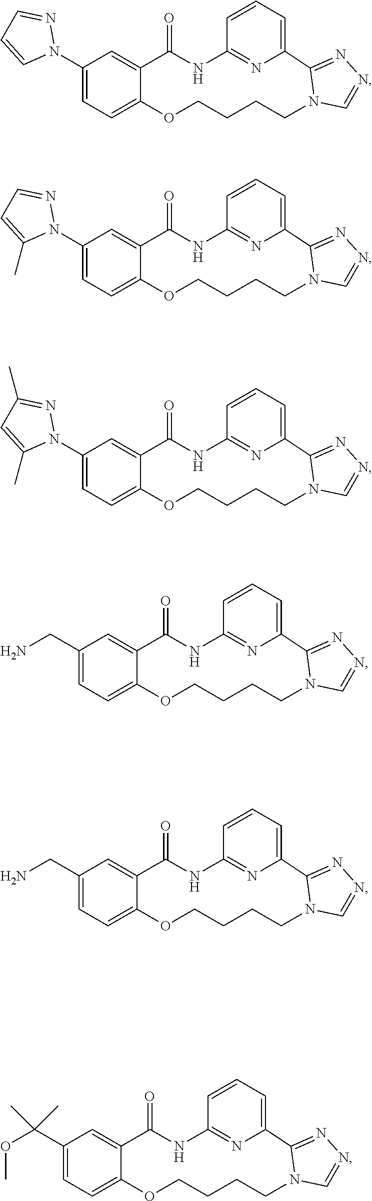 [SMILES: O=C1Nc2cccc(n2)-c2nncn2CCCCOc2ccc(-n3cccn3)cc21 ; Cc1ccnn1-c1ccc2c(c1)C(=O)Nc1cccc(n1)-c1nncn1CCCCO2 ; Cc1cc(C)n(-c2ccc3c(c2)C(=O)Nc2cccc(n2)-c2nncn2CCCCO3)n1 ; NCc1ccc2c(c1)C(=O)Nc1cccc(n1)-c1nncn1CCCCO2 ; NCc1ccc2c(c1)C(=O)Nc1cccc(n1)-c1nncn1CCCCO2 ; COC(C)(C)c1ccc2c(c1)C(=O)Nc1cccc(n1)-c1nncn1CCCCO2]

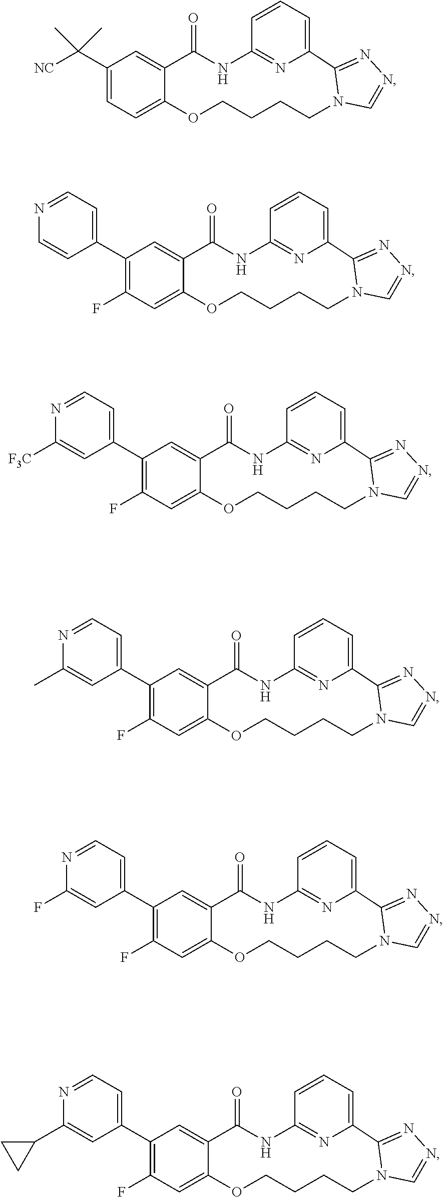 [SMILES: CC(C)(C#N)c1ccc2c(c1)C(=O)Nc1cccc(n1)-c1nncn1CCCCO2 ; O=C1Nc2cccc(n2)-c2nncn2CCCCOc2cc(F)c(-c3ccncc3)cc21 ; O=C1Nc2cccc(n2)-c2nncn2CCCCOc2cc(F)c(-c3ccnc(C(F)(F)F)c3)cc21 ; Cc1cc(-c2cc3c(cc2F)OCCCCn2cnnc2-c2cccc(n2)NC3=O)ccn1 ; O=C1Nc2cccc(n2)-c2nncn2CCCCOc2cc(F)c(-c3ccnc(F)c3)cc21 ; O=C1Nc2cccc(n2)-c2nncn2CCCCOc2cc(F)c(-c3ccnc(C4CC4)c3)cc21]

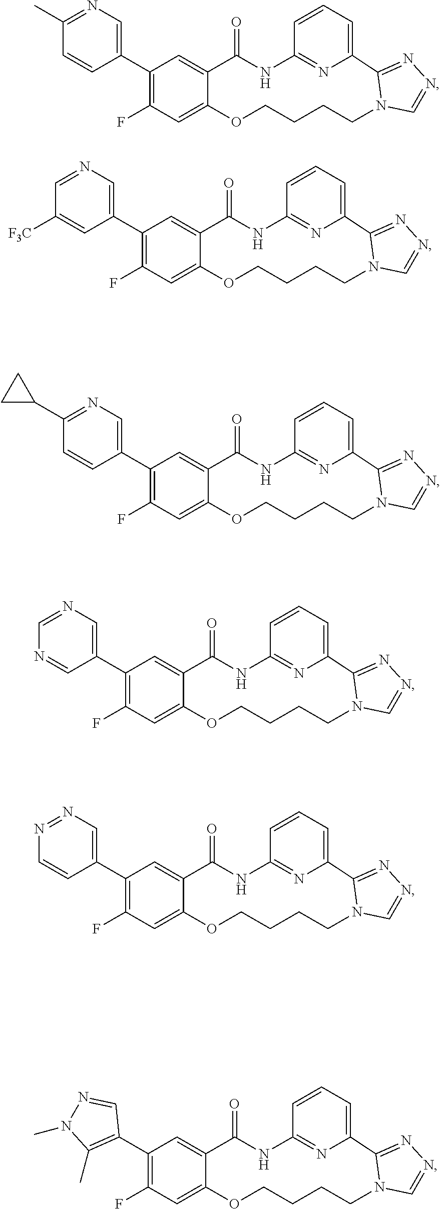 [SMILES: Cc1ccc(-c2cc3c(cc2F)OCCCCn2cnnc2-c2cccc(n2)NC3=O)cn1 ; O=C1Nc2cccc(n2)-c2nncn2CCCCOc2cc(F)c(-c3cncc(C(F)(F)F)c3)cc21 ; O=C1Nc2cccc(n2)-c2nncn2CCCCOc2cc(F)c(-c3ccc(C4CC4)nc3)cc21 ; O=C1Nc2cccc(n2)-c2nncn2CCCCOc2cc(F)c(-c3cncnc3)cc21 ; O=C1Nc2cccc(n2)-c2nncn2CCCCOc2cc(F)c(-c3ccnnc3)cc21 ; Cc1c(-c2cc3c(cc2F)OCCCCn2cnnc2-c2cccc(n2)NC3=O)cnn1C]

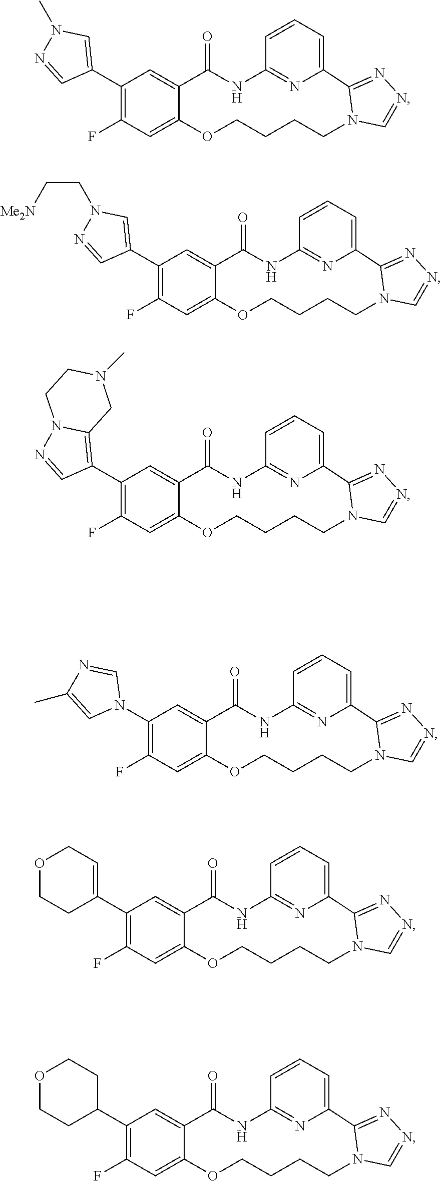

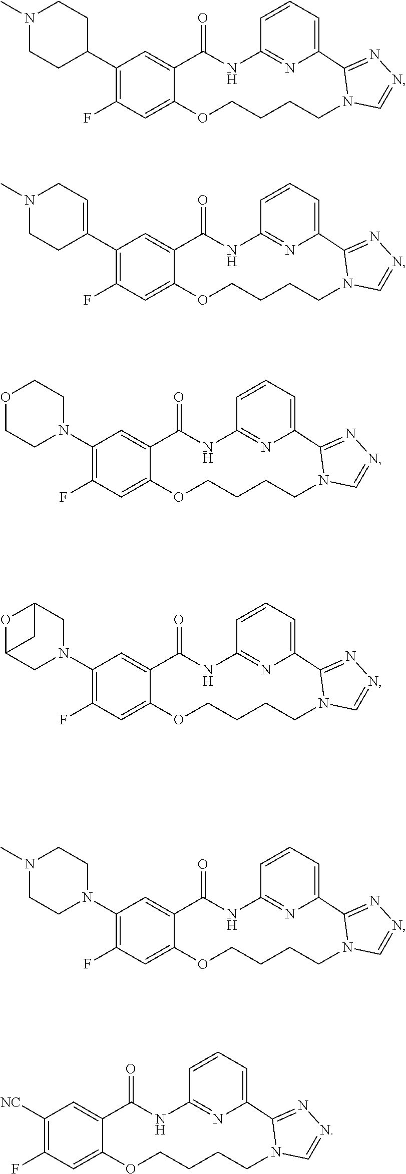 [SMILES: CN1CCC(c2cc3c(cc2F)OCCCCn2cnnc2-c2cccc(n2)NC3=O)CC1 ; CN1CC=C(c2cc3c(cc2F)OCCCCn2cnnc2-c2cccc(n2)NC3=O)CC1 ; O=C1Nc2cccc(n2)-c2nncn2CCCCOc2cc(F)c(N3CCOCC3)cc21 ; O=C1Nc2cccc(n2)-c2nncn2CCCCOc2cc(F)c(N3CC4CC(C3)O4)cc21 ; CN1CCN(c2cc3c(cc2F)OCCCCn2cnnc2-c2cccc(n2)NC3=O)CC1 ; N#Cc1cc2c(cc1F)OCCCCn1cnnc1-c1cccc(n1)NC2=O]

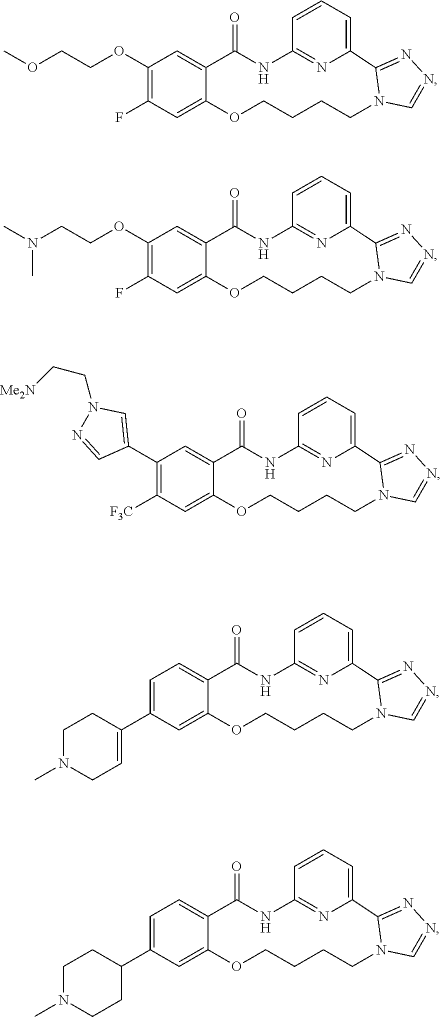 [SMILES: COCCOc1cc2c(cc1F)OCCCCn1cnnc1-c1cccc(n1)NC2=O ; CN(C)CCOc1cc2c(cc1F)OCCCCn1cnnc1-c1cccc(n1)NC2=O ; CN(C)CCn1cc(-c2cc3c(cc2C(F)(F)F)OCCCCn2cnnc2-c2cccc(n2)NC3=O)cn1 ; CN1CC=C(c2ccc3c(c2)OCCCCn2cnnc2-c2cccc(n2)NC3=O)CC1 ; CN1CCC(c2ccc3c(c2)OCCCCn2cnnc2-c2cccc(n2)NC3=O)CC1]

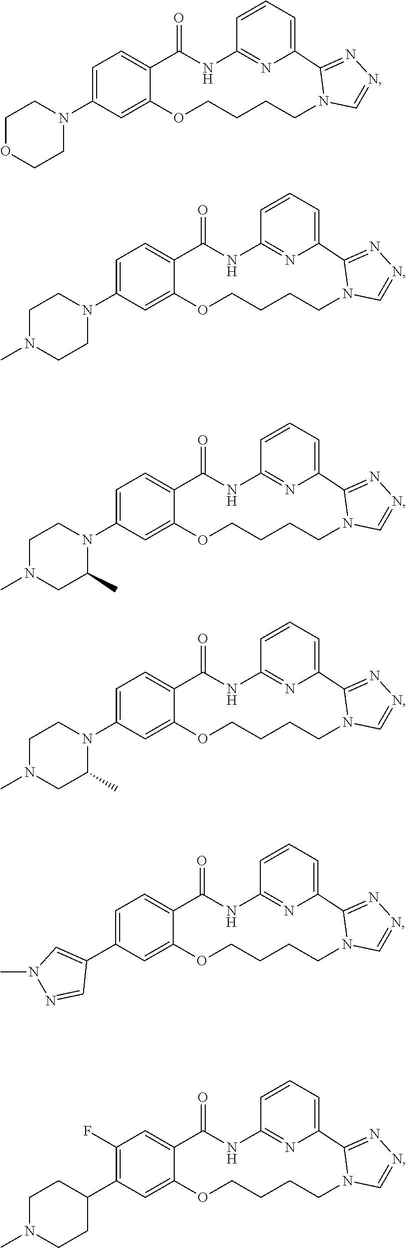 [SMILES: O=C1Nc2cccc(n2)-c2nncn2CCCCOc2cc(N3CCOCC3)ccc21 ; CN1CCN(c2ccc3c(c2)OCCCCn2cnnc2-c2cccc(n2)NC3=O)CC1 ; C[C@H]1CN(C)CCN1c1ccc2c(c1)OCCCCn1cnnc1-c1cccc(n1)NC2=O ; C[C@@H]1CN(C)CCN1c1ccc2c(c1)OCCCCn1cnnc1-c1cccc(n1)NC2=O ; Cn1cc(-c2ccc3c(c2)OCCCCn2cnnc2-c2cccc(n2)NC3=O)cn1 ; CN1CCC(c2cc3c(cc2F)C(=O)Nc2cccc(n2)-c2nncn2CCCCO3)CC1]

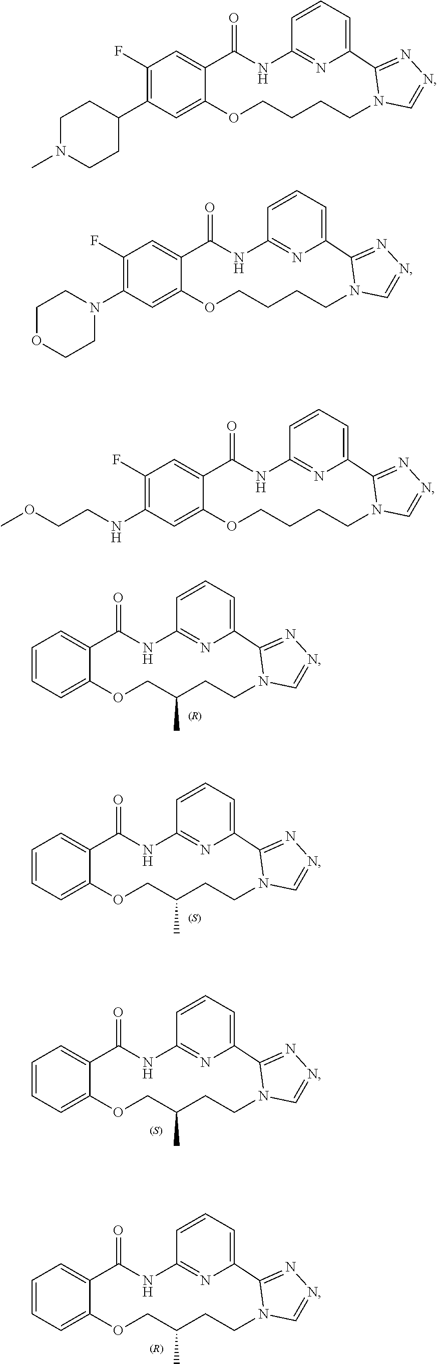

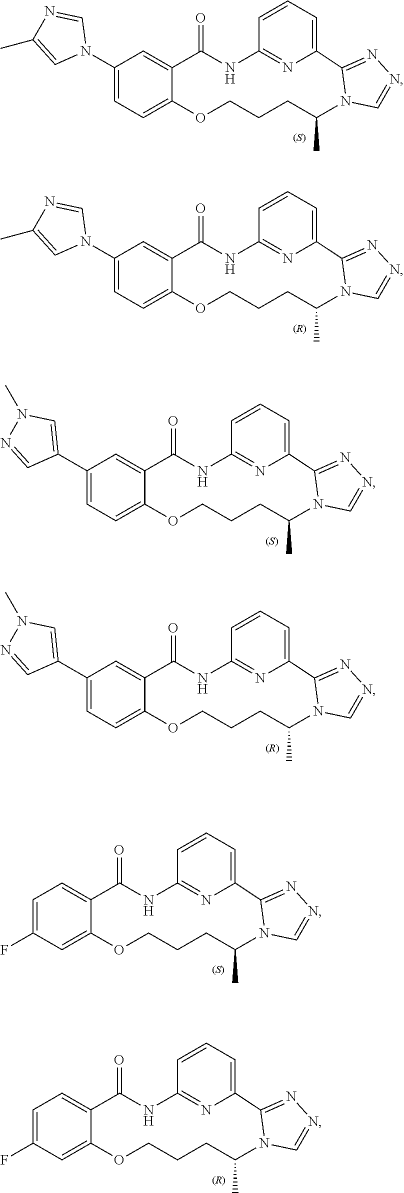

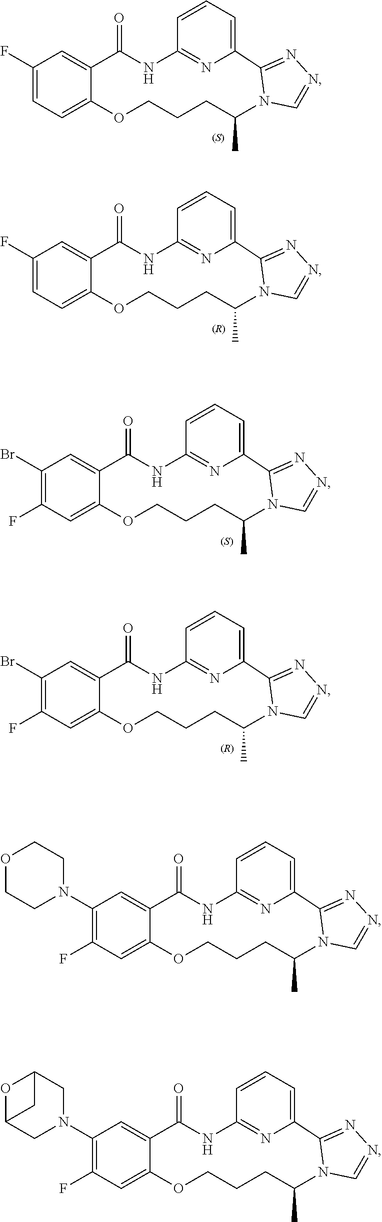

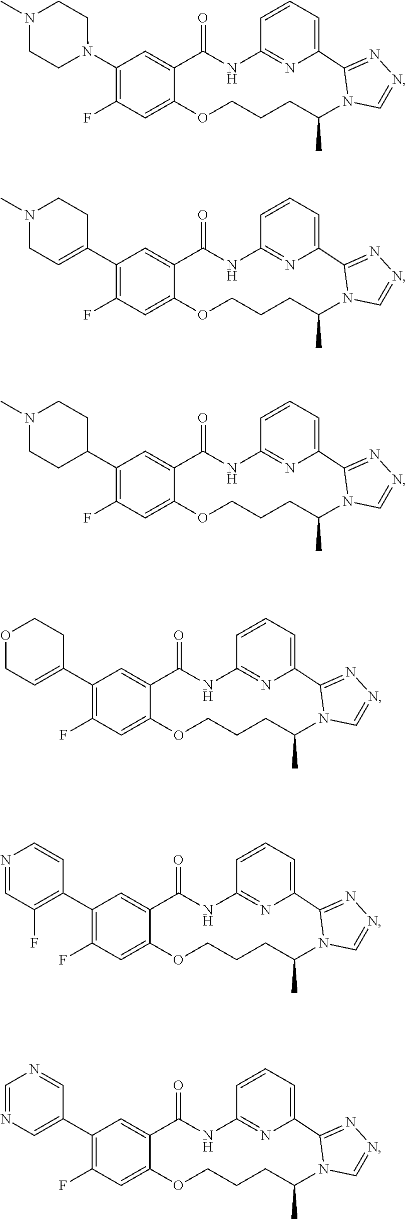 [SMILES: C[C@H]1CCCOc2cc(F)c(N3CCN(C)CC3)cc2C(=O)Nc2cccc(n2)-c2nncn21 ; C[C@H]1CCCOc2cc(F)c(C3=CCN(C)CC3)cc2C(=O)Nc2cccc(n2)-c2nncn21 ; C[C@H]1CCCOc2cc(F)c(C3CCN(C)CC3)cc2C(=O)Nc2cccc(n2)-c2nncn21 ; C[C@H]1CCCOc2cc(F)c(C3=CCOCC3)cc2C(=O)Nc2cccc(n2)-c2nncn21 ; C[C@H]1CCCOc2cc(F)c(-c3ccncc3F)cc2C(=O)Nc2cccc(n2)-c2nncn21 ; C[C@H]1CCCOc2cc(F)c(-c3cncnc3)cc2C(=O)Nc2cccc(n2)-c2nncn21]

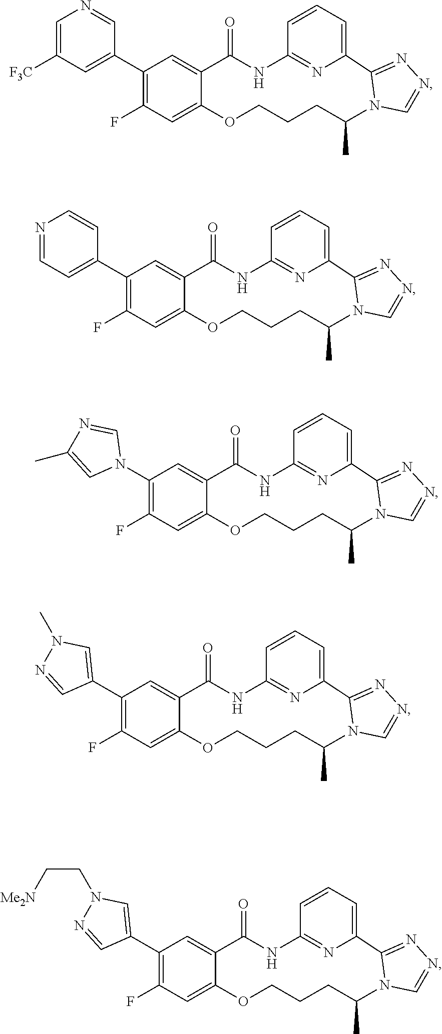 [SMILES: C[C@H]1CCCOc2cc(F)c(-c3cncc(C(F)(F)F)c3)cc2C(=O)Nc2cccc(n2)-c2nncn21 ; C[C@H]1CCCOc2cc(F)c(-c3ccncc3)cc2C(=O)Nc2cccc(n2)-c2nncn21 ; Cc1cn(-c2cc3c(cc2F)OCCC[C@H](C)n2cnnc2-c2cccc(n2)NC3=O)cn1 ; C[C@H]1CCCOc2cc(F)c(-c3cnn(C)c3)cc2C(=O)Nc2cccc(n2)-c2nncn21 ; C[C@H]1CCCOc2cc(F)c(-c3cnn(CCN(C)C)c3)cc2C(=O)Nc2cccc(n2)-c2nncn21]

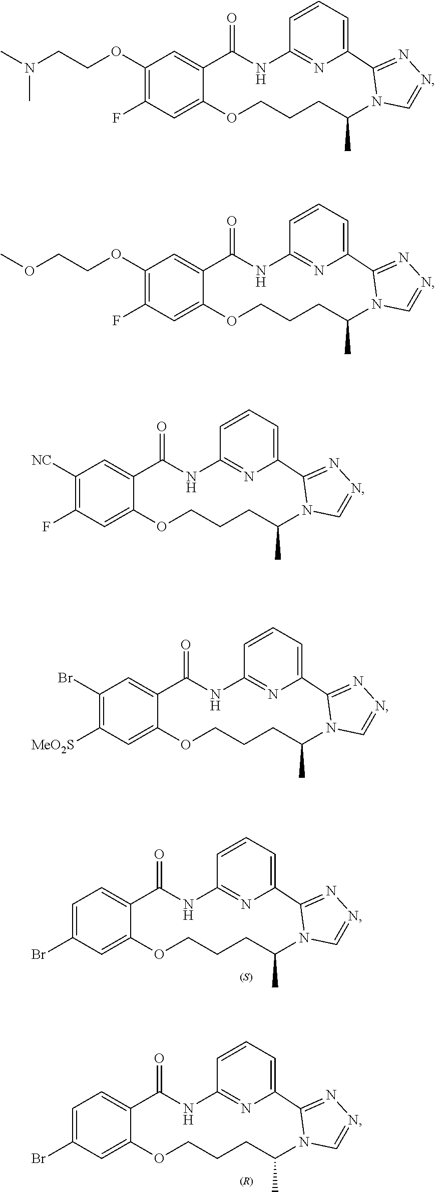

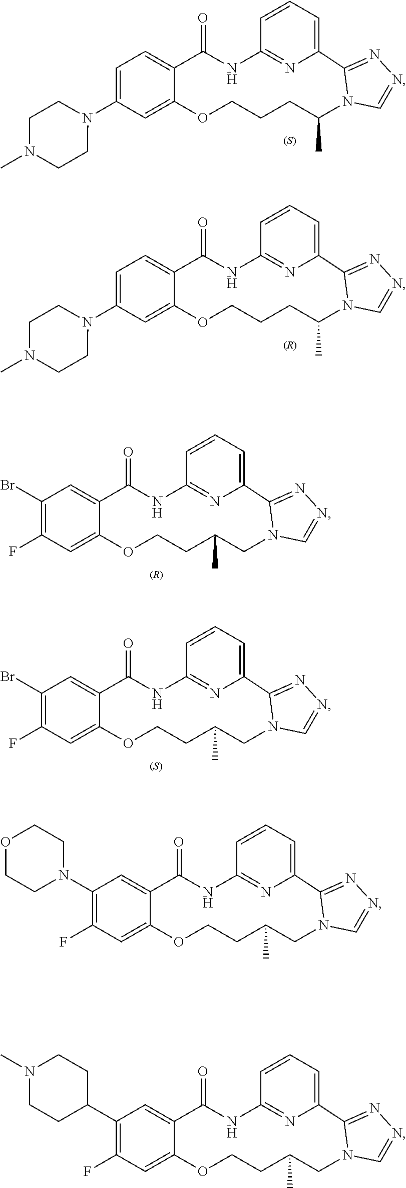

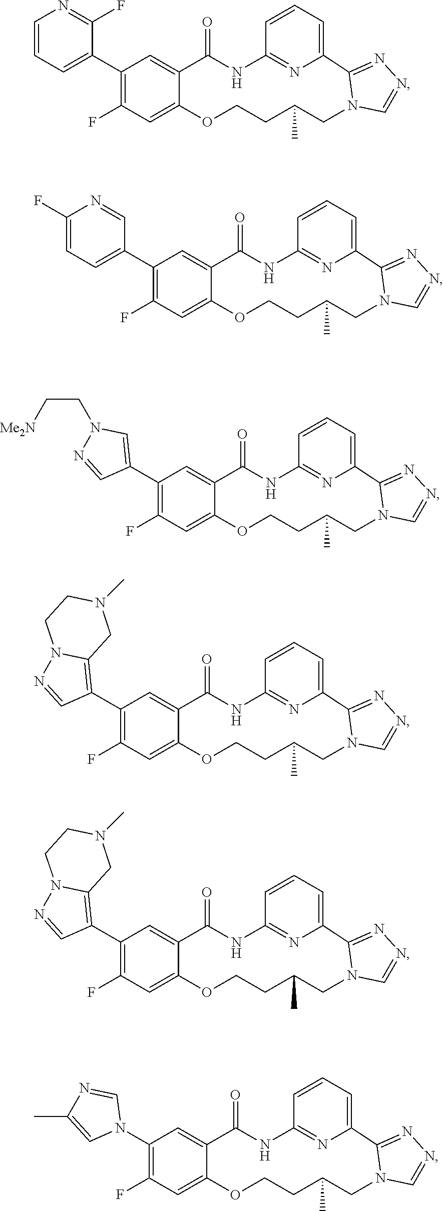 [SMILES: C[C@H]1CCOc2cc(F)c(-c3cccnc3F)cc2C(=O)Nc2cccc(n2)-c2nncn2C1 ; C[C@H]1CCOc2cc(F)c(-c3ccc(F)nc3)cc2C(=O)Nc2cccc(n2)-c2nncn2C1 ; C[C@H]1CCOc2cc(F)c(-c3cnn(CCN(C)C)c3)cc2C(=O)Nc2cccc(n2)-c2nncn2C1 ; C[C@H]1CCOc2cc(F)c(-c3cnn4c3CN(C)CC4)cc2C(=O)Nc2cccc(n2)-c2nncn2C1 ; C[C@@H]1CCOc2cc(F)c(-c3cnn4c3CN(C)CC4)cc2C(=O)Nc2cccc(n2)-c2nncn2C1 ; Cc1cn(-c2cc3c(cc2F)OCC[C@H](C)Cn2cnnc2-c2cccc(n2)NC3=O)cn1]

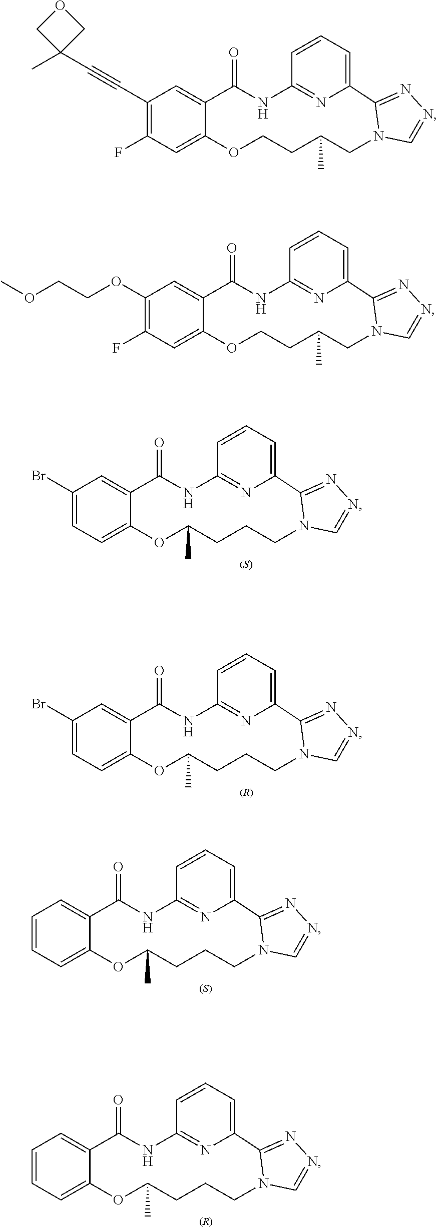

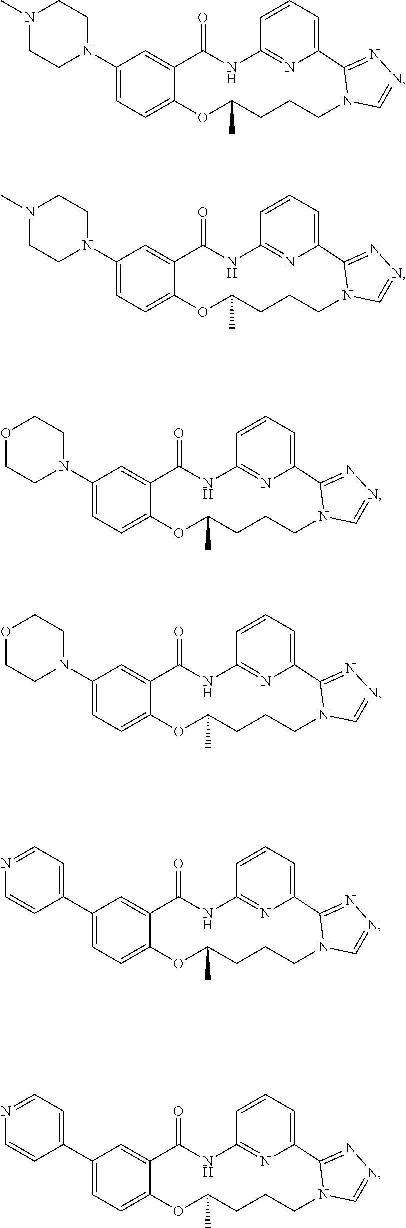 [SMILES: C[C@H]1CCCn2cnnc2-c2cccc(n2)NC(=O)c2cc(N3CCN(C)CC3)ccc2O1 ; C[C@@H]1CCCn2cnnc2-c2cccc(n2)NC(=O)c2cc(N3CCN(C)CC3)ccc2O1 ; C[C@H]1CCCn2cnnc2-c2cccc(n2)NC(=O)c2cc(N3CCOCC3)ccc2O1 ; C[C@@H]1CCCn2cnnc2-c2cccc(n2)NC(=O)c2cc(N3CCOCC3)ccc2O1 ; C[C@H]1CCCn2cnnc2-c2cccc(n2)NC(=O)c2cc(-c3ccncc3)ccc2O1 ; C[C@@H]1CCCn2cnnc2-c2cccc(n2)NC(=O)c2cc(-c3ccncc3)ccc2O1]

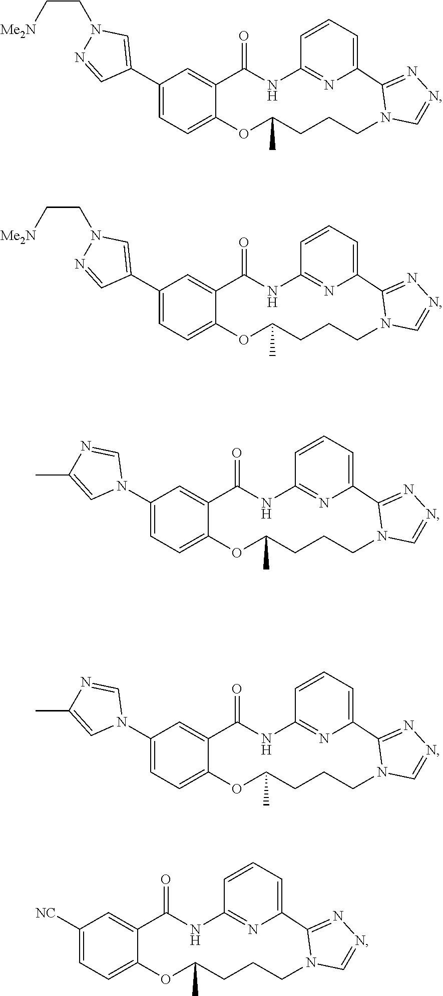 [SMILES: C[C@H]1CCCn2cnnc2-c2cccc(n2)NC(=O)c2cc(-c3cnn(CCN(C)C)c3)ccc2O1 ; C[C@@H]1CCCn2cnnc2-c2cccc(n2)NC(=O)c2cc(-c3cnn(CCN(C)C)c3)ccc2O1 ; Cc1cn(-c2ccc3c(c2)C(=O)Nc2cccc(n2)-c2nncn2CCC[C@H](C)O3)cn1 ; Cc1cn(-c2ccc3c(c2)C(=O)Nc2cccc(n2)-c2nncn2CCC[C@@H](C)O3)cn1 ; C[C@H]1CCCn2cnnc2-c2cccc(n2)NC(=O)c2cc(C#N)ccc2O1]

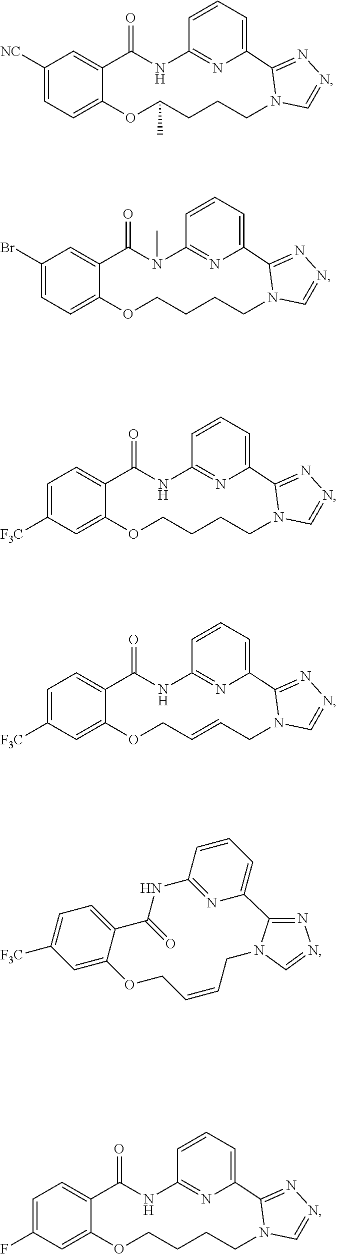 [SMILES: C[C@@H]1CCCn2cnnc2-c2cccc(n2)NC(=O)c2cc(C#N)ccc2O1 ; CN1C(=O)c2cc(Br)ccc2OCCCCn2cnnc2-c2cccc1n2 ; O=C1Nc2cccc(n2)-c2nncn2CCCCOc2cc(C(F)(F)F)ccc21 ; O=C1Nc2cccc(n2)-c2nncn2C/C=C/COc2cc(C(F)(F)F)ccc21 ; O=C1Nc2cccc(n2)-c2nncn2C/C=C\COc2cc(C(F)(F)F)ccc21 ; O=C1Nc2cccc(n2)-c2nncn2CCCCOc2cc(F)ccc21]

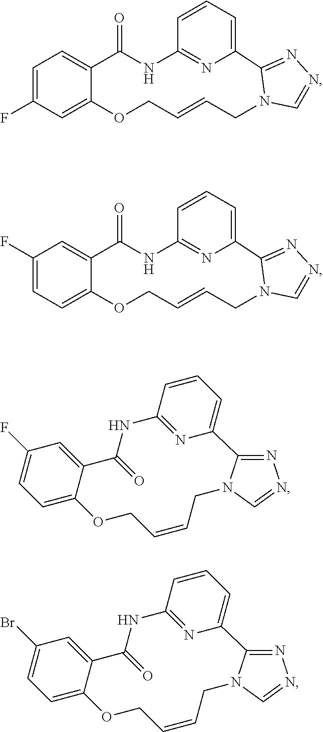 [SMILES: O=C1Nc2cccc(n2)-c2nncn2C/C=C/COc2cc(F)ccc21 ; O=C1Nc2cccc(n2)-c2nncn2C/C=C/COc2ccc(F)cc21 ; O=C1Nc2cccc(n2)-c2nncn2C/C=C\COc2ccc(F)cc21 ; O=C1Nc2cccc(n2)-c2nncn2C/C=C\COc2ccc(Br)cc21]

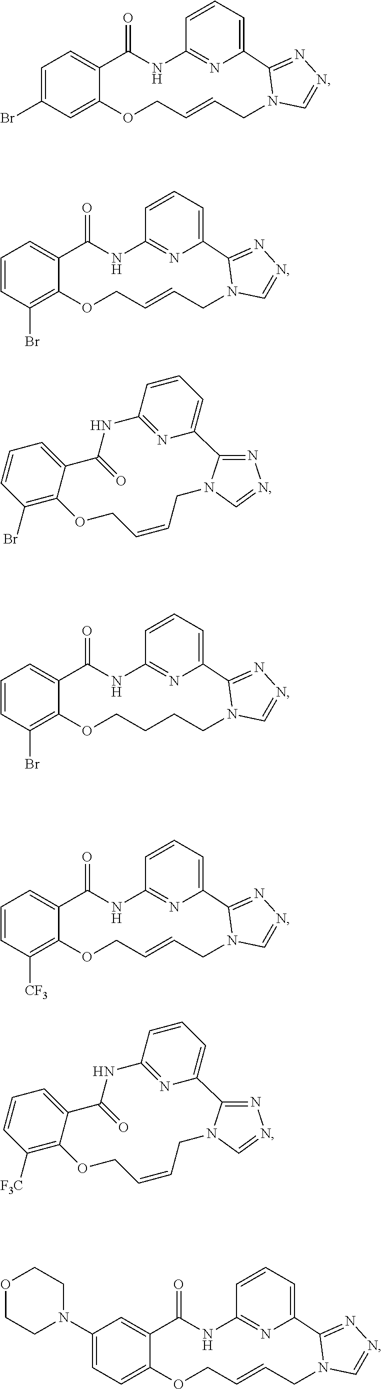

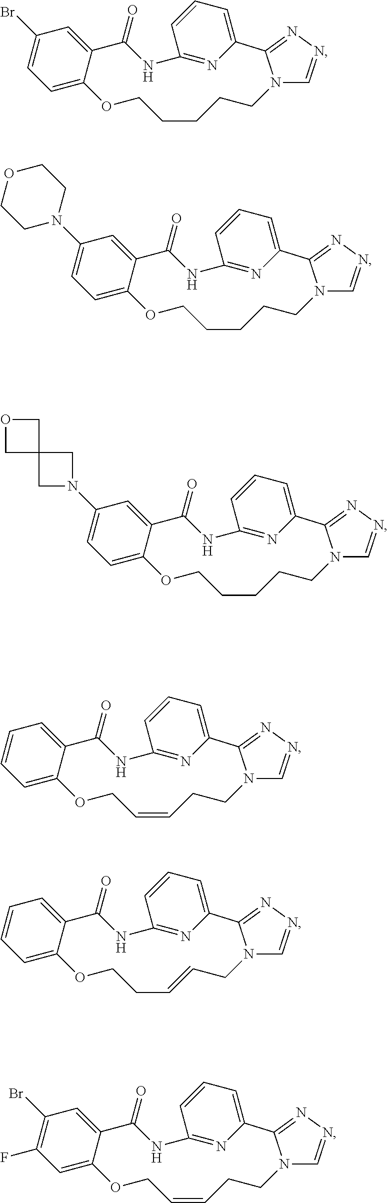 [SMILES: O=C1Nc2cccc(n2)-c2nncn2CCCCCOc2ccc(Br)cc21 ; O=C1Nc2cccc(n2)-c2nncn2CCCCCOc2ccc(N3CCOCC3)cc21 ; O=C1Nc2cccc(n2)-c2nncn2CCCCCOc2ccc(N3CC4(COC4)C3)cc21 ; O=C1Nc2cccc(n2)-c2nncn2CC/C=C\COc2ccccc21 ; O=C1Nc2cccc(n2)-c2nncn2C/C=C/CCOc2ccccc21 ; O=C1Nc2cccc(n2)-c2nncn2CC/C=C\COc2cc(F)c(Br)cc21]

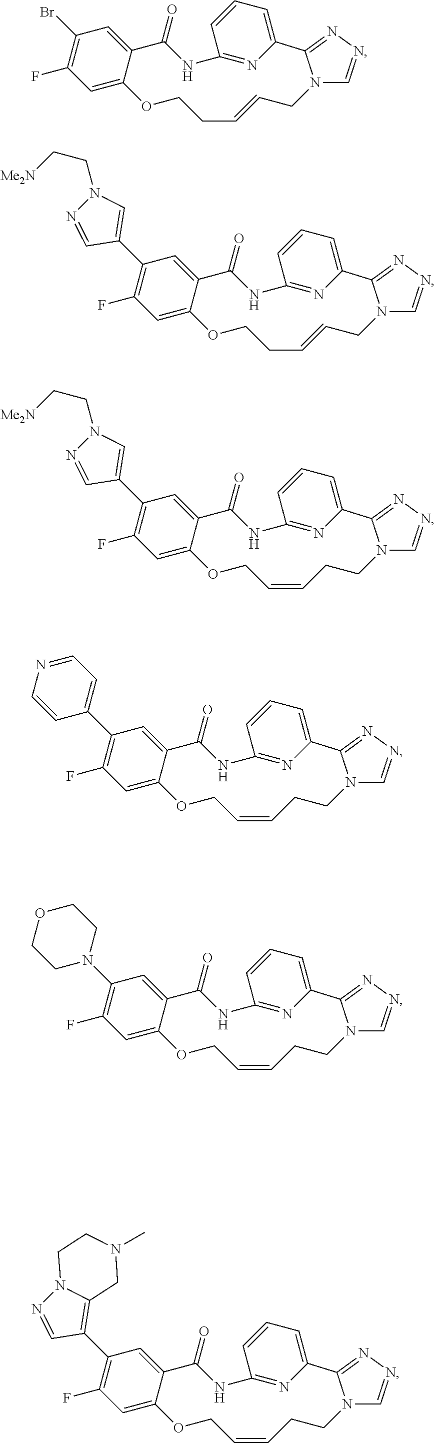 [SMILES: O=C1Nc2cccc(n2)-c2nncn2C/C=C/CCOc2cc(F)c(Br)cc21 ; CN(C)CCn1cc(-c2cc3c(cc2F)OCC/C=C/Cn2cnnc2-c2cccc(n2)NC3=O)cn1 ; CN(C)CCn1cc(-c2cc3c(cc2F)OC/C=C\CCn2cnnc2-c2cccc(n2)NC3=O)cn1 ; O=C1Nc2cccc(n2)-c2nncn2CC/C=C\COc2cc(F)c(-c3ccncc3)cc21 ; O=C1Nc2cccc(n2)-c2nncn2CC/C=C\COc2cc(F)c(N3CCOCC3)cc21 ; CN1CCn2ncc(-c3cc4c(cc3F)OC/C=C\CCn3cnnc3-c3cccc(n3)NC4=O)c2C1]

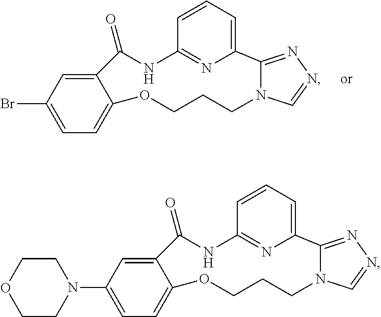 [SMILES: O=C1Nc2cccc(n2)-c2nncn2CCCOc2ccc(Br)cc21 ; O=C1Nc2cccc(n2)-c2nncn2CCCOc2ccc(N3CCOCC3)cc21]

or a pharmaceutically acceptable salt or solvate thereof.

In another aspect presented herein, is a method for treating a cognitive impairment in a subject in need thereof, comprising administering to the subject a dual inhibitor of ASK1 and DYRK1A, wherein the dual inhibitor is a compound having the structure of Formula (VIII), or a pharmaceutically acceptable salt or solvate thereof:

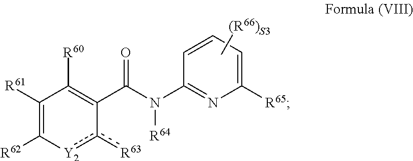

wherein:

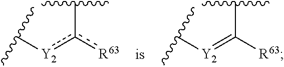

wherein:
Y² is N or CR^{Y2};
R⁶³ is hydrogen, halogen, —CN, —OR⁶⁷, —SR⁶⁷, —S(═O)R⁶⁹, —NO₂, —NR⁶⁷R⁶⁸, —S(═O)₂R⁶⁹, —NR⁶⁷S(═O)₂R⁶⁹, —S(═O)₂NR⁶⁷R⁶⁸, —C(═O)R⁶⁹, —OC(═O)R⁶⁹, —CO₂R⁶⁷, —OCO₂R⁶⁷, —C(═O)NR⁶⁷R⁶⁸, —OC(═O)NR⁶⁷R⁶⁸, —NR⁶⁷C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl; and
R^{Y2} is hydrogen, halogen, —CN, —OR⁶⁷, —SR⁶⁷, —S(═O)R⁶⁹, —NO₂, —NR⁶⁷R⁶⁸, —S(═O)₂R⁶⁹, —NR⁶⁷S(═O)₂R⁶⁹, —S(═O)₂NR⁶⁷R⁶⁸, —C(═O)R⁶⁹, —OC(═O)R⁶⁹, —CO₂R⁶⁷, —OCO₂R⁶⁷, —C(═O)NR⁶⁷R⁶⁸, —OC(═O)NR⁶⁷R⁶⁸, —NR⁶⁷C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl; or

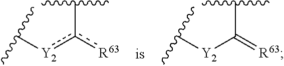

wherein:
Y² is NR^{Y3};
R⁶³ is O or S; and
R^{Y3} is hydrogen or optionally substituted alkyl;
R⁶⁰, R⁶¹, and R⁶² are independently hydrogen, halogen, —CN, —OR⁶⁷, —SR⁶⁷, —S(═O)R⁶⁹, —NO₂, —NR⁶⁷R⁶⁸, —S(═O)₂R⁶⁹, —NR⁶⁷S(═O)₂R⁶⁹, —S(═O)₂NR⁶⁷R⁶⁸, —C(═O)R⁶⁹, —OC(═O)R⁶⁹, -CO₂R⁶⁷, —OCO₂R⁶⁷, —C(═O)NR⁶⁷R⁶⁸, —OC(═O)NR⁶⁷R⁶⁸, —NR⁶⁷C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl;
R⁶⁴ is hydrogen, optionally substituted alkyl, optionally substituted alkenyl, optionally substituted alkynyl, optionally substituted heteroalkyl, optionally substituted cycloalkyl, optionally substituted heterocycloalkyl, optionally substituted aryl, or optionally substituted heteroaryl;
or R⁶³ and R⁶⁴ are taken together with the atoms to which they are attached to form an optionally substituted heterocycloalkyl or an optionally substituted heteroaryl;
R⁶⁵ is optionally substituted fused bicyclic heterocycloalkyl or optionally substituted fused bicyclic heteroaryl;
each R⁶⁶ is independently halogen, —CN, —OR⁶⁷, —SR⁶⁷, —S(═O)R⁶⁹, —NO₂, —NR⁶⁷R⁶⁸, —S(═O)₂R⁶⁹, —NR⁶⁷S(═O)₂R⁶⁹, —S(═O)₂NR⁶⁷R⁶⁸, —C(═O)R⁶⁹, —OC(═O)R⁶⁹, —CO₂R⁶⁷, —OCO₂R⁶⁷, —C(═O)NR⁶⁷R⁶⁸, —OC(═O)N R⁶⁷R⁶⁸, —NR⁶⁷C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl;
each R⁶⁷ and R⁶⁸ is independently hydrogen, optionally substituted —CN, —OR⁶⁷, —SR⁶⁷, —S(═O)R⁶⁹, —NO₂, —NR⁶⁷R⁶⁸, —S(═O)₂R⁶⁹, —NR⁶⁷S(═O)₂R⁶⁹, —S(═O)₂NR⁶⁷R⁶⁸, —C(═O)R⁶⁹, —OC(═O)R⁶⁹, —CO₂R⁶⁷, —OCO₂R⁶⁷, —C(═O)NR⁶⁷R⁶⁸, —OC(═O)N R⁶⁷R⁶⁸, —NR⁶⁷C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl;
or R⁶⁷ and R⁶⁸, together with the nitrogen atom to which they are attached, form an optionally substituted heterocycloalkyl or optionally substituted heteroaryl;
R⁶⁹ is optionally substituted alkyl, optionally substituted alkynyl, optionally substituted heteroalkyl, optionally substituted cycloalkyl, optionally substituted heterocycloalkyl, optionally substituted aryl, or optionally substituted heteroaryl; and
s3 is 0-3.

In some embodiments of the dual inhibitor of ASK1 and DYRK1A is:

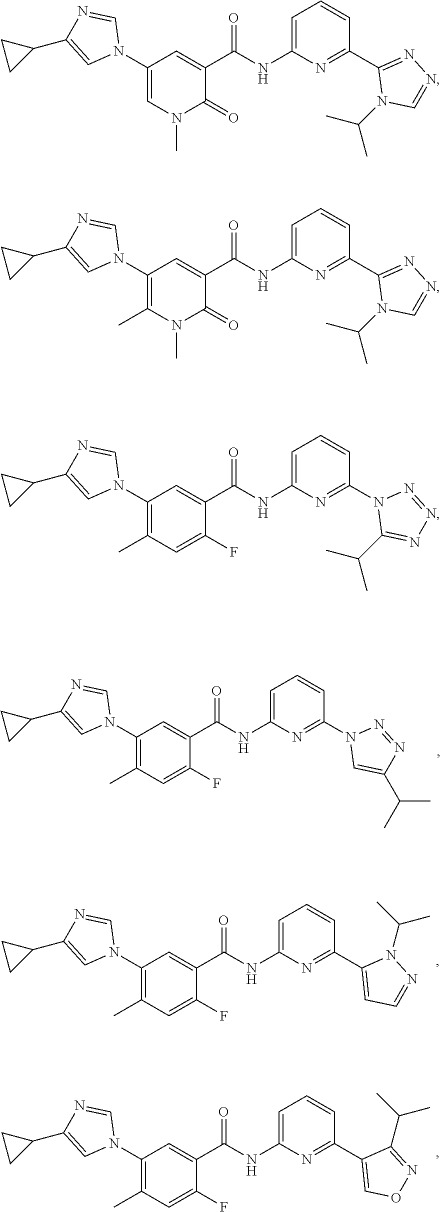 [SMILES: CC(C)n1cnnc1-c1cccc(NC(=O)c2cc(-n3cnc(C4CC4)c3)cn(C)c2=O)n1 ; Cc1c(-n2cnc(C3CC3)c2)cc(C(=O)Nc2cccc(-c3nncn3C(C)C)n2)c(=O)n1C ; Cc1cc(F)c(C(=O)Nc2cccc(-n3nnnc3C(C)C)n2)cc1-n1cnc(C2CC2)c1 ; Cc1cc(F)c(C(=O)Nc2cccc(-n3cc(C(C)C)nn3)n2)cc1-n1cnc(C2CC2)c1 ; Cc1cc(F)c(C(=O)Nc2cccc(-c3ccnn3C(C)C)n2)cc1-n1cnc(C2CC2)c1 ; Cc1cc(F)c(C(=O)Nc2cccc(-c3conc3C(C)C)n2)cc1-n1cnc(C2CC2)c1]

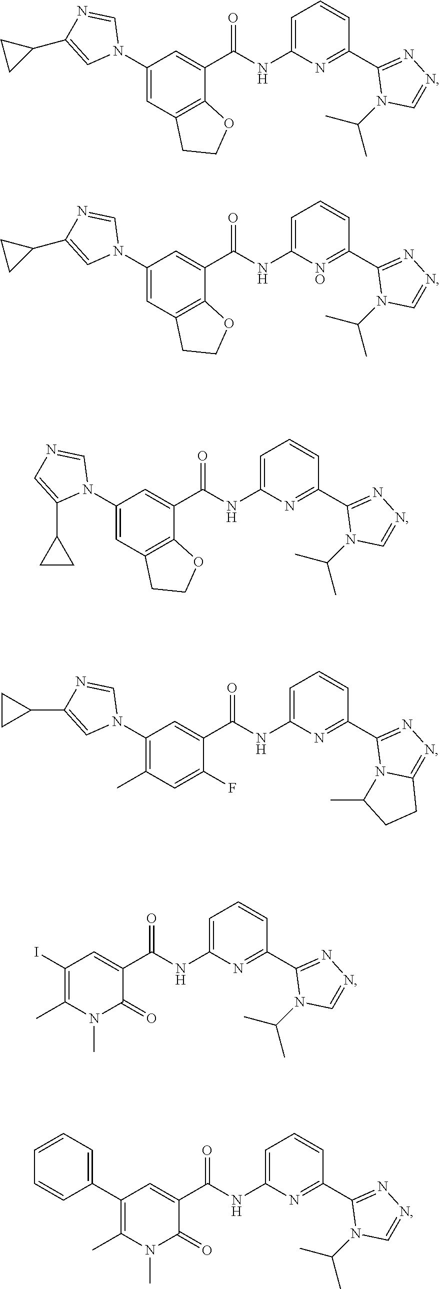

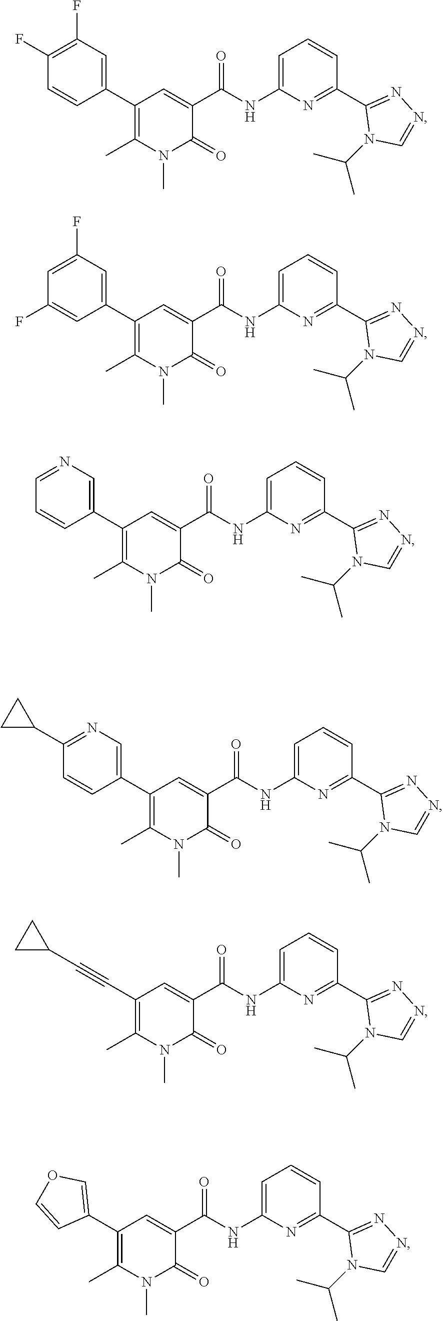 [SMILES: Cc1c(-c2ccc(F)c(F)c2)cc(C(=O)Nc2cccc(-c3nncn3C(C)C)n2)c(=O)n1C ; Cc1c(-c2cc(F)cc(F)c2)cc(C(=O)Nc2cccc(-c3nncn3C(C)C)n2)c(=O)n1C ; Cc1c(-c2cccnc2)cc(C(=O)Nc2cccc(-c3nncn3C(C)C)n2)c(=O)n1C ; Cc1c(-c2ccc(C3CC3)nc2)cc(C(=O)Nc2cccc(-c3nncn3C(C)C)n2)c(=O)n1C ; Cc1c(C#CC2CC2)cc(C(=O)Nc2cccc(-c3nncn3C(C)C)n2)c(=O)n1C ; Cc1c(-c2ccoc2)cc(C(=O)Nc2cccc(-c3nncn3C(C)C)n2)c(=O)n1C]

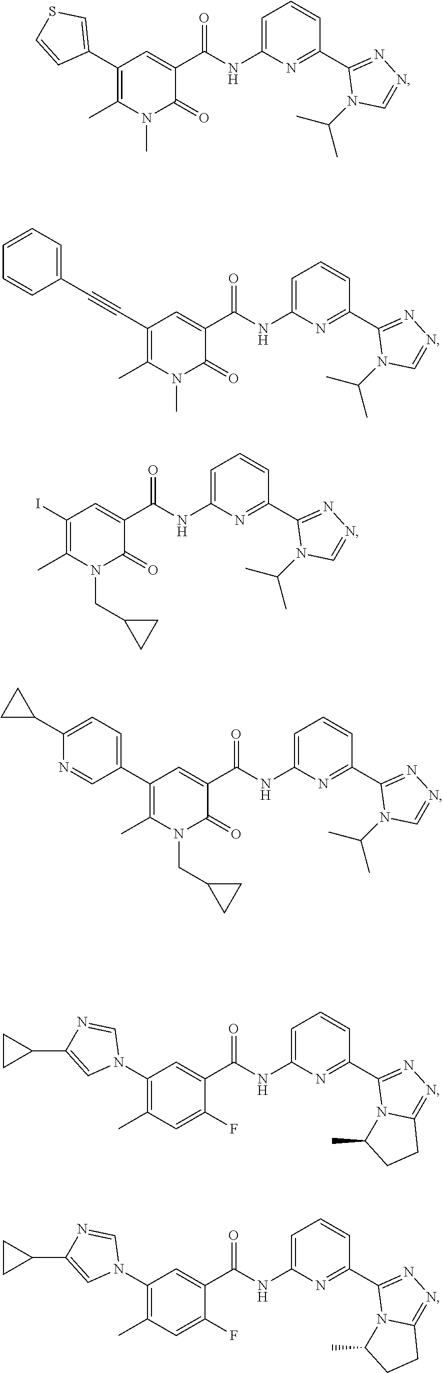 [SMILES: Cc1c(-c2ccsc2)cc(C(=O)Nc2cccc(-c3nncn3C(C)C)n2)c(=O)n1C ; Cc1c(C#Cc2ccccc2)cc(C(=O)Nc2cccc(-c3nncn3C(C)C)n2)c(=O)n1C ; Cc1c(I)cc(C(=O)Nc2cccc(-c3nncn3C(C)C)n2)c(=O)n1CC1CC1 ; Cc1c(-c2ccc(C3CC3)nc2)cc(C(=O)Nc2cccc(-c3nncn3C(C)C)n2)c(=O)n1CC1CC1 ; Cc1cc(F)c(C(=O)Nc2cccc(-c3nnc4n3[C@H](C)CC4)n2)cc1-n1cnc(C2CC2)c1 ; Cc1cc(F)c(C(=O)Nc2cccc(-c3nnc4n3[C@@H](C)CC4)n2)cc1-n1cnc(C2CC2)c1]

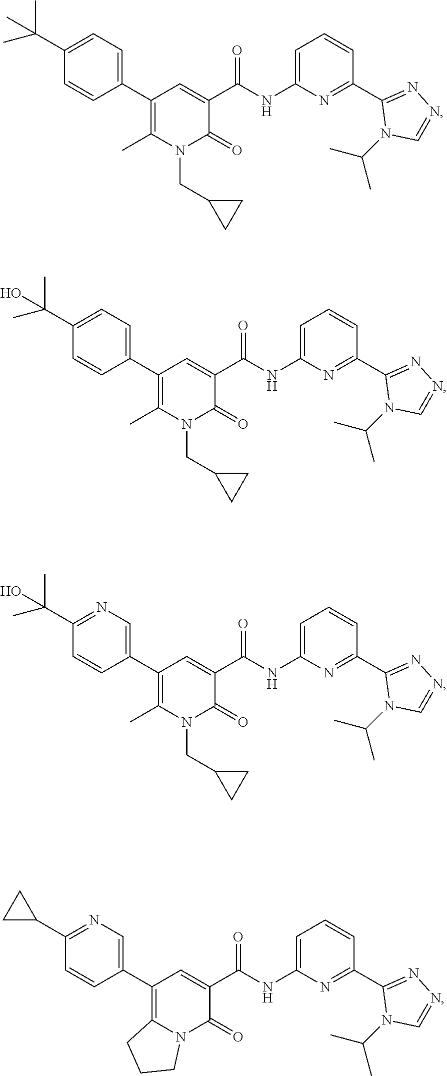 [SMILES: Cc1c(-c2ccc(C(C)(C)C)cc2)cc(C(=O)Nc2cccc(-c3nncn3C(C)C)n2)c(=O)n1CC1CC1 ; Cc1c(-c2ccc(C(C)(C)O)cc2)cc(C(=O)Nc2cccc(-c3nncn3C(C)C)n2)c(=O)n1CC1CC1 ; Cc1c(-c2ccc(C(C)(C)O)nc2)cc(C(=O)Nc2cccc(-c3nncn3C(C)C)n2)c(=O)n1CC1CC1 ; CC(C)n1cnnc1-c1cccc(NC(=O)c2cc(-c3ccc(C4CC4)nc3)c3n(c2=O)CCC3)n1]

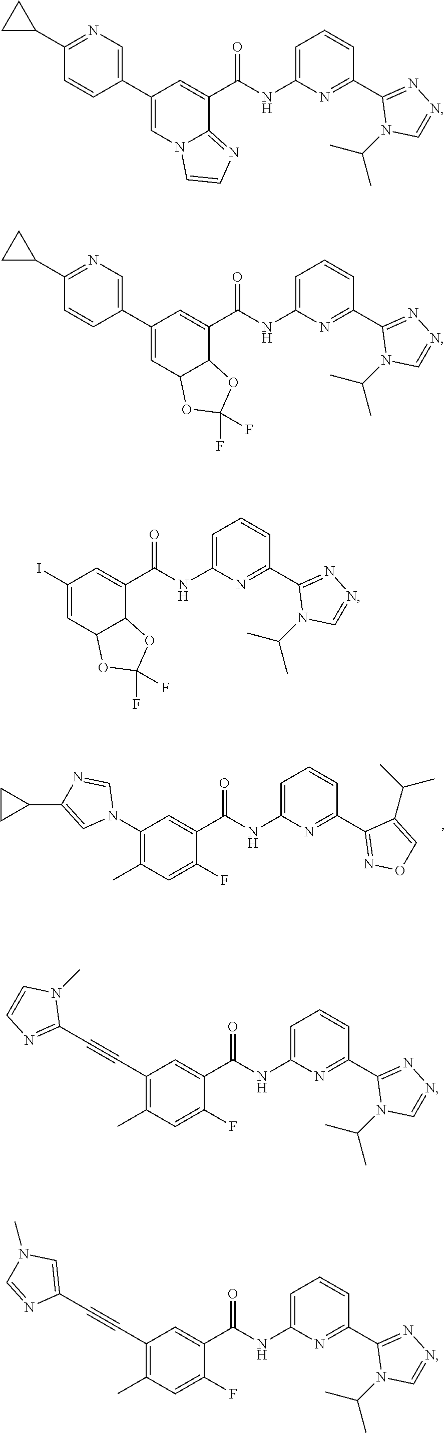 [SMILES: CC(C)n1cnnc1-c1cccc(NC(=O)c2cc(-c3ccc(C4CC4)nc3)cn3ccnc23)n1 ; CC(C)n1cnnc1-c1cccc(NC(=O)C2=CC(c3ccc(C4CC4)nc3)=CC3OC(F)(F)OC23)n1 ; CC(C)n1cnnc1-c1cccc(NC(=O)C2=CC(I)=CC3OC(F)(F)OC23)n1 ; Cc1cc(F)c(C(=O)Nc2cccc(-c3nocc3C(C)C)n2)cc1-n1cnc(C2CC2)c1 ; Cc1cc(F)c(C(=O)Nc2cccc(-c3nncn3C(C)C)n2)cc1C#Cc1nccn1C ; Cc1cc(F)c(C(=O)Nc2cccc(-c3nncn3C(C)C)n2)cc1C#Cc1cn(C)cn1]

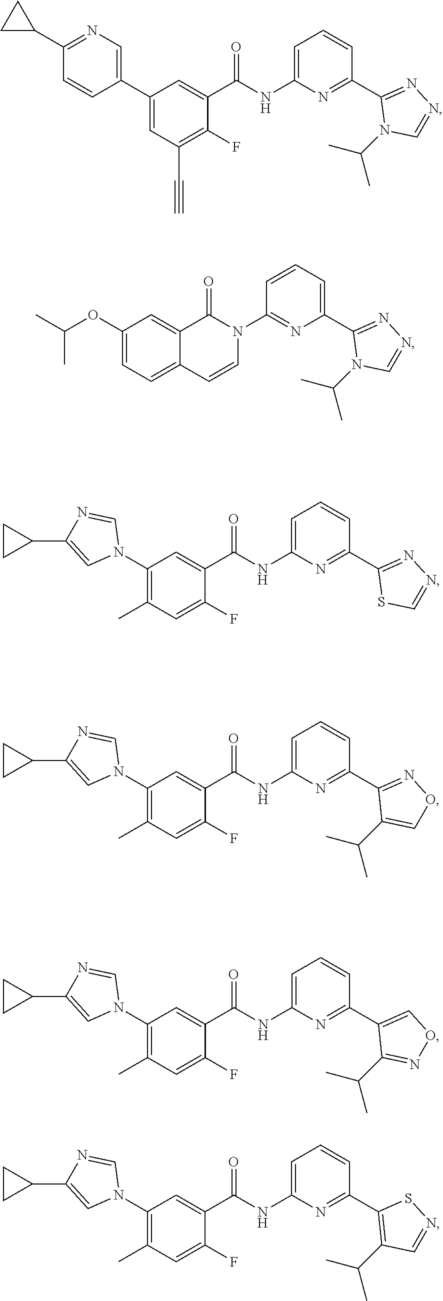 [SMILES: C#Cc1cc(-c2ccc(C3CC3)nc2)cc(C(=O)Nc2cccc(-c3nncn3C(C)C)n2)c1F ; CC(C)Oc1ccc2ccn(-c3cccc(-c4nncn4C(C)C)n3)c(=O)c2c1 ; Cc1cc(F)c(C(=O)Nc2cccc(-c3nncs3)n2)cc1-n1cnc(C2CC2)c1 ; Cc1cc(F)c(C(=O)Nc2cccc(-c3nocc3C(C)C)n2)cc1-n1cnc(C2CC2)c1 ; Cc1cc(F)c(C(=O)Nc2cccc(-c3conc3C(C)C)n2)cc1-n1cnc(C2CC2)c1 ; Cc1cc(F)c(C(=O)Nc2cccc(-c3sncc3C(C)C)n2)cc1-n1cnc(C2CC2)c1]

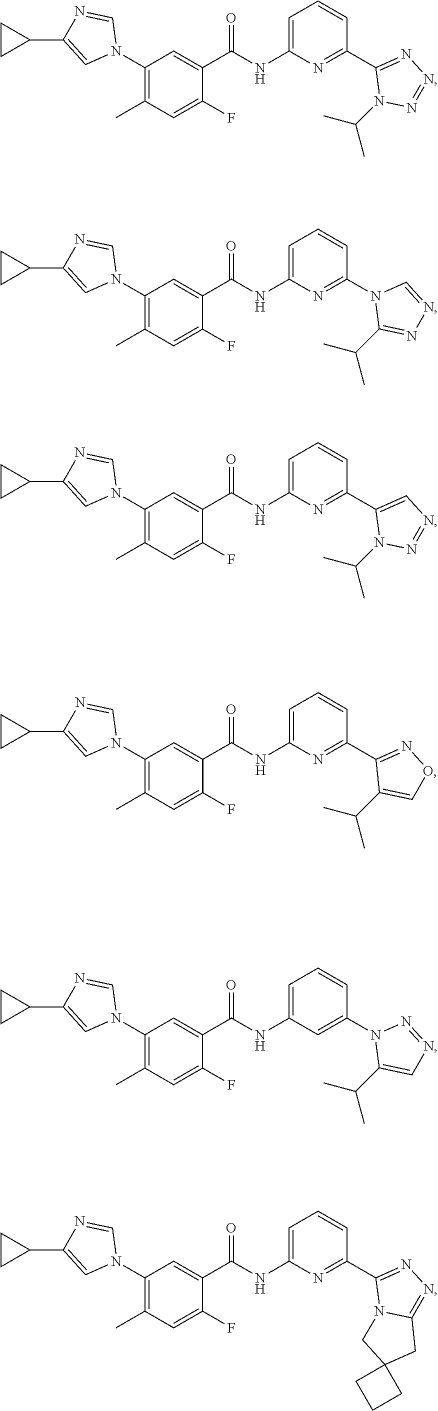 [SMILES: Cc1cc(F)c(C(=O)Nc2cccc(-c3nnnn3C(C)C)n2)cc1-n1cnc(C2CC2)c1 ; Cc1cc(F)c(C(=O)Nc2cccc(-n3cnnc3C(C)C)n2)cc1-n1cnc(C2CC2)c1 ; Cc1cc(F)c(C(=O)Nc2cccc(-c3cnnn3C(C)C)n2)cc1-n1cnc(C2CC2)c1 ; Cc1cc(F)c(C(=O)Nc2cccc(-c3nocc3C(C)C)n2)cc1-n1cnc(C2CC2)c1 ; Cc1cc(F)c(C(=O)Nc2cccc(-n3nncc3C(C)C)c2)cc1-n1cnc(C2CC2)c1 ; Cc1cc(F)c(C(=O)Nc2cccc(-c3nnc4n3CC3(CCC3)C4)n2)cc1-n1cnc(C2CC2)c1]

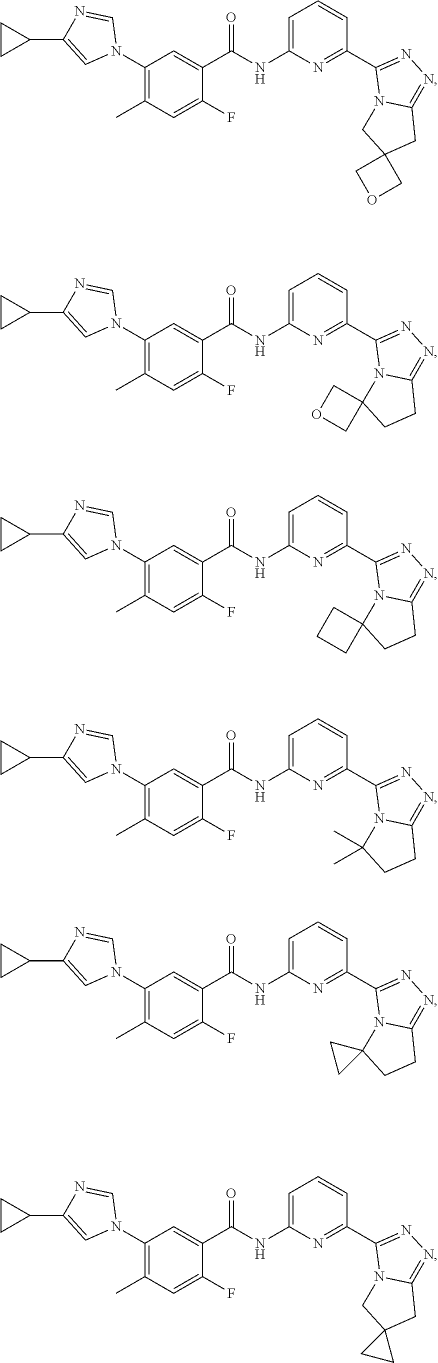 [SMILES: Cc1cc(F)c(C(=O)Nc2cccc(-c3nnc4n3CC3(COC3)C4)n2)cc1-n1cnc(C2CC2)c1 ; Cc1cc(F)c(C(=O)Nc2cccc(-c3nnc4n3C3(CC4)COC3)n2)cc1-n1cnc(C2CC2)c1 ; Cc1cc(F)c(C(=O)Nc2cccc(-c3nnc4n3C3(CCC3)CC4)n2)cc1-n1cnc(C2CC2)c1 ; Cc1cc(F)c(C(=O)Nc2cccc(-c3nnc4n3C(C)(C)CC4)n2)cc1-n1cnc(C2CC2)c1 ; Cc1cc(F)c(C(=O)Nc2cccc(-c3nnc4n3C3(CC4)CC3)n2)cc1-n1cnc(C2CC2)c1 ; Cc1cc(F)c(C(=O)Nc2cccc(-c3nnc4n3CC3(CC3)C4)n2)cc1-n1cnc(C2CC2)c1]

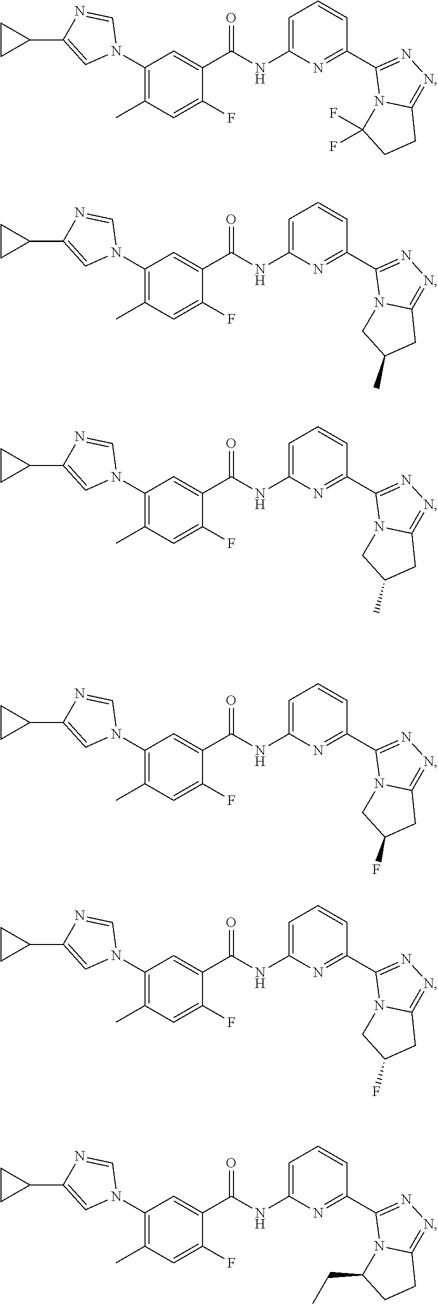 [SMILES: Cc1cc(F)c(C(=O)Nc2cccc(-c3nnc4n3C(F)(F)CC4)n2)cc1-n1cnc(C2CC2)c1 ; Cc1cc(F)c(C(=O)Nc2cccc(-c3nnc4n3C[C@H](C)C4)n2)cc1-n1cnc(C2CC2)c1 ; Cc1cc(F)c(C(=O)Nc2cccc(-c3nnc4n3C[C@@H](C)C4)n2)cc1-n1cnc(C2CC2)c1 ; Cc1cc(F)c(C(=O)Nc2cccc(-c3nnc4n3C[C@H](F)C4)n2)cc1-n1cnc(C2CC2)c1 ; Cc1cc(F)c(C(=O)Nc2cccc(-c3nnc4n3C[C@@H](F)C4)n2)cc1-n1cnc(C2CC2)c1 ; CC[C@@H]1CCc2nnc(-c3cccc(NC(=O)c4cc(-n5cnc(C6CC6)c5)c(C)cc4F)n3)n21]

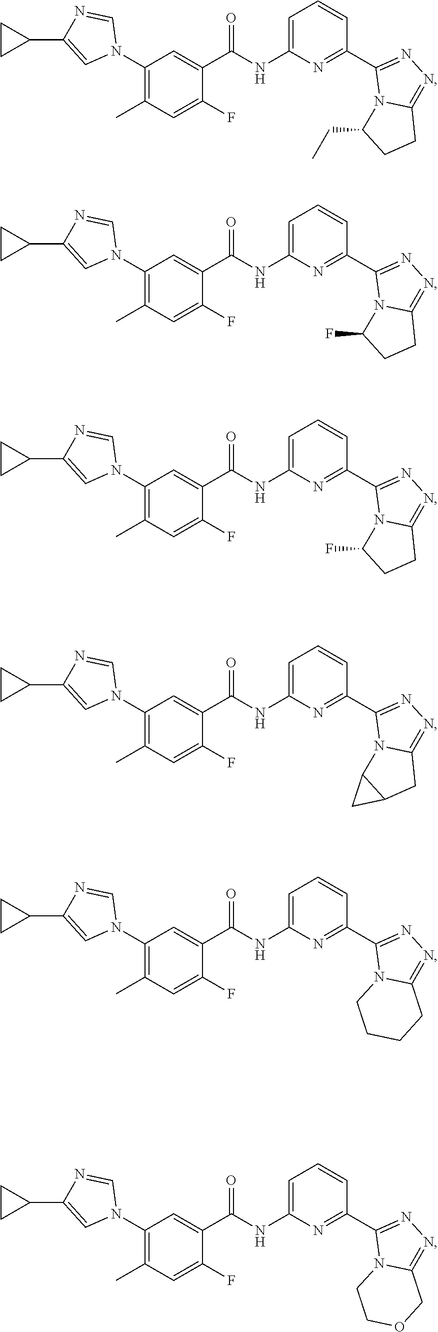 [SMILES: CC[C@H]1CCc2nnc(-c3cccc(NC(=O)c4cc(-n5cnc(C6CC6)c5)c(C)cc4F)n3)n21 ; Cc1cc(F)c(C(=O)Nc2cccc(-c3nnc4n3[C@H](F)CC4)n2)cc1-n1cnc(C2CC2)c1 ; Cc1cc(F)c(C(=O)Nc2cccc(-c3nnc4n3[C@@H](F)CC4)n2)cc1-n1cnc(C2CC2)c1 ; Cc1cc(F)c(C(=O)Nc2cccc(-c3nnc4n3C3CC3C4)n2)cc1-n1cnc(C2CC2)c1 ; Cc1cc(F)c(C(=O)Nc2cccc(-c3nnc4n3CCCC4)n2)cc1-n1cnc(C2CC2)c1 ; Cc1cc(F)c(C(=O)Nc2cccc(-c3nnc4n3CCOC4)n2)cc1-n1cnc(C2CC2)c1]

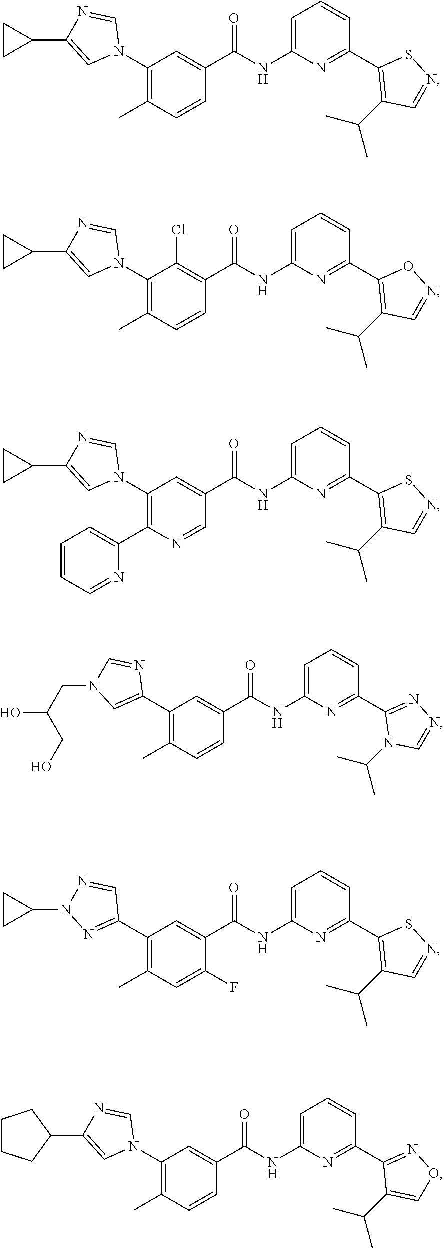 [SMILES: Cc1ccc(C(=O)Nc2cccc(-c3sncc3C(C)C)n2)cc1-n1cnc(C2CC2)c1 ; Cc1ccc(C(=O)Nc2cccc(-c3oncc3C(C)C)n2)c(Cl)c1-n1cnc(C2CC2)c1 ; CC(C)c1cnsc1-c1cccc(NC(=O)c2cnc(-c3ccccn3)c(-n3cnc(C4CC4)c3)c2)n1 ; Cc1ccc(C(=O)Nc2cccc(-c3nncn3C(C)C)n2)cc1-c1cn(CC(O)CO)cn1 ; Cc1cc(F)c(C(=O)Nc2cccc(-c3sncc3C(C)C)n2)cc1-c1cnn(C2CC2)n1 ; Cc1ccc(C(=O)Nc2cccc(-c3nocc3C(C)C)n2)cc1-n1cnc(C2CCCC2)c1]

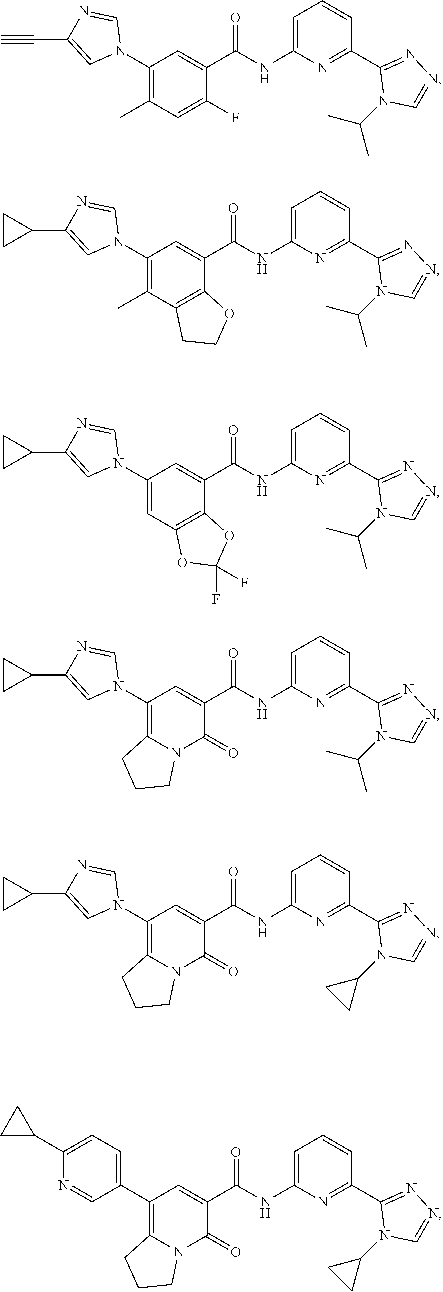 [SMILES: C#Cc1cn(-c2cc(C(=O)Nc3cccc(-c4nncn4C(C)C)n3)c(F)cc2C)cn1 ; Cc1c(-n2cnc(C3CC3)c2)cc(C(=O)Nc2cccc(-c3nncn3C(C)C)n2)c2c1CCO2 ; CC(C)n1cnnc1-c1cccc(NC(=O)c2cc(-n3cnc(C4CC4)c3)cc3c2OC(F)(F)O3)n1 ; CC(C)n1cnnc1-c1cccc(NC(=O)c2cc(-n3cnc(C4CC4)c3)c3n(c2=O)CCC3)n1 ; O=C(Nc1cccc(-c2nncn2C2CC2)n1)c1cc(-n2cnc(C3CC3)c2)c2n(c1=O)CCC2 ; O=C(Nc1cccc(-c2nncn2C2CC2)n1)c1cc(-c2ccc(C3CC3)nc2)c2n(c1=O)CCC2]

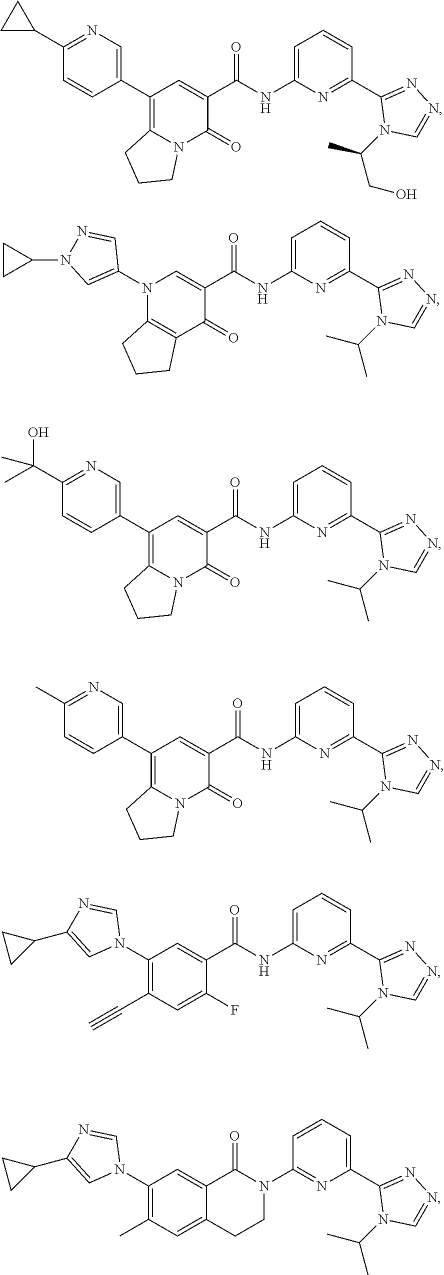 [SMILES: C[C@H](CO)n1cnnc1-c1cccc(NC(=O)c2cc(-c3ccc(C4CC4)nc3)c3n(c2=O)CCC3)n1 ; CC(C)n1cnnc1-c1cccc(NC(=O)c2cn(-c3cnn(C4CC4)c3)c3c(c2=O)CCC3)n1 ; CC(C)n1cnnc1-c1cccc(NC(=O)c2cc(-c3ccc(C(C)(C)O)nc3)c3n(c2=O)CCC3)n1 ; Cc1ccc(-c2cc(C(=O)Nc3cccc(-c4nncn4C(C)C)n3)c(=O)n3c2CCC3)cn1 ; C#Cc1cc(F)c(C(=O)Nc2cccc(-c3nncn3C(C)C)n2)cc1-n1cnc(C2CC2)c1 ; Cc1cc2c(cc1-n1cnc(C3CC3)c1)C(=O)N(c1cccc(-c3nncn3C(C)C)n1)CC2]

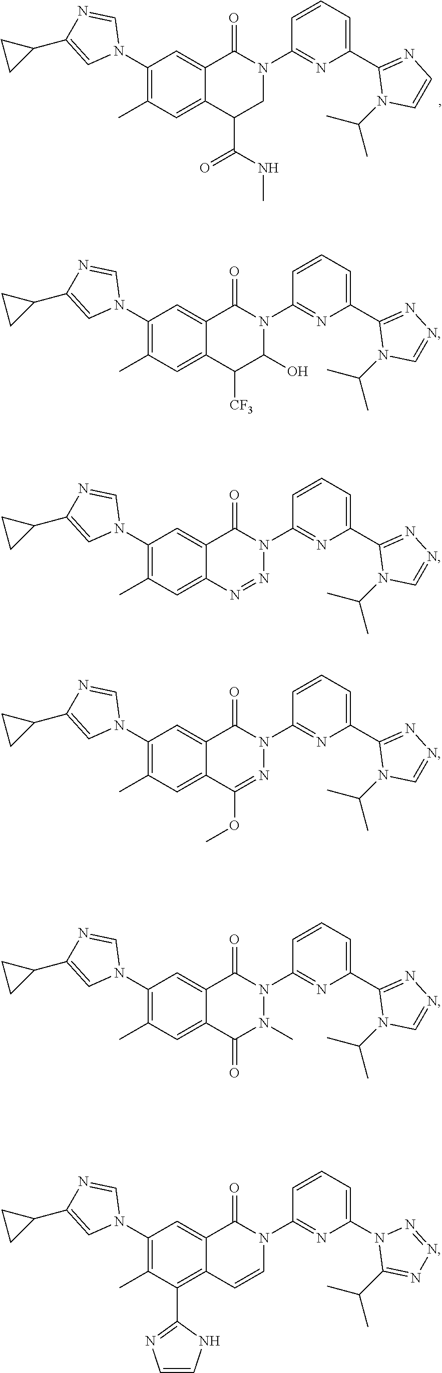 [SMILES: CNC(=O)C1CN(c2cccc(-c3nccn3C(C)C)n2)C(=O)c2cc(-n3cnc(C4CC4)c3)c(C)cc21 ; Cc1cc2c(cc1-n1cnc(C3CC3)c1)C(=O)N(c1cccc(-c3nncn3C(C)C)n1)C(O)C2C(F)(F)F ; Cc1cc2nnn(-c3cccc(-c4nncn4C(C)C)n3)c(=O)c2cc1-n1cnc(C2CC2)c1 ; COc1nn(-c2cccc(-c3nncn3C(C)C)n2)c(=O)c2cc(-n3cnc(C4CC4)c3)c(C)cc12 ; Cc1cc2c(=O)n(C)n(-c3cccc(-c4nncn4C(C)C)n3)c(=O)c2cc1-n1cnc(C2CC2)c1 ; Cc1c(-n2cnc(C3CC3)c2)cc2c(=O)n(-c3cccc(-n4nnnc4C(C)C)n3)ccc2c1-c1ncc[nH]1]

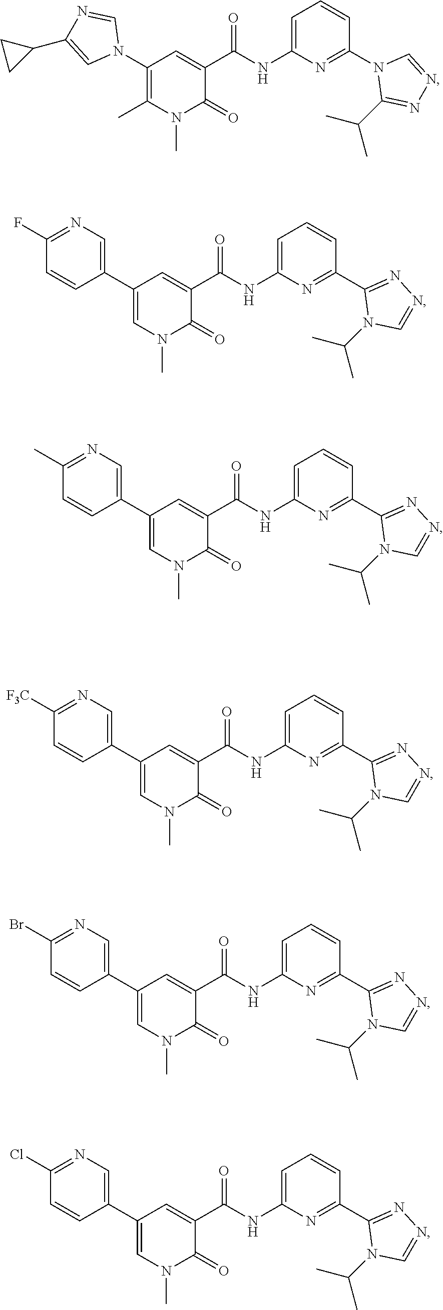 [SMILES: Cc1c(-n2cnc(C3CC3)c2)cc(C(=O)Nc2cccc(-n3cnnc3C(C)C)n2)c(=O)n1C ; CC(C)n1cnnc1-c1cccc(NC(=O)c2cc(-c3ccc(F)nc3)cn(C)c2=O)n1 ; Cc1ccc(-c2cc(C(=O)Nc3cccc(-c4nncn4C(C)C)n3)c(=O)n(C)c2)cn1 ; CC(C)n1cnnc1-c1cccc(NC(=O)c2cc(-c3ccc(C(F)(F)F)nc3)cn(C)c2=O)n1 ; CC(C)n1cnnc1-c1cccc(NC(=O)c2cc(-c3ccc(Br)nc3)cn(C)c2=O)n1 ; CC(C)n1cnnc1-c1cccc(NC(=O)c2cc(-c3ccc(Cl)nc3)cn(C)c2=O)n1]

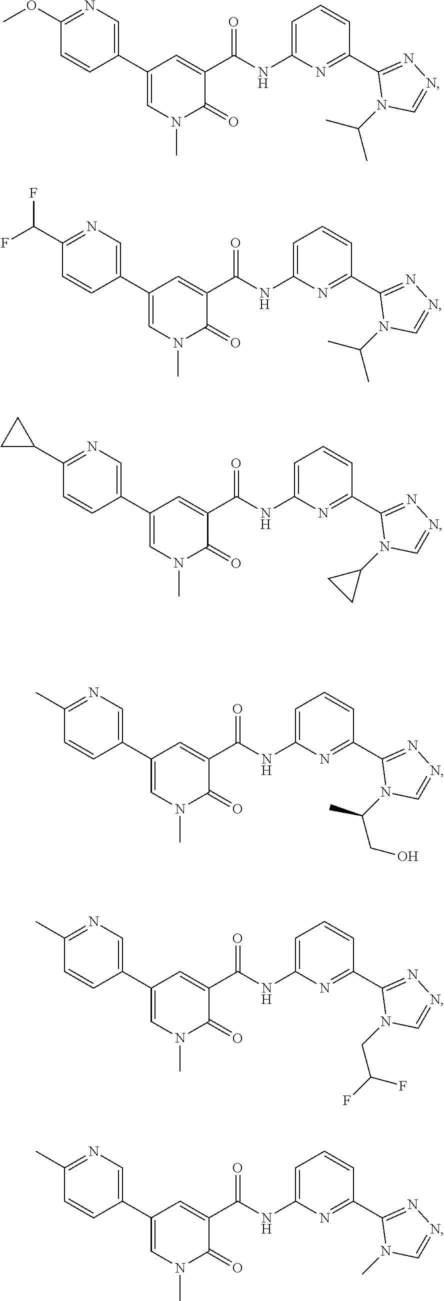 [SMILES: COc1ccc(-c2cc(C(=O)Nc3cccc(-c4nncn4C(C)C)n3)c(=O)n(C)c2)cn1 ; CC(C)n1cnnc1-c1cccc(NC(=O)c2cc(-c3ccc(C(F)F)nc3)cn(C)c2=O)n1 ; Cn1cc(-c2ccc(C3CC3)nc2)cc(C(=O)Nc2cccc(-c3nncn3C3CC3)n2)c1=O ; Cc1ccc(-c2cc(C(=O)Nc3cccc(-c4nncn4[C@H](C)CO)n3)c(=O)n(C)c2)cn1 ; Cc1ccc(-c2cc(C(=O)Nc3cccc(-c4nncn4CC(F)F)n3)c(=O)n(C)c2)cn1 ; Cc1ccc(-c2cc(C(=O)Nc3cccc(-c4nncn4C)n3)c(=O)n(C)c2)cn1]

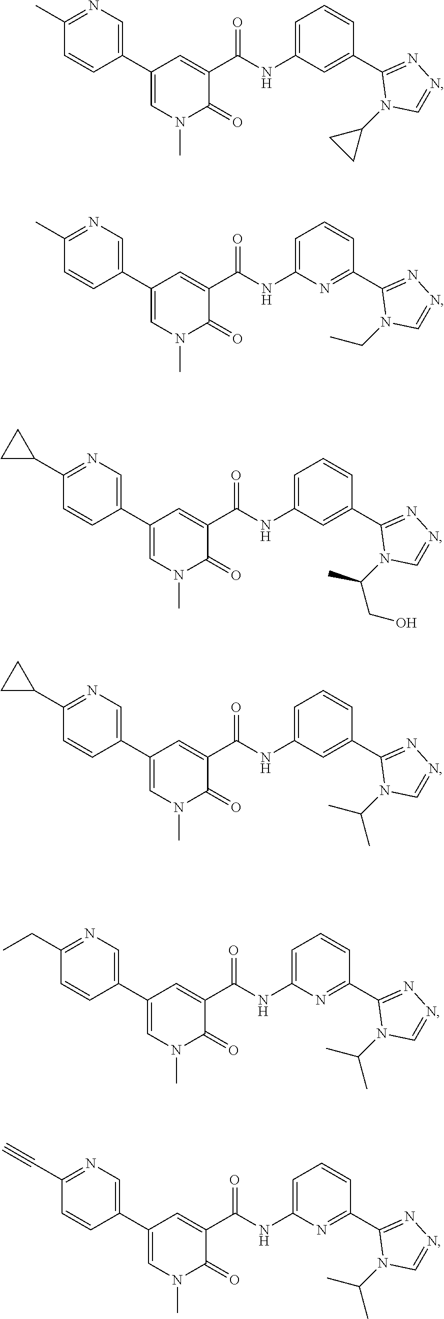 [SMILES: Cc1ccc(-c2cc(C(=O)Nc3cccc(-c4nncn4C4CC4)c3)c(=O)n(C)c2)cn1 ; CCn1cnnc1-c1cccc(NC(=O)c2cc(-c3ccc(C)nc3)cn(C)c2=O)n1 ; C[C@H](CO)n1cnnc1-c1cccc(NC(=O)c2cc(-c3ccc(C4CC4)nc3)cn(C)c2=O)c1 ; CC(C)n1cnnc1-c1cccc(NC(=O)c2cc(-c3ccc(C4CC4)nc3)cn(C)c2=O)c1 ; CCc1ccc(-c2cc(C(=O)Nc3cccc(-c4nncn4C(C)C)n3)c(=O)n(C)c2)cn1 ; C#Cc1ccc(-c2cc(C(=O)Nc3cccc(-c4nncn4C(C)C)n3)c(=O)n(C)c2)cn1]

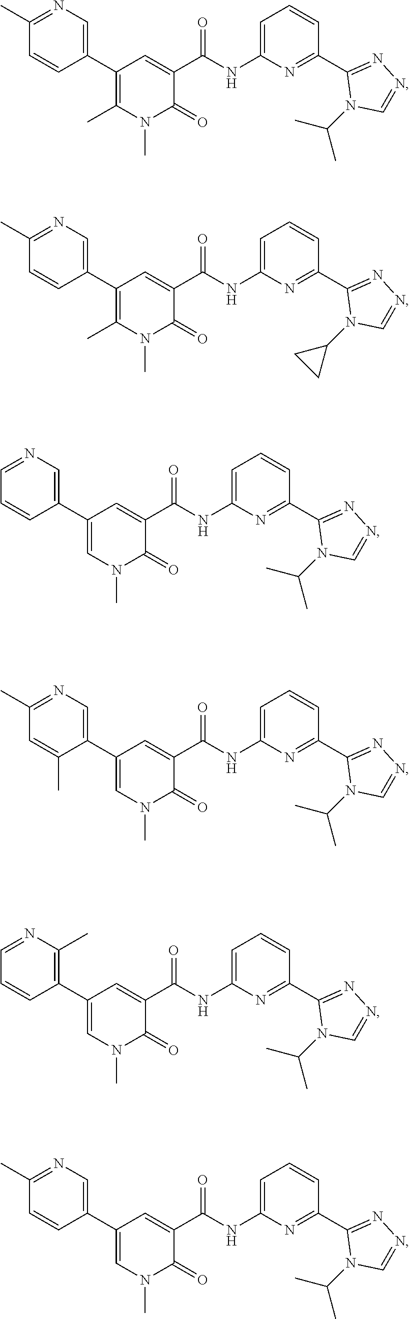 [SMILES: Cc1ccc(-c2cc(C(=O)Nc3cccc(-c4nncn4C(C)C)n3)c(=O)n(C)c2C)cn1 ; Cc1ccc(-c2cc(C(=O)Nc3cccc(-c4nncn4C4CC4)n3)c(=O)n(C)c2C)cn1 ; CC(C)n1cnnc1-c1cccc(NC(=O)c2cc(-c3cccnc3)cn(C)c2=O)n1 ; Cc1cc(C)c(-c2cc(C(=O)Nc3cccc(-c4nncn4C(C)C)n3)c(=O)n(C)c2)cn1 ; Cc1ncccc1-c1cc(C(=O)Nc2cccc(-c3nncn3C(C)C)n2)c(=O)n(C)c1 ; Cc1ccc(-c2cc(C(=O)Nc3cccc(-c4nncn4C(C)C)n3)c(=O)n(C)c2)cn1]

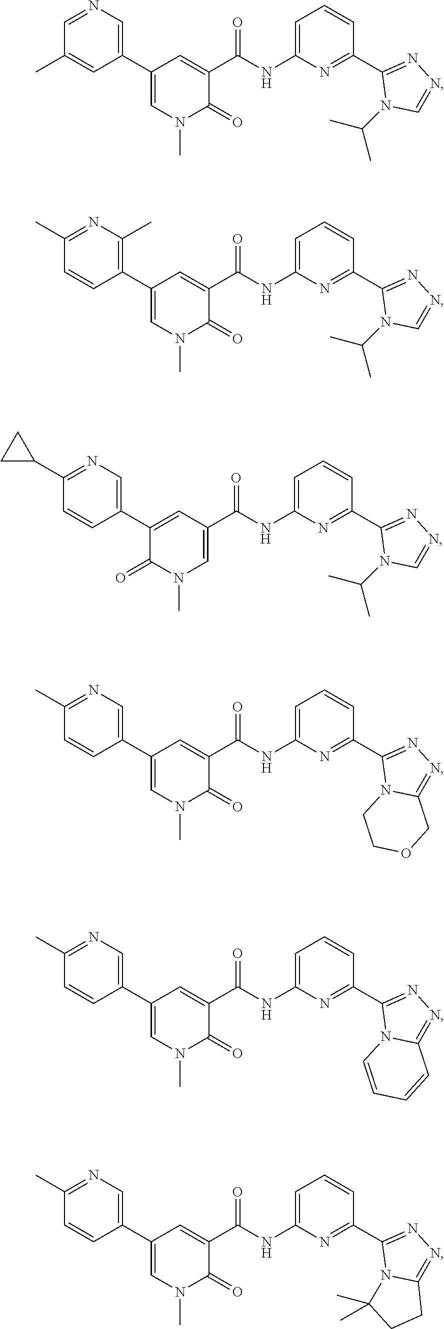 [SMILES: Cc1cncc(-c2cc(C(=O)Nc3cccc(-c4nncn4C(C)C)n3)c(=O)n(C)c2)c1 ; Cc1ccc(-c2cc(C(=O)Nc3cccc(-c4nncn4C(C)C)n3)c(=O)n(C)c2)c(C)n1 ; CC(C)n1cnnc1-c1cccc(NC(=O)c2cc(-c3ccc(C4CC4)nc3)c(=O)n(C)c2)n1 ; Cc1ccc(-c2cc(C(=O)Nc3cccc(-c4nnc5n4CCOC5)n3)c(=O)n(C)c2)cn1 ; Cc1ccc(-c2cc(C(=O)Nc3cccc(-c4nnc5ccccn45)n3)c(=O)n(C)c2)cn1 ; Cc1ccc(-c2cc(C(=O)Nc3cccc(-c4nnc5n4C(C)(C)CC5)n3)c(=O)n(C)c2)cn1]

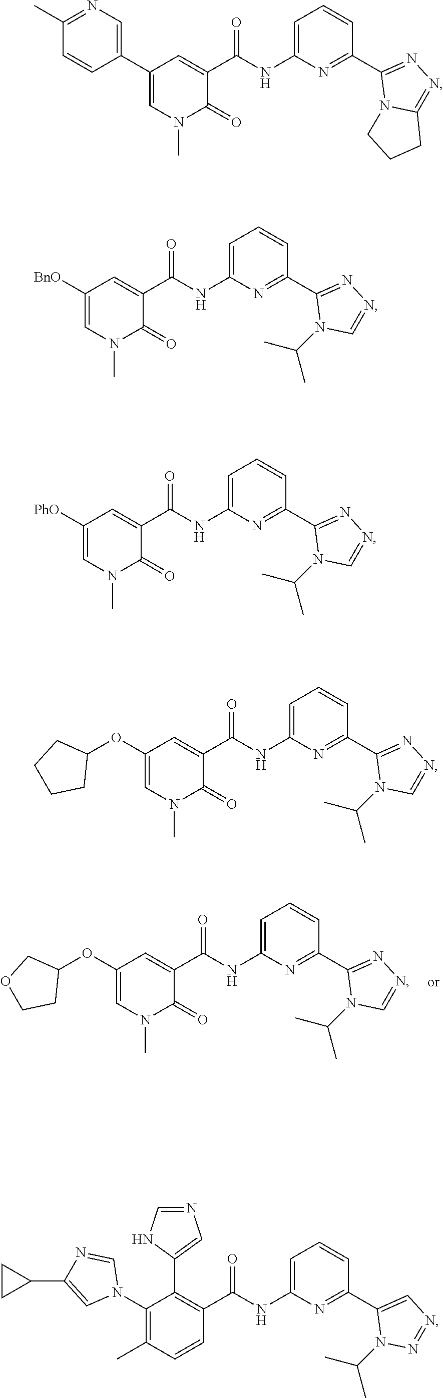 [SMILES: Cc1ccc(-c2cc(C(=O)Nc3cccc(-c4nnc5n4CCC5)n3)c(=O)n(C)c2)cn1 ; CC(C)n1cnnc1-c1cccc(NC(=O)c2cc(OCc3ccccc3)cn(C)c2=O)n1 ; CC(C)n1cnnc1-c1cccc(NC(=O)c2cc(Oc3ccccc3)cn(C)c2=O)n1 ; CC(C)n1cnnc1-c1cccc(NC(=O)c2cc(OC3CCCC3)cn(C)c2=O)n1 ; CC(C)n1cnnc1-c1cccc(NC(=O)c2cc(OC3CCOC3)cn(C)c2=O)n1 ; Cc1ccc(C(=O)Nc2cccc(-c3cnnn3C(C)C)n2)c(-c2cnc[nH]2)c1-n1cnc(C2CC2)c1]

or a pharmaceutically acceptable salt or solvate thereof.

In another aspect, presented herein is a method for treating cognitive impairment in a subject in need thereof, comprising administering to the subject a dual inhibitor of ASK1 and DYRK1A, wherein the dual inhibitor is a compound having the structure of Formula (IX), or a pharmaceutically acceptable salt or solvate thereof:

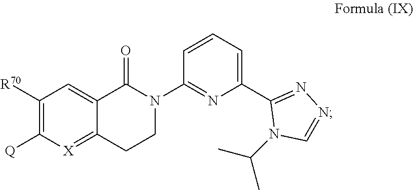

wherein:
X is selected from the group consisting of CH and N;
Q is selected from the group consisting of CH₃ and H; and

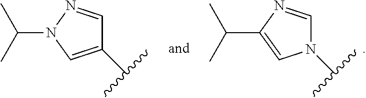

R⁷⁰ is selected from the group consisting of

In some embodiments, the dual inhibitor of Formula (IX) is 3-(4-cyclopropylimidazol-1-yl)-6-[6-(4-isopropyl-1,2,4-triazol-3-yl)-2-pyridyl]-7,8-dihydro-1,6-naphthyridin-5-one or 7-(4-cyclopropylimidazol-1-yl)-2-[6-(4-isopropyl-1,2,4-triazol-3-yl)-2-pyridyl]-6-methyl-3,4-dihydroisoquinolin-1-one, or a pharmaceutically acceptable salt or solvate thereof.

In another aspect, presented herein is a method for treating cognitive impairment in a subject in need thereof, comprising administering to the subject a dual inhibitor of ASK1 and DYRK1A, wherein the dual inhibitor is a compound having the structure of Formula (X), or a pharmaceutically acceptable salt or solvate thereof:

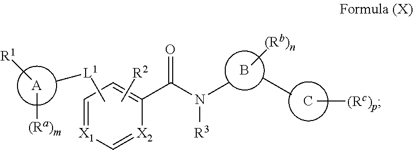

wherein:
Ring C is phenyl, 6-membered hetroaryl, or a 5-membered heteroaryl;
each R^{a} is independently hydrogen, deuterium, halogen, —CN, —OR⁵, —SR⁵, —S(═O)R⁴, —S(═O)₂R⁴, —S(═O)₂N(R⁵)₂, —R⁵S(═O)₂R⁴, —C(═O)R⁴, —OC(═O)R⁴, —CO₂R⁵, —OCO₂R⁴, —N(R⁵)₂, —OC(═O)N(R⁵)₂, —C(═O)N(R⁵)₂, —R⁵C(═O)R₄, —R⁵C(═O)OR⁴, —R⁵C(═O)N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, substituted or unsubstituted C₃-C₆cycloalkyl;
m is 0, 1, 2, or 3;
R¹ is hydrogen, deuterium, halogen, —CN, —OR⁵, —SR⁵, —S(═O)R⁴, —S(═O)₂R⁴, —N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, or substituted or unsubstituted —C₁-C₄alkylene-N(R⁵)₂;
L¹ is linker that is —X²—, L², -L²-X²—, —X²-L³-, or -L²-X²-L³-;
   X² is —O—, —S—, —S(═O)—, —S(═O)₂—, —S(═O)₂NR⁶—, —C(═O)—, —C(═O)O—, —C(═O)NR⁶—, —OC(═O)NR⁶—, —NR⁶C(═O)O, —NR⁶C(═O)NR⁶—, —OC(═O)—, —NR⁶C(═O)—, —NR⁶S(═O)₂—, or —NR⁶—; R⁶ is H, C₁-C₆alkyl, C₁-C₆fluoroalkyl, or C₁-C₆deuteroalkyl;
L² is substituted or unsubstituted C₁-C₄alkylene, substituted or unsubstituted C₂-C₄alkenylene or substituted or unsubstituted C₂-C₄alkynylene;
L³ is C₁-C₄alkylene;
X¹ is CR² or N;
X² is CR² or N;
each R² is independently hydrogen, deuterium, halogen, —CN, —OR⁵, —SR⁵, —S(═O)R⁴, —S(═O)₂R⁴, —N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, substituted or unsubstituted C3-C6cycloalkyl;
R³ is H, substituted or unsubstituted C₁-C₆alkyl, substituted or unsubstituted C₁-C₆fluoroalkyl, or substituted or unsubstituted C₁-C₆deuteroalkyl;
Ring D is a 6-membered heteroaryl, phenyl, or a 5-membered heteroaryl;
each R^{b} is independently hydrogen, deuterium, halogen, —CN, —OR⁵, —SR^{S}, —S(═O)R⁴, —S(═O)₂R⁴, —S(═O)₂N(R⁵)₂, —R⁵S(═O)₂R⁴, —C(═O)R⁴, —OC(═O)R⁴, —CO₂R⁵, —OCO₂R⁴, —N(R⁵)₂, —OC(═O)N(R⁵)₂, —R⁵C(═O)R⁴, —R²C(═O)OR⁴, —C(═O)N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, substituted or unsubstituted C₃-C₆cycloalkyl;
n is 0, 1, 2, 3, or 4;
Ring E is a 5-membered heteroaryl;
each R^{c} is independently hydrogen, deuterium, halogen, —CN, —OR⁵, —SR⁵, —S(═O)R⁴, —S(═O)₂R⁴, —S(═O)₂N(R⁵)₂, —NR⁵S(═O)₂R⁴, —C(═O)R⁴, —OC(═O)R⁴, —CO₂R⁵, —OCO₂R⁴, —N(R⁵)₂, —OC(═O)N(R⁵)₂, —NR⁵C(═O)R⁴, —NR⁵C(═O)OR⁴, —C(═O)N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, or substituted or unsubstituted C₃-C₆cycloalkyl;
p is 0, 1, 2, or 3;
each R⁴ is independently selected from C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, substituted or unsubstituted benzyl and substituted or unsubstituted heteroaryl;
each R⁵ is independently selected from hydrogen, C₁-C₆alkyl, C₁-C₆fluoroalkyl, C₁-C₆deuteroalkyl, C₁-C₆heteroalkyl, substituted or unsubstituted C₃-C₁ₒcycloalkyl, substituted or unsubstituted C₂-C₁₀heterocycloalkyl, substituted or unsubstituted aryl, substituted or unsubstituted benzyl and substituted or unsubstituted heteroaryl;
or two R⁵ on the same N atom are taken together with the N atom to which they are attached to a substituted or unsubstituted N-containing heterocycle.

In some embodiments of the dual inhibitor of ASK1 and DYRK1A is:
N-(6-(4-Isopropyl-4H-1,2,4-triazol-3-yl)pyridin-2-yl)-3-phenoxybenzamide;
N-(6-(4-Isopropyl-4H-1,2,4-triazol-3-yl)pyridin-2-yl)-3-(pyridin-2-yloxy)benzamide;
N-(6-(4-Isopropyl-4H-1,2,4-triazol-3-yl)pyridin-2-yl)-3-(pyridin-3-yloxy)benzamide;
N-(6-(4-isopropyl-4H-1,2,4-triazol-3-yl)pyridin-2-yl)-3-(pyridin-4-yloxy)benzamide;
N-(6-(4-isopropyl-4H-1,2,4-triazol-3-yl)pyridin-2-yl)-3-(pyrimidin-4-yloxy)benzamide;
N-(6-(4-Isopropyl-4H-1,2,4-triazol-3-yl)pyridin-2-yl)-3-(pyrimidin-5-yloxy)benzamide;
N-(6-(4-Isopropyl-4H-1,2,4-triazol-3-yl)pyridin-2-yl)-3-(pyrimidin-2-yloxy)benzamide;
N-(6-(4-Isopropyl-4H-1,2,4-triazol-3-yl)pyridin-2-yl)-3-(pyrazin-2-yloxy)benzamide;
N-(6-(4-Isopropyl-4H-1,2,4-triazol-3-yl)pyridin-2-yl)-3-(pyridazin-3-yloxy)benzamide;
N-(6-(4-Isopropyl-4H-1,2,4-triazol-3-yl)pyridin-2-yl)-3-(pyridazin-4-yloxy)benzamide;
3-((6-Cyclopropylpyridin-2-yl)oxy)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-((6-(trifluoromethyl)pyridin-2-yl)oxy)benzamide;
N-(6-(4-Isopropyl-4H-1,2,4-triazol-3-yl)pyridin-2-yl)-3-((6-methoxypyridin-2-yl)oxy)benzamide;
N-(6-(4-Isopropyl-4H-1,2,4-triazol-3-yl)pyridin-2-yl)-3-((6-(pyrrolidin-1-yl)pyridin-2-yl)oxy)benzamide;
3-46-(Dimethylamino)pyridin-2-yl)oxy)-N-(6-(4-isopropyl-4H-1,2,4-triazol-3-yl)pyridin-2-yl)benzamide;
3-((6-(Dimethylamino)pyridin-3-yl)oxy)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-((5,6,7,8-tetrahydro-1,8-naphthyridin-2-yl)oxy)benzamide;
6-(3-46-(4-Isopropyl-4H-1,2,4-triazol-3-yl)pyridin-2-yl)carbamoyl)phenoxy)picolinic acid;
3-((5-Cyclopropylpyridin-2-yl)oxy)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-((5-(trifluoromethyl)pyridin-2-yl)oxy)benzamide;
N-(6-(4-Isopropyl-4H-1,2,4-triazol-3-yl)pyridin-2-yl)-3-((5-methoxypyridin-2-yl)oxy)benzamide;
6-(3-46-(4-Isopropyl-4H-1,2,4-triazol-3-yl)pyridin-2-yl)carbamoyl)phenoxy)nicotinic acid;
3-((4-Cyano-6-(trifluoromethyl)pyridin-2-yl)oxy)-N-(6-(4-isopropyl-4H-1,2,4-triazol-3-yl)pyridin-2-yl)benzamide;
2-(3-46-(4-Isopropyl-4H-1,2,4-triazol-3-yl)pyridin-2-yl)carbamoyl)phenoxy)-6-(trifluoromethypisonicotinic acid;
3-((2-Cyclopropylpyrimidin-4-yl)oxy)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-((2-(trifluoromethyl)pyrimidin-4-yl)oxy)benzamide;
N-(6-(4-Isopropyl-4H-1,2,4-triazol-3-yl)pyridin-2-yl)-3-((2-methoxypyrimidin-4-yl)oxy)benzamide;
N-(6-(4-Isopropyl-4H-1,2,4-triazol-3-yl)pyridin-2-yl)-3-42-(2-(pyrrolidin-1-yl)ethoxy)pyrimidin-4-yl)oxy)benzamide;
N-(6-(4-Isopropyl-4H-1,2,4-triazol-3-yl)pyridin-2-yl)-3-((2-(pyrrolidin-1-yl)pyrimidin-4-yl)oxy)benzamide;
4-(3-46-(4-Isopropyl-4H-1,2,4-triazol-3-yl)pyridin-2-yl)carbamoyl)phenoxy)pyrimidine-2-carboxylic acid;
3-((2-Cyclopropylpyrimidin-5-yl)oxy)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-((2-(trifluoromethyl)pyrimidin-5-yl)oxy)benzamide;
N-(6-(4-Isopropyl-4H-1,2,4-triazol-3-yl)pyridin-2-yl)-3-((2-methoxypyrimidin-5-yl)oxy)benzamide;
N-(6-(4-Isopropyl-4H-1,2,4-triazol-3-yl)pyridin-2-yl)-3-42-(2-(pyrrolidin-1-yl)ethoxy)pyrimidin-5-yl)oxy)benzamide;
N-(6-(4-Isopropyl-4H-1,2,4-triazol-3-yl)pyridin-2-yl)-3-((2-(pyrrolidin-1-yl)pyrimidin-5-yl)oxy)benzamide;
5-(3-46-(4-Isopropyl-4H-1,2,4-triazol-3-yl)pyridin-2-yl)carbamoyl)phenoxy)pyrimidine-2-carboxylic acid;
N-(6-(4-Isopropyl-4H-1,2,4-triazol-3-yl)pyridin-2-yl)-3-(phenylamino)benzamide;
N-(6-(4-Isopropyl-4H-1,2,4-triazol-3-yl)pyridin-2-yl)-3-(pyridin-2-ylamino)benzamide;
N-(6-(4-Isopropyl-4H-1,2,4-triazol-3-yl)pyridin-2-yl)-3-(pyridin-3-ylamino)benzamide;
N-(6-(4-Isopropyl-4H-1,2,4-triazol-3-yl)pyridin-2-yl)-3-(pyridin-4-ylamino)benzamide;
N-(6-(4-Isopropyl-4H-1,2,4-triazol-3-yl)pyridin-2-yl)-3-(pyrimidin-4-ylamino)benzamide;
N-(6-(4-Isopropyl-4H-1,2,4-triazol-3-yl)pyridin-2-yl)-3-(pyrimidin-5-ylamino)benzamide;
N-(6-(4-Isopropyl-4H-1,2,4-triazol-3-yl)pyridin-2-yl)-3-(pyrimidin-2-ylamino)benzamide;
N-(6-(4-Isopropyl-4H-1,2,4-triazol-3-yl)pyridin-2-yl)-3-(pyrazin-2-ylamino)benzamide;
N-(6-(4-Isopropyl-4H-1,2,4-triazol-3-yl)pyridin-2-yl)-3-(pyridazin-3-ylamino)benzamide;
N-(6-(4-Isopropyl-4H-1,2,4-triazol-3-yl)pyridin-2-yl)-3-(pyridazin-4-ylamino)benzamide;
3-((6-Cyclopropylpyridin-2-yl)amino)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-((6-(trifluoromethyl)pyridin-2-yl)amino)benzamide;
N-(6-(4-Isopropyl-4H-1,2,4-triazol-3-yl)pyridin-2-yl)-3-((6-methoxypyridin-2-yl)amino)benzamide;
N-(6-(4-Isopropyl-4H-1,2,4-triazol-3-yl)pyridin-2-yl)-3-((6-(pyrrolidin-1-yl)pyridin-2-yl)amino)benzamide;
N-(6-(4-Isopropyl-4H-1,2,4-triazol-3-yl)pyridin-2-yl)-3-((5,6,7,8-tetrahydro-1,8-naphthyridin-2-yl)amino)benzamide;
6-43-46-(4-Isopropyl-4H-1,2,4-triazol-3-yl)pyridin-2-yl)carbamoyl)phenyl)amino)picolinic acid;
3-((5-Cyclopropylpyridin-2-yl)amino)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-((5-(trifluoromethyl)pyridin-2-yl)amino)benzamide;
N-(6-(4-Isopropyl-4H-1,2,4-triazol-3-yl)pyridin-2-yl)-3-((5-methoxypyridin-2-yl)amino)benzamide;
6-43-46-(4-Isopropyl-4H-1,2,4-triazol-3-yl)pyridin-2-yl)carbamoyl)phenyl)amino)nicotinic acid;
3-((2-Cyclopropylpyrimidin-4-yl)amino)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-((2-(trifluoromethyl)pyrimidin-4-yl)amino)benzamide;
N-(6-(4-Isopropyl-4H-1,2,4-triazol-3-yl)pyridin-2-yl)-3-((2-methoxypyrimidin-4-yl)amino)benzamide;
N-(6-(4-Isopropyl-4H-1,2,4-triazol-3-yl)pyridin-2-yl)-3-((2-(2-(pyrrolidin-1-yl)ethoxy)pyrimidin-4-yl)amino)benzamide;
N-(6-(4-Isopropyl-4H-1,2,4-triazol-3-yl)pyridin-2-yl)-3-((2-(pyrrolidin-1-yl)pyrimidin-4-yl)amino)benzamide;
4-43-46-(4-Isopropyl-4H-1,2,4-triazol-3-yl)pyridin-2-yl)carbamoyl)phenyl)amino)pyrimidine-2-carboxylic acid;
3-((2-Cyclopropylpyrimidin-5-yl)amino)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-((2-(trifluoromethyl)pyrimidin-5-yl)amino)benzamide;
N-(6-(4-Isopropyl-4H-1,2,4-triazol-3-yl)pyridin-2-yl)-3-((2-methoxypyrimidin-5-yl)amino)benzamide;
N-(6-(4-Isopropyl-4H-1,2,4-triazol-3-yl)pyridin-2-yl)-3-((2-(2-(pyrrolidin-1-yl)ethoxy)pyrimidin-5-yl)amino)benzamide;
N-(6-(4-Isopropyl-4H-1,2,4-triazol-3-yl)pyridin-2-yl)-3-((2-(pyrrolidin-1-yl)pyrimidin-5-yl)amino)benzamide;
5-43-46-(4-Isopropyl-4H-1,2,4-triazol-3-yl)pyridin-2-yl)carbamoyl)phenyl)amino)pyrimidine-2-carboxylic acid;
3-((1H-Pyrrol-2-yl)amino)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-((1-methyl-1H-pyrrol-2-yl)amino)benzamide; 3-(Furan-2-ylamino)-N-(6-(4-isopropyl-4H-1,2,4-triazol-3-yl)pyridin-2-yl)benzamide;
N-(6-(4-Isopropyl-4H-1,2,44riazol-3-yl)pyridin-2-yl)-3-(thiophen-2-ylamino)benzamide;
N-(6-(4-Isopropyl-4H-1,2,4-triazol-3-yl)pyridin-2-yl)-3-((1-methyl-1H-pyrazol-5-yl)amino)benzamide;
N-(6-(4-Isopropyl-4H-1,2,4-triazol-3-yl)pyridin-2-yl)-3-((1-methyl-1H-pyrazol-3-yl)amino)benzamide;
3-((1H midazol-5-yl)amino)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-((1-methyl-1H-imidazol-5-yl)amino)benzamide;
N-(6-(4-Isopropyl-4H-1,2,4-triazol-3-yl)pyridin-2-yl)-3-((1-methyl-1H-imidazol-4-yl)amino)benzamide;
N-(6-(4-Isopropyl-4H-1,2,4-triazol-3-yl)pyridin-2-yl)-3-(oxazol-4-ylamino)benzamide;
N-(6-(4-Isopropyl-4H-1,2,4-triazol-3-yl)pyridin-2-yl)-3-(oxazol-5-ylamino)benzamide;
N-(6-(4-Isopropyl-4H-1,2,4-triazol-3-yl)pyridin-2-yl)-3-(isoxazol-5-ylamino)benzamide;
N-(6-(4-Isopropyl-4H-1,2,4-triazol-3-yl)pyridin-2-yl)-3-(isoxazol-3-ylamino)benzamide;
3-((1,3,4-Oxadiazol-2-yl)amino)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-(thiazol-5-ylamino)benzamide;
3-((1,3,4-Thiadiazol-2-yl)amino)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-(phenylthio)benzamide;
N-(6-(4-Isopropyl-4H-1,2,4-triazol-3-yl)pyridin-2-yl)-3-(pyridin-2-ylthio)benzamide;
N-(6-(4-Isopropyl-4H-1,2,4-triazol-3-yl)pyridin-2-yl)-3-(pyridin-3-ylthio)benzamide;
N-(6-(4-Isopropyl-4H-1,2,4-triazol-3-yl)pyridin-2-yl)-3-(pyridin-4-ylthio)benzamide;
N-(6-(4-Isopropyl-4H-1,2,4-triazol-3-yl)pyridin-2-yl)-3-(pyrimidin-4-ylthio)benzamid;
N-(6-(4-Isopropyl-4H-1,2,4-triazol-3-yl)pyridin-2-yl)-3-(pyrimidin-5-ylthio)benzamide;
N-(6-(4-Isopropyl-4H-1,2,4-triazol-3-yl)pyridin-2-yl)-3-(pyrimidin-2-ylthio)benzamide;
N-(6-(4-Isopropyl-4H-1,2,4-triazol-3-yl)pyridin-2-yl)-3-(pyrazin-2-ylthio)benzamide;
N-(6-(4-Isopropyl-4H-1,2,4-triazol-3-yl)pyridin-2-yl)-3-(pyridazin-3-ylthio)benzamide;
N-(6-(4-Isopropyl-4H-1,2,4-triazol-3-yl)pyridin-2-yl)-3-(pyridazin-4-ylthio)benzamide;
3-((6-Cyclopropylpyridin-2-yl)thio)-N-(6-(4 sopropyl-4H-1,2,4-triazol-3-yppyridin-2-yl)benzamide;
N-(6-(4-Isopropyl-4H-1,2,4-triazol-3-yl)pyridin-2-yl)-3-((6-(trifluoromethyl)pyridin-2-yl)thio)benzamide;
N-(6-(4-Isopropyl-4H-1,2,4-triazol-3-yl)pyridin-2-yl)-3-((6-methoxypyridin-2-yl)thio)benzamide;
N-(6-(4-Isopropyl-4H-1,2,4-triazol-3-yl)pyridin-2-yl)-3-((6-(pyrrolidin-1-yl)pyridin-2-yl)thio)benzamide;
N-(6-(4-Isopropyl-4H-1,2,4-triazol-3-yl)pyridin-2-yl)-3-((5,6,7,8-tetrahydro-1,8-naphthyridin-2-yl)thio)benzamide;
6-43-46-(4-Isopropyl-4H-1,2,4-triazol-3-yl)pyridin-2-yl)carbamoyl)phenyl)thio)picolinic acid;
3-((5-Cyclopropylpyridin-2-yl)thio)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-((5-(trifluoromethyl)pyridin-2-yl)thio)benzamide;
N-(6-(4-Isopropyl-4H-1,2,4-triazol-3-yl)pyridin-2-yl)-3-((5-methoxypyridin-2-yl)thio)benzamide;
6-43-46-(4-Isopropyl-4H-1,2,4-triazol-3-yl)pyridin-2-yl)carbamoyl)phenyl)thio)nicotinic acid;
3-((2-Cyclopropylpyrimidin-4-yl)thio)-N-(6-(4-isopropyl-4H-1,2,4-triazol-3-yl)pyridin-2-yl)benzamide;
N-(6-(4-Isopropyl-4H-1,2,4-triazol-3-yl)pyridin-2-yl)-3-(2-(trifluoromethyl)pyrimidin-4-yl)thio)benzamide;
N-(6-(4-Isopropyl-4H-1,2,4-triazol-3-yl)pyridin-2-yl)-3-(2-methoxypyrimidin-4-yl)thio)benzamide;
N-(6-(4-Isopropyl-4H-1,2,4-triazol-3-yl)pyridin-2-yl)-3-(2-(2-(pyrrolidin-1-yl)ethoxy)pyrimidin-4-yl)thio)benzamide;
N-(6-(4-Isopropyl-4H-1,2,4-triazol-3-yl)pyridin-2-yl)-3-(2-(pyrrolidin-1-yl)pyrimidin-4-yl)thio)benzamide;
4-43-46-(4-Isopropyl-4H-1,2,4-triazol-3-yl)pyridin-2-yl)carbamoyl)phenyl)thio)pyrimidine-2-carboxylic acid;
3-((2-Cyclopropylpyrimidin-5-yl)thio)-N-(6-(4-isopropy-4H-1 ,2,4-triazol-3-yl)pyridin-2-yl)benzamide;
N-(6-(4-Isopropyl-4H-1,2,4-triazol-3-yl)pyridin-2-yl)-3-(2-(trifluoromethyl)pyrimidin-5-yl)thio)benzamide;
N-(6-(4-Isopropyl-4H-1,2,4-triazol-3-yl)pyridin-2-yl)-3-(2-methoxypyrimidin-5-yl)thio)benzamide;
N-(6-(4-Isopropyl-4H-1,2,4-triazol-3-yl)pyridin-2-yl)-3-(2-(2-(pyrrolidin-1-yl)ethoxy)pyrimidin-5-yl)thio)benzamide;
N-(6-(4-Isopropyl-4H-1,2,4-triazol-3-yl)pyridin-2-yl)-3-(2-(pyrrolidin-1-yl)pyrimidin-5-yl)thio)benzamide;
5-43-46-(4-Isopropyl-4H-1,2,4-triazol-3-yl)pyridin-2-yl)carbamoyl)phenyl)thio)pyrimidine-2-carboxylic acid;
N-(6-(4-Isopropyl-4H-1,2,4-triazol-3-yl)pyridin-2-yl)-3-phenylsulfonyl)benzamide;
N-(6-(4-Isopropyl-4H-1,2,4-triazol-3-yl)pyridin-2-yl)-3-pyridin-2-ylsulfonyl)benzamide;
N-(6-(4-Isopropyl-4H-1,2,4-triazol-3-yl)pyridin-2-yl)-3-pyridin-3-ylsulfonyl)benzamide;
N-(6-(4-Isopropyl-4H-1,2,4-triazol-3-yl)pyridin-2-yl)-3-pyridin-4-ylsulfonyl)benzamide;
N-(6-(4-Isopropyl-4H-1,2,4-triazol-3-yl)pyridin-2-yl)-3-pyrimidin-4-ylsulfonyl)benzamide;
N-(6-(4-Isopropyl-4H-1,2,4-triazol-3-yl)pyridin-2-yl)-3-pyrimidin-5-ylsulfonyl)benzamide;
N-(6-(4-Isopropyl-4H-1,2,4-triazol-3-yl)pyridin-2-yl)-3-(pyrimidin-2-ylsulfonyl)benzamide;
N-(6-(4-Isopropyl-4H-1,2,4-triazol-3-yl)pyridin-2-yl)-3-(pyrazin-2-ylsulfonyl)benzamide;
N-(6-(4-Isopropyl-4H-1,2,4-triazol-3-yl)pyridin-2-yl)-3-(pyridazin-3-ylsulfonyl)benzamide;
N-(6-(4-Isopropyl-4H-1,2,4-triazol-3-yl)pyridin-2-yl)-3-(pyridazin-4-ylsulfonyl)benzamide;
N1-(6-(4-Isopropyl-4H-1,2,4-triazol-3-yl)pyridin-2-yl)-V3-phenylisophthal amide;
N1-(6-(4-Isopropyl-4H-1,2,4-triazol-3-yl)pyridin-2-yl)-N3-(pyridin-2-yl)isophthal amide;
N1-(6-(4-Isopropyl-4H-1,2,4 riazol-3-yl)pyridin-2-yl)-N1-(pyridin-3-yl)isophthalamide;
N1-(6-(4-Isopropyl-4H-1,2,4-triazol-3-yl)pyridin-2-yl)-N3-(pyridin-4-yl)isophthal amide;
N1-(6-(4-Isopropyl-4H-1,2,4-triazol-3-yl)pyridin-2-yl)-N3-(pyrimidin-4-yl)isophthal amide;
N1-(6-(4-Isopropyl-4H-1,2,4-triazol-3-yl)pyridin-2-yl)-N3-(pyrimidin-5-yl)isophthal amide;
N1-(6-(4-Isopropyl-4H-1,2,4-triazol-3-yl)pyridin-2-yl)-N3-(pyrazin-2-yl)isophthal amide;
N1-(6-(4-Isopropyl-4H-1,2,4 riazol-3-yl)pyridin-2-yl)-N1-(pyridazin-4-yl)isophthalamide;
N1-(6-(4 sopropyl-4H-1,2,4-triazol-3-yppyridin-2-yl)-N3-0H-pyrrol-2-ypisophthalamide;
N1 6 4 sopropyl-4H-1,2,4-triazol-3-yl)pyridin-2-yl)-N3-(1-methyl-1H-pyrrol-2-yl)isophthalamide;
N1-(Furan-2-yl)-N3-(6-(4-isopropyl-4H-1,2,4-triazol-3-yl)pyridin-2-yl)isophthalamide;
N1-(1H-Imidazol-5-yl)-N3-(6-(4-i sopropyl-4H-1,2,4-triazol-3-yl)pyridin-2-yl)isophthalamide;
N1-(6-(4-Isopropyl-4H-1,2,4-triazol-3-yl)pyridin-2-yl)-N3-(1-methyl-1H-imidazol -4-yl)isophthalamide;
N1-(6-(4-Isopropyl-4H-1,2,4-triazol-3-yl)pyridin-2-yl)-N3-(oxazol-4-yl)isophthal amide;
N1-(6-(4-Isopropyl-4H-1,2,4-triazol-3-yl)pyridin-2-yl)-N3-(oxazol-5-yl)isophthal amide;
N1-(6-(4-Isopropyl-4H-1,2,4-triazol-3-yl)pyridin-2-yl)-N3-(isoxazol-5-yl)isophthal amide;
N1-(6-(4-Isopropyl-4H-1,2,4-triazol-3-yl)pyridin-2-yl)-N3-(isoxazol-3-yl)isophthal amide; or
N1-(6-(4-Isopropyl-4H-1,2,4-triazol-3-yl)pyridin-2-yl)-N3-(1,3 ,4-oxadiazol-2-yl)isophthalamide; or a
   pharmaceutically acceptable salt or solvate thereof.

In another aspect, presented herein is a method for treating cognitive impairment in a subject in need thereof, comprising administering to the subject a dual inhibitor of ASK1 and DYRK1A, wherein the dual inhibitor is a compound having the structure of Formula (XI), or a pharmaceutically acceptable salt or solvate thereof:

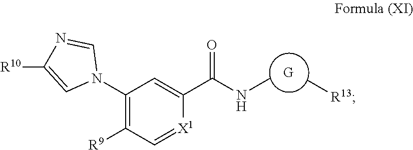

wherein:
Ring G is selected from

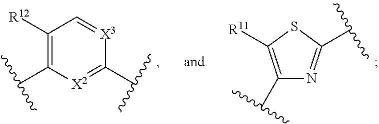

X¹, X² and X³ are each independently selected from N or)C(R¹⁰);
R¹⁰, R¹¹ and R¹² are each independently selected from the group consisting of: hydrogen, halogen, cyano, optionally substituted —C₁-C₆ alkyl, optionally substituted —C₃-C₈ cycloalkyl, optionally substituted 3- to 8-membered heterocycloalkyl, and optionally substituted —C₁-C₆ alkoxyl;
R¹³ is selected from:

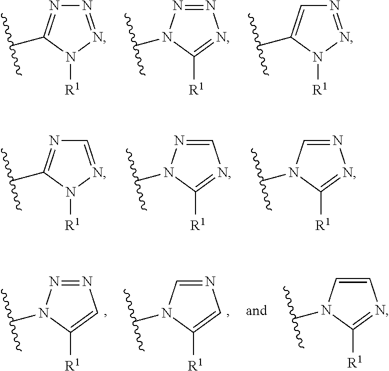

each of which is optionally substituted when possible;
R¹ is selected from the group consisting of: hydrogen, optionally substituted —C₁-C₆ alkyl, optionally substituted —C₂-C₈ alkenyl, optionally substituted —C₂-C₈ alkynyl, optionally substituted —C₃-C₈ cycloalkyl, optionally substituted aryl, optionally substituted arylalkyl, optionally substituted 3- to 8-membered heterocycloalkyl, optionally substituted heteroaryl, optionally substituted heteroarylalkyl, and —N(R¹⁶)(R¹⁷);
provided that when le is R¹ is

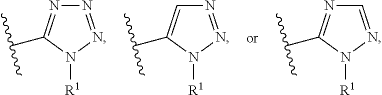

R¹ not —N(R¹⁶)(R¹⁷);
R⁹ is selected from the group consisting of: hydrogen, halogen, cyano, optionally substituted —C₁-C₆ alkyl, optionally substituted —C₂-C₈ alkenyl, optionally substituted —C₂-C₈ alkynyl, optionally substituted —C₃-C₈ cycloalkyl, optionally substituted aryl, optionally substituted arylalkyl, optionally substituted 3- to 8-membered heterocycloalkyl, optionally substituted heteroaryl, optionally substituted heteroarylalkyl, —N(R¹⁶)(R¹⁷), —S(O)₂N(R ¹⁶)(R¹⁷), —N(R¹⁶)C(O)(R¹⁷), and —N(R¹⁶)S(O)₂(R¹⁷);
   wherein R¹⁶ and R¹⁷ are independently selected from the group consisting of hydrogen, —C₁-C₁₅ alkyl, preferably C₁-C₆-alkyl; cycloalkyl, heterocycloalkyl, aryl, and heteroaryl, wherein each alkyl, cycloalkyl, heterocycloalkyl, aryl and heteroaryl is optionally substituted with 1-3 substituents independently selected from halo, alkyl,alkylamino, dialkylamino, alkyl-C(O)NH—, aryl-C(O)NH—, heteroaryl-C(O)—NH, —CN, alkoxy, —CF₃, aryl, and heteroaryl;
   or R¹⁷ and R¹⁶ are taken together with the nitrogen to which they are attached to form a heterocyclic.

In some embodiments, the dual inhibitor of ASK1 and DYRK1A is:

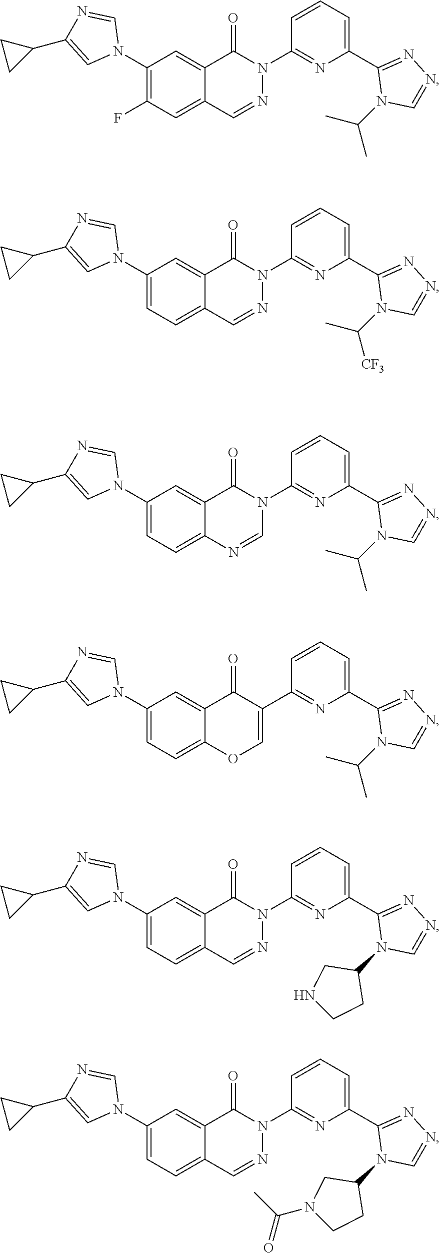

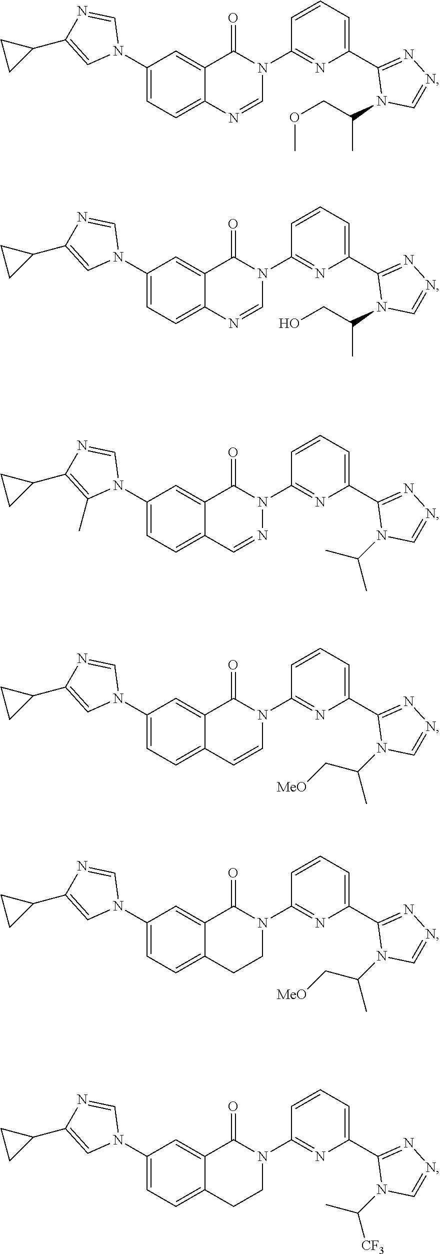

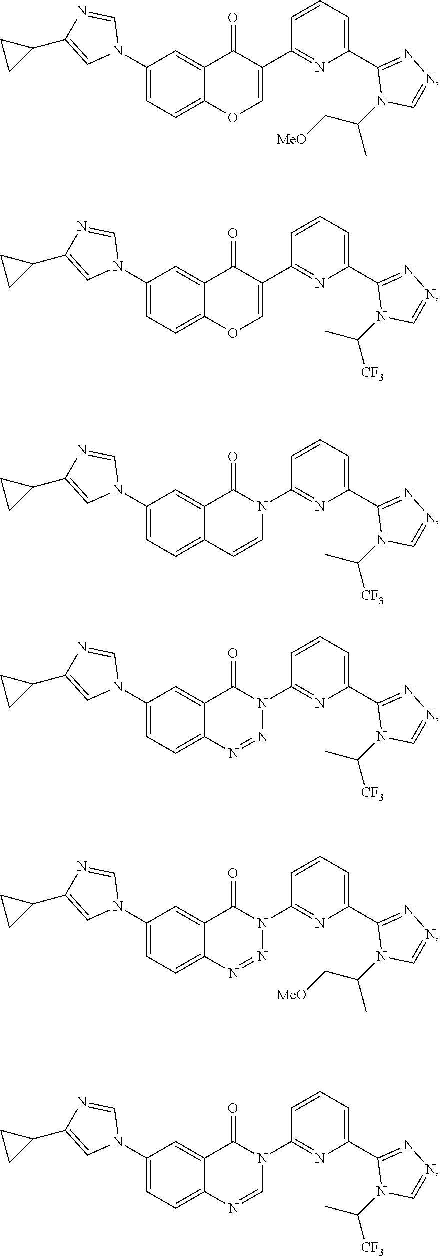 [SMILES: COCC(C)n1cnnc1-c1cccc(-c2coc3ccc(-n4cnc(C5CC5)c4)cc3c2=O)n1 ; CC(n1cnnc1-c1cccc(-c2coc3ccc(-n4cnc(C5CC5)c4)cc3c2=O)n1)C(F)(F)F ; CC(n1cnnc1-c1cccc(-n2ccc3ccc(-n4cnc(C5CC5)c4)cc3c2=O)n1)C(F)(F)F ; CC(n1cnnc1-c1cccc(-n2nnc3ccc(-n4cnc(C5CC5)c4)cc3c2=O)n1)C(F)(F)F ; COCC(C)n1cnnc1-c1cccc(-n2nnc3ccc(-n4cnc(C5CC5)c4)cc3c2=O)n1 ; CC(n1cnnc1-c1cccc(-n2cnc3ccc(-n4cnc(C5CC5)c4)cc3c2=O)n1)C(F)(F)F]

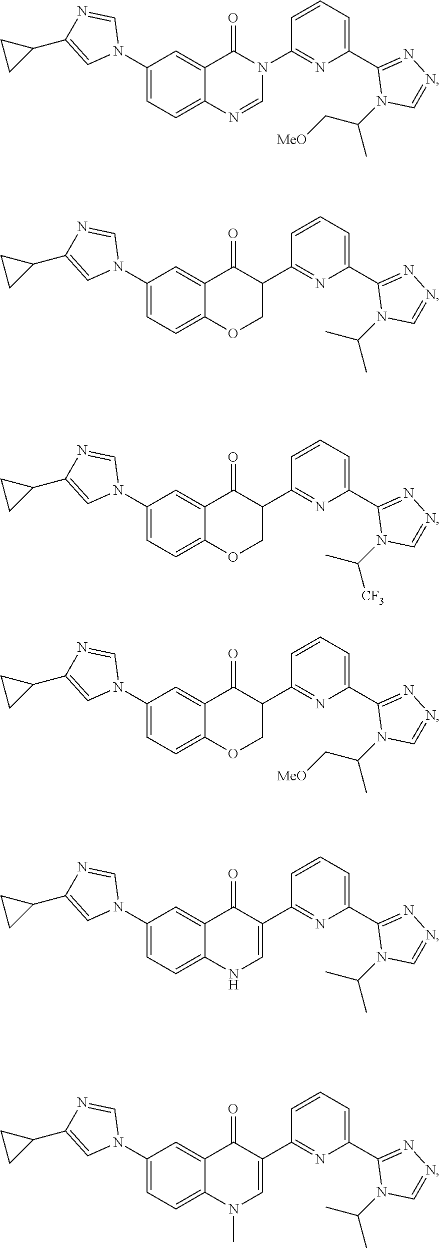 [SMILES: COCC(C)n1cnnc1-c1cccc(-n2cnc3ccc(-n4cnc(C5CC5)c4)cc3c2=O)n1 ; CC(C)n1cnnc1-c1cccc(C2COc3ccc(-n4cnc(C5CC5)c4)cc3C2=O)n1 ; CC(n1cnnc1-c1cccc(C2COc3ccc(-n4cnc(C5CC5)c4)cc3C2=O)n1)C(F)(F)F ; COCC(C)n1cnnc1-c1cccc(C2COc3ccc(-n4cnc(C5CC5)c4)cc3C2=O)n1 ; CC(C)n1cnnc1-c1cccc(-c2c[nH]c3ccc(-n4cnc(C5CC5)c4)cc3c2=O)n1 ; CC(C)n1cnnc1-c1cccc(-c2cn(C)c3ccc(-n4cnc(C5CC5)c4)cc3c2=O)n1]

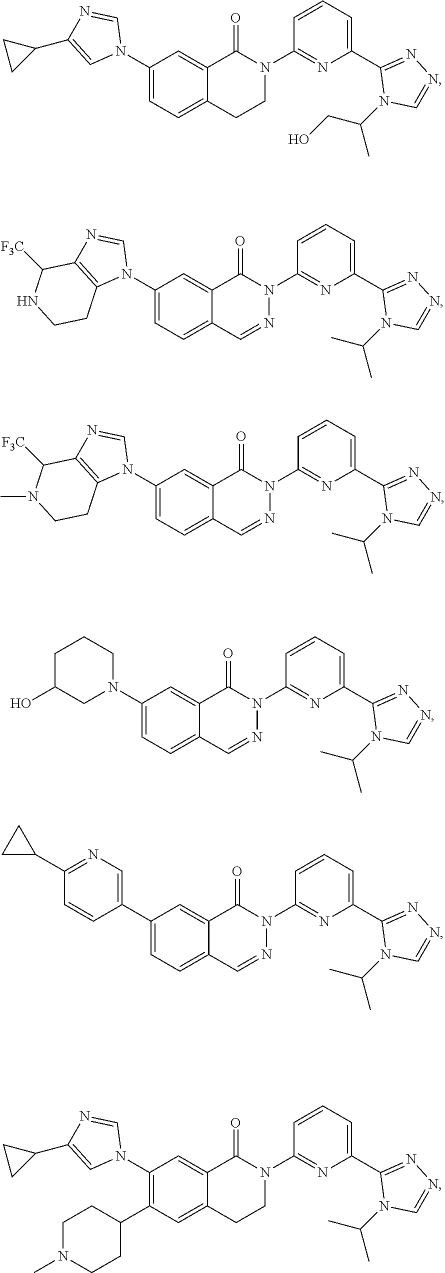 [SMILES: CC(CO)n1cnnc1-c1cccc(N2CCc3ccc(-n4cnc(C5CC5)c4)cc3C2=O)n1 ; CC(C)n1cnnc1-c1cccc(-n2ncc3ccc(-n4cnc5c4CCNC5C(F)(F)F)cc3c2=O)n1 ; CC(C)n1cnnc1-c1cccc(-n2ncc3ccc(-n4cnc5c4CCN(C)C5C(F)(F)F)cc3c2=O)n1 ; CC(C)n1cnnc1-c1cccc(-n2ncc3ccc(N4CCCC(O)C4)cc3c2=O)n1 ; CC(C)n1cnnc1-c1cccc(-n2ncc3ccc(-c4ccc(C5CC5)nc4)cc3c2=O)n1 ; CC(C)n1cnnc1-c1cccc(N2CCc3cc(C4CCN(C)CC4)c(-n4cnc(C5CC5)c4)cc3C2=O)n1]

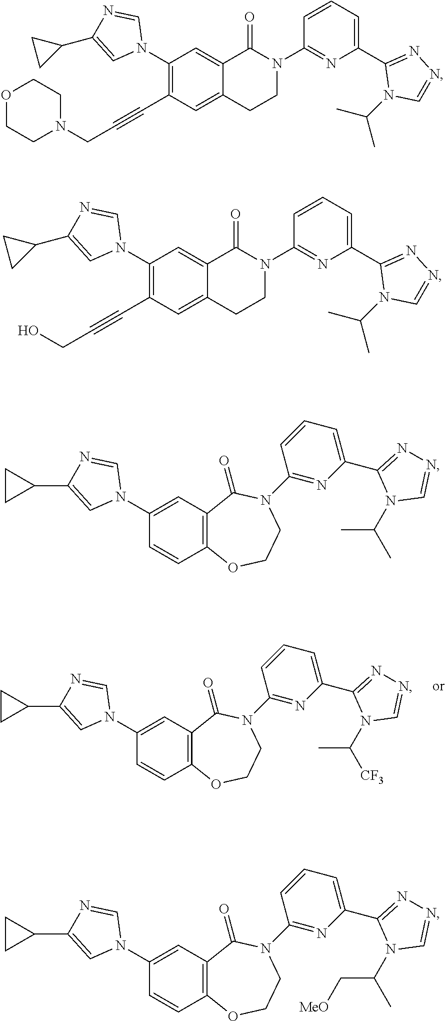 [SMILES: CC(C)n1cnnc1-c1cccc(N2CCc3cc(C#CCN4CCOCC4)c(-n4cnc(C5CC5)c4)cc3C2=O)n1 ; CC(C)n1cnnc1-c1cccc(N2CCc3cc(C#CCO)c(-n4cnc(C5CC5)c4)cc3C2=O)n1 ; CC(C)n1cnnc1-c1cccc(N2CCOc3ccc(-n4cnc(C5CC5)c4)cc3C2=O)n1 ; CC(n1cnnc1-c1cccc(N2CCOc3ccc(-n4cnc(C5CC5)c4)cc3C2=O)n1)C(F)(F)F ; COCC(C)n1cnnc1-c1cccc(N2CCOc3ccc(-n4cnc(C5CC5)c4)cc3C2=O)n1]

or a pharmaceutically acceptable salt or solvate thereof.

In some embodiments, the dual inhibitor of ASK1 and DYRK1A is:

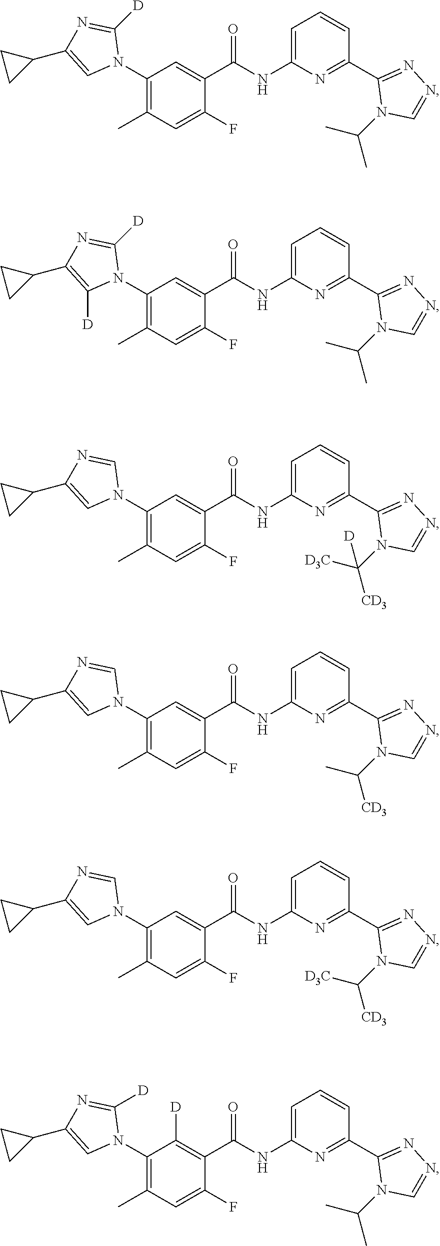 [SMILES: [2H]c1nc(C2CC2)cn1-c1cc(C(=O)Nc2cccc(-c3nncn3C(C)C)n2)c(F)cc1C ; [2H]c1nc(C2CC2)c([2H])n1-c1cc(C(=O)Nc2cccc(-c3nncn3C(C)C)n2)c(F)cc1C ; [2H]C([2H])([2H])C([2H])(n1cnnc1-c1cccc(NC(=O)c2cc(-n3cnc(C4CC4)c3)c(C)cc2F)n1)C([2H])([2H])[2H] ; [2H]C([2H])([2H])C(C)n1cnnc1-c1cccc(NC(=O)c2cc(-n3cnc(C4CC4)c3)c(C)cc2F)n1 ; [2H]C([2H])([2H])C(n1cnnc1-c1cccc(NC(=O)c2cc(-n3cnc(C4CC4)c3)c(C)cc2F)n1)C([2H])([2H])[2H] ; [2H]c1c(C(=O)Nc2cccc(-c3nncn3C(C)C)n2)c(F)cc(C)c1-n1cc(C2CC2)nc1[2H]]

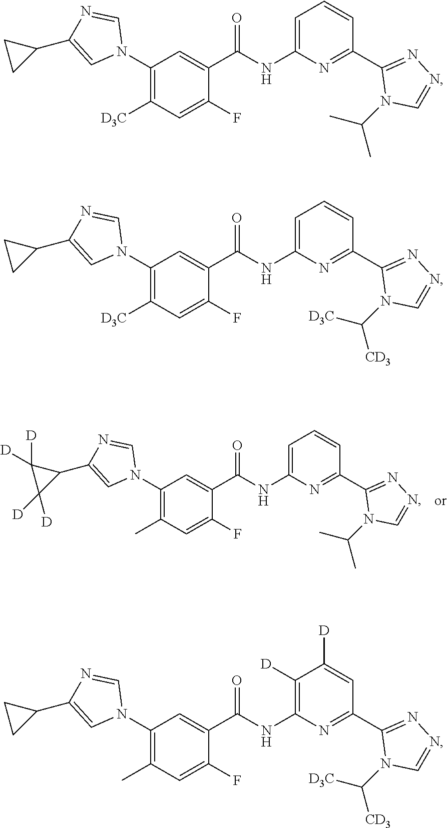 [SMILES: [2H]C([2H])([2H])c1cc(F)c(C(=O)Nc2cccc(-c3nncn3C(C)C)n2)cc1-n1cnc(C2CC2)c1 ; [2H]C([2H])([2H])c1cc(F)c(C(=O)Nc2cccc(-c3nncn3C(C([2H])([2H])[2H])C([2H])([2H])[2H])n2)cc1-n1cnc(C2CC2)c1 ; [2H]C1([2H])C(c2cn(-c3cc(C(=O)Nc4cccc(-c5nncn5C(C)C)n4)c(F)cc3C)cn2)C1([2H])[2H] ; [2H]c1cc(-c2nncn2C(C([2H])([2H])[2H])C([2H])([2H])[2H])nc(NC(=O)c2cc(-n3cnc(C4CC4)c3)c(C)cc2F)c1[2H]]

or a pharmaceutically acceptable salt or solvate thereof.

### Methods of Use

In one aspect presented herein, is a method for the treatment of prevention of an ASK1 or DYRK1A associated condition, disease, or disorder in a subject in need thereof, which is not a cardio-metabolic disease, comprising administering to the subject a therapeutically effective amount of a dual inhibitor of ASK1 and DYRK1A having the structure of Formula (IV), (IVa), (IVb), (IVc), (V), (VI), (VII), (IX), (X), or (XI), or a pharmaceutically acceptable salt or solvate thereof

In some embodiments, the dual inhibitor of Formula (IV), (IVa), (IVb), (IVc), (V), (VI), (VII), (VIII), (IX), (X), or (XI), or a pharmaceutically acceptable salt or solvate thereof, can be used to decrease the activity of ASK1 and DYRK1A, or otherwise be used to affect the properties and/or behavior of ASK1 and DYRK1A (e.g., stability, phosphorylation, kinase activity, and interactions with other proteins).

In some embodiments, the ASK1 or DYRK1A associated condition is a cognitive impairment. In some embodiments, the ASK1 or DYRK1A associated condition is a cognitive dysfunction.

In one aspect, disclosed herein is a method for the treatment of prevention of a cognitive impairment in a subject in need thereof, comprising administering to the subject a therapeutically effective amount of a dual inhibitor of ASK1 and DYRK1A, wherein the dual inhibitor is a compound having the structure of Formula (IV), (IVa), (IVb), (IVc), (V), (VI), (VII), (VIII), (IX), (X), or (XI), or a pharmaceutically acceptable salt or solvate thereof

In some embodiments, the cognitive impairment is a neurodegenerative disease or a neurodevelopmental disorder. In some embodiments, the disease or disorder is a promotion of neuroprotection.

In some embodiments, the dual inhibitors of the present disclosure have utility in the reduction one or more physiological conditions or symptom associated with a neurodegenerative disease, thereby treating the neurodegenerative disease. In some embodiments, the dual inhibitors of the present disclosure have utility in the prevention of one or more physiological conditions or symptoms associated with a neurodegenerative disease, thereby providing a neuroprotection.

In some embodiments, the dual inhibitors of the present disclosure have utility to reduce one or more physiological conditions or symptom associated with a neurodevelopmental disorder, thereby treating the neurodevelopmental disorder.

In some embodiments, the neurodegenerative disease or disorder is an Alexander disease, an Alper's disease, Alzheimer's disease, an amyotrophic lateral sclerosis (ALS), an ataxia telangiectasia, a Canavan disease, a chronic traumatic encephalopathy, a Cockayne syndrome, a corticobasal degeneration, a Creutzfeldt-Jakob disease, a Guillain-Barre syndrome, a Huntington disease, a Kennedy's disease, a Krabbe disease, a Lewy body dementia, a Lytico-Bodig disease, a Machado-Joseph disease, a multiple sclerosis, a Parkinson's disease, a Pelizaeus-Merzbacher disease, a Pick's disease, a primary lateral sclerosis, a Refsum's disease, a Sandhoff disease, a Schilder's disease, a spinal cord injury, a Steele-Richardson-Olszewski disease, a stroke, a tabes dorsalis, and/or a traumatic brain injury. In some embodiments, ALS is also known as Lou Gerhrig's disease. In some embodiments, Pick's disease is also known as frontotemporal dimentia (FTD) or frontotemporal lobar degeneration (FTLD).

In some embodiments, the neurodevelopmental disorder is an Asperger syndrome, an attention deficient hyperactivity disorder, an autism disorder, an autism spectrum disorder, an autosomal dominant 1 mental retardation, a childhood disintegrative disorder, a Down syndrome, a Heller's syndrome, a mental retardation, a mental retardation 7, a mosaic Down syndrome, a pervasive developmental disorder not otherwise specified (PDD-NOS), and/or a trisomy 21.

In some embodiments, the dual inhibitors have utility for the treatment of at least one symptom associated with a neurodegenerative disorder including, but not limited to, abnormal inhibition of axon growth, abnormal axonal transport, aberrant synaptic function, synaptic transmission loss, impaired synaptic plasticity, synaptic loss, neuronal degeneration, motor neuron degeneration, motor neuron loss, poor neuronal survival, beta-amyloid plaque deposits, tau protein deposits, tau hyperphosphorylation, aberrant neurofilament accumulation, dementia, memory loss, loss of cognitive function, compulsive behavior, chorea, general restlessness, lack of impulse restraint, reactive astroglia, and/or reactive microglia.

In some embodiments, the dual inhibitors of the present disclosure are effective in the treatment or prevention of neurodegenerative diseases (e.g., ALS, Alzheimer's disease, multiple sclerosis). Neurodegenerative diseases are conditions that affect brain function. They result from the deterioration of neurons and are characterized by progressive central or peripheral nervous system dysfunction. ASK1 and DYRK1A play a role in neurodegenerative disease pathology. For example, oxidative stress related to ASK1 activation and hyperphoshorylation of tau protein substrates related to DYRK1A activity. Thus, inhibitors of ASK1 and DYRK1A have utility in the treatment and/or prevention of neurodegenerative diseases and thereby in the symptoms associated with neurodegenerative processes. A neurodegenerative disease include, without limitation, an Alexander disease, an Alper's disease, Alzheimer's disease, an amyotrophic lateral sclerosis, an ataxia telangiectasia, a Canavan disease, a chronic traumatic encephalopathy, a Cockayne syndrome, a corticobasal degeneration, a Creutzfeldt-Jakob disease, a Guillain-Barre Syndrome a HIV-induced neurodegeneration, a Huntington disease, a Kennedy's disease, a Krabbe disease, a Lewy body dementia, a Machado-Joseph disease, a multiple sclerosis, a Parkinson's disease, a Pelizaeus-Merzbacher disease, a Pick's disease, a primary lateral sclerosis, a Refsum's disease, a Sandhoff disease, a Schilder's disease, a spinal cord injury, a Steele-Richardson-Olszewski disease, a stroke, a tabes dorsalis, and/or a traumatic brain injury. Symptoms associated with a neurodegenerative disease include, without limitation, abnormal movement, abnormal sensation, limb grasping, muscle weakness, atrophy, paralysis, abnormal inhibition of axon growth, abnormal axonal transport, aberrant synaptic function, synaptic transmission loss, impaired synaptic plasticity, synaptic loss, neuronal degeneration, motor neuron degeneration, motor neuron loss, poor neuronal survival, memory loss, impaired learning, chorea, impaired cognition, dementia, ABeta-amyloid plaque deposits, aberrant neurofilament accumulation, tau protein deposit, reactive astroglia and/or reactive microglia.

In some embodiments, the dual inhibitors of the present disclosure are effective in the treatment and/or prevention of neurodevelopmental disorders (e.g., autism spectrum disorder and Down syndrome). Neurodevelopmental disorders are conditions that affect brain development. They have many causes including environmental and genetic. Incomplete development of the brain, the central nervous system, and neuron networks has far reaching affect associated with cognition and intelligence. ASK1 and DYRK1A play a role in brain function and brain development. Thus, the dual inhibitors presented herein have utility for the treatment or the prevention of the symptoms associated with a neurodevelopmental disorder. A neurodevelopmental disorder includes, without limitation, an Asperger syndrome, an attention deficient hyperactivity disorder, an autism disorder, an autism spectrum disorder, an autosomal dominant 1 mental retardation, a childhood disintegrative disorder, a Down syndrome, a Heller's syndrome, a mental retardation, a mental retardation 7, a mosaic Down syndrome, a pervasive developmental disorder not otherwise specified (PDD-NOS), and/or a trisomy 21. Symptoms associated with neurodevelopmental disorders include, without limitation abnormal movement, muscle weakness, abnormal inhibition of axon growth, abnormal axonal transport, aberrant synaptic function, impaired memory, impaired learning, impaired cognition, impaired cognitive flexibility, psychomotor retardation including slowing down of physical and emotional reaction, impulse control, lack of restraint, hyperactivity, and/or problems with speaking.

In some embodiments, is a method for the treatment or prevention of a neurodegenerative disease in a subject in need thereof, comprising administering to the subject a therapeutically effective amount of a dual inhibitor of ASK1 and DYRK1A, wherein the dual inhibitor is a compound having the structure of Formula (IV), (IVa), (IVb), (IVc), (V), (VI), (VII), (VIII), (IX), (X), or (XI), or a pharmaceutically acceptable salt or solvate thereof.

In some embodiments, is a method for the treatment or prevention of Alzheimer's disease in a subject in need thereof, comprising administering to the subject a therapeutically effective amount of a dual inhibitor of ASK1 and DYRK1A, wherein the dual inhibitor is a compound having the structure of Formula (IV), (IVa), (IVb), (IVc), (V), (VI), (VII), (VIII), (IX), (X), or (XI), or a pharmaceutically acceptable salt or solvate thereof.

In some embodiments, is a method for the treatment or prevention of Parkinson's disease in a subject in need thereof, comprising administering to the subject a therapeutically effective amount of a dual inhibitor of ASK1 and DYRK1A, wherein the dual inhibitor is a compound having the structure of Formula (IV), (IVa), (IVb), (IVc), (V), (VI), (VII), (VIII), (IX), (X), or (XI), or a pharmaceutically acceptable salt or solvate thereof.

In some embodiments, is a method for the treatment or prevention of a neurodevelopmental disorder in a subject in need thereof, comprising administering to the subject a therapeutically effective amount of a dual inhibitor of ASK1 and DYRK1A, wherein the dual inhibitor is a compound having the structure of Formula (IV), (IVa), (IVb), (IVc), (V), (VI), (VII), (VIII), (IX), (X), or (XI), or a pharmaceutically acceptable salt or solvate thereof.

In some embodiments, is a method for the treatment or prevention of a Down syndrome in a subject in need thereof, comprising administering to the subject a therapeutically effective amount of a dual inhibitor of ASK1 and DYRK1A, wherein the dual inhibitor is a compound having the structure of Formula (IV), (IVa), (IVb), (IVc), (V), (VI), (VII), (VIII), (IX), (X), or (XI), or a pharmaceutically acceptable salt or solvate thereof.

In some embodiments, a “dual inhibitor of ASK1 and DYRK1A” refers to a compound displaying less than 10-fold selectivity between ASK1 and DYRK1A. In some embodiments, the compound described herein displays less than 10-fold selectivity for ASK1 over DYRK1A. In some embodiments, the compound described herein displays less than 10-fold selectivity for DYRK1A over ASK1.

In some embodiments, there is less than 5-fold, less than 6-fold, less than 7-fold, less than 8-fold, less than 9-fold, or less than 10-fold selectivity between ASK1 and DYRK1A. In some embodiments, there is less than 5-fold selectivity between the two enzymes. In some embodiments, there is less than 6-fold selectivity between the two enzymes. In some embodiments, there is less than 7-fold selectivity between the two enzymes. In some embodiments, there is less than 8-fold selectivity between the two enzymes. In some embodiments, there is less than 9-fold selectivity between the two enzymes. In some embodiments, there is less than 10-fold selectivity between the two enzymes.

In some embodiments, the compound simultaneously acts on ASK1 and DYRK1A with an IC₅₀ of less than 500 nM, less than 400 nM, less than 300 nM, less than 200 nM, less than 100 nM, or less than 50 nM. In some embodiments, both enzymes are inhibited with an IC₅₀ of less than 500 nM, less than 400 nM, less than 300 nM, less than 200 nM, less than 100 nM, or less than 50 nM. In some embodiments, both enzymes are inhibited with an IC₅₀ of less than 500 nM. In some embodiments, both enzymes are inhibited with an IC₅₀ of less than 400 nM. In some embodiments, both enzymes are inhibited with an IC₅₀ of less than 300 nM. In some embodiments, both enzymes are inhibited with an IC₅₀ of less than 200 nM. In some embodiments, both enzymes are inhibited with an IC₅₀ of less than 100 nM. In some embodiments, both enzymes are inhibited with an IC₅₀ of less than 50 nM.

In one aspect, the dual inhibitor described herein is selected from a compound in table Table 1, or a pharmaceutically acceptable salt or solvate thereof.

TABLE 1

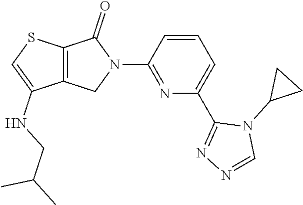 [SMILES: CC(C)CNc1csc2c1CN(c1cccc(-c3nncn3C3CC3)n1)C2=O]

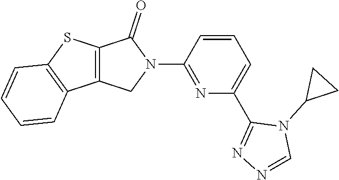 [SMILES: O=C1c2sc3ccccc3c2CN1c1cccc(-c2nncn2C2CC2)n1]

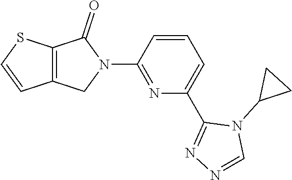 [SMILES: O=C1c2sccc2CN1c1cccc(-c2nncn2C2CC2)n1]

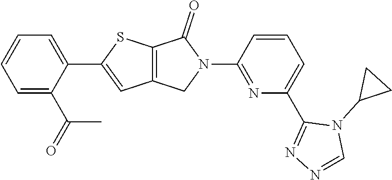 [SMILES: CC(=O)c1ccccc1-c1cc2c(s1)C(=O)N(c1cccc(-c3nncn3C3CC3)n1)C2]

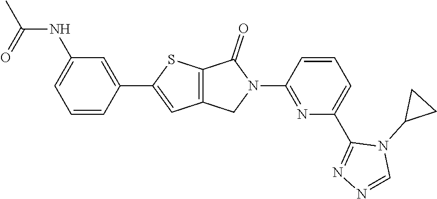 [SMILES: CC(=O)Nc1cccc(-c2cc3c(s2)C(=O)N(c2cccc(-c4nncn4C4CC4)n2)C3)c1]

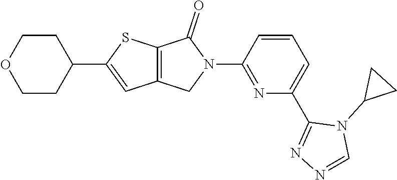 [SMILES: O=C1c2sc(C3CCOCC3)cc2CN1c1cccc(-c2nncn2C2CC2)n1]

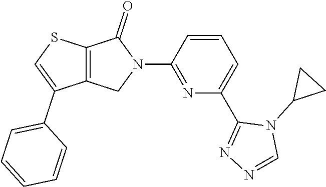 [SMILES: O=C1c2scc(-c3ccccc3)c2CN1c1cccc(-c2nncn2C2CC2)n1]

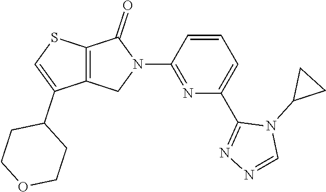 [SMILES: O=C1c2scc(C3CCOCC3)c2CN1c1cccc(-c2nncn2C2CC2)n1]

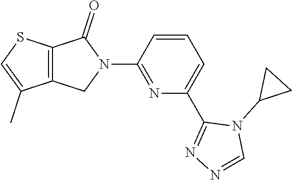 [SMILES: Cc1csc2c1CN(c1cccc(-c3nncn3C3CC3)n1)C2=O]

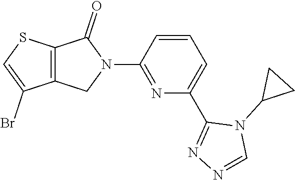 [SMILES: O=C1c2scc(Br)c2CN1c1cccc(-c2nncn2C2CC2)n1]

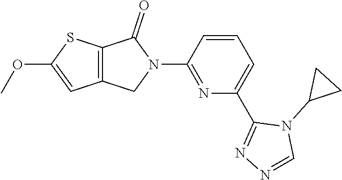 [SMILES: COc1cc2c(s1)C(=O)N(c1cccc(-c3nncn3C3CC3)n1)C2]

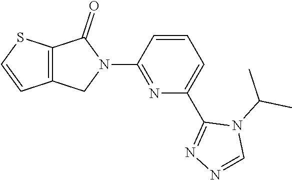 [SMILES: CC(C)n1cnnc1-c1cccc(N2Cc3ccsc3C2=O)n1]

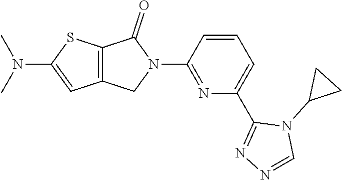 [SMILES: CN(C)c1cc2c(s1)C(=O)N(c1cccc(-c3nncn3C3CC3)n1)C2]

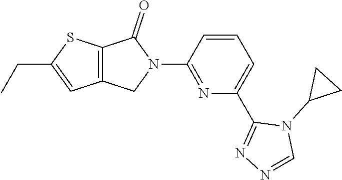 [SMILES: CCc1cc2c(s1)C(=O)N(c1cccc(-c3nncn3C3CC3)n1)C2]

### Synthesis of Compounds

Compounds described herein are synthesized using standard synthetic techniques or using methods known in the art in combination with methods described herein. In addition, solvents, temperatures and other reaction conditions presented herein may vary.

The starting material used for the synthesis of the compounds described herein are either synthesized or obtained from commercial sources, such as, but not limited to, Sigma-Aldrich, Fluka, Acros Organics, Alfa Aesar, and the like. The compounds described herein, and other related compounds having different substituents are synthesized using techniques and materials described herein or otherwise known, including those found in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999). General methods for the preparation of compounds can be modified by the use of appropriate reagents and conditions for the introduction of the various moieties found in the formulae as provided herein. A detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

In one aspect, compounds described herein are synthesized as outlined in the Examples. Throughout the specification, groups and substituents thereof are chosen by one skilled in the field to provide stable moieties and compounds.

### Further Forms of Compounds

In another aspect, compounds described herein possess one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis-, trans-, syn-, anti-, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

“Pharmaceutically acceptable,” as used herein, refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound described herein with acids. Pharmaceutically acceptable salts are also obtained by reacting a compound described herein with a base to form a salt.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid to form a salt such as, for example, a hydrochloric acid salt, a hydrobromic acid salt, a sulfuric acid salt, a phosphoric acid salt, a metaphosphoric acid salt, and the like; or with an organic acid to form a salt such as, for example, an acetic acid salt, a propionic acid salt, a hexanoic acid salt, a cyclopentanepropionic acid salt, a glycolic acid salt, a pyruvic acid salt, a lactic acid salt, a malonic acid salt, a succinic acid salt, a malic acid salt, a maleic acid salt, a fumaric acid salt, a trifluoroacetic acid salt, a tartaric acid salt, a citric acid salt, a benzoic acid salt, a 3-(4-hydroxybenzoyl)benzoic acid salt, a cinnamic acid salt, a mandelic acid salt, a methanesulfonic acid salt, an ethanesulfonic acid salt, a 1,2-ethanedisulfonic acid salt, a 2-hydroxyethanesulfonic acid salt, a benzenesulfonic acid salt, a toluenesulfonic acid salt, a 2-naphthalenesulfonic acid salt, a 4-methylbicyclo42.2.2loct-2-ene-1-carboxylic acid salt, a glucoheptonic acid salt, a 4,4′-methylenebis-(3-hydroxy-2-ene-1-carboxylic acid) salt, a 3-phenylpropionic acid salt, a trimethylacetic acid salt, a tertiary butylacetic acid salt, a lauryl sulfuric acid salt, a gluconic acid salt, a glutamic acid salt, a hydroxynaphthoic acid salt, a salicylic acid salt, a stearic acid salt, a muconic acid salt, a butyric acid salt, a phenylacetic acid salt, a phenylbutyric acid salt, a valproic acid salt, and the like; (2) salts formed when an acidic proton present in the parent compound is replaced by a metal ion, e.g., an alkali metal ion (e.g. a lithium salt, a sodium salt, or a potassium salt), an alkaline earth ion (e.g. a magnesium salt, or a calcium salt), or an aluminum ion (e.g. an aluminum salt). In some cases, compounds described herein may coordinate with an organic base to form a salt, such as, but not limited to, an ethanolamine salt, a diethanolamine salt, a triethanolamine salt, a tromethamine salt, a N-methylglucamine salt, a dicyclohexylamine salt, or a tris(hydroxymethyl)methylamine salt. In other cases, compounds described herein may form salts with amino acids such as, but not limited to, an arginine salt, a lysine salt, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In some embodiments, a dual inhibitor as described herein is administered in a local rather than systemic manner, for example, via injection of the dual inhibitor directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the dual inhibitor as described herein is provided in the form of a rapid release formulation, in the form of an extended release formulation, or in the form of an intermediate release formulation. In yet other embodiments, the dual inhibitor described herein is administered topically.

The dual inhibitor of the present disclosure can be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

### Pharmaceutical Compositions/Formulations

In some embodiments, the dual inhibitors described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

Provided herein are pharmaceutical compositions that include a dual inhibitor of ASK1 and DYRK1A, wherein the dual inhibitor is a compound having the structure of Formula (IV), (IVa), (IVb), (IVc), (V), (VI), (VII), (VIII), (IX), (X), or (XI), or a pharmaceutically acceptable salt thereof, and at least one pharmaceutically acceptable inactive ingredient, for example an excipient. In some embodiments, the dual inhibitor is administered as a pharmaceutical composition in which the dual inhibitor is a compound having the structure of Formula (IV), (IVa), (IVb), (IVc), (V), (VI), (VII), (VIII), (IX), (X), or (XI), or a pharmaceutically acceptable salt thereof, is mixed with other active ingredients, as in combination therapy. In other embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In yet other embodiments, the pharmaceutical compositions include other therapeutically valuable substances.

A pharmaceutical composition, as used herein, refers to a mixture of a dual inhibitor having the structure of Formula (IV), (IVa), (IVb), (IVc), (V), (VI), (VII), (IX), (X), or (XI), or a pharmaceutically acceptable salt thereof, mixed with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the dual inhibitor to a mammal.

A therapeutically effective amount can vary widely depending on the severity of the disease, the age and relative health of the subject, the potency of the compound used and other factors. The dual inhibitors can be used singly or in combination with one or more therapeutic agents as components of mixtures.

The pharmaceutical compositions described herein are administered to a subject by appropriate administration routes, including but not limited to, oral, parenteral (e.g., intravenous, subcutaneous, intramuscular), intranasal, buccal, topical, rectal, or transdermal administration routes. The pharmaceutical compositions described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a dual inhibitor having the structure of Formula (W), (IVa), (IVb), (IVc), (V), (VI), (VII), (VIII), (IX), (X), or (XI), or a pharmaceutically acceptable salt thereof, are manufactured in a conventional manner, such as, by way of example only, by means of conventional mixing, dissolving, granulating, dragee-making, levigating, emulsifying, encapsulating, entrapping or compression processes.

The pharmaceutical compositions will include at least one dual inhibitor of Formula (IV), (IVa), (IVb), (IVc), (V), (VI), (VII), (VIII), (IX), (X), or (XI), as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, compounds described herein exist in unsolvated form or in solvated forms with pharmaceutically acceptable solvents such as water, ethanol, and the like. The solvated forms of the compounds presented herein are also considered to be disclosed herein.

The pharmaceutical compositions described herein, which include a dual inhibitor of Formula (IV), (IVa), (IVb), (IVc), (V), (VI), (VII), (VIII), (IX), (X), or (XI), or a pharmaceutically acceptable salt thereof, are formulated into any suitable dosage form, including but not limited to, aqueous oral dispersions, liquids, gels, syrups, elixirs, slurries, suspensions, solid oral dosage forms, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In some embodiments, the push-fit capsules do not include any other ingredient besides the capsule shell and the active ingredient. In soft capsules, the active compounds are dissolved or suspended in suitable liquids, such as fatty oils, liquid paraffin, or liquid polyethylene glycols. In some embodiments, stabilizers are added.

All formulations for oral administration are in dosages suitable for such administration.

In one aspect, solid oral dosage forms are prepared by mixing a dual inhibitor of Formula (IV), (IVa), (IVb), (IVc), (V), (VI), (VII), (VIII), (IX), or (X), or a pharmaceutically acceptable salt thereof, with one or more of the following: antioxidants, flavoring agents, and carrier materials such as binders, suspending agents, disintegration agents, filling agents, surfactants, solubilizers, stabilizers, lubricants, wetting agents, and diluents.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. In still other embodiments, the pharmaceutical formulation is in the form of a tablet. In other embodiments, pharmaceutical formulation is in the form of a capsule.

In some embodiments, solid dosage forms, e.g., tablets, effervescent tablets, and capsules, are prepared by mixing particles of a dual inhibitor of Formula (IV), (IVa), (IVb), (IVc), (V), (VI), (VII), (VIII), (IX), or (X), or a pharmaceutically acceptable salt thereof, with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

In some embodiments, tablets will include a film surrounding the final compressed tablet. In some embodiments, the film coating can provide a delayed release of the dual inhibitor of Formula (IV), (IVa), (IVb), (IVc), (V), (VI), (VII), (VIII), (IX), or (X), or a pharmaceutically acceptable salt thereof, from the formulation. In other embodiments, the film coating aids in subject compliance (e.g., Opadry® coatings or sugar coating). Film coatings including Opadry® typically range from about 1% to about 3% of the tablet weight.

A capsule may be prepared, for example, by placing the bulk blend of the formulation of the compound described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

In various embodiments, the particles of the dual inhibitor of Formula (W), (IVa), (IVb), (IVc), (V), (VI), (VII), (VIII), (IX), or (X), or a pharmaceutically acceptable salt thereof, and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In still other embodiments, effervescent powders are also prepared. Effervescent salts have been used to disperse medicines in water for oral administration.

In some embodiments, the pharmaceutical solid oral dosage forms are formulated to provide a controlled release of the active compound. Controlled release refers to the release of the active compound from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules.

Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. In one embodiment, the pulsatile dosage form includes at least two groups of particles, (i.e. multiparticulate) each containing the formulation described herein. The first group of particles provides a substantially immediate dose of the active compound upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or in the art.

In some embodiments, pharmaceutical compositions are provided that include particles of a dual inhibitor of Formula (W), (IVa), (IVb), (IVc), (V), (VI), (VII), (VIII), (IX), (X), or (XI), or a pharmaceutically acceptable salt thereof, and at least one dispersing agent or suspending agent for oral administration to a subject. The compositions may be a powder and/or granules for suspension, and upon admixture with water, a substantially uniform suspension is obtained.

In one aspect, liquid formulation dosage forms for oral administration are in the form of aqueous suspensions selected from the group including, but not limited to, pharmaceutically acceptable aqueous oral dispersions, emulsions, solutions, elixirs, gels, and syrups. See, e.g., Singh et al.., Encyclopedia of Pharmaceutical Technology, 2nd Ed., pp. 754-757 (2002). In addition to the particles of the dual inhibitor of Formula (IV), (IVa), (IVb), (IVc), (V), (VI), (VII), (VIII), (IX), (X), or (XI), the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

Buccal formulations that include the dual inhibitor of Formula (IV), (IVa), (IVb), (IVc), (V), (VI), (VII), (VIII), (IX), (X), or (XI), or a pharmaceutically acceptable salt thereof, are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

In some embodiments, the dual inhibitor of Formula (IV), (IVa), (IVb), (IVc), (V), (VI), (VII), (VIII), (IX), (X), or (XI), or a pharmaceutically acceptable salt thereof, are prepared as transdermal dosage forms. In one embodiment, the transdermal formulations described herein include at least three components: (1) a formulation of a compound of Formula (W), (IVa), (IVb), (IVc), (V), (VI), (VII), (VIII), (IX), (X), or (XI), or a pharmaceutically acceptable salt thereof; (2) a penetration enhancer; and (3) an aqueous adjuvant. In some embodiments the transdermal formulations include additional components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of compounds described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the compounds described herein can be accomplished by means of iontophoretic patches and the like. In one aspect, transdermal patches provide controlled delivery of the active compound. In one aspect, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In one aspect, the dual inhibitor of Formula (IV), (IVa), (IVb), (IVc), (V), (VI), (VII), (VIII), (IX), (X), or (XI), or a pharmaceutically acceptable salt thereof, is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), vegetable oils and organic esters, such as ethyl oleate. In some embodiments, formulations suitable for subcutaneous injection contain additives such as preserving, wetting, emulsifying, and dispensing agents. Prolonged absorption of the injectable pharmaceutical form can be brought about by the use of agents delaying absorption, such as aluminum monostearate and gelatin.

For intravenous injections, compounds described herein are formulated in aqueous solutions, preferably in physiologically compatible buffers such as Hank's solution, Ringer's solution, or physiological saline buffer.

For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

### EXAMPLES

Compounds disclosed herein are made by the methods depicted in the reaction schemes shown below. Procedures are provided herein that, in combination with the knowledge of the synthetic organic chemist of ordinary skill in the art, are in some embodiments used to prepare the full range of compounds as disclosed and claimed herein. Some of the compounds herein were made by the synthetic procedures presented in PCT/US2018/26134, filed on Apr. 4, 2018, which is herein incorporate by reference in its entirety.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds disclosed herein are in some embodiments synthesized, and various modifications to these schemes can be made and will be suggested to one skilled in the art having referred to this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data. Proton nuclear magnetic resonance spectra were obtained on a Bruker 400 MHz spectrometer. Spectra are given in ppm and coupling constants, J values, are reported in hertz (Hz). Mass spectra analyses were performed on Agilent 6120 Mass Spectrometer in ESI or APCI mode when appropriate. Some abbreviations used herein are as follows:

DCM: dichloromethane

DMAP: 4-dimethylaminopyridine

DMF: dimethyl formamide

DMF-DMA: N,N-dimethylformamide dimethyl acetal

EDCI: 1-ethyl-3-(3-dimethylaminopropyl) carbodiimide

EtOAc: ethyl acetate

EtOH: ethanol

Me OH: methanol

PE: petroleum ether.

### Example 1

### Preparation of 2-(6-(4-cyclopropyl-4H-1,2,4-triazol-3-yl)pyridin-2-yl)-1,2-dihydro-3H-indazol-3-one (compound 87)

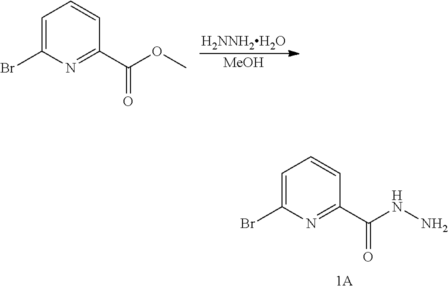

Step 1: Hydrazine hydrate (1.16 mg, 23.1 mmol, 10 eq) was added to a solution of methyl 6-bromopicolinate (500 mg, 2.31 mmol, 1.0 eq) in MeOH (15 mL) at room temperature, then the reaction was stirred for 1 hour. The reaction mixture was concentrated under reduced pressure to give the desired hydrazide product 1A which was used without further purification: ¹H NMR (400 MHz, CDCl₃) δ 8.84 (s, 1H), 8.12 (d, J=7.5 Hz, 1H), 7.71 (t, J=7.7 Hz, 1H), 7.62 (d, J=7.9 Hz, 1H), 4.10 (br s, 2H).

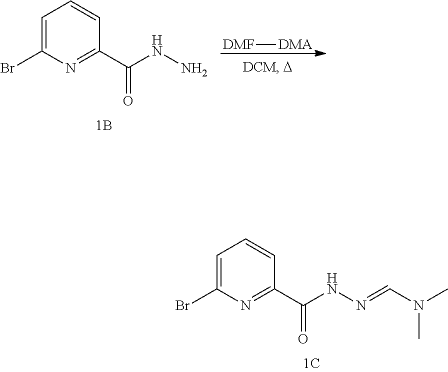

Step 2: A solution of 1B (500 mg, 2.31 mmol, 1.0 eq) and DMF-DMA (1.38 g, 11.6 mmol, 5.0 eq) in DCM (10 mL) was refluxed for 6 hours. After cooling, the reaction mixture was concentrated under reduced pressure to give the desired product 1C which was used in the subsequent step without any further purification.

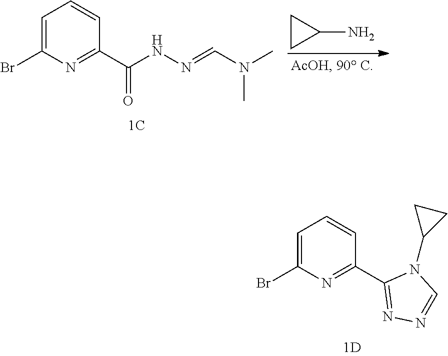

Step 3: Cyclopropylamine (396 mg, 6.93 mmol, 3.0 eq) was added to a stirred solution of 1C (630 mg, 2.31 mmol, 1.0 equiv) in glacial acetic acid (15 ml) at room temperature. After stirring at 90° C. for 3 hours, the reaction mixture was allowed to cool to room temperature. The solvent was removed under reduced pressure and the residue was purified by column chromatography (30%400% EtOAc in PE) to give 500 mg of 1D (>85% purity). The partially purified material was then used directly in the next step. ¹H NMR (400 MHz, CDCl₃) δ 8.29 (s, 1H), 8.24 (d, J=7.7 Hz, 1H), 7.70 (t, J=7.8 Hz, 1H), 7.55 (d, J=7.9 Hz, 1H), 3.91-3.83 (m, 1H), 1.20 (q, J=6.9 Hz, 2H), 0.93 (q, J=6.6 Hz, 2H).

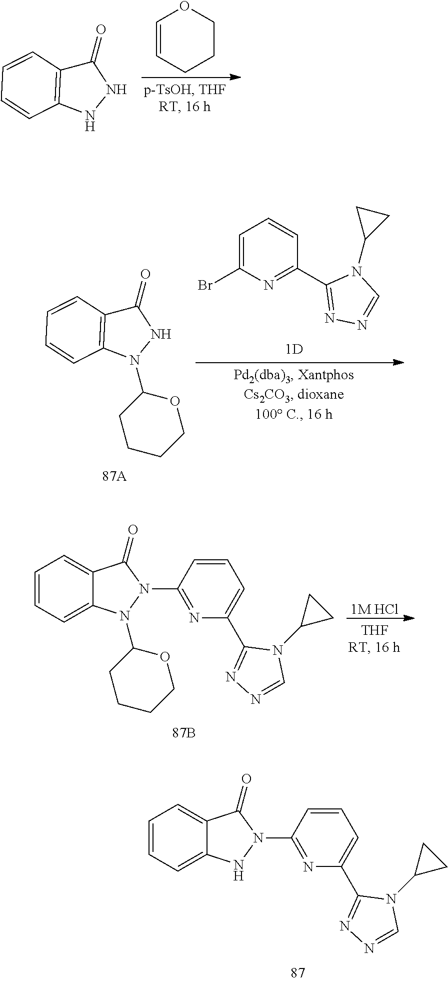

Step 4: A mixture of 3,4-dihydro-2H-pyran (2.1 g, 24.6 mmol), 1H-indazol-3(2H)-one (3 g, 22.4 mmol), toluene-4-sulfonic acid (775 mg, 4.5 mmol) in THF (25 mL) was stirred at RT overnight. The mixture was concentrated under vacuum and purified by silica gel column chromatography (1%-5% EtOAc in pet. ether) to afford 87A (2 g, 41% yield) as a white solid: ¹H NMR (400 MHz, DMSO-d₆) δ 10.79 (s, 1H), 7.62 (d, J=8 Hz, 1H), 7.52 (d, J=8.4 Hz, 1H), 7.36 (t, J=7.6 Hz, 1H), 7.07-7.03 (m, 1H), 5.58 (dd, J=10 Hz, 1 Hz, 1H), 3.87-3.84 (m, 1H), 3.69-3.62 (m, 1H), 2.34-2.24 (m, 1H), 2.01-1.98 (m, 1H), 1.91-1.87 (m, 1H), 1.76-1.65 (m, 1H), 1.55-1.48 (m, 2H); ESI m/z 219.1 [M+H]⁺.

Step 5: A mixture of 87A (550 mg, 2.52 mmol), 1D (668 mg, 2.52 mmol) Pd₂(dba)₃ (74 mg, 0.08 mmol), Xantphos (75 mg, 0.13 mmol) and Cs₂CO₃ (985 mg, 3.0 mmol) in dioxane (50 mL) was heated to 100° C. overnight. After cooling to room temperature, the reaction mixture was filtered. The filtrate was concentrated under reduced pressure and purified by column chromatography (1%-50% EtOAc in pet. ether) to give 87B (600 mg, 60% yield) as a yellow solid: ESI m/z 403.1 [M+H]⁺.

Step 6: A mixture of 87B (600 mg, 1.49 mmol) and HCl (1.0 M, 20 mL) in THF (10 mL) was stirred at room temperature overnight. The mixture was poured into water and extracted with EtOAc (100 mL×3). The combined organic fractions were washed with water and brine and dried over sodium sulfate. The solvent was removed under vacuum and the residue was purified by column chromatography on silica gel (1%-5% MeOH in DCM) to provide compound 87 (230 mg, 48% yield) as a white solid: ¹H NMR (400 MHz, DMSO-d₆) δ 12.69 (s, 1H), 8.42 (s, 1H), 8.13-8.09 (m, 1H), 7.91 (d, J=7.2 Hz, 1H), 7.51 (d, J=8.4 Hz, 1H), 7.44-7.36 (m, 3H), 7.06 (t, J=7.2 Hz, 1H), 2.85-2.79 (m, 1H), 0.65-0.60 (m, 2H), 0.15-0.10 (m, 2H); ESI m/z 319.0 [M+H]⁺.

### Example 2

### Preparation of 2-(6-(4-cyclopropyl-4H-1,2,4-triazol-3-yl)pyridin-2-yl)-1-methyl-1,2-dihydro-3H-indazol-3-one (compound 88)

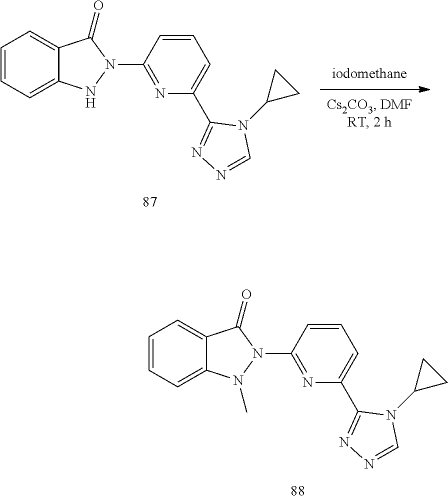 [SMILES: O=c1c2ccccc2[nH]n1-c1cccc(-c2nncn2C2CC2)n1 ; Cn1c2ccccc2c(=O)n1-c1cccc(-c2nncn2C2CC2)n1]

To a mixture of compound 87 (100 mg, 0.31 mmol) and Cs₂CO₃ (206 mg, 0.63 mmol) in DMF (10 mL) was added iodomethane (67 mg, 0.47 mmol). After stirring at room temperature for 2 h, the mixture was poured into water and extracted with EtOAc (50 mL×3), the organic layer was washed with water and brine, dried over Na₂SO₄, filtered, concentrated and purified by silica gel column chromatography (MeOH/DCM=1/100 to 1/30, v/v) and Prep-TLC (MeOH/DCM=1/15, v/v) to afford the product (13 mg, 13% yield) as a white solid: ¹H NMR (400 MHz, DMSO-d₆) δ 8.43 (s, 1H), 8.11 (t, J=8 Hz, 1H), 7.91 (d, J=7.6 Hz, 1H), 7.64 (d, J=8.8 Hz, 1H), 7.45-7.38 (m, 3H), 7.11-7.07 (m, 1H), 3.97 (s, 3H), 2.87-2.81 (m, 1H), 0.66-0.62 (m, 2H), 0.17-0.12 (m, 2H); ESI m/z 333.0 [M+H]⁺.

### Example 3

### Preparation of 2-(6-(4-cyclopropyl-4H-1,2,4-triazol-3-yl)pyridin-2-yl)benzo[d]isothiazol-3(2H)-one (compound 89)

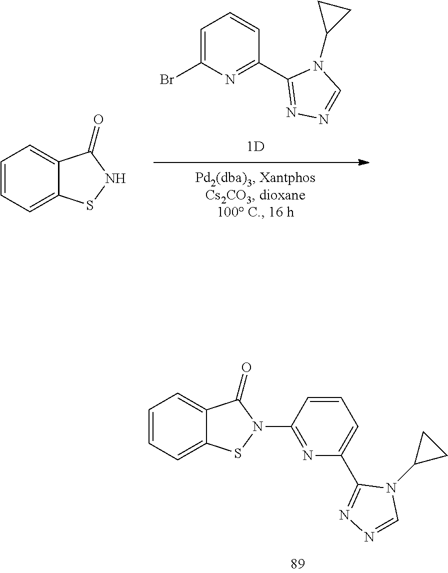 [SMILES: O=c1[nH]sc2ccccc12 ; Brc1cccc(-c2nncn2C2CC2)n1 ; O=c1c2ccccc2sn1-c1cccc(-c2nncn2C2CC2)n1]

A stirred mixture of benzokIlisothiazol-3(2H)-one (113 mg, 0.75 mmol), 1D (200 mg, 0.75 mmol), Pd₂(dba)₃ (34 mg, 0.0375 mmol), Cs₂CO₃ (733 mg, 2.25 mmol) and Xantphos (30 mg, 0.0525 mmol) in 1,4-dioxane (20 mL) was heated to 100° C. overnight. The reaction mixture was allowed to cool to room temperature and filtered. The filtrate was concentrated under reduced pressure and the residue was purified by silica column chromatography (1%-5% MeOH in DCM) to afford compound 89 (20 mg, 8% yield) as a yellow solid: ¹H NMR (400 MHz, CDCl₃) δ 8.86 (d, J=8.4 Hz, 1H), 8.27 (s, 1H), 8.10 (dd, J=7.6 Hz, 3.2 Hz, 1H), 7.97 (t, J=8 Hz, 1H), 7.68 (t, J=7.6 Hz, 1H), 7.57 (d, J=8 Hz, 1H), 7.44 (t, J=7.6 Hz, 1H), 4.16-4.11 (m, 1H), 1.28-1.23 (m, 2H), 0.96-0.92 (m, 2H); ESI m/z 336.0 [M+H]⁺.

### Example 4

### Preparation of 5-(6-(4-Cyclopropyl-4H-1,2,4-triazol-3-yl)pyridin-2-yl)-4,5-dihydro-6H-thieno[2,3-c]pyrrol-6-one (compound 90)

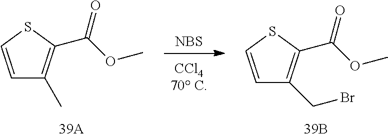

Step 1: A mixture of methyl 3-methylthiophene-2-carboxylate (5.00 g, 32.0 mmol), 2,2′-azobisisobutyronitrile (526 mg, 3.20 mmol) and N-bromosuccinimide (5.70 g, 32.0 mmol) in CCl₄ (300 mL) was heated to 70° C. After 3 hr, the mixture was allowed to cool to room temperature and was filtered. The resulting filter cake was washed with carbon tetrachloride (2×50 mL). The filtrate was diluted with ethyl acetate (300 mL) and washed with water (40 mL), saturated aqueous sodium bicarbonate (40 mL) and saturated brine (40 mL). The organic layer was dried over anhydrous Na₂SO₄, filtered and concentrated under reduced pressure. The crude product was purified by silica gel chromatography (1%-2% EtOAc in PE) to afford compound 39B (4.0 g, 54% yield) as a colorless oil. ESI m/z 234.9 [M+H]⁺.

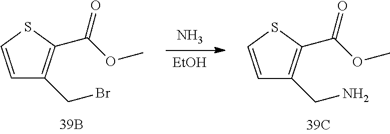

Step 2: A mixture of 39B (4.0 g, 17.1 mmol) in 2N NH₃/EtOH (23 mL) was stirred at room temperature overnight. The reaction mixture was concentrated at reduced pressure and the residue was purified by silica gel chromatography (2.5%-10% MeOH in DCM) to give compound 39C (1.2 g, 41% yield) as a white solid. ESI m/z 172.0 [M+H]⁺.

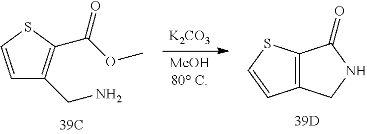

Step 3: A mixture of 39C (500 mg, 2.9 mmol) and anhydrous potassium carbonate (801 mg, 5.8 mmol) in methanol (40 mL) was stirred at 80° C. After 16 hr the reaction mixture was concentrated under reduced pressure. The residue was diluted with water (50 mL) and extracted with ethyl acetate (3×100 mL). The organic layers were combined, dried over anhydrous sodium sulfate and concentrated under reduced pressure. The residue was purified by silica gel chromatography (5%-10% MeOH in DCM) to afford compound 39D (400 mg, 50% yield) as a white solid. ESI m/z 177.9 [M+K]⁺.

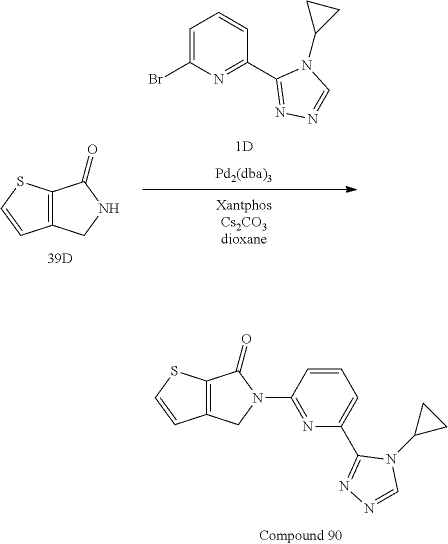 [SMILES: Brc1cccc(-c2nncn2C2CC2)n1 ; O=C1NCc2ccsc21 ; O=C1c2sccc2CN1c1cccc(-c2nncn2C2CC2)n1]

Step 4: A stirred mixture of compound 39D (200 mg, 1.4 mmol), compound 1D (371 mg, 1.4 mmol), Pd₂(dba)₃ (64 mg, 0.07 mmol), Cs₂CO₃ (1.4 g, 4.2 mmol) and Xantphos (58 mg, 0.1 mmol) in 1,4-dioxane (30 mL) was heated to 100° C. overnight. The reaction mixture was allowed to cool to room temperature and filtered. The resulting filtrate was concentrated under reduced pressure and the crude material was purified by silica gel chromatography (1.5% MeOH in DCM) to give compound 90 (80 mg, 18%) as a white powder: ¹H NMR (400 MHz, CDCl₃) δ 8.63 (d, J=9.2 Hz, 1H), 8.22 (s, 1H), 7.95-7.86 (m, 2H), 7.76 (d, J=4.8 Hz, 1H), 7.13 (d, J=4.8 Hz, 1H), 5.0 (s, 2H), 3.91 (m, 1H), 1.15-1.09 (m, 2H), 0.97-0.93 (m, 2H); ESI m/z 324.0 [M+H]⁺.

### Example 5

### Preparation of 2-(2-Acetylphenyl)-5-(6-(4-cyclopropyl-4H-1,2,4-triazol-3-yl) pyridin-2-yl)-4,5-dihydro-6H-thieno[2,3-c]pyrrol-6-one (compound 91)

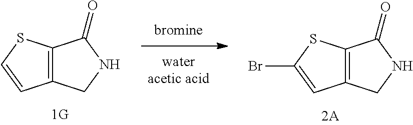 [SMILES: O=C1NCc2ccsc21 ; O=C1NCc2cc(Br)sc21]

Step 1: Bromine (3.4 g, 20 mmol) was added to a solution of 1G (1.5 g, 10 mmol) in acetic acid (20 mL) and water (10 mL) at 0° C. After stirring overnight, the reaction mixture was diluted with water (50 mL) and the mixture was extracted with EtOAc (2×100 mL). The combined organic fractions were washed with 5% aq. Na₂SO₃ (100 mL), sat. aq. NaHCO₃ (100 mL) and brine (100 mL). The reaction mixture was dried over Na₂SO₄, filtered and concentrated under vacuum. The residue was purified by flash chromatography over silica gel (5% MeOH in DCM) to yield 2A (1.2 g, 51% yield) as a yellow solid: ¹H NMR (400 MHz, MeOD) δ 7.27 (s, 1H), 4.38 (s, 3H); ESI m/z 218.0, 220.0 [M+H]⁺.

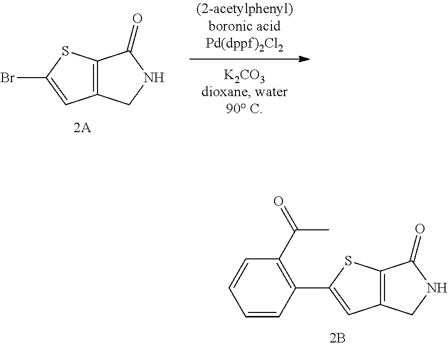 [SMILES: O=C1NCc2cc(Br)sc21 ; CC(=O)c1ccccc1-c1cc2c(s1)C(=O)NC2]

Step 2: A mixture of compound 2A (200 mg, 0.92 mmol), (2-acetylphenyl)boronic acid (226 mg, 1.38 mmol), potassium carbonate (381 mg, 2.76 mmol) and Pd(dppf)₂Cl₂ (34 mg, 0.046 mmol) in 1,4-dioxane (20 mL) and water (5 mL) was stirred at 90° C. for 10 hours under a nitrogen atmosphere. The mixture was poured into water and extracted with EtOAc (100 mL×3). The combined organic fractions were washed with water and brine, dried over anhydrous Na₂SO₄ and concentrated under reduced pressure. The residue was purified by chromatography on silica gel (1%-5% MeOH in DCM) to give 2B (200 mg, 84% yield) as a white solid: ESI m/z 258.1 [M+H]⁺.

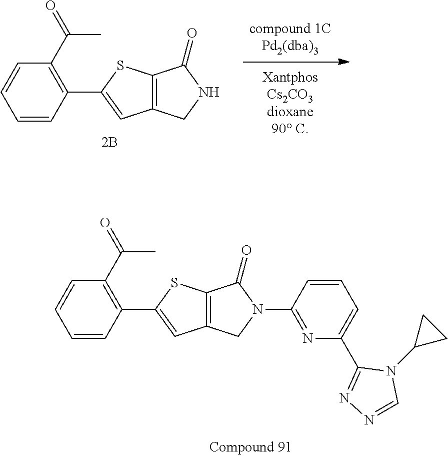 [SMILES: CC(=O)c1ccccc1-c1cc2c(s1)C(=O)NC2 ; CC(=O)c1ccccc1-c1cc2c(s1)C(=O)N(c1cccc(-c3nncn3C3CC3)n1)C2]

Step 3: A mixture of 2B (200 mg, 0.78 mmol), 1C (206 mg, 0.78 mmol), Cs₂CO₃ (762 mg, 2.34 mmol), Xantphos (32 mg, 0.055 mmol) and Pd₂(dba)₃ (36 mg, 0.039 mmol) in dioxane (25 mL) was stirred at 90° C. overnight under nitrogen. The mixture was concentrated under reduced pressure and purified by chromatography on silica gel (1%-5% MeOH in DCM) to give Compound 91 (80 mg, 23% yield) as a white solid: ¹H NMR (400 MHz, CDCl₃) δ 8.65 (dd, J=8 Hz, 0.8 Hz, 1H), 8.24 (s, 1 H), 7.97-7.89 (m, 2H), 7.57-7.49 (m, 4H), 7.03 (s, 1H), 5.02 (s, 2H), 3.96-3.90 (m, 1H), 2.32 (s, 3H), 1.18-1.13 (m, 2H), 0.98-0.94 (m, 2H); ESI m/z 442.0 [M+H]⁺.

### Example 6

### Preparation of N-(3-(5-(6-(4-Cyclopropyl-4H-1,2,4-triazol-3-yl)pyridin-2-yl)-6-oxo-5,6-dihydro-4H-thieno[2,3-c]pyrrol-2-yl)phenypacetamide (compound 92)

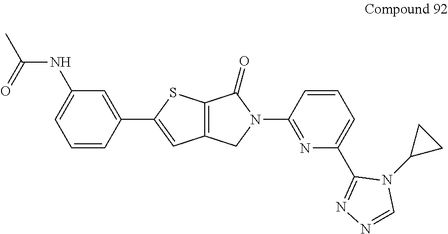 [SMILES: CC(=O)Nc1cccc(-c2cc3c(s2)C(=O)N(c2cccc(-c4nncn4C4CC4)n2)C3)c1]

Compound 92 was prepared according to the procedure for Example 2 substituting (3-acetamidophenyl)boronic acid in place of (2-acetylphenyl) boronic acid. ¹H NMR (400 MHz, DMSO-d₆) δ 8.72-8.66 (m, 3H), 8.13-8.05 (m, 3H), 7.98 (d, J=8 Hz, 1H), 7.92 (d, J=7.6 Hz, 1H), 7.81 (d, J=7.2 Hz, 1H), 7.66 (d, J=7.6 Hz, 1H), 7.40-7.33 (m, 2H), 5.30 (s, 2H), 4.16-4.10 (m, 1H), 1.17-1.12 (m, 2H), 1.02-0.98 (m, 2H); ESI m/z 434.0 [M+H]⁺.

### Example 7

### Preparation of 5-(6-(4-cyclopropyl-4H-1,2,4-triazol-3-yl)pyridin-2-yl)-2-(tetrahydro-2H-pyran-4-yl)-4,5-dihydro-6H-thieno[2,3-c]pyrrol-6-one (compound 93)

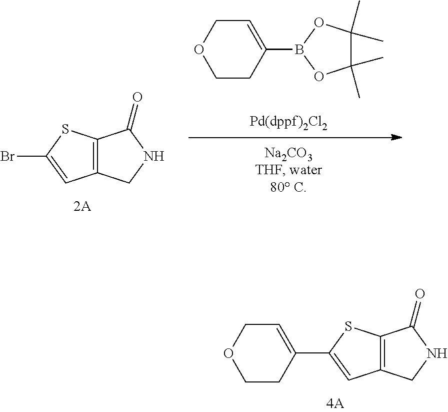

Step 1: A mixture of 2A (600 mg, 2.75 mmol), 2-(3,6-dihydro-2H-pyran-4-yl)-4,4,5,5-tetramethyl-1,3,2-dioxaborolane (867 mg, 4.13 mmol), Pd(dppf)C1₂ (402 mg, 0.55 mmol) and Na₂CO₃ (875 mg, 8.25 mmol) in THF (15 mL) and water (3 mL) was stirred at 80° C. overnight. The mixture was extracted with EtOAc (100 mL×3). The organic fractions were dried over anhydrous Na₂SO₄, filtered and concentrated under reduced pressure. The residue was purified by chromatography on silica gel (1%-1.25% MeOH in DCM) to give 4A (212 mg, 34% yield) as a yellow solid: ESI m/z 222.0 [M+H]⁺.

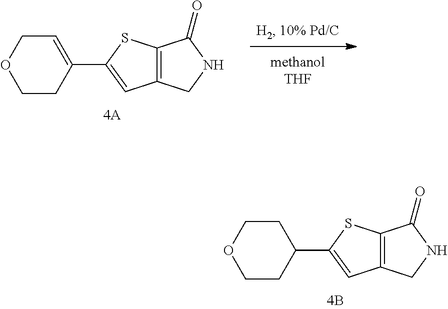 [SMILES: O=C1NCc2cc(C3=CCOCC3)sc21 ; O=C1NCc2cc(C3CCOCC3)sc21]

Step 2: A mixture of 4A (215 mg, 0.97 mmol) and 10% palladium on carbon (100 mg) was stirred in methanol (20 mL) and THF (20 mL) under a hydrogen atmosphere overnight. The reaction mixture was filtered through Celite and the filtrate was concentrated under reduced pressure to give 4B (230 mg, 100% yield) as a yellow solid: ¹H NMR (400 MHz, CDCl₃) δ 6.83 (s, 1H), 6.11 (s, 1H), 4.35 (s, 2H), 4.09 (dd, J=11.2 Hz, 3.2 Hz, 2H), 3.55 (td, J=11.7 Hz, 2.0 Hz, 2H), 3.13 (s, 1H), 2.04-1.94 (m, 2H), 1.88 (td, J=11.8, 3.7 Hz, 2H); ESI m/z 224.1 [M+H]⁺.

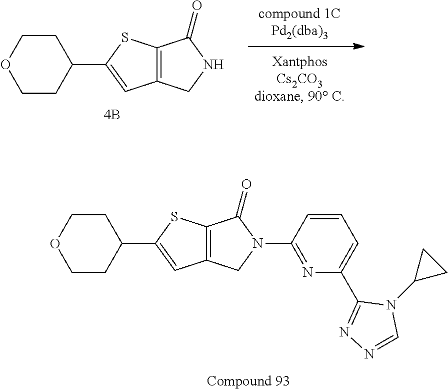 [SMILES: O=C1NCc2cc(C3CCOCC3)sc21 ; O=C1c2sc(C3CCOCC3)cc2CN1c1cccc(-c2nncn2C2CC2)n1]

Step 3: A mixture of 4B (200 mg, 0.90 mmol), 1C (239 mg, 0.90 mmol), Cs₂CO₃ (880 mg, 2.70 mmol), Xantphos (104 mg, 0.18 mmol) and Pd₂(dba)₃ (82 mg, 0.09 mmol) in dioxane (15 mL) was stirred at 90° C. overnight under nitrogen. The mixture was poured into water and extracted with EtOAc (100 mL×3). The organic fractions were dried over anhydrous Na₂SO₄, filtered and concentrated under reduced pressure. The residue was purified by chromatography on silica gel (1%-2% MeOH in DCM) to give compound 93 (70 mg, 20% yield) as a yellow oil: ¹H NMR (400 MHz, CDCl₃) δ 8.62 (dd, J=1.2 Hz 1H), 8.22 (s, 1H), 7.94-7.86 (m, 2H), 6.89 (s, 1H), 4.94 (s, 2H), 4.10-4.07 (m, 2H), 3.93-3.87 (m, 1H), 3.54 (t, J=12 Hz, 2H), 3.18-3.10 (m, 1H), 2.03-1.95 (m, 2H), 1.92-1.81 (m, 2H), 1.11 (q, J=6.8 Hz, 2H), 0.97-0.93 (m, 2H); ESI m/z 408.1 [M+H]⁺.

### Example 8

### Preparation of 5-(6-(4-cyclopropyl-4H-1,2,4-triazol-3-yl)pyridin-2-yl)-2-methyl-4,5-dihydro-6H-thieno[2,3-c]pyrrol-6-one (compound 96)

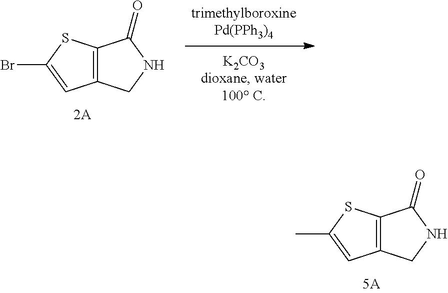

Step 1: A stirred mixture of 2A (500 mg, 2.29 mmol), trimethylboroxine (4.3 g, 34.39 mmol), Pd(PPh₃)₄ (265 mg, 0.23 mmol), and K₂CO₃ (1.6 g, 11.45 mmol) in 1,4-dioxane (40 mL) and water (5 mL) was heated to 100° C. for 6 hours under nitrogen. The reaction mixture was allowed to cool to room temperature, poured into water, and extracted with EtOAc (3×80 mL). The combined organic fractions were washed with brine, dried with anhydrous sodium sulfate, filtered and concentrated under reduced pressure. The residue was purified by column chromatography on silica gel (0.5%-30% EtOAc in PE) to give compound 5A (220 mg, 63% yield) as a white solid: ESI m/z 154.0 [M+H]⁺.

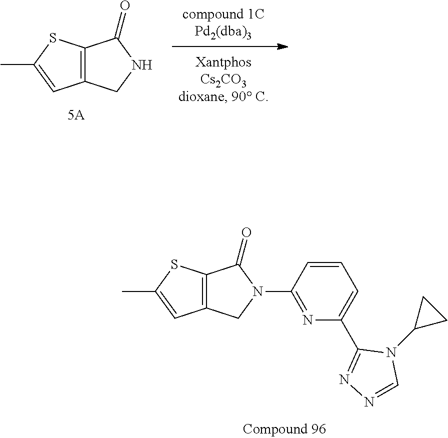

Step 2: A stirred mixture of 5A (220 mg, 1.44 mmol), compound 1C (381 mg, 1.44 mmol), Pd₂(dba)₃ (66 mg, 0.072 mmol), Xantphos (58 mg, 58 mmol) and Cs₂CO₃ (1.41 g, 4.32 mmol) in 1,4-dioxane (40 mL) was heated to 100° C. overnight under nitrogen. The reaction mixture was allowed to cool to room temperature and filtered. The filtrate was concentrated under reduced pressure and the residue was purified by silica column chromatography (0.5%-2.5% MeOH in DCM). The product was further purified by preparative HPLC to give Compound 96 (28 mg, 6% yield) as a white solid: ¹H NMR (400 MHz, CDCl₃) δ 8.62-8.60 (m, 1H), 8.22 (s, 1H), 7.91-7.84 (m, 2H), 6.80 (s, 1H), 4.91 (s, 2H), 3.93-3.88 (m, 1H), 2.61 (s, 3H), 1.13-1.08 (m, 2H), 0.96-0.92 (m, 2H); ESI m/z 338.1 [M+H]⁺.

### Example 9

### Preparation of 5-(6-(4-cyclopropyl-4H-1,2,4-triazol-3-yl)pyridin-2-yl)-2-ethyl-4,5-dihydro-6H-thieno[2,3-c]pyrrol-6-one (compound 97)

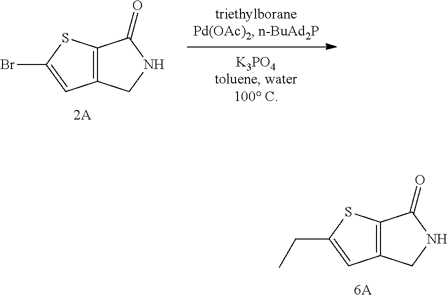

Step 1: A stirred mixture of 2A (600 mg, 2.75 mmol), triethylborane (1M in THF, 8.25 mL, 8.25 mmol), Pd(OAc)₂ (273 mg, 0.83 mmol), butyl di-1-adamantylphosphine (179 mg, 0.50 mmol) and K₃PO₄.3H₂O (2.2 g, 8.25 mmol) in 1,4-dioxane (40 mL) and water (4 mL) was heated to 100° C. overnight under nitrogen. The reaction mixture was allowed to cool to room temperature, poured into water and extracted with EtOAc (3×80 mL). The combined organic fractions were washed with brine, dried with anhydrous sodium sulfate, filtered and concentrated under reduced pressure. The residue was purified by column chromatography on silica gel (0.5%-50% EtOAc in PE) to give a mixture (240 mg) containing 6A: ESI m/z 167.8 [M+H]⁺.

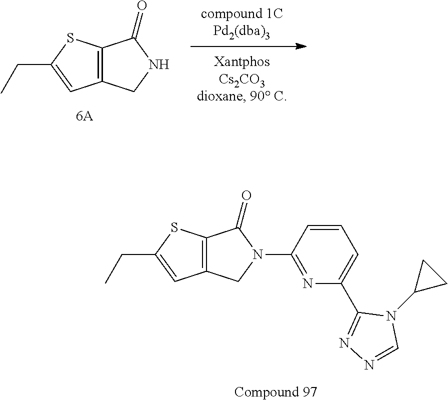 [SMILES: CCc1cc2c(s1)C(=O)NC2 ; CCc1cc2c(s1)C(=O)N(c1cccc(-c3nncn3C3CC3)n1)C2]

Step 2: A mixture of 6A (240 mg, 1.44 mmol), compound 1C (381 mg, 1.44 mmol), Pd₂(dba)₃ (66 mg, 0.072 mmol), Xantphos (58 mg, 58 mmol) and Cs₂CO₃ (1.41 g, 4.32 mmol) in 1,4-dioxane (40 mL) was stirred at 100° C. overnight under nitrogen. The reaction mixture was allowed to cool to room temperature and filtered. The filtrate was concentrated under reduced pressure and the residue was purified by silica gel column chromatography (0.5%-2.5% MeOH in DCM). The product was further purified by preparative reverse phase HPLC to afford Compound 97 (35 mg, 7% yield) as a white solid: ¹H NMR (400 MHz, CDCl₃) δ 8.62-8.60 (m, 1H), 8.22 (s, 1H), 7.91-7.84 (m, 2H), 6.84 (s, 1H), 4.91 (s, 2H), 3.93-3.88 (m, 1H), 2.68-2.92 (m, 2H), 1.37 (m, 3H), 1.13-1.08 (m, 2H), 0.96-0.92 (m, 2H); ESI m/z 352.1 [M+H]⁺.

### Example 10

### Preparation of 5-(6-(4-cyclopropyl-4H-1,2,4-triazol-3-yl)pyridin-2-yl)-3-phenyl-4,5-dihydro-6H-thieno[2,3-c]pyrrol-6-one (compound 94)

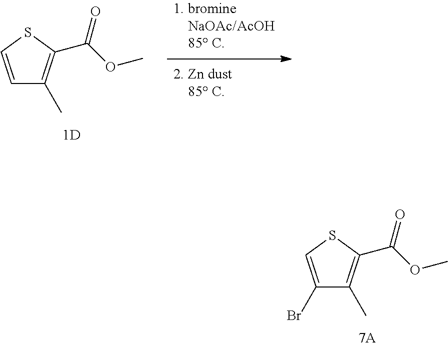

Step 1: A solution of 1D (20 g, 103 mmol) and sodium hydroxide (12.3 g, 307 mmol) in acetic acid (75 mL) was heated to 60° C. Bromine (46.9 g, 294 mmol) was added dropwise in order to keep the reaction temperature below 85° C. After stirring at 85° C. for 6 h, the reaction mixture was allowed to cool to 50° C. Zinc dust (15.4 g, 236 mmol) was added in 3 gram portions to the mixture to keep the reaction temperature below 85° C. After stirring at 85° C. for 1 h, the hot reaction mixture was filtered through a small bed of Celite. The filtrate was diluted with water (300 mL) and the mixture was extracted with hexane (300 mL). The organic fraction was washed with water and concentrated under vacuum to give 7A (27 g, 89% yield) as an off-white solid: ¹H NMR (400 MHz, CDCl₃) δ 7.42 (s, 1H), 3.86 (s, 3H), 2.54 (s, 3H); ESI m/z 234.9, 236.9 [M+H]⁺.

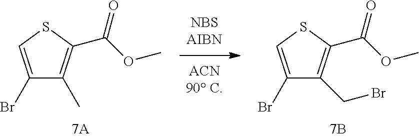

Step 2: A mixture of 7A (0.5 g, 2.14 mmol), N-bromosuccinimide (0.392 g, 2.2 mmol) and 2,2′-azobis(2-methylpropionitrile) (0.175 g, 1.05 mmol) in ACN (20 mL) was stirred overnight at 90° C. under nitrogen. The mixture was then filtered, evaporated and purified by column chromatography on silica gel (petroleum ether) to give 7B (0.4 g, 60% yield) as a yellow oil: ¹H NMR (400 MHz, CDCl₃) δ 7.48 (s, 1H), 4.90 (s, 2H), 3.93 (s, 3H); ESI m/z 314.7 [M+H]⁺.

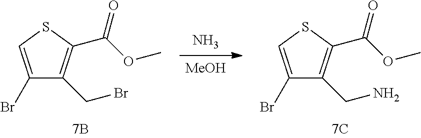

Step 3: A mixture of 7B (18 g, 58.3 mmol) and ammonia in methanol (7.0 M, 230 mL) was stirred at room temperature overnight. The mixture was concentrated under reduced pressure and purified by column chromatography on silica (1%-5% EtOAc in PE) to afford compound 7C (10.7 g, 74% yield) as an off-white solid: ESI m/z 249.9, 251.8 [M+H]⁺.

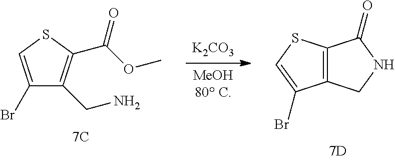

Step 4: A mixture of 7C (10 g, 40.2 mmol) and potassium carbonate (16.7 g, 120.5 mmol) in methanol (120 mL) was heated at 80° C. overnight under nitrogen. The mixture was concentrated under reduced pressure and purified by column chromatography on silica (1%-50% EtOAc in PE) to afford compound 7D (2.8 g, 32% yield) as a yellow solid: ¹H NMR (400 MHz, CDCl₃) δ 7.57 (s, 1H), 6.98 (s, 1H), 4.30 (s, 2H); ESI m/z 217.8, 219.8 [M+H]⁺.

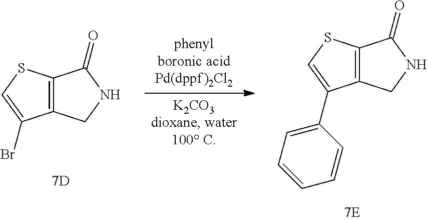 [SMILES: O=C1NCc2c(Br)csc21 ; "K2CO3dioxane, water100° C." ; "phenylboronic acidPd(dppf)2Cl2" ; O=C1NCc2c(-c3ccccc3)csc21]

Step 5: A mixture of 7D (300 mg, 1.38 mmol), phenylboronic acid (252 mg, 2.06 mmol), potassium carbonate (572 mg, 4.14 mmol) and Pd(dppf)₂C1₂ (63 mg, 0.069 mmol) in 1,4-dioxane (20 mL) and water (10 mL) was stirred at 100° C. overnight under a nitrogen atmosphere. The mixture was poured into water and extracted with EtOAc (100 mL×3). The organic fraction was washed with water and brine, dried over anhydrous sodium sulfate and concentrated under reduced pressure. The residue was purified by chromatography on silica gel (1%-5% MeOH in DCM) to give compound 7E (197 mg, 66% yield) as a white solid: ESI m/z 216.2 [M+H]⁺.

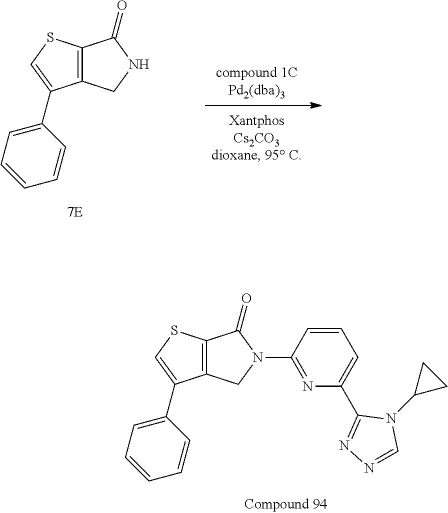 [SMILES: O=C1NCc2c(-c3ccccc3)csc21 ; O=C1c2scc(-c3ccccc3)c2CN1c1cccc(-c2nncn2C2CC2)n1]

Step 6: A mixture of 7E (130 mg, 0.60 mmol), compound 1C (160 mg, 0.60 mmol), cesium carbonate (586 mg, 1.80 mmol), Xantphos (24 mg, 0.042 mmol) and Pd(dba)₃ (27 mg, 0.037 mmol) in 1,4-dioxane (20 mL) was stirred at 95° C. overnight under nitrogen. The mixture was concentrated under reduced pressure and purified by chromatography on silica gel (1%-5% MeOH in DCM) to give Compound 94 (50 mg, 21% yield) as a white solid: ¹H NMR (400 MHz, CDCl₃) δ 8.70 (s, 1H), 8.51 (d, J=8 Hz, 1H), 8.43 (s, 1H), 8.06 (t, J=8 Hz, 1H), 7.88 (d, J=7.2 Hz, 1H), 7.71 (d, J=7.2 Hz, 1H), 7.51-7.38 (m, 3H), 5.41 (s, 2H), 4.21-4.14 (m, 1H), 1.11-1.06 (m, 4H); ESI m/z 400.0 [M+H]⁺.

### Example 11

### Preparation of 5-(6-(4-cyclopropyl-4H-1,2,4-triazol-3-yl)pyridin-2-yl)-3-(tetrahydro-2H-pyran-4-yl)-4,5-dihydro-6H-thieno[2,3-c]pyrrol-6-one (compound 98)

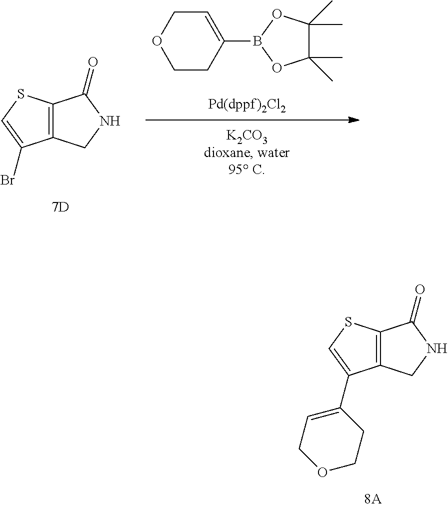

Step 1: A mixture of 7D (600 mg, 2.75 mmol), 2-(3,6-dihydro-2H-pyran-4-yl)-4,4,5,5-tetramethyl-1,3,2-dioxaborolane (867 mg, 4.13 mmol), potassium carbonate (1.14 g, 8.25 mmol) and Pd(dppf)₂Cl₂ (126 mg, 0.138 mmol) in 1,4-dioxane (40 mL) and water (10 mL) was stirred at 95° C. overnight under nitrogen. The mixture was poured into water and extracted with EtOAc (100 mL×3), the organic fractions were washed with water and brine, dried over Na₂SO₄ and concentrated under reduced pressure. The residue was purified by chromatography on silica gel (1%-5% MeOH in DCM) to give compound 8A (330 mg, 54% yield) as a white solid: ESI m/z 222.1 [M+H]⁺.

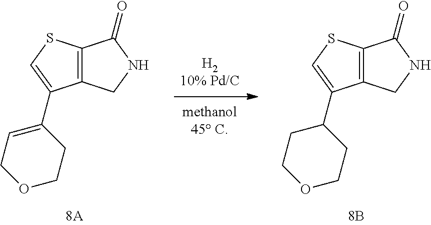 [SMILES: O=C1NCc2c(C3=CCOCC3)csc21 ; O=C1NCc2c(C3CCOCC3)csc21]

Step 2: A mixture of 8A (300 mg, 1.36 mmol) and 10% palladium on carbon (300 mg) in methanol (20 mL) was heated to 45° C. overnight under one atmosphere of hydrogen. The reaction mixture was filtered through Celite and the filtrate was concentrated under vacuum. The residue was purified by silica column chromatography (5% MeOH in DCM) to give compound 8B (80 mg, 59% yield) as a white solid: ESI m/z 224.1 [M+H]⁺.

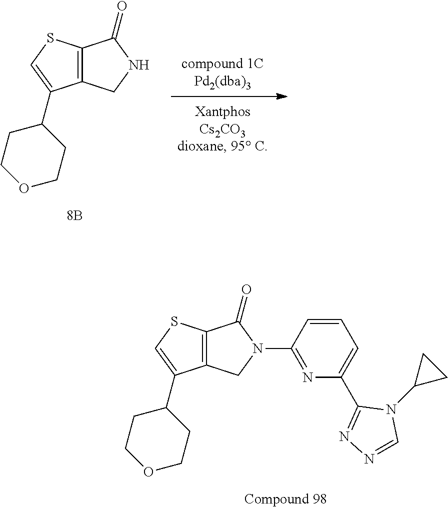 [SMILES: O=C1NCc2c(C3CCOCC3)csc21 ; O=C1c2scc(C3CCOCC3)c2CN1c1cccc(-c2nncn2C2CC2)n1]

Step 3: A mixture of 8B (170 mg, 0.76 mmol), compound 1C (155 mg, 0.76 mmol), cesium carbonate (743 mg, 2.28 mmol), Xantphos (31 mg, 0.053 mmol) and Pd₂(dba)₃ (35 mg, 0.038 mmol) in 1,4-dioxane (15 mL) was stirred at 95° C. overnight under nitrogen. The mixture was concentrated under reduced pressure and purified by chromatography on silica gel (1%-5% MeOH in DCM) to give Compound 98 (45 mg, 15% yield) as a white solid: ¹H NMR (400 MHz, CDCl₃) δ 8.62 (dd, J=7.2 Hz, 2 Hz, 1H), 8.23 (s, 1H), 7.92-7.87 (m, 2H), 7.40 (s, 1H), 5.00 (s, 2H), 4.08 (dd, J=11.2 Hz, 2.8 Hz, 2H), 3.91-3.85 (m, 1H), 3.56-3.50 (m, 2H), 2.97-2.89 (m, 1H), 1.89-1.77 (m, 4H), 1.13-1.08 (m , 2H), 0.99-0.95 (m,2H); ESI m/z 408.0 [M+H]⁺.

### Example 12

### Preparation of 5-(6-(4-cyclopropyl-4H-1,2,4-triazol-3-yl)pyridin-2-yl)-3-methyl-4,5-dihydro-6H-thieno[2,3-c]pyrrol-6-one (compound 99)

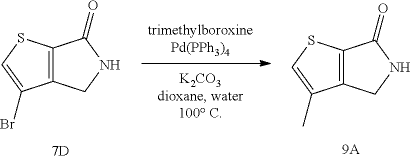

Step 1: A mixture of 7D (350 mg, 1.60 mmol), trimethylboroxine (3.0 g, 24.1 mmol), Pd(PPh₃)₄ (185 mg, 0.16 mmol) and potassium carbonate (1.1 g, 8.02 mmol) in 1,4-dioxane (30 mL) and water (2 mL) was heated to 100° C. for 6 hours under nitrogen After cooling to room temperature, the reaction mixture was diluted with water and extracted with EtOAc (3×80 mL). The organic fractions were washed with brine, dried with sodium sulfate and concentrated under reduced pressure. The residue was purified by column chromatography on silica gel (0.5%-1% MeOH in DCM) to give the compound 9A (240 mg, 98% yield) as a yellow solid: ¹H NMR (400 MHz, DMSO-d₆) δ 8.33 (s, 1H), 7.54 (s, 1H), 4.23 (s, 2H), 2.19 (s, 3H); ESI m/z 154.0 [M+H]⁺.

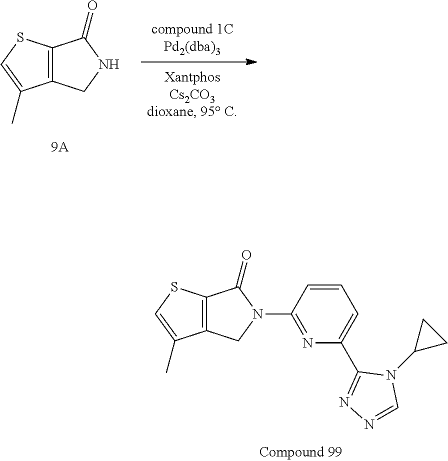

Step 2: A mixture of 9A (240 mg, 1.63 mmol), 1C (432 mg, 1.63 mmol), Pd₂(dba)₃ (100 mg, 0.11 mmol), Xantphos (91 mg, 0.16 mmol) and cesium carbonate (1.5 g, 4.70 mmol) in 1,4-dioxane (30 mL) was heated to 95° C. overnight under nitrogen. The reaction mixture was allowed to cool to room temperature and filtered. The resulting filtrate was concentrated under reduced pressure and the residue was purified by silica column chromatography (0.5%-1.25% MeOH in DCM) to give the Compound 99 (120 mg, 23% yield) as an off-white solid: ¹H NMR (400 MHz, CDCl₃) δ 8.65-8.61 (m, 1H), 8.24 (s, 1H), 7.90-7.88 (m, 2H), 7.35 (d, J=0.8 Hz, 1H), 4.89 (s, 2H), 3.92-3.86 (m, 1H), 2.29 (s, 3H), 1.14-1.09 (m, 2H), 0.97-0.93 (m, 2H); ESI m/z 338.0 [M+H]⁺.

### Example 13

### Preparation of 3-bromo-5-(6-(4-cyclopropyl-4H-1,2,4-triazol-3-yl)pyridin-2-yl)-4,5-dihydro-6H-thieno[2,3-c]pyrrol-6-one (compound 100)

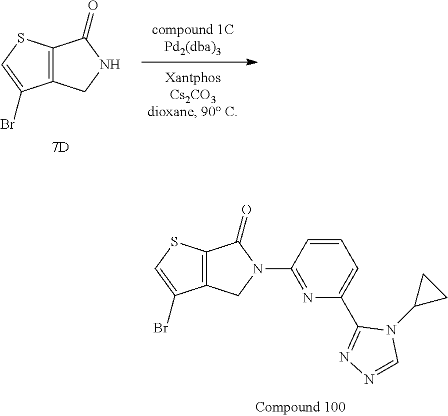 [SMILES: O=C1NCc2c(Br)csc21 ; O=C1c2scc(Br)c2CN1c1cccc(-c2nncn2C2CC2)n1]

A mixture of 7D (500 mg, 2.29 mmol), 1C (608 mg, 2.29 mmol), Pd₂(dba)₃ (147 mg, 0.16 mmol), Xantphos (132 mg, 0.22 mmol) and cesium carbonate (2.23 g, 6.82 mmol) in 1,4-dioxane (50 mL) was heated to 90° C. overnight under nitrogen. The reaction mixture was allowed to cool to room temperature and filtered. The resulting filtrate was concentrated under reduced pressure and the residue was purified by silica column chromatography (1%-1.25% MeOH in DCM) to give compound 100 (200 mg, 22% yield) as a yellow solid: ¹H NMR (400 MHz, DMSO-d₆) δ 8.69 (s, 1H), 8.47 (d, J=8 Hz, 1H), 8.26 (s, 1H), 8.06 (t, J=8 Hz, 1H), 7.85 (d, J=7.2 Hz, 1H), 5.07 (s, 2H), 4.06-4.02 (m, 1H), 1.10-0.99 (m, 4H); ESI m/z 401.9, 403.9 [M+H]⁺.

### Example 14

### Preparation of 5-(6-(4-cyclopropyl-4H-1,2,4-triazol-3-yl)pyridin-2-yl)-3-(isobutylamino)-4,5-dihydro-6H-thieno[2,3-c] pyrrol-6-one (compound 101)

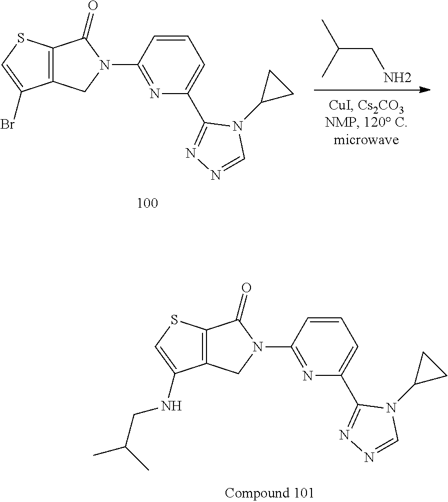

A mixture of 100 (100 mg, 0.25 mmol), 2-methylpropan-1-amine (72 mg, 1.0 mmol), CuI (48 mg, 0.25 mmol), and cesium carbonate (162 mg, 0.50 mmol) in NMP (6 mL) was heated at 120° C. for 40 m by microwave. The mixture was concentrated under vacuum and purified by column chromatography on silica gel (1%-2% MeOH in DCM) to afford compound 101 (3 mg, 3% yield) as a yellow solid: ¹H NMR (400 MHz, DMSO-d₆) δ 8.74 (s, 1H), 8.50 (d, J=8.4 Hz, 1H), 8.04 (t, J=8 Hz, 1H), 7.78 (d, J=7.2 Hz, 1H), 6.47 (s, 1H), 4.94 (s, 2H), 2.86 (d, J=8.4 Hz, 1H), 1.92-1.86 (m, 1H), 1.08-1.04 (m, 2H), 0.96-0.95 (m, 6H), 0.88-0.84 (m, 2H); ESI m/z 395.0 [M+H]⁺.

### Example 15

### Preparation of 2-(6-(4-cyclopropyl-4H-1,2,4-triazol-3-yl)pyridin-2-yl)-1,2-dihydro-3H-benzo[4,5]thieno[2,3-c]pyrrol-3-one (compound 95)

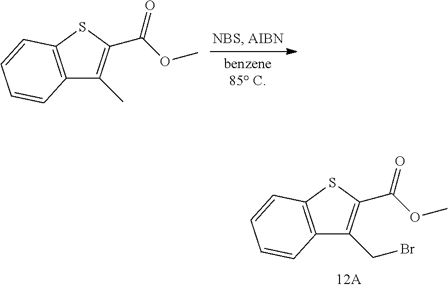

Step 1: A mixture of methyl 3-methylbenzo[b]thiophene-2-carboxylate (2.26 g, 11.1 mmol), N-bromosuccinimide (1.96 g, 11.10 mmol) and 2,2′-azobisisobutyronitrile (903 mg, 5.5 mmol) in benzene (30 mL) was stirred at 85° C. overnight. The mixture was concentrated under reduced pressure and purified by chromatography on silica gel (2%-25% EtOAc in PE) to give compound 12A (1.68 g, 54% yield) as a white solid: ESI m/z 285.4, 287.4 [M+H]⁺.

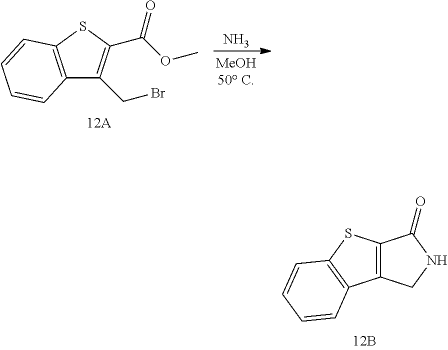

Step 2: Compound 12A (1.6 g, 5.61 mmol) was stirred in a solution of ammonia in methanol (7.0 M, 50 mL) at 50° C. overnight in a sealed tube. The mixture was concentrated under reduced pressure and purified by chromatography on silica gel (2%-50% EtOAc in PE) to give 12B (3 g, 80% yield) as an off-white solid: ESI m/z 190.1 [M+H]⁺.

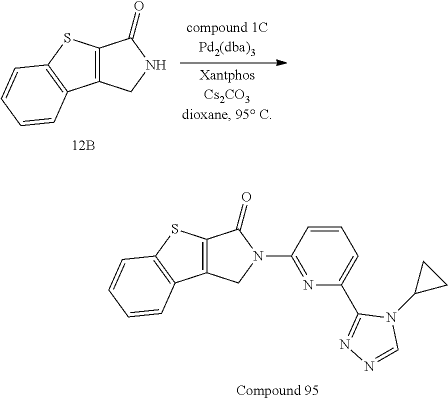 [SMILES: O=C1NCc2c1sc1ccccc21 ; O=C1c2sc3ccccc3c2CN1c1cccc(-c2nncn2C2CC2)n1]

Step 3: A mixture of 12B (160 mg, 0.85 mmol), 1C (173 mg, 0.85 mmol), cesium carbonate (831 mg, 2.55 mmol), Xantphos (34 mg, 0.0595 mmol) and Pd₂(dba)₃ (39 mg, 0.0425 mmol) in 1,4-dioxane (20 mL) was stirred at 95° C. overnight under nitrogen. The mixture was concentrated under reduced pressure and purified by chromatography on silica gel (1%-5% MeOH in DCM) to give compound 95 (50 mg, 16% yield) as a white solid: ¹H NMR (400 MHz, DMSO-d₆) δ 8.73 (s, 1H), 8.53 (dd, J=8.4 Hz, 1H), 8.21-8.15 (m, 2H), 8.07 (t, J=8.4 Hz, 1H), 7.85 (d, J=7.6 Hz, 1H), 7.59-7.57 (m, 2H), 5.37 (s, 2H), 4.16-4.10 (m, 1H), 1.15-1.11 (m, 2H), 1.05-0.95 (m, 2H); ESI m/z 374.0 [M+H]⁺.

### Example 16

### Preparation of 5-(6-(4-cyclopropyl-4H-1,2,4-triazol-3-yl)pyridin-2-yl)-2-methoxy-4,5-dihydro-6H-thieno[2,3-c]pyrrol-6-one (compound 102)

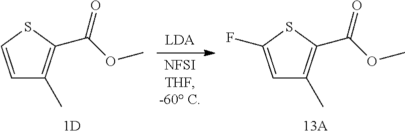

Step 1: n-BuLi (2.5 M in n-hexane, 6.7 mL, 16.7 mmol) was added to a stirred solution of diisopropylamine (1.56 g, 15.37 mmol) in THF (40 mL) at −78° C. The reaction mixture was stirred at 0° C. for 10 minutes and then cooled to −60° C. A solution of 1D (2.0 g, 12.81 mmol) in THF (40 mL) was added dropwise and the reaction was stirred at −60° C. for 30 min. A solution of N-fluorobenzenesulfonimide (5.25, 16.65 mmol) in THF (5 mL) was added and the reaction was stirred at −60° C. for 2 hours. The reaction mixture was quenched with a solution of saturated ammonium chloride and extracted with dichloromethane (2×100 mL). The combined organic fractions were dried over anhydrous sodium sulfate, filtered and concentrated under vacuum to a volume of ˜50 mL. The mixture was then purified by flash silica gel column chromatography (1% DCM in PE). The collected fractions were concentrated to a volume of ˜50 mL. Since the product was volatile, the solution of 13A was used directly in the next step: ¹H NMR (400 MHz, CDCl₃) δ 6.35 (d, J=1.2 Hz, 1H), 3.83 (s, 3H), 2.49 (s, 3H).

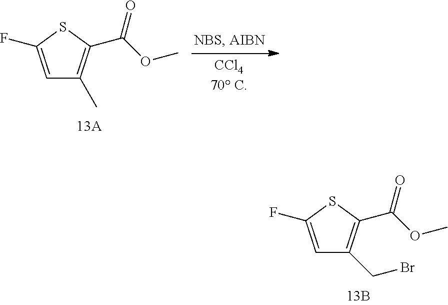

Step 2: A stirred mixture of 13A (estimated 3.44 mmol), 2,2′-azobisisobutyronitrile (113 mg, 0.688 mmol) and N-bromosuccinimide (613 mg, 3.44 mmol) in carbon tetrachloride (20 mL) was heated to 80° C. for 4 hours. Then the reaction mixture was cooled to room temperature and filtered. The filter cake was washed with carbon tetrachloride (2×20 mL). The filtrate was diluted with ethyl acetate (200 mL) and washed with water (40 mL), saturated aqueous sodium bicarbonate (40 mL) and brine (40 mL). The organic fraction was dried over anhydrous sodium sulfate, filtered and concentrated under reduced pressure to afford 13B (1 g) as a crude oil. The product was used without further purification.

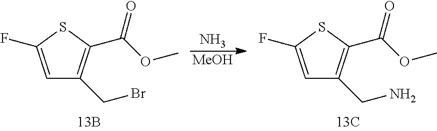

Step 3: 13B (800 mg, 3.16 mmol) was stirred in a solution of ammonia in methanol (7N, 30 mL) at room temperature overnight. The reaction mixture was concentrated at reduced pressure and the residue was purified by silica gel column chromatography (2%-10% MeOH in DCM) to give a mixture containing 13C (330 mg). The mixture was used in the next step. ESI m/z 190.1 [M+H]⁺.

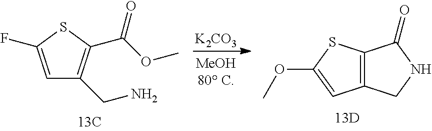

Step 4: A stirred mixture of 13C (300 mg, 1.59 mmol) and potassium carbonate (438 mg, 3.17 mmol) in methanol (30 mL) was stirred for overnight at 80° C. After cooling, the reaction mixture was concentrated under reduced pressure and purified by silica gel column chromatography (2%-5% MeOH in DCM) to afford a mixture containing 13D (110 mg): ¹H NMR (400 MHz, CDCl₃) δ 6.24 (s, 1H), 4.31 (s, 2H), 3.96 (s, 3H); ESI m/z 170.2 [M+H]⁺.

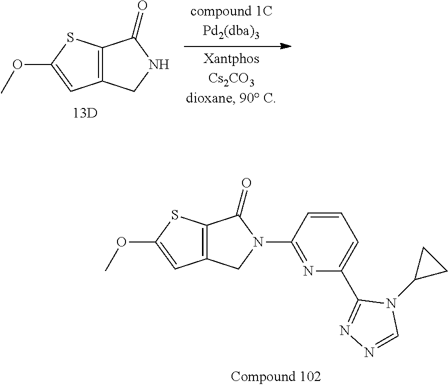 [SMILES: COc1cc2c(s1)C(=O)NC2 ; COc1cc2c(s1)C(=O)N(c1cccc(-c3nncn3C3CC3)n1)C2]

Step 5: A stirred mixture of 13D (110 mg, 0.65 mmol), compound 1C (172 mg, 0.65 mmol), Cs₂CO₃ (741 mg, 2.28 mmol), Xantphos (53 mg, 0.091 mmol) and Pd₂(dba)₃ (60 mg, 0.065 mmol) in 1,4-dioxane (6 mL) was stirred at 90° C. overnight under nitrogen. The mixture was concentrated under reduced pressure and purified by silica gel column chromatography (1%-5% MeOH in DCM) and preparative reverse phase HPLC to give compound 102 (15 mg, 7%) as a white solid: ¹H NMR (400 MHz, CDCl₃) δ 8.58 (dd, J=8 Hz, 1.2 Hz, 1H), 8.21 (s, 1H), 7.89-7.82 (m, 2H), 6.30 (s, 1H), 4.89 (s, 2H), 3.99 (s, 3H), 3.92-3.86 (m, 1H), 1.13-1.08 (m, 2H), 0.96-0.91 (m, 2H); ESI m/z 354.1 [M+H]⁺.

### Example 17

### Preparation of 5-(6-(4-isopropyl-4H-1,2,4-triazol-3-yl)pyridin-2-yl)-4,5-dihydro-6H-thieno[2,3-c]pyrrol-6-one (compound 103)

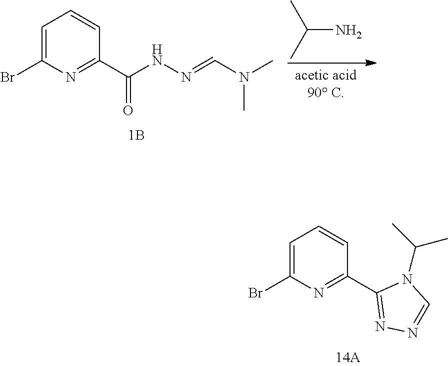

Step 1: A mixture of 1B (2.1 g, 7.75 mmol), propan-2-amine hydrochloride (3.71 g, 38.8 mmol) and DIPEA (5.0 g, 38.8 mmol) in ACN (32 mL) and acetic acid (8 mL) was heated to 90° C. overnight. The mixture was concentrated and purified by column chromatography on silica gel (1%-50% EtOAc in PE) to afford 14A (1.7 g, 82% yield) as a white solid: ¹H NMR (400 MHz, DMSO-d₆) δ 8.92 (s, 1), 8.15 (d, J=8 Hz, 1H), 7.96-7.92 (m, 1H), 7.77 (d, J=8 Hz, 1H), 5.34-5.24 (m, 1H), 1.48 (d, J=6.8 Hz, 6H); ESI m/z 266.9, 268.9 [M+H]⁺.

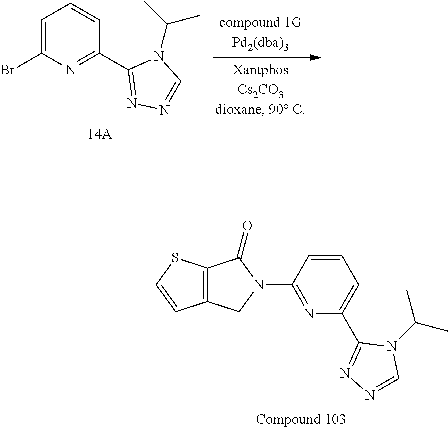 [SMILES: CC(C)n1cnnc1-c1cccc(Br)n1 ; CC(C)n1cnnc1-c1cccc(N2Cc3ccsc3C2=O)n1]

Step 2: A stirred mixture of 14A (286 mg, 108 mmol), 1G (150 mg, 1.08 mmol), Cs₂CO₃ (1.06 g, 3.24 mmol), Xantphos (87 mg, 0.15 mmol) and Pd₂(dba)₃ (99 mg, 0.108 mmol) in 1,4-dioxane (10 mL) was heated at 90° C. overnight under nitrogen. The mixture was concentrated under reduced pressure and purified by chromatography on silica gel (1%-5% MeOH in DCM) followed by preparative reverse phase HPLC to give the title compound 103 (80 mg, 23% yield) as a white solid: ¹H NMR (400 MHz, CDCl₃) δ 8.62 (d, J=8.4 Hz, 1H), 8.42 (s, 1H), 8.02 (d, J=7.6 Hz, 1H), 7.89 (t, J=8 Hz, 1H), 7.69 (d, J=4.8 Hz, 1H), 7.16 (d, J=4.8 Hz, 1H), 5.63-5.53 (m, 1H), 4.97 (s, 2H), 1.62 (d, J=6.8 Hz, 6H); ESI m/z 326.1 [M+H]⁺.

### Example 18

### Preparation of 5-(6-(4-cyclopropyl-4H-1,2,4-triazol-3-yl)pyridin-2-yl)-2-(dimethylamino)-4,5-dihydro-6H-thieno[2,3-c] pyrrol-6-one (compound 104)

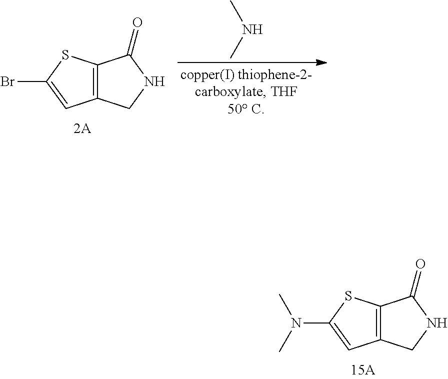

Step 1: A stirred mixture of 2A (500 mg, 2.29 mmol), dimethylamine (2M in THF, 40 mL) and copper (I) thiophene-2-carboxylate (219 mg, 1.15 mmol) was heated to 50° C. for 10 hours under a nitrogen atmosphere. After cooling to room temperature, the reaction mixture was poured into water and extracted with EtOAc (3×80 mL). The combined organic fractions were washed with brine, dried with anhydrous sodium sulfate, and concentrated under reduced pressure. The residue was purified by column chromatography on silica gel (0.5%-50% EtOAc in PE) and then further purified by prep-TLC (silica gel, 5% MeOH in DCM) to give compound 15A (50 mg, 12% yield) as a yellow solid: ¹H NMR (400 MHz, CDCl₃) δ 6.15 (s, 1H), 5.77 (s, 1H), 4.24 (s, 2H), 3.0 (s, 6H); ESI m/z 183.0 [M+H]⁺.

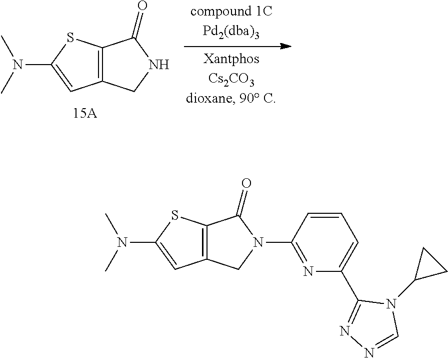 [SMILES: CN(C)c1cc2c(s1)C(=O)NC2 ; CN(C)c1cc2c(s1)C(=O)N(c1cccc(-c3nncn3C3CC3)n1)C2]

Step 2: A mixture of 15A (50 mg, 0.27 mmol), compound 1C (73 mg, 0.27 mmol), Pd₂(dba)₃ (25 mg, 0.027 mmol), Xantphos (22 mg, 0.038 mmol) and Cs₂CO₃ (312 g, 0.96 mmol) in 1,4-dioxane (6 mL) was stirred at 90° C. overnight under nitrogen. The reaction mixture was allowed to cool to room temperature and filtered. The filtrate was concentrated under reduced pressure and the residue was purified by silica gel column chromatography (0.5%-2.5% MeOH in DCM) to afford 104 (35 mg, 35% yield) as a yellow solid: ¹H NMR (400 MHz, DMSO-d₆) δ 8.67 (s, 1H), 8.46 (d, J=8.4, 1H), 7.95 (m, 1H), 7.73 (d, J=7.2, 1H), 6.07 (s, 1H), 4.95 (s, 2H), 4.09-4.02 (m, 1H), 3.03 (s, 6H), 1.10-1.05 (m, 2H), 0.96-0.94 (m, 2H); ESI m/z 367.1 [M+H]⁺.

### Example 19

ASK1 Kinase Assay. The ASK1 enzymatic assay was run following Promega ASK1 Kinase Enzyme System (Cat #V3881). The kit provides the protocol, enzymes and all reagents necessary to run an assay.

Firstly, the compounds, enzyme, substrate and ATP were diluted in provided assay buffer. The final concentration of the enzyme was 50 nM, substrate (Myelin basic protein) 1μg/ml and ATP 10 μM. The compound and the enzyme were pre-incubated in a 384 well white solid bottom plate (Greiner, Cat #784075) for 10 minutes. After incubation, the substrate and ATP were added and incubated for further 60 minutes. After 60 minutes, ADP-gloTM was added and plate was incubated for another 40 minutes. After 40 minutes, Kinase Detection Reagent was added and the plate was incubated for 45 minutes. After 45 minutes, plate was read on Perkin Elmer EnVision using luminescence read (0.5 seconds/well). IC₅₀ data for compounds are shown in Table 2.

### Example 20

### DYRK1A Kinase Assay

The DYRK1A kinase assay was performed using recombinant human full-length DYRK1A protein (GST-tagged, expressed in insect cells), in a 12-concentration IC₅₀ format with singlicate wells (12 pt IC₅₀), using microfluidics detection technology. Kinase reactions were assembled in 384 well plates (Greiner) in a total volume of 20 uL as follows: kinase protein was pre-diluted in assay buffer comprising: 100 mM HEPES, pH 7.5, 0.1% BSA, 0.01% Triton X-100, 1mM DTT, 5mM MgCl₂ and dispensed into 384 well plate (10 μL per well). All compounds and reference standards were serially pre-diluted in DMSO and added to the protein samples by acoustic dispensing (Labcyte Echo). DMSO concentration was equalized to 1% in all samples. Control samples (0%-inhibition in the absence of inhibitor, DMSO only) and 100%-inhibition (in the absence of enzyme) are assembled in replicates of four and used to calculate the %-inhibition in the presence of compounds. Reactions were initiated by addition of 104 of FAM-labeled DYRKtide peptide substrate and ATP prepared in the same assay buffer. Final concentrations of enzyme, ATP and substrate peptide were 0.01 nM, 20 μM and 1 μM, respectively. The kinase reactions are allowed to proceed for 3 h at room temperature. Following incubation, the reactions were quenched by addition of 50 μL of termination buffer (100 mM HEPES, pH7.5, 0.01% Triton X-100, 50 mM EDTA). Terminated plates are analyzed on a microfluidic electrophoresis instrument (Caliper LabChip® 3000, Caliper Life Sciences/Perkin Elmer), enabling electrophoretic separation and detection of product and substrate. A change in the relative intensity of the peptide substrate and phosphorylated product was the parameter measured. Activity in each test sample was determined as the product to sum ratio (PSR): P/(S+P), where P is the peak height of the product, and S is the peak height of the substrate. Percent inhibition (Pinh) was determined using the following equation: Pinh=(PSR0% inh—PSR compound)/(PSR0% inh—PSR100% inh)*100, in which: PSR compound is the product/sum ratio in the presence of compound, PSR0% inh is the product/sum ratio in the absence of compound and the PSR100% inh is the product/sum ratio in the absence of the enzyme. The compound IC₅₀ (50%-inhibition) was determined by the %-inh data (Pinh versus compound concentration) fitted by a 4-parameter sigmoid dose-response model using XLfit software (IDBS). IC₅₀ data for compounds are shown in Table 2.

Activity data for compounds are shown in Table 2.

TABLE 2

| | ASK1 Kinase | DYRK1A Kinase |
|---|---|---|
| Compound | IC₅₀ | IC₅₀ |
| 90 | +++ | ++ |
| 91 | ++ | ++ |
| 92 | ++ | +++ |
| 93 | ++ | +++ |
| 94 | ++ | + |
| 95 | ++ | + |
| 96 | +++ | ++ |
| 97 | ++ | +++ |
| 98 | + | + |
| 99 | ++ | + |
| 100 | ++ | + |
| 101 | + | + |
| 102 | +++ | ++ |
| 103 | +++ | +++ |
| 104 | ++ | ++ |

For ASK1 Kinase Assay: +++ = IC₅₀ <200 nM; ++ = IC₅₀ 200 nM-<1 μM; + = IC₅₀ 1-10 μM.
For DYRK1A Kinase Assays: +++ = IC₅₀ <200 nM; ++ = IC₅₀ 200 nM-<1 μM; + = IC₅₀ 1-10 μM.

### Example 21

### Clinical Trial of Dual Inhibitor of ASK1 and DYRK1A in AD Patient Selection/Management

Patient inclusion criteria are: men and women >55 with probably AD diagnosis, mild-to-moderate Alzheimer's disease, imaging study (CT or MRI) compatible with AD or age-related changes, Clinical Dementia Rating-Global Score (CDR-GS) of 0.5, 1.0 or 2.0, and a community dwelling with caregiver able and willing to accompany the participant to all visits if necessary.

Patient exclusion criteria are: no significant neurological disease other than AD that may affect cognition, no current clinically-significant systemic illness that is likely to result in deterioration of the patient's condition, no history of clinically-evident stroke, no myocardial infarction, no history of drug or alcohol abuse as defined by DSM-IV criteria within the last two years, imaging CT or MRI absent of significant abnormalities that may explain cognitive decline, such as multiple lacunar infarct or a single prior infarct >1 cubic cm, microhemorrhages or evidence of a prior hemorrhage >1 cubic cm, evidence of cerebral contusion, encephalomalacia, aneurysm, vascular malformation, or space occupying lesion such as an arachnoid cyst or brain tumor.

### Cohort Design/Drug Administration

The study is randomized, double-blind, parallel-group, and placebo-controlled. Subjects are randomly assigned to one of four parallel treatment groups: placebo or three escalating doses of any of the dual inhibitors of Formula (IV), (IVa), (IVb), (IVc), (V), (VI), (VII), (VIII), (IX), (X), or (XI), described herein. The inhibitor is administered orally once daily for 4 weeks. On completion of treatment, subjects are followed for 4 weeks.

### Measures of Drug Efficacy

Concentrations of the dual inhibitor are determined in plasma by using a validated bioanalytical assay to assess drug concentration. Steady state analysis of pharmacokinetic parameters (e.g. (Cₘₐₓ), time of Cₘₐₓ (Tₘₐₓ), half-life (T_{1/2}), and area under the plasma concentration versus time curve over the dosing interval (AUCₜₐᵤ)) occurs between weeks 2 and 4.

Brain MRI will be performed to explore the effects of changes in brain network, in functional and structural connectivity, and in assessing perfusion after administration of study drug.

### Safety Analysis

Safety monitoring includes clinical laboratory tests, physical examinations, vital signs measurements, 12-lead electrocardiograms, and documentation of adverse events (AEs).

### Efficacy Endpoints

Cerebral spinal fluid (CSF) obtained through lumbar puncture, will be analyzed for changes to concentration of beta amyloid (1-42), total tau, phosphorylated tau, tau isoforms and fragments, and tau phosphopeptides from baseline concentrations to week 4.

Changes in cerebral glucose metabolism as a proxy for mitochondrial respiration will be measured at baseline and at 4 weeks. PET scan will be performed. Correlations will be sought with assays of oxidative stress reduction to see if greater reductions in brain oxidative stress are reflected in elevations of cortical 2-fluorodeoxyglucose from baseline levels.

Plasma concentration—time data for each subject are analyzed using standard noncompartmental methods to compute pharmacokinetic parameters. Exposure and response relationships for the dual inhibitors are determined by fitting Cₘₐₓ or AUCₜₐᵤ to time-weighted absolute changes in phosphorylated tau, and beta amyloid (1-42).

Qualitative assessment of cognitive status will be taken at baseline, week 4, and week 8 post dosing. This will consist of administering the Alzheimer's Disease Assessment Scale—Cognitive subscale (ADAS-Cog) and the Mini Mental State Examination (MMSE).

### Example 22

### Clinical Trial of Dual Inhibitors of ASK1 and DYRK1A in DS

### Patient Selection/Management

Patient inclusion criteria are: age 10-17 at time of screening, proven trisomy 21 (Down syndrome) documented by chromosomal analysis (karytyping), distribution of both males and females, participant ability to complete the Clinical Evaluation of Language Fundamentals (CELF)-preschool 2 word classes task, Vineland-II Adaptive Behavior Scales (VAB-II)/Parent/Caregiver Rating Form (PCRF) receptive sub-domain raw score of >=25 and expressive sub-domain raw score of >=61.

Study participant must have a parent or a caregiver who will agree to accompany the study participant to all clinical visits during study. The parent or caregiver must be a reliable informant with sufficient contact with the participant to have detailed knowledge of the participant's adaptive functioning in order to be able to complete the VABS-II/PCRF accurately.

Patient exclusion criteria are: <10 or >17 years at the screening visit, participants without a reliable parent or caregiver, any current primary psychiatric diagnosis other the DS (as per DSM-IV).

### Cohort Design/Drug Administration

The study is randomized, double-blind, parallel-group, and placebo-controlled. Subjects are randomly assigned to one of three parallel treatment groups: placebo or two escalating doses of any of the dual inhibitors of Formula (IV), (IVa), (IVb), (IVc), (V), (VI), (VII), (VIII), (IX), (X), or (XI), described herein. The inhibitor is administered orally once daily for 10 weeks. On completion of treatment, subjects are followed for 4 weeks.

### Measures of Drug Efficacy

Concentrations of the dual inhibitors are determined in plasma by using a validated bioanalytical assay to assess drug concentration. Steady state analysis of pharmacokinetic parameters (e.g. (Cₘₐₓ), time of Cₘₐₓ (Tₘₐₓ), half-life (T_{1/2}), and area under the plasma concentration versus time curve over the dosing interval (AUCₜₐᵤ)) occurs between weeks 2 and 4.

### Safety Analysis

Safety monitoring includes clinical laboratory tests, physical examinations, vital signs measurements, 12-lead electrocardiograms, and documentation of adverse events (AEs).

### Efficacy Endpoints

Mean change in Vineland-II Adaptive Behavior Scale (VABS-II) Parent/Caregiver Rating Form (PCRF) Score using the last observed carried forward (LOCF) from time frame baseline (Day 0) to week 10 or at early termination. This will be a sum of the 9 sub-domain v-scores (3 scores for each of the communication, daily living skills, and socialization domains). Mean change in the Test of Verbal Expression and Reasoning (TOVER) from baseline (Day 0) to week 10 or early termination. TOVER is a subject performance-based measure of expressive language function.

The examples and embodiments described herein are for illustrative purposes only and various modifications or changes suggested to persons skilled in the art to be included within the spirit and purview of this application and scope of the appended claims.

What is claimed is:

1. A method for the treatment or prevention of a condition associated with ASK1 or DYRK1A which is not a cardio-metabolic disease, comprising administering to a subject in need thereof a therapeutically effective amount of a dual inhibitor of ASK1 and DYRK 1A, wherein the dual inhibitor displays less than 10-fold selectivity between ASK1 and DYRK1A and/or simultaneously acts on ASK1 and DYRK1A with an IC₅₀ lower than 100 nM on each.

2. The method of claim 1, wherein the ASK1 and DYRK1A associated condition is a cognitive impairment.

3. The method of claim 2, wherein the cognitive impairment is associated with a neurodegenerative disease or a neurodevelopmental disorder.

4. The method of claim 3, wherein the neurodegenerative disease or disorder is a Alzheimer's disease (AD), a Parkinson's disease (PD), a Huntington disease (HD), dementia, an Alexander disease, an Alper's disease, an amyotrophic lateral sclerosis (ALS), an ataxia telangiectasia, a Canavan disease, a Cockayne syndrome, a corticobasal degeneration, a Creutzfeld-Jakob disease, a Guillai-Barre Syndrome, a Kennedy's disease, a Krabbe disease, a Pelizaeus-Merzbacher disease, a Pick's disease, a Refsum's disease, a Sandhoff disease, a Schilder's disease, a spinal cord injury, a Steele-Richardson-Olszewski disease, a tabes dorsalis, multiple sclerosis, and/or a traumatic brain injury.

5. The method of claim 4, wherein the neurodegenerative disease is Alzheimer's disease (AD).

6. The method of claim 4, wherein the neurodegenerative disease is Parkinson's disease (PD).

7. The method of claim 2, wherein the cognitive impairment is associated with a neurodevelopmental disorder.

8. The method of claim 7, wherein the neurodevelopmental disorder is a Down syndrome, a mental retardation 7 (MRD7), and/or an Autism Spectrum disorder (ASD).

9. The method of claim 8, wherein the neurodevelopmental disorder is Down syndrome.

10. The method of any one of claims 1-9, wherein the dual inhibitor is a compound having the structure of Formula (IV), or a pharmaceutically acceptable salt or solvate thereof: 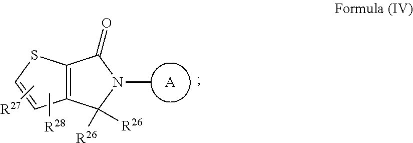
wherein: 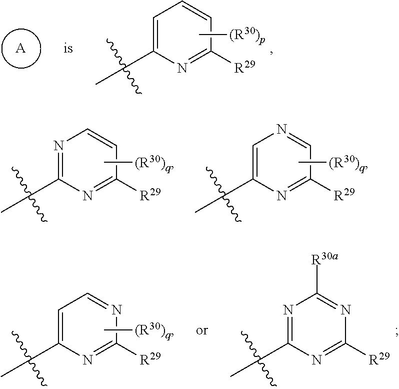
each R²⁶ is independently selected from a group consisting of H, halo, and C₁-C₆alkyl;
R²⁷ is selected from a group consisting of hydrogen, halogen, —CN, —OH, —OR³¹, —SR³¹, —S(═O)R³², —NO₂, —N(R³¹)₂, —S(═O)₂R³², —NHS(═O)₂R³², —S(═O)₂N(R³¹)₂, —C(═O)R³², —C(═O)OR³¹, —OC(═O)R³², —C(═O)N(R³¹)₂, —OC(═O)N(R³¹)₂, —NR³¹C(═O)N(R³¹)₂, —NR³¹C(═O)R³², —NR³¹C(═O)OR³¹, C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂—N(R³⁸)₂, —N(R³⁸)₂, —N(R³⁸)C(═O)R³⁹, and —N(R³⁸)S(═O)₂R³⁸;
R²⁸ is selected from a group consisting of H, halo, —CN, and C₁₋₆alkyl;
or R²⁷ and R²⁸, are combined to form a phenyl ring optionally substituted with one, two, or three R³³ substituents;
R²⁹ is selected from a group consisting of H, halo, —CN, —OH, —OR³¹, —SR³¹, —S(═O)R³², —NO₂, —N(R³¹)₂, —S(═O)₂R³², —NHS(═O)₂R³², —S(═O)₂N(R³¹)₂, —C(═O)R³², —C(═O)OR³¹, —OC(═O)R³², —C(═O)N(R³¹)₂, —OC(═O)N(R³¹)₂, —NR³¹C(═O)N(R³¹)₂, —NR³¹C(═O)R³², —NR³¹C(═O)OR³¹, C₁₋₆alkyl, C₂₋₆alkenyl, C₂₋₆alkynyl, C₃₋₈cycloalkyl, C₂₋₉heterocycle, C₆₋ₘaryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋ₘaryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸;
each R³⁰ is independently selected from a group consisting of halo, —CN, and C₁₋₆alkyl;
R^{30a} is selected from the group consisting of H and C₁-C₆alkyl;
each R³¹ is independently selected from the group consisting of H, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle;
or two R³¹ on the same heteroatom are taken together with that heteroatom to which they are attached to form a C₂₋₉heterocycle or a C₂₋₉heteroaryl;
each R³² is independently selected from the group consisting of C₁-C₆alkyl, C₃-C₈cycloalkyl, and C₂₋₉heterocycle;
each R³³ is independently selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸;
each R³⁸ is independently selected from the group consisting of H, C₁-C₆alkyl, and C₃-C₈cycloalkyl; or two R³⁸ on the same heteroatom are taken together with that heteroatom to which they are attached to form a C₂₋₉heterocycle;
each R³⁹ is independently selected from the group consisting of C₁-C₆alkyl and C₃-C₈cycloalkyl;
p is 0, 1, 2, or 3; and
q is 0, 1, or 2.

11. The method of any one of claims 1-9, wherein the dual inhibitor having the structure of Formula (IV), has the structure of Formula (IVa), or a pharmaceutically acceptable salt or solvate thereof:

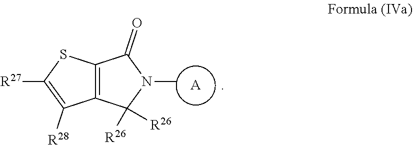

12. The method of any one of claims 1-9, wherein the dual inhibitor having the structure of Formula (IV), has structure of Formula (IVb), or a pharmaceutically acceptable salt or solvate thereof:

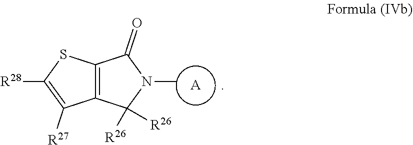

13. The method of any one of claims 10-12, or a pharmaceutically acceptable salt or solvate thereof, wherein:
R²⁷ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸.

14. The method of any one of claims 10-13, or a pharmaceutically acceptable salt or solvate thereof, wherein:
R²⁷ is selected from a group consisting of C₂₋₉heterocycle and C₁₋₉heteroaryl; wherein C₂₋₉heterocycle and C₁₋₉heteroaryl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂—N(R³⁸)₂, —N(R³⁸)₂, —N(R³⁸)C(═O)R³⁹, and —N(R³⁸)S(═O)₂R³⁸.

15. The method of any one of claims 10-14, or a pharmaceutically acceptable salt or solvate thereof, wherein:
R²⁷ is selected from a group consisting of C₂₋₉heterocycle and C₁₋₉heteroaryl; wherein C₂₋₉heterocycle and C₁₋₉heteroaryl are optionally substituted with one or two substituents selected from the group consisting of halo, —CN, C₁₋₆alkyl, —C₁₋₆alkyl-OH, C₁₋₆haloalkyl, C₃₋₈ cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂—N(R³⁸)₂, —N(R³⁸)₂, —N(R³⁸)C(═O)R³⁹, and —N(R³⁸)S(═O)₂R³⁸.

16. The method of any one of claims 10-15, or a pharmaceutically acceptable salt or solvate thereof, wherein:
R²⁷ is selected from a group consisting of pyrazole, imidazole, thiazole, and pyridine; wherein pyrazole, imidazole, thiazole, and pyridine are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂-N(R³⁸)₂, —N(R³⁸)₂, —N(R³⁸)C(═O)R³⁹, and —N(R³⁸)S(═O)₂R³⁸.

17. The method of any one of claims 10-16, or a pharmaceutically acceptable salt or solvate thereof, wherein:
R²⁷ is selected from a group consisting of pyrazole, imidazole, thiazole, and pyridine; wherein pyrazole, imidazole, thiazole, and pyridine are optionally substituted with one or two substituents selected from the group consisting of halo, C₁₋₆alkyl, and C₃₋₈cycloalkyl.

18. The method of any one of claims 10-17, or a pharmaceutically acceptable salt or solvate thereof, wherein:
R²⁷ is 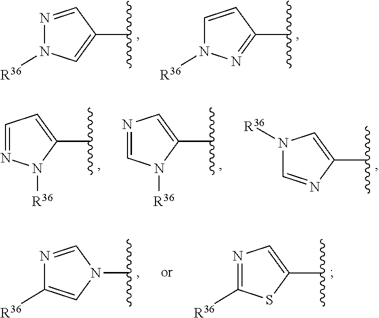
wherein R³⁶ is C₁-C₆alkyl or C₃-C₆cycloalkyl.

19. The method of any one of claims 10-18, or a pharmaceutically acceptable salt or solvate thereof, wherein:
R²⁷ is 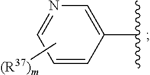
wherein each R³⁷ is independently H, halo, CN, C₁-C₆alkyl, or C₃-C₆cycloalkyl; and
m is 1 or 2.

20. The method of any one of claims 10-19, or a pharmaceutically acceptable salt or solvate thereof, wherein:
R²⁷ is selected from a group consisting of unsubstituted pyrazole, unsubstituted imidazole, unsubstituted thiazole, and unsubstituted pyridine.

21. The method of any one of claims 10-20, or a pharmaceutically acceptable salt or solvate thereof, wherein:
R²⁷ is C₆₋₁₀aryl optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂—N(R³⁸)₂, —N(R³⁸)₂, —N(R³⁸)C(═O)R³⁹, and —N(R³⁸)S(═O)₂R³⁸.

22. The method of any one of claim 21, or a pharmaceutically acceptable salt or solvate thereof, wherein:
R²⁷ is phenyl optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)_{R}³⁹, —C(═O)OR³⁸, —C(═O)N(R³⁸)₂, —S(═O)_{R}³⁹, —S(═O)₂R³⁸, —S(═O)₂—N(R³⁸)₂, —N(R³⁸)₂, —N(R³⁸)C(═O)R³⁹, and —N(R³⁸)S(═O)₂R³⁸.

23. The method of any one of claims 10-20, or a pharmaceutically acceptable salt or solvate thereof, wherein:
R²⁷ is —C(═O)N(R³¹)₂ and each R³¹ is independently selected from the group consisting of H, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle; or two R³¹ on the same heteroatom are taken together with that heteroatom to which they are attached to form a C₂₋₉heterocycle or a C₂₋₉heteroaryl.

24. The method of claim 23, or a pharmaceutically acceptable salt or solvate thereof wherein:
R²⁷ is

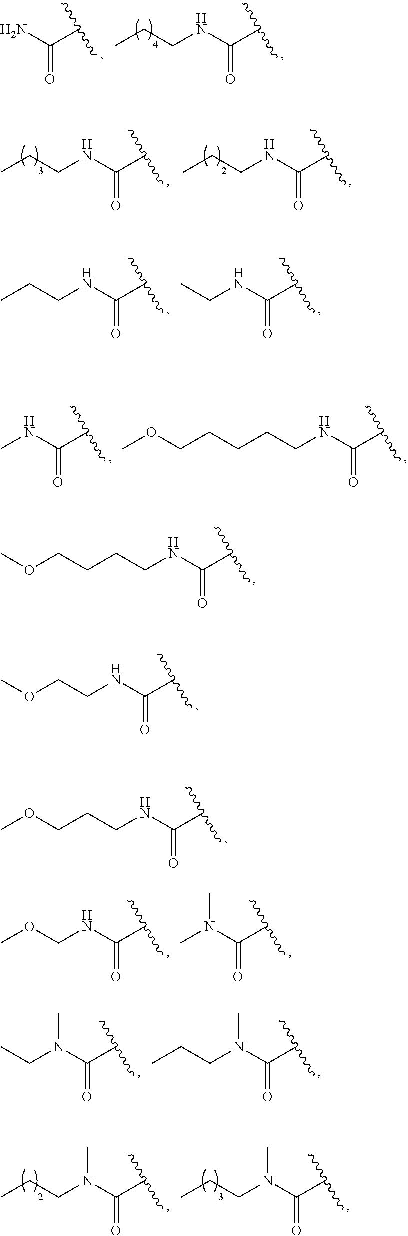

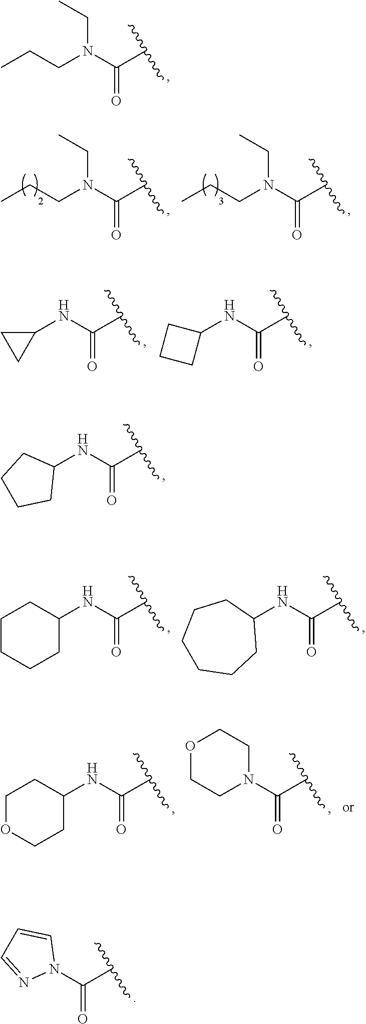

25. The method of claim 23, or a pharmaceutically acceptable salt or solvate thereof wherein:
R²⁷ is 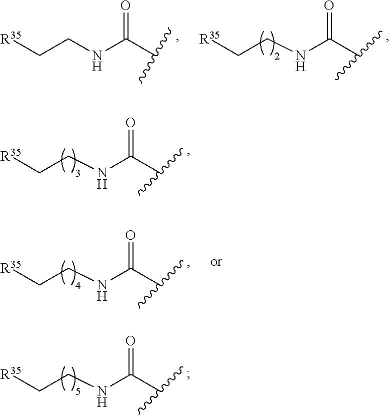
wherein R³⁵ is C₁₋₉heteroaryl.

26. The method of any one of claims 10-20, or a pharmaceutically acceptable salt or solvate thereof, wherein R²⁷ is —NHC(═O)R³².

27. The method of claim 26, or a pharmaceutically acceptable salt or solvate thereof wherein:
R²⁷ is

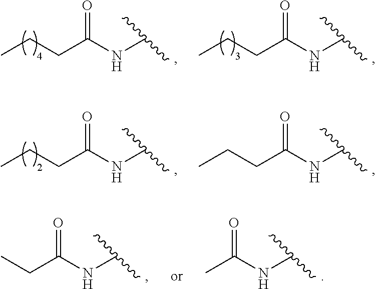

28. The method of any one of claims 10-20, or a pharmaceutically acceptable salt or solvate thereof, wherein R²⁷ is —C(═O)R³².

29. The method of claim 28, or a pharmaceutically acceptable salt or solvate thereof wherein:
R²⁷ is

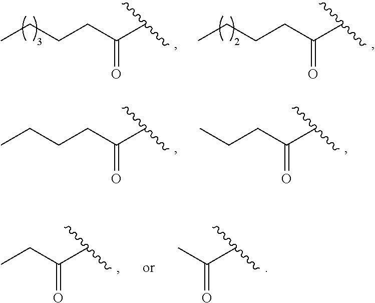

30. The method of any one of claims 10-29, or a pharmaceutically acceptable salt or solvate thereof, wherein R²⁸ is H.

31. The method of any one of claims 10-29, or a pharmaceutically acceptable salt or solvate thereof, wherein R²⁸ is C₁-C₆alkyl.

32. The method of any one of claims 1-9, wherein the dual inhibitor having the structure of Formula (IV) has the structure of Formula (IVc), or a pharmaceutically acceptable salt or solvate thereof: 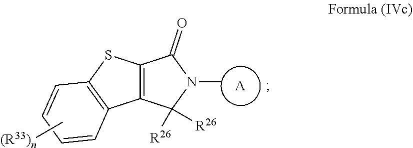
wherein n is 0, 1, 2, or 3.

33. The method of claim 32, or a pharmaceutically acceptable salt or solvate thereof wherein n is 0.

34. The method of claim 32, or a pharmaceutically acceptable salt or solvate thereof wherein n is 1.

35. The method of claim 32, or a pharmaceutically acceptable salt or solvate thereof wherein n is 2.

36. The method of any one of claims 32-35, or a pharmaceutically acceptable salt or solvate thereof, wherein:
R²⁹ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸.

37. The method of any one of claims 32-36, or a pharmaceutically acceptable salt or solvate thereof, wherein:
R²⁹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸.

38. The method of any one of claims 32-37, or a pharmaceutically acceptable salt or solvate thereof, wherein:
R²⁹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂, —N(R³⁸)₂, —N(R³⁸)₂, —N(R³⁸)C(═O)R³⁹, and —N(R³⁸)S(═O)₂R³⁸.

39. The method of any one of claims 32-38, or a pharmaceutically acceptable salt or solvate thereof, wherein:
R²⁹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R³⁹, —C(═O)OR³⁸, —C(═O)N(R³⁸)₂, —S(═O)R³⁹, —S(═O)₂R³⁸, —S(═O)₂⁻N(R³⁸)₂, —N(R³⁸)₂, —N(R³⁸)C(═O)R³⁹, and —N(R³⁸)S(═O)₂R³⁸.

40. The method of any one of claims 32-39, or a pharmaceutically acceptable salt or solvate thereof, wherein:
R²⁹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halo, C₁₋₆alkyl, and C₃₋₈cycloalkyl.

41. The method of claim 40, or a pharmaceutically acceptable salt or solvate thereof, wherein:
R²⁹ is

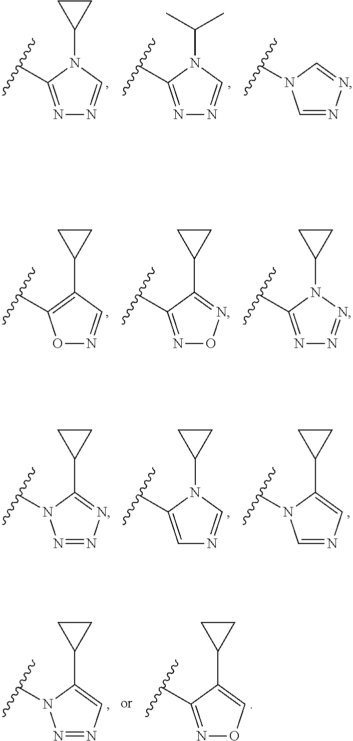

42. The method of claim 38, or a pharmaceutically acceptable salt or solvate thereof, wherein:
R²⁹ is

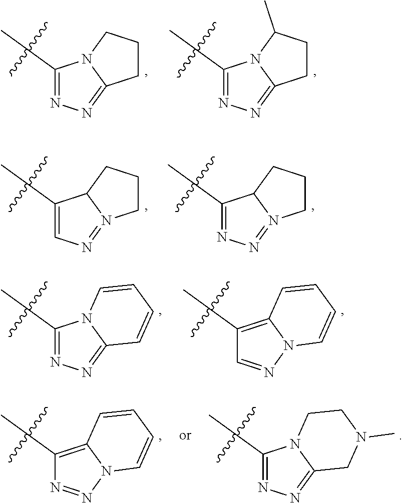

43. The method of any one of claims 32-42, or a pharmaceutically acceptable salt or solvate thereof, wherein

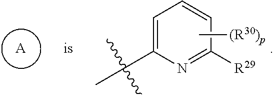

44. The method of any one of claims 32-43, or a pharmaceutically acceptable salt or solvate thereof, wherein p is 0.

45. The method of any one of claims 32-44, or a pharmaceutically acceptable salt or solvate thereof, wherein

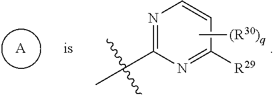

46. The method of any one of claims 32-45, or a pharmaceutically acceptable salt or solvate thereof, wherein

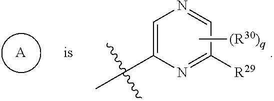

47. The method of any one of claims 32-46, or a pharmaceutically acceptable salt or solvate thereof, wherein

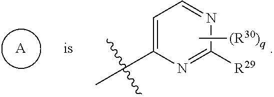

48. The method of any one of claims 32-47, or a pharmaceutically acceptable salt or solvate thereof, wherein q is 0.

49. The method of any one of claims 32-48, or a pharmaceutically acceptable salt or solvate thereof, wherein

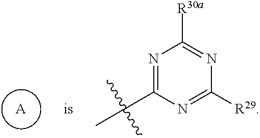

50. The method of claim 49, or a pharmaceutically acceptable salt or solvate thereof, wherein R^{30a} is H.

51. The method of any one of claims 32-50, or a pharmaceutically acceptable salt or solvate thereof, wherein each R²⁶ is H.

52. The method of any one of claims 10-51, or a pharmaceutically acceptable salt thereof, wherein the dual inhibitor is:

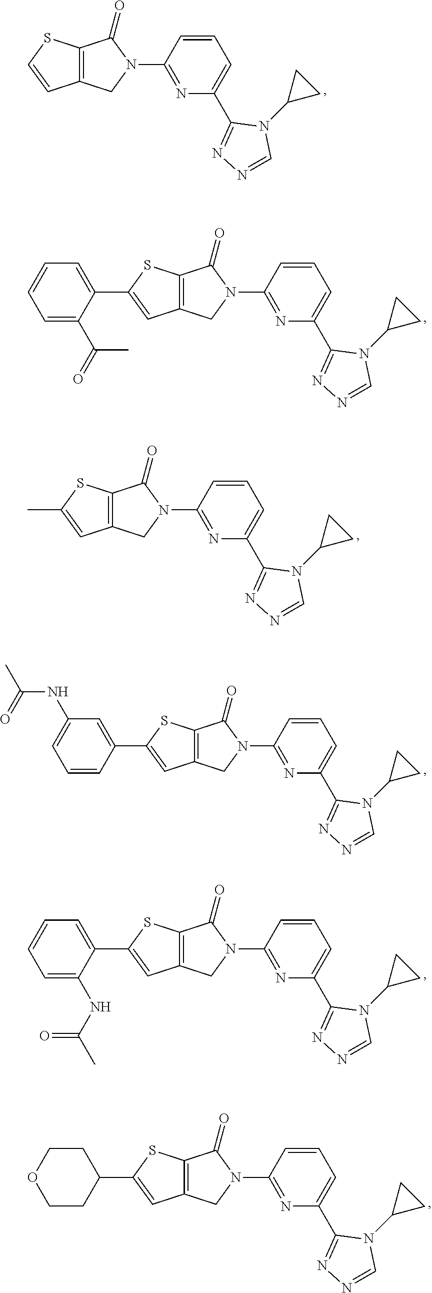

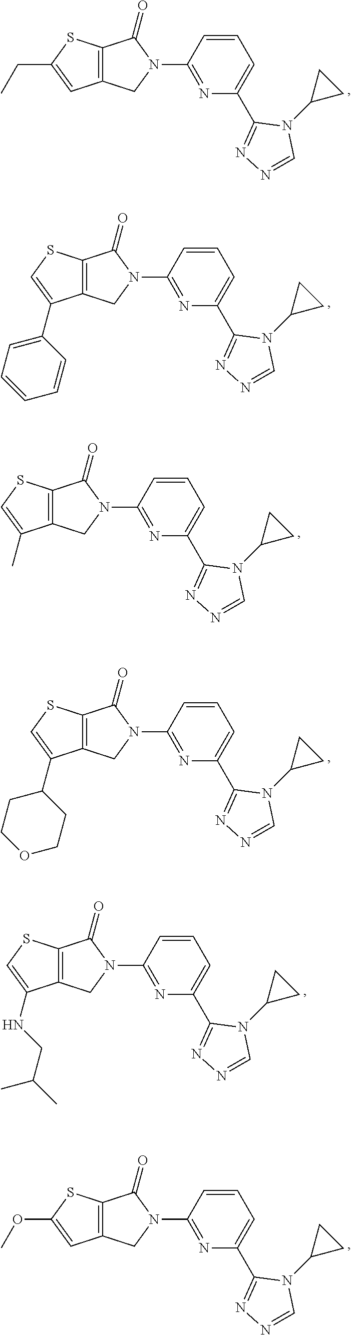

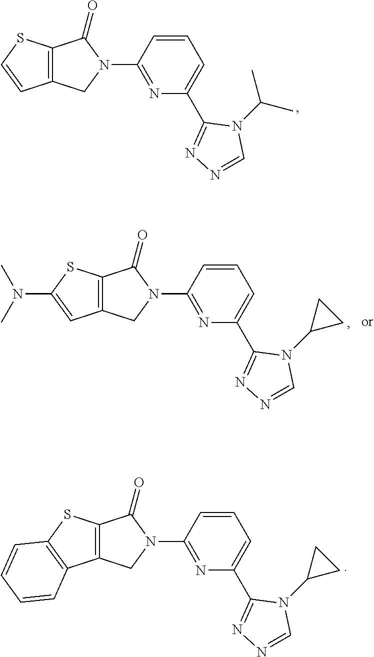

53. The method of any one of claims 1-9, wherein the dual inhibitor is a compound having the structural of Formula (V), or a pharmaceutically acceptable salt or solvate thereof: 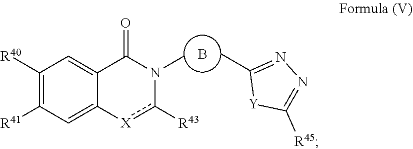
wherein: [symbol]
is a single bond or a double bond;
X is C(R⁴²), CH(R⁴²), N, or N(R⁴²);
Y is N(R⁴⁴) or O; 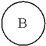
is selected from a group consisting of phenyl and a 5 or 6-membered heteroaryl;
R⁴⁰ is a C₅₋₁₀heteroaryl optionally substituted with one, two, or three R⁴⁶ groups;
R⁴¹ is selected from a group consisting of H, halo, OH, or NH₂, or is selected from a group consisting of C₁₋₆ alkyl, C₁₋₃ heteroalkyl, a 5 or 6-membered heterocycloalkyl, phenyl, and a 5 or 6-membered heteroaryl, optionally substituted with one, two, or three R⁴⁶ groups;
R⁴² is selected from a group consisting of H, halo, OH, and NH₂;
R⁴³ is selected from a group consisting of H, C₁₋₃ alkyl, and C₁₋₃ alkoxy;
R⁴⁴ is selected from a group consisting of H, C₁,₈ alkyl, C₃₋₇ cycloalkyl, and a 3 to 6-membered heterocycloalkyl, optionally substituted with one, two, or three R⁴⁶ groups;
R⁴⁵ is selected from a group consisting of H and C₁₋₆ alkyl;
or R⁴⁴ and R⁴⁵ are joined together to form a 5- or 6-membered ring;
R⁴⁶ is selected from the group consisting of H, halo, CN, OH, NH₂, NH₂—(C═O)—, C₁₋₃alkyl, C₁₋₃ alkoxy, C₁₋₃alkyl-NH—(C═O)—, C₁₋₃alkyl-S(═O)₂-, C₃₋₆cycloalkyl, a 3 to 6-membered heterocycloalkyl, and phenyl; the “hetero” moieties of the 5 to 10-membered heteroaryl, C₁₋₃ heteroalkyl, 5 or 6-membered heterocycloalkyl, 5 or 6-membered heteroaryl, and 3-6-membered heterocycloalkyl are each independently selected from the group consisting of —NH—, N, —O—, —S—, —S(═O)₂—, and —NH—C(═O)—; and the numbers of the heteroatoms or heteroatom groups in any of the above cases are each independently one, two, or three.

54. The method of any one of claims 1-9, wherein the dual inhibitor is a compound having the structural of Formula (VI),or a pharmaceutically acceptable salt or solvate thereof: 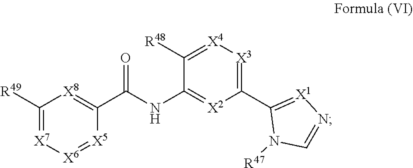
wherein:
R⁴⁷ is selected from a group consisting of alkyl, alkenyl, alkynyl, cycloalkyl, aryl, heteroaryl, or heterocyclyl, optionally substituted with one, two, or three substituents individually selected from halogen, oxo, alkyl, cycloalkyl, heterocyclyl, aryl, aryloxy, —NO2, R⁵², —C(O)—R⁵², —OC(O)—R⁵²—C(O)—O—R⁵², —C(O)—N(R⁵²)(R⁵³), —OC(O)—N(R⁵²)(R⁵³), —S—R⁵², —S(═O)—R⁵², —S(═O)₂R⁵², —S(═O)₂—N(R⁵²)(R⁵³), —S(═O)₂—O—R⁵², —N(R⁵²)(R⁵³), —N(R⁵²)-C(O)—R⁵³, —N(R⁵²)—C(O)—O—R⁵³, —N(R⁵²)—C(O)—N(R⁵²)(R⁵³), —N(R⁵²)—S(═O)₂—R⁵², —CN, and —O—R⁵², wherein alkyl, cycloalkyl, heterocyclyl, phenyl, and phenoxy are optionally substituted with one, two, or three substituents selected from alkyl, cycloalkyl, alkoxy, hydroxyl, and halo;
wherein R⁵² and R⁵³ are independently selected from the group consisting of H, C₁₋₁₅alkyl, cycloalkyl, heterocyclyl, aryl, and heteroaryl, all of which are optionally substituted with one, two, or three substituents selected from halo, alkyl, mono- or dialkylamino, alkyl or aryl or heteroaryl amide, —CN, alkoxy, —CF₃, aryl, and heteroaryl; or
R₅₂ and R₅₃ when taken together with the nitrogen to which they are attached form a heterocycle;
R⁴⁸ is H, halo, cyano, alkoxy, or alkyl optionally substituted by halo;
R⁴⁹ is aryl, heteroaryl, or heterocyclyl, optionally substituted with one or more substituents selected from alkyl, alkoxy, cycloalkyl, cycloalkylalkyl, aryl, arylalkyl, heteroaryl, heteroarylalkyl, heterocyclyl, heterocyclylalkyl, halo, oxo, —NO₂, haloalkyl, haloalkoxy, —CN, —O—R⁵², —O—C(O)—R⁵², —O—C(O)—N(R⁵²)(R⁵³), —S—R⁵², —N(R⁵²)(R⁵³), —S(═O)—R⁵², —S(═O)₂R⁵², —S(═O)₂—N(R⁵²)(R⁵³), —S(═O)₂—O—R⁵², —N(R⁵²)—C(O)—R⁵³, —N(R⁵²)—C(O)—O—R⁵³, —N(R⁵²)—C(O)—N(R⁵²)(R⁵³), —C(O)—R⁵², —C(O)—O—R⁵², —C(O)—N(R⁵²)(R⁵³), and —N(R⁵²)—S(═O)₂—R⁵³, wherein alkyl, alkoxy, cycloalkyl, aryl, heteroaryl ,or heterocyclyl, is further optionally substituted with one or more groups selected from halo, oxo, —NO₂, alkyl, haloalkyl, haloalkoxy, —N(R⁵²)(R⁵³), —C(O)—R⁵², —C(O)—O—R⁵², —C(O)—N(R⁵²)(R⁵³), —CN, —O—R⁵², cycloalkyl, aryl, heteroaryl, and heterocyclyl;
with the proviso that the heteroaryl or heterocyclyl moiety includes at least one ring nitrogen atom; X¹, X², X³, X⁴, X⁵, X⁶, X⁷, and X⁸ are independently C(R⁵⁰) or N, in which each R⁵⁰ is independently H, alkyl, alkoxy, cycloalkyl, aryl, heteroaryl, heterocyclyl, halo, —NO₂, haloalkyl, haloalkoxy, —CN, —O—R⁵², —S—R⁵², —N(R⁵²)(R⁵³), —S(═O)—R⁵², —S(═O)₂R⁵², —S(═O)₂—N(R⁵²)(R⁵³), —S(═O)₂—O—R⁵², —N(R⁵²)—C(O)—R⁵³, —N(R⁵²)—C(O)—O—R⁵³, —N(R⁵²)—C(O)—N(R⁵²)(R⁵³), —C(O)—R⁵², —C(O)—O—R⁵², —C(O)—N(R⁵²)(R⁵³), or —N(R⁵²)—S(═O)₂—R⁵³, wherein the alkyl, cycloalkyl, aryl, heteroaryl, and heterocyclyl is further optionally substituted with one or more groups selected from halo, oxo, —NO₂, —CF₃, —O—CF₃, —N(R⁵²)(R⁵³), —C(O)—R⁵², —C(O)—O—R⁵³, —C(O)—N(R⁵²)(R⁵³), —CN, —O—R⁵²; or X⁵ and X⁶ or X⁶ and X⁷ are joined to provide optionally substituted fused aryl or optionally substituted fused heteroaryl; and
with the proviso that at least one of X², X³, and X⁴ is C(R⁵⁰);
at least two of X⁵, X⁶, X⁷, and X⁸ are C(R⁵⁰); and
at least one of X², X³, X⁴, X⁵, X⁶, X⁷, and X⁸ is N.

55. The method of claim 54, wherein the dual inhibitor is selonsertib (GS-4997), or a pharmaceutically acceptable salt or solvate thereof

56. The method of any one of claims 1-9, wherein the dual inhibitor is a compound having the structure of Formula (VII),or a pharmaceutically acceptable salt or solvate thereof: 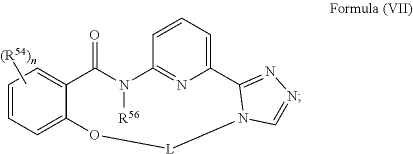
wherein:
L is selected from C₃₋₅alkylene and C₃₋₅alkenylene, wherein C₃₋₅alkylene and C₃₋₅alkenylene are optionally substituted with one or two R⁵⁵ groups;
each R⁵⁴ is independently selected from C₁₋₆alkyl, C₂-₆alkenyl, C₂₋₆alkynyl, carbocyclyl, heterocyclyl, halo, —CN, —C(O)R^{54a}, —C(O)₂R^{54a}, —C(O)N(R^{54a})₂, —N(R^{54a})₂, —N(R^{54a})C(O)R^{54a}, —N(R^{54a})C(O)₂R^{54a}, —N(R^{54a})C(O)N(R^{54a})₂, —N(R^{54a})S(O)₂R^{54a}, —OR^{54a}, —OC(O)R^{54a}, —OC(O)N(R^{54a})₂, —SR^{54a}, —S(O)R^{54a}, —S(O)₂R^{54a}, —S(O)N(R^{54a})₂, and —S(O)₂N(R^{54a})₂, wherein C₁₋₆alkyl, C₂₋₆alkenyl, C₂₋₆alkynyl, carbocyclyl, and heterocyclyl, are optionally substituted with one or more R⁵⁸ groups;
each R^{54a} is independently selected from H, C₁₋₆alkyl, C₂₋₆alkenyl, C₂₋₆alkynyl, carbocyclyl, and heterocyclyl, wherein C₁₋₆alkyl, C₂₋₆alkenyl, C₂₋₆alkynyl, carbocyclyl, and heterocyclyl are each optionally and independently substituted with one or more R⁵⁸ groups;
each R⁵⁸ is independently selected from C₁₋₆alkyl, C₂₋₆alkenyl, C₂₋₆alkynyl, carbocyclyl, heterocyclyl, halo, —CN, —C(O)R^{58a}, —C(O)₂R^{58a}, —C(O)N(R^{58a})₂, —N(R^{58a})₂, —N(R^{58a})C(O)R^{58a}, —N(R^{58a})C(O)₂R^{58a}, —N(R^{58a})C(O)N(R^{58a})₂, —N(R^{58a})S(O)₂R^{58a}, —OR^{58a}, —OC(O)R^{58a}, —OC(O)N(R^{58a})₂, —SR^{58a}, —S(O)R^{58a}, —S(O)₂R^{58a}, —S(O)N(R^{58a})₂, and —S(O)₂N(R^{58a})₂, wherein C₁₋₆alkyl, C₂₋₆alkenyl, C₂₋₆alkynyl, carbocyclyl, and heterocyclyl are optionally and independently substituted with one or more groups selected from halo, —CN, —C(O)R^{58a}, —C(O)₂R^{58a}, —C(O)N(R^{58a})₂, —N(R^{58a}), —N(R^{58a})C(O)R^{58a}, —N(R^{58a})C(O)₂R^{58a}, —N(R^{58a})C(O)N(R^{58a})₂, —N(R^{58a})S(O)₂R^{58a}, —OR^{58a}, —OC(O)R^{58a}, —OC(O)N(R^{58a})₂, —SR^{58a}, —S(O)R^{58a}, —S(O)₂R^{58a}, —S(O)N(R^{58a})₂, and —S(O)₂N(R^{58a})₂;
each R^{58a} is independently selected from H, C₁₋₆alkyl, C₂₋₆alkenyl, C₂₋₆alkynyl, carbocyclyl, and heterocyclyl;
each R⁵⁵ is independently selected from C₁₋₆alkyl, —CN, —C(O)R^{55a}, —C(O)₂R^{55a}, —C(O)N(R^{55a})₂, —NO₂, —N(R^{55a})₂, —N(R^{55a})C(O)R^{55a}, —N(R^{55a})C(O)₂R^{55a}, —N(R^{55a})C(O)N(R^{55a})₂, —N(R^{55a})S(O)₂R^{55a}, —OR^{55a}, —OC(O)R^{55a}, —OC(O)N(R^{55a})₂, —SR^{55a}, —S(O)R^{55a}, —S(O)₂R^{55a}, —S(O)N(R^{55a})₂, and —S(O)₂N(R⁵⁴)₂, wherein C₁₋₆alkyl is optionally substituted with one or more R⁵⁹ groups;
each R^{55a} is independently selected from H, C₁₋₆alkyl, C₂₋₆alkenyl, C₂₋₆alkynyl, carbocyclyl, and heterocyclyl wherein C₁₋₆alkyl, C₂₋₆alkenyl, C₂₋₆alkynyl, carbocyclyl, and heterocyclyl is optionally and independently substituted with one or more R⁵⁹ groups; and
each R⁵⁹ is independently selected from C₁₋₆alkyl, halo, and —OR^{59a};
each R^{59a} is independently selected from H, C₁₋₆alkyl, C₂₋₆alkenyl, C₂₋₆alkynyl, carbocyclyl, and heterocyclyl;
R⁵⁶ is H or C₁₋₄alkyl; and
n is selected from 0, 1, and 2.

57. The method of claim 56, wherein the dual inhibitor of Formula (VII) is a compound having the structure of Formula (VIIa),or a pharmaceutically acceptable salt or solvate thereof: 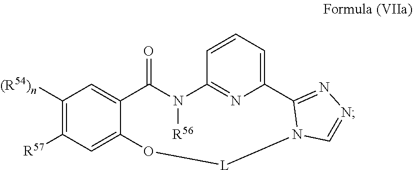
wherein:
L is —(CH₂)₄—, —CH₂—CH₂—CH₂—CH(CH₃)—, or —CH(CH₃)—CH₂—CH₂—CH₂—;
R⁵⁴ is —CN, halogen, or heterocyclyl, selected from imidazolyl, pyrazolyl, pyridine, piperazine, 1,2,3,6-tetrahydropyridine, piperidine, pyrimidine, and 6-oxa-3-azabicyclo[3.1.1]heptanyl, wherein each heterocyclyl is optionally substituted with one R⁵⁸ group;
R⁵⁶ is H;
R⁵⁷ is halogen;
R⁵⁸ is C₁₋₄lkyl or C₃₋₆cycloalkyl, wherein C₁₋₄alkyl is optionally substituted with one —N(R^{58a})₂;
R^{58a} is H or C₁₋₄alkyl; and
n is 0 or 1.

58. The method of any one of claims 1-9, wherein the dual inhibitor is a compound having the structure of Formula (VIII),or a pharmaceutically acceptable salt or solvate thereof: 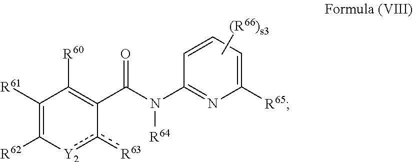
wherein: 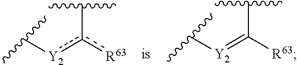
wherein:
Y² is N or CR^{Y2};
R⁶³ is H, halogen, —CN, —OR⁶⁷, —SR⁶⁷, —S(═O)R⁶⁹, —NO₂, —NR⁶⁷R⁶⁸, —S(═O)₂R⁶⁹, —NR⁶⁷S(═O)₂R⁶⁹, —S(═O)₂NR⁶⁷R⁶⁸, —C(═O)R⁶⁹, —OC(═O)R⁶⁹, —CO R⁶⁷, —OCO₂R⁶⁷, —C(═O)NR⁶⁷R⁶⁸, —OC(═O)NR⁶⁷R⁶⁸, —NR⁶⁷C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl; and
R^{Y2} is H, halogen, —CN, —OR⁶⁷, —SR⁶⁷, —S(═O)R⁶⁹, —NO₂, —NR⁶⁷R⁶⁸, —S(═O)₂R⁶⁹, —NR⁶⁷S(═O)₂R⁶⁹, —S(═O)₂NR⁶⁷R⁶⁸, —C(═O)R⁶⁹, —OC(═O)R⁶⁹, -CO₂R⁶⁷, —OCO₂R⁶⁷, —C(═O)NR⁶⁷R⁶⁸, —OC(═O)NR⁶⁷R⁶⁸, —NR⁶⁷C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl; or 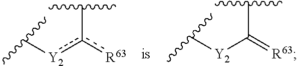
wherein:
Y² is NR^{Y3};
R⁶³ is O or S; and
R^{Y3} is H or optionally substituted alkyl;
R⁶⁰, R⁶¹, and R⁶² are independently H, halogen, —CN, —OR⁶⁷, —SR⁶⁷, —S(═O)R⁶⁹, —NO₂, —NR⁶⁷R⁶⁸, —S(═O)₂R⁶⁹, —NR⁶⁷S(═O)₂R⁶⁹, —S(═O)₂NR⁶⁷R⁶⁸, —C(═O)R⁶⁹, —OC(═O)R⁶⁹, —CO₂R⁶⁷, —OCO₂R⁶⁷, —C(═O)NR⁶⁷R⁶⁸, —OC(═O)N R⁶⁷R⁶⁸, —NR⁶⁷C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl;
R⁶⁴ is H, optionally substituted alkyl, optionally substituted alkenyl, optionally substituted alkynyl, optionally substituted heteroalkyl, optionally substituted cycloalkyl, optionally substituted heterocycloalkyl, optionally substituted aryl, or optionally substituted heteroaryl;
or R⁶³ and R⁶⁴ are taken together with the atoms to which they are attached to form an optionally substituted 6-membered heterocycloalkyl or an optionally substituted heteroaryl;
R⁶⁵ is optionally substituted fused bicyclic heterocycloalkyl or optionally substituted fused bicyclic heteroaryl;
each R⁶⁶ is independently halogen, —CN, —OR⁶⁷, —SR⁶⁷, —S(═O)R⁶⁹, —NO₂, —NR⁶⁷R⁶⁸, —S(═O)₂R⁶⁹, —NR⁶⁷S(═O)₂R⁶⁹, —S(═O)₂NR⁶⁷R⁶⁸, —C(═O)R⁶⁹, —OC(═O)R⁶⁹, —CO₂R⁶⁷, —OCO₂R⁶⁷, —C(═O)NR⁶⁷R⁶⁸, —OC(═O)N R⁶⁷R⁶⁸, —NR⁶⁷C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl;
each R⁶⁷ and R⁶⁸ is independently H, optionally substituted —CN, —OR⁶⁷, —SR⁶⁷, —S(═O)R⁶⁹, —NO₂, —NR⁶⁷R⁶⁸, —S(═O)₂R⁶⁹, —NR⁶⁷S(═O)₂R⁶⁹, —S(═O)₂NR⁶⁷R⁶⁸, —C(═O)R⁶⁹, —OC(═O)R⁶⁹, —CO₂R⁶⁷, —OCO₂R⁶⁷, —C(═O)NR⁶⁷R⁶⁸, —OC(═O)N R⁶⁷R⁶⁸, —NR67C(═O)NR⁶⁷R⁶⁸, —NR⁶⁷C(═O)NR⁶⁹, —NR⁶⁷C(═O)OR⁶⁷, optionally substituted alkyl, optionally substituted alkenyl, optionally substituted aklynyl, optionally substituted heteroalkyl, optionally substituted cycloalkyl, optionally substituted heterocycloalkyl, optionally substituted aryl, or optionally substituted heteroaryl;
or R⁶⁷ and R⁶⁸ are together with the nitrogen atom to which they are attached, form an optionally substituted heterocycloalkyl or optionally substituted heteroaryl;
R⁶⁹ is optionally substituted alkyl, optionally substituted alkynyl, optionally substituted heteroalkyl, optionally substituted cycloalkyl, optionally substituted heterocycloalkyl, optionally substituted aryl, or optionally substituted heteroaryl; and
s3 is 0-3.

59. The method of any one of claims 1-9, wherein the dual inhibitor is a compound having the structure of Formula (IX), or a pharmaceutically acceptable salt or solvate thereof: 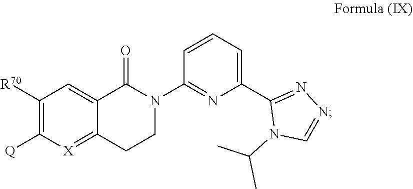
wherein:
X is selected from the group consisting of CH and N;
Q is selected from the group consisting of CH₃ and H; and
R⁷⁰ is selected from the group consisting of

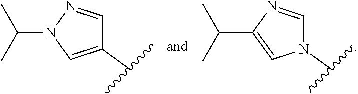

60. The method of any one of claims 1-9, wherein the dual inhibitor is a compound of structural Formula (X), or a pharmaceutically acceptable salt or solvate thereof: 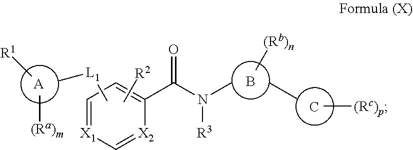
wherein:
Ring C is phenyl, 6-membered hetroaryl, or a 5-membered heteroaryl;
each R^{a} is independently H, D, halogen, —CN, —OR⁵, —SR⁵, —S(═O)R⁴, —S(═O)₂R⁴, —S(═O)₂N(R⁵)₂, —R⁵S(═O)₂R⁴, —C(═O)R⁴, —OC(═O)R⁴, —CO₂R⁵, —OCO₂R⁴, —N(R⁵)₂, —OC(═O)N(R⁵)₂, —C(═O)N(R⁵)₂, —R⁵C(═O)R₄, —R⁵C(═O)OR⁴, —R⁵C(═O)N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, substituted or unsubstituted C₃-C₆cycloalkyl;
m is 0, 1, 2, or 3;
R¹ is H, D, halogen, —CN, —OR⁵, —SR⁵, —S(═O)R⁴, —S(═O)₂R⁴, —N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, or substituted or unsubstituted —C₁-C₄alkylene-N(R⁵)₂;
L¹ is linker that is —X²—, L², -L²-X²—, —X²-L³-, or -L²-X²-L³-;
X² is —O—, —S—, —S(═O)—, —S(═O)₂—, —S(═O)₂NR⁶—, —C(═O)—, —C(═O)O—, —C(═O)NR⁶—, —OC(═O)NR⁶—, —NR⁶C(═O)O, ≥NR⁶C(═O)NR⁶≥, —OC(═O)—, —NR⁶C(═O)—, —NR⁶S(═O)₂—, or —NR⁶—;
R⁶ is H, C₁-C₆alkyl, C₁-C₆fluoroalkyl, or C₁-C₆deuteroalkyl;
L² is substituted or unsubstituted C₁-C₄alkylene, substituted or unsubstituted C₂-C₄alkenylene or substituted or unsubstituted C₂-C₄alkynylene;
L³ is C₁-C₄alkylene;
X¹ is CR² or N;
X² is CR² or N;
each R² is independently H, D, halogen, —CN, —OR⁵, —SR^{S}, —S(═O)R⁴, —S(═O)₂R⁴, —N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, substituted or unsubstituted C3-C6cycloalkyl;
R³ is H, substituted or unsubstituted C₁-C₆alkyl, substituted or unsubstituted C₁-C₆fluoroalkyl, or substituted or unsubstituted C₁-C₆deuteroalkyl;
Ring D is a 6-membered heteroaryl, phenyl, or a 5-membered heteroaryl;
each R^{b} is independently H, D, halogen, —CN, —OR⁵, —SR^{S}, —S(═O)R⁴, —S(═O)₂R⁴, —S(═O)₂N(R⁵)₂, —R⁵S(═O)₂R⁴, —C(═O)R⁴, —OC(═O)R⁴, —CO₂R⁵, —OCO₂R⁴, —N(R⁵)₂, —OC(═O)N(R⁵)₂, —R⁵C(═O)R⁴, —R²C(═O)OR⁴, —C(═O)N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, substituted or unsubstituted C₃-C₆cycloalkyl;
n is 0, 1, 2, 3, or 4;
Ring E is a 5-membered heteroaryl;
each R^{c} is independently H, D, halogen, —CN, —OR⁵, —SR^{S}, —S(═O)R⁴, —S(═O)₂R⁴, —S(═O)₂N(R⁵)₂, —NR⁵S(═O)₂R⁴, —C(═O)R⁴, —OC(═O)R⁴, —CO₂R⁵, —OCO₂R⁴, —N(R⁵)₂, —OC(═O)N(R⁵)₂, —NR⁵C(═O)R⁴, —NR⁵C(═O)OR⁴, —C(═O)N(R⁵)₂, substituted or unsubstituted C₁-C₆alkyl, substituted or unsubstituted C₁-C₆fluoroalkyl, substituted or unsubstituted C₁-C₆deuteroalkyl, substituted or unsubstituted C₁-C₆heteroalkyl, or substituted or unsubstituted C₃-C₆cycloalkyl;
p is 0, 1, 2, or 3;
each R⁴ is independently selected from C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, substituted or unsubstituted benzyl and substituted or unsubstituted heteroaryl;
each R⁵ is independently selected from H, C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, substituted or unsubstituted benzyl and substituted or unsubstituted heteroaryl;
or two R⁵ on the same N atom are taken together with the N atom to which they are attached to a substituted or unsubstituted N-containing heterocycle.

61. The method of any one of claims 1-9, wherein the dual inhibitor is a compound having the structure of Formula (X), or a pharmaceutically acceptable salt or solvate thereof: 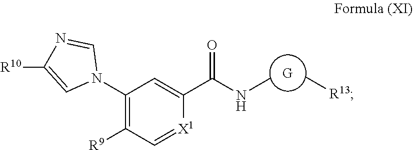
wherein:
Ring G is selected from 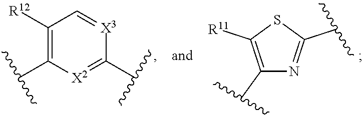
X¹, X² and X³ are each independently selected from N or)C(R¹⁰);
R¹⁰, R¹¹ and R¹² are each independently selected from the group consisting of: hydrogen, halogen, cyano, optionally substituted —C₁-C₆ alkyl, optionally substituted —C₃-C₈ cycloalkyl, optionally substituted 3- to 8-membered heterocycloalkyl, and optionally substituted —C₁-C₆ alkoxyl;
R¹³ is selected from: 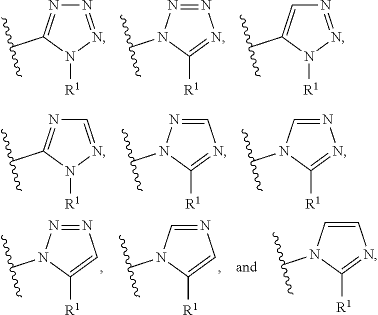
each of which is optionally substituted when possible;
R¹ is selected from the group consisting of: hydrogen, optionally substituted —C₁-C₆ alkyl, optionally substituted —C₂-C₈ alkenyl, optionally substituted —C₂-C₈ alkynyl, optionally substituted —C₃-C₈ cycloalkyl, optionally substituted aryl, optionally substituted arylalkyl, optionally substituted 3- to 8-membered heterocycloalkyl, optionally substituted heteroaryl, optionally substituted heteroarylalkyl, and —N(R⁶)(R⁷);
provided that when R¹³ is 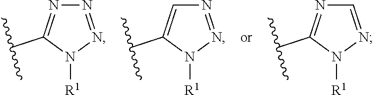
R¹ is not —N(R¹⁶)(R¹⁷);
R⁹ is selected from the group consisting of: hydrogen, halogen, cyano, optionally substituted —C₁-C₆ alkyl, optionally substituted —C₂-C₈ alkenyl, optionally substituted —C₂-C₈ alkynyl, optionally substituted —C₃-C₈ cycloalkyl, optionally substituted aryl, optionally substituted arylalkyl, optionally substituted 3- to 8-membered heterocycloalkyl, optionally substituted heteroaryl, optionally substituted heteroarylalkyl, —N(R¹⁶)(R¹⁷), —S(O)₂N(R¹⁶)(R¹⁷), —N(R¹⁶)C(O)(R¹⁷), and —N(R¹⁶)S(O)₂(R¹⁷);
wherein R¹⁶ and R¹⁷ are independently selected from the group consisting of hydrogen, —C₁-C₁₅ alkyl, cycloalkyl, heterocycloalkyl, aryl, and heteroaryl, wherein each alkyl, cycloalkyl, heterocycloalkyl, aryl and heteroaryl is optionally substituted with 1-3 substituents independently selected from halo, alkyl,alkylamino, dialkylamino, alkyl-C(O)NH—, aryl-C(O)NH—, heteroaryl-C(O)-NH, —CN, alkoxy, —CF₃, aryl, and heteroaryl, or R¹⁷ and le are taken together with the nitrogen to which they are attached to form a heterocyclic ring.

62. The method of any one of claims 1-9, wherein the dual inhibitor is a pharmaceutical composition comprising a compound of any one of claims 10-61, or a pharmaceutically acceptable salt or solvate thereof, and at least one pharmaceutically acceptable excipient.
